(19) United States
(12) Patent Application Publication
(10) Pub. No.: US 2016/0319374 A1
(43) Pub. Date: Nov. 3, 2016

(54) DETECTION OF ENTAMOEBA NUCLEIC ACIDS

(71) Applicant: BECTON, DICKINSON AND COMPANY, Franklin Lakes, NJ (US)

(72) Inventors: Steven Knapp, Apex, NC (US); Charlotte Brown, Chapel Hill, NC (US); Karen Lenz, Durham, NC (US); Melissa Adams, Chapel Hill, NC (US); Robert Swan, Durham, NC (US)

(21) Appl. No.: 15/108,771

(22) PCT Filed: Dec. 30, 2014
(86) PCT No.: PCT/US14/72709

§ 371 Date: Jun. 28, 2016

Related U.S. Application Data
(60) Provisional application No. 61/923,086, filed on Jan. 2, 2014.

(51) Int. Cl. C12Q 1/68

(52) U.S. Cl. CPC C12Q 1/6893; C12Q 2600/16

(57) ABSTRACT

Provided herein are compositions, methods, and kits for detection of Entamoeba nucleic acids. Some embodiments relate to detection of E. histolytica but not E. dispar. Some embodiments relate to quantification of levels of E. histolytica.

### CROSS REFERENCE TO RELATED APPLICATIONS

The present application claims priority to U.S. Provisional App. No. 61/923,086 filed Jan. 2, 2014, which is hereby incorporated by reference in its entirety.

### REFERENCE TO SEQUENCE LISTING, TABLE, OR COMPUTER PROGRAM LISTING

The present application is being filed along with a Sequence Listing in electronic format. The Sequence Listing is provided as a file entitled GENOM123WOSEQUENCE.TXT, created and last saved on Dec. 22, 2014, which is 7,503 bytes in size. The information is incorporated herein by reference in its entirety.

### FIELD

Embodiments herein relate generally to methods and compositions that are useful for detecting the presence of Entamoeba nucleic acids.

### BACKGROUND

Amebiasis is a disease that can be caused by infection with the protozoan Entamoeba histolytica. E. histolytica infection is typically in the intestinal tract, and can cause colitis, and amoebic dysentery. E. histolytica infection can also spread to other organs, including the liver, the lungs, or central nervous system. E. dispar is a non-pathogenic species, and is morphologically indistinguishable from the pathogenic E. histolytica (Verweij et al., J. Clin. Microbiol. 42: 1220-23, 2004). Moreover, E. dispar and E. histolytica genomes have a high degree of nucleic acid sequence homology. It has been estimated that E. histolytica and/or E. dispar parasitize 10% of the world's population (Verweij et al., J. Clin. Microbiol. 42: 1220-23, 2004). However, it has been estimated that only about 10% of these Entamoeba infections are pathogenic (e.g. infection by E. histolytica) so as to require treatment (Gonin et al., J. Clin. Microbiol. 41: 237-42, 2003). Thus, distinguishing between E. dispar and E. histolytica infection is useful in guiding clinical decisions.

Quantitative nucleic acid amplification reactions can be useful for quantifying the relative and/or absolute amount of target nucleic acid sequences present in a sample. Due to the highly sensitive nature of quantitative nucleic acid amplification reactions, in order to avoid false positives, false negatives, overestimation of target or product quantity, or underestimation of target or product quantity, extreme care must be taken when selecting reagents and methods for quantitative nucleic acid amplification. Ribosomal DNA (rDNA) genes are highly conserved. The high degree of conservation of rDNA sequences can result in little variability between different organisms of the same species, a feature that can make rDNA genes useful for nucleic-acid-based detection assays directed to the detection of a desired species. However, the high degree of homology between E. histolytica and E. dispar rDNA genes can complicate quantitative nucleic acid amplification for the specific detection of the different species. For example, it has been reported that multi-template PCR amplification or rDNA genes can be subject to bias, and can produce various artifacts (Kanagawa, J. Bioscience and Bioengineering 96: 317-23, 2003; Wang et al., Microbiology 142: 1107-14, 1996).

### SUMMARY

According to some embodiments, a method of detecting the presence of an E. histolytica polynucleotide sequence in a sample. The method can comprise contacting the sample with a first primer consisting essentially of SEQ ID NO: 1 (GTACAAAATGGCCAATTCATTCAATG). The method can comprise contacting the sample with a second primer consisting essentially of SEQ ID NO: 2 (ACTACCAACTGATTGATAGATCAG). The method can comprise extending the first and second primer, thereby producing at least one amplicon if the E. histolytica polynucleotide sequence is present in the sample. The method can comprise contacting the sample with an oligonucleotide probe comprising a polynucleotide consisting essentially of SEQ ID NO: 3 (ATTGTCGTGGCATCCTAACTCA) or its complement. In some emboidmnents, the probe provides detectable signal when it is bound to a substantially complementary nucleic acid, but does not provide detectable signal when it is single-stranded. The method can comprise detecting the signal, if the amplicon is present. In some embodiments, if used under standard amplification conditions, the first primer and second primer amplify the E. histolytica polynucleotide sequence, but do not substantially amplify any E. dispar polynucleotide sequence. In some embodiments, the first primer hybridizes to the E. histolytica polynucleotide sequence if contacted with the E. histolytica polynucleotide sequence at a temperature of at least about 50° C. in 5 mM MgCl2, 100 mM Tris, 10 mM NaOH, 0.019% ProClin300, 0.010% Tween-20, 1.96% Trehalose, 0.6 mg/ml BSA, but does not hybridize to any E. dispar polynucleotide sequence if contacted with any E. dispar polynucleotide sequence at a temperature of at least about 60° C. in 5 mM MgCl₂, 100 mM Tris, 10 mM NaOH, 0.019% ProClin300, 0.010% Tween-20, 1.96% Trehalose, 0.6 mg/ml BSA. In some embodiments, the second primer hybridizes to the E. histolytica polynucleotide sequence if contacted with E. histolytica polynucleotide sequence at a temperature of at least about 60° C. in 5 mM MgCl2, 100 mM Tris, 10 mM NaOH, 0.019% ProClin300, 0.010% Tween-20, 1.96% Trehalose, 0.6 mg/ml BSA1.96% Trehalose, 0.6 mg/ml BSA, and hybridizes to an E. dispar polynucleotide sequence if contacted with the E. dispar polynucleotide sequence at a temperature of at least about 60° C. in 5 mM MgCl2, 100 mM Tris, 10 mM NaOH, 0.019% ProClin300, 0.010% Tween-20, 1.96% Trehalose, 0.6 mg/ml BSA1.96% Trehalose, 0.6 mg/ml BSA. In some embodiments, each of the first primer and second primer hybridizes to the E. histolytica polynucleotide sequence if contacted with the E. histolytica polynucleotide sequence at a temperature of at least about 60° C. in in 5 mM MgCl2, 100 mM Tris, 10 mM NaOH, 0.019% ProClin300, 0.010% Tween-20, 1.96% Trehalose, 0.6 mg/ml BSA1.96% Trehalose, 0.6 mg/ml BSA, but the second primer does not hybridize to any E. dispar polynucleotide sequence if contacted with any E. dispar polynucleotide sequence at a temperature of at least about 60° C. in 5 mM MgCl2, 100 mM Tris, 10 mM NaOH, 0.019% ProClin300, 0.010% Tween-20, 1.96% Trehalose, 0.6 mg/ml BSA. In some embodiments, the sample comprises E. histolytica and E. dispar. In some embodiments, the sample comprises fecal material of a human. In some embodiments, the sample comprises fixed material. In some embodiments, the sample is non-fixed. In some embodiments, a 95% limit of detection for E. histolytica comprises no more than about 17 E. histolytica genomes per milliliter. In some embodiments, if used under standard amplification conditions, the primers and probes do not cross-react with any of the following organisms, if present in the sample: Abiotrophia defectiva, Acinetobacter baumannii, Acinetobacter Iwoffii, Aeromonas hydrophila, Alcaligenes faecalis subsp. faecalis, Anaerococcus tetradius, Arcobacter butzleri, Arcobacter cryaerophilus, Bacillus cereus, Bacteroides caccae, Bacteroides merdae, Bacteroides stercoris, Bifidobacterium adolescentis, Bifidobacterium longum, Camplylobacter coli, Campylobacter concisus, Campylobacter curvus, Campylobacter fetus subsp. fetus, Campylobacter fetus subsp. venerealis, Campylobacter gracilis, Campylobacter hominis, Camplylobacter jejuni, Campylobacter lari, Campylobacter rectus, Campylobacter upsaliensis, Candida albicans, Candida catenulate, Cedecea davisae, Chlamydia trachomatis, Citrobacter amalonaticus, Citrobacter fruendii, Citrobacter koseri, Citrobacter sedlakii, Clostridium difficile 17858, Clostridium difficile 43598, Clostridium difficile CCUG 8864-9689, Clostridium difficile 43255, Clostridium difficile BAA-1805, Clostridium difficile 43593, Clostridium perfringens, Collinsella aerofaciens, Corynebacterium genitalium, Desulfovibrio piger, Edwardsiella tarda, Eggerthella lenta, Enterobacter aerogenes, Enterobacter cloacae, Enterococcus casseliflavus, Enterococcus cecorum, Enterococcus dispar, Enterococus faecalis, Enterococcus gallinarum, Enterococcus hirae, Enterococcus raffinosus, Escherichia coli, Escherichia fergusonii, Escherichia hermannii, Escherichia vulneris, Fusobacterium varium, Gardnerella vaginalis, Gemella morbillorum, Hafnia alvei, Helicobacter fennelliae, Helicobacter pylori, Klebsiella oxytoca, Klebsiella pneumonia, Lactobacillus acidophilus, Lactobacillus reuteri, Lactococcus lactis, Leminorella grimontii, Listeria grayi, Listeria innocua, Listeria monocytogenes, Morganella morganii, Peptoniphilus asaccharolyticus, Peptostreptococcus anaerobius, Plesiomonas shigelloides, Porphyromonas asaccharolytica, Prevotella melaninogenica, Proteus mirabilis, Proteus penneri, Proteus vulgaris, Providencia alcalifaciens, Providencia rettgeri, Providencia stuartii, Pseudomonas aeruginosa, Pseudomonas fluorescens, Ruminococcus bromii, Salmonella typhimurium, Salmonella enteriditis, Serratia liquefaciens, Serratia marcescens, Shigella sonnei, Shigella flexneri, Staphylococcus aureus, Staphylococcus epidermidis, Stenotrophomonas maltophilia, Streptococcus agalactiae, Streptococcus dysgalactiae, Streptococcus intermedius, Streptococcus uberis, Trabulsiella guamensis, Veillonella parvula, Vibrio cholera, Vibrio parahaemolyticus, Yersinia bercovieri, Yersinia enterocolitica, Yersinia rohdei, Adenovirus type 2, Adenovirus type 14, Adenovirus type 40, Adenovirus type 41, Coxsackie A9, Coxsackie B1, HHV-5, Cytomegalovirus, Enterovirus type 69, Human Papillomavirus Type 16, Human Papillomavirus Type 18, Herpes Simplex Virus I, Herpes Simplex Virus II, Norovirus Norovirus II, Rotavirus, Blastocystis hominis, Encephalitozoon intestinalis, Encephalitozoon helium, Encephalitozoon cuniculi, Pentatrichomonas hominis, Entamoeba barrette, Entamoeba dispar, Entamoeba gigivalis, Entamoeba invadens, Entamoeba moshkovskii, Entamobea ranarum, Citrobacter fruendii (rpt), Enterobacter cloacae (rpt), Cryptosporidium parvum, Giardia lamblia, or Cryptosporidium meleagridis. In some embodiments, if used under standard amplification conditions, the primers and probes do not cross-react with any of the following organisms, if present in the sample: Entamoeba coli, Entamoeba dispar, Entamoeba polecki, Entamoeba muris, Entamoeba nuttalli, Entamoeba hartmanni, and Entamoeba bovis. In some embodiments, if used under standard amplification conditions, the primers and probes produce fewer than 1 in 1600 false positives for samples that do not comprise E. histolytica. In some embodiments, E. dispar, if present, does not inhibit production of the amplicon if the E. histolytica polynucleotide sequence is present in the sample. In some embodiments, E. dispar, if present, does not inhibit determining the presence or absence of E. histolytica.

According to some embodiments, a kit is provided. The kit can comprise a first primer. The kit can comprise a second primer. In some embodiments, if used under standard amplification conditions, the first primer and second primer amplify a E. histolytica polynucleotide sequence, thereby producing an amplicon, but do not substantially amplify any E. dispar polynucleotide sequence. The kit can comprise a probe, wherein the probe comprises a polynucleotide consisting essentially of a sequence, wherein the sequence or its complement is present in each of the amplicon, a polynucleotide sequence of E. histolytica, and a polynucleotide sequence of E. dispar. In some embodiments, the probe comprises a fluorophore; and a quencher. In some embodiments, the primers and probes amplify an E. histolytica polynucleotide sequence with a 95% limit of detection of no more than about 17 E. histolytica organisms per mililiter. In some embodiments, if used under standard amplification conditions, the primers and probes do not cross-react with any of the following organisms, if present in the sample: Abiotrophia defectiva, Acinetobacter baumannii, Acinetobacter Iwoffii, Aeromonas hydrophila, Alcaligenes faecalis subsp. faecalis, Anaerococcus tetradius, Arcobacter butzleri, Arcobacter cryaerophilus, Bacillus cereus, Bacteroides caccae, Bacteroides merdae, Bacteroides stercoris, Bifidobacterium adolescentis, Bifidobacterium longum, Camplylobacter coli, Campylobacter concisus, Campylobacter curvus, Campylobacter fetus subsp. fetus, Campylobacter fetus subsp. venerealis, Campylobacter gracilis, Campylobacter hominis, Camplylobacter jejuni, Campylobacter lari, Campylobacter rectus, Campylobacter upsaliensis, Candida albicans, Candida catenulate, Cedecea davisae, Chlamydia trachomatis, Citrobacter amalonaticus, Citrobacter fruendii, Citrobacter koseri, Citrobacter sedlakii, Clostridium difficile 17858, Clostridium difficile 43598, Clostridium difficile CCUG 8864-9689, Clostridium difficile 43255, Clostridium difficile BAA-1805, Clostridium difficile 43593, Clostridium perfringens, Collinsella aerofaciens, Corynebacterium genitalium, Desulfovibrio piger, Edwardsiella tarda, Eggerthella lenta, Enterobacter aerogenes, Enterobacter cloacae, Enterococcus casseliflavus, Enterococcus cecorum, Enterococcus dispar, Enterococus faecalis, Enterococcus gallinarum, Enterococcus hirae, Enterococcus raffinosus, Escherichia coli, Escherichia fergusonii, Escherichia hermannii, Escherichia vulneris, Fusobacterium varium, Gardnerella vaginalis, Gemella morbillorum, Hafnia alvei, Helicobacter fennelliae, Helicobacter pylori, Klebsiella oxytoca, Klebsiella pneumonia, Lactobacillus acidophilus, Lactobacillus reuteri, Lactococcus lactis, Leminorella grimontii, Listeria grayi, Listeria innocua, Listeria monocytogenes, Morganella morganii, Peptomphilus asaccharolyticus, Peptostreptococcus anaerobius, Plesiomonas shigelloides, Porphyromonas asaccharolytica, Prevotella melaninogenica, Proteus mirabilis, Proteus penneri, Proteus vulgaris, Providencia alcalifaciens, Providencia rettgeri, Providencia stuartii, Pseudomonas aeruginosa, Pseudomonas fluorescens, Ruminococcus bromii, Salmonella typhimurium, Salmonella enteriditis, Serratia liquefaciens, Serratia marcescens, Shigella sonnei, Shigella flexneri, Staphylococcus aureus, Staphylococcus epidermidis, Stenotrophomonas maltophilia, Streptococcus agalactiae, Streptococcus dysgalactiae, Streptococcus intermedius, Streptococcus uberis, Trabulsiella guamensis, Veillonella parvula, Vibrio cholera, Vibrio parahaemolyticus, Yersinia bercovieri, Yersinia enterocolitica, Yersinia rohdei, Adenovirus type 2, Adenovirus type 14, Adenovirus type 40, Adenovirus type 41, Coxsackie A9, Coxsackie B1, HHV-5, Cytomegalovirus, Enterovirus type 69, Human Papillomavirus Type 16, Human Papillomavirus Type 18, Herpes Simplex Virus I, Herpes Simplex Virus II, Norovirus I, Norovirus II, Rotavirus, Blastocystis hominis, Encephalitozoon intestinalis, Encephalitozoon helium, Encephalitozoon cuniculi, Pentatrichomonas hominis, Entamoeba barrette, Entamoeba dispar, Entamoeba gigivalis, Entamoeba invadens, Entamoeba moshkovskii, Entamobea ranarum, Citrobacter fruendii (rpt), Enterobacter cloacae (rpt), Cryptosporidium parvum, Giardia lamblia, or Cryptosporidium meleagridis. In some embodiments, the first primer comprises a polynucleotide having at least about 90% identity to SEQ ID NO: 1 (GTACAAAATGGCCAATTCATTCAATG) or its complement. In some embodiments, the first primer consists essentially of SEQ ID NO: 1 (GTACAAAATGGCCAATTCATTCAATG) or its complement. In some embodiments, the second primer comprises a polynucleotide having at least about 90% identity to SEQ ID NO: 2 (ACTACCAACTGATTGATAGATCAG) or its complement. In some embodiments, the second primer comprises a polynucleotide having the sequence of SEQ ID NO: 2 (ACTACCAACTGATTGATAGATCAG) or its complement. In some embodiments, the probe comprises a polynucleotide having at least about 90% identity to SEQ ID NO: 3 (ATTGTCGTGGCATCCTAACTCA) or its complement. In some embodiments, the probe comprises a polynucleotide having the sequence of SEQ ID NO: 3 (ATTGTCGTGGCATCCTAACTCA) or its complement. In some embodiments, if used under standard amplification conditions, the primers and probes do not cross-react with any of the following organisms, if present in the sample: Entamoeba coli, Entamoeba dispar, Entamoeba polecki, Entamoeba muris, Entamoeba nuttalli, Entamoeba hartmanni, and Entamoeba bovis. In some embodiments, if used under standard amplification conditions, the primers and probes produce fewer than 1 in 1600 false positives for samples that do not comprise E. histolytica. In some embodiments, E. dispar, if present, does not inhibit production of the amplicon if the E. histolytica polynucleotide sequence is present in the sample. In some embodiments, E. dispar, if present, does not inhibit determining the presence or absence of E. histolytica. [0006]

According to some embodiments, a kit is provided. The kit can comprise a first primer comprising a polynucleotide having at least about 90% identity to SEQ ID NO: 1 (GTACAAAATGGCCAATTCATTCAATG). The kit can comprise a second primer comprising polynucleotide having at least about 90% identity to SEQ ID NO: 2 (ACTACCAACTGATTGATAGATCAG). The kit can comprise a probe comprising a polynucleotide having at least about 90% identity to SEQ ID NO: 3 (ATTGTCGTGGCATCCTAACTCA) or its complement; a flurophore; and a quencher. In some embodiments, the first primer consists essentially of SEQ ID NO: 1 (GTACAAAATGGCCAATTCATTCAATG). In some embodiments, the second primer consists essentially of SEQ ID NO: 2 (ACTACCAACTGATTGATAGATCAG). In some embodiments, the probe comprises a polynucleotide consisting essentially of SEQ ID NO: 3 (ATTGTCGTGGCATCCTAACTCA) or its complement. In some embodiments, if used under standard amplification conditions, the primers and probes do not cross-react with any of the following organisms, if present in the sample: Entamoeba coli, Entamoeba dispar, Entamoeba polecki, Entamoeba muris, Entamoeba nuttalli, Entamoeba hartmanni, and Entamoeba bovis. In some embodiments, if used under standard amplification conditions, the primers and probes produce fewer than 1 in 1600 false positives for samples that do not comprise E. histolytica. In some embodiments, E. dispar, if present, does not inhibit production of the amplicon if the E. histolytica polynucleotide sequence is present in the sample. In some embodiments, E. dispar, if present, does not inhibit determining the presence or absence of E. histolytica.

In some embodiments, a method of detecting the presence of an E. histolytica polynucleotide sequence in a sample. The method can comprise contacting the sample with a first primer. The method can comprise contacting the sample with a second primer. In some embodiments, if used standard amplification conditions, the first primer and second primer amplify the E. histolytica polynucleotide sequence, but do not substantially amplify any E. dispar polynucleotide sequence. The method can comprise extending the first and second primer, thereby producing at least one amplicon if the E. histolytica polynucleotide sequence is present in the sample. The method can comprise contacting the sample with an oligonucleotide probe. In some embodiments, the probe provides detectable signal when it is bound to a substantially complementary nucleic acid, but does not provide detectable signal when it is single-stranded. In some embodiments, the probe comprises a polynucleotide consisting essentially of sequence that is a portion of the E. histolytica polynucleotide sequence, a polynucleotide sequence of E. dispar, and a sequence of the amplicon. The method can comprise detecting the signal, if the amplicon is present. In some embodiments, the first primer hybridizes to the E. histolytica polynucleotide sequence if contacted with the E. histolytica polynucleotide sequence at a temperature of at least about 50° C. in 5 mM MgCl2, 100 mM Tris, 10 mM NaOH, 0.019% ProClin300, 0.010% Tween-20, 1.96% Trehalose, 0.6 mg/ml BSA, but does not hybridize to any E. dispar polynucleotide sequence if contacted with any E. dispar polynucleotide sequence at a temperature of at least about 60° C. in 5 mM MgCl₂, 100 mM Tris, 10 mM NaOH, 0.019% ProClin300, 0.010% Tween-20, 1.96% Trehalose, 0.6 mg/ml BSA. In some embodiments, the second primer hybridizes to the E. histolytica polynucleotide sequence if contacted with E. histolytica polynucleotide sequence at a temperature of at least about 60° C. in 5 mM MgCl2, 100 mM Tris, 10 mM NaOH, 0.019% ProClin300, 0.010% Tween-20, 1.96% Trehalose, 0.6 mg/ml BSA, and hybridizes to an E. dispar polynucleotide sequence if contacted with the E. dispar polynucleotide sequence at a temperature of at least about 60° C. in 5 mM MgCl2, 100 mM Tris, 10 mM NaOH, 0.019% ProClin300, 0.010% Tween-20, 1.96% Trehalose, 0.6 mg/ml BSA. In some embodiments, each of the first primer and second primer hybridizes to the E. histolytica polynucleotide sequence if contacted with the E. histolytica polynucleotide sequence at a temperature of at least about 60° C. in in 5 mM MgCl2, 100 mM Tris, 10 mM NaOH, 0.019% ProClin300, 0.010% Tween-20, 1.96% Trehalose, 0.6 mg/ml BSA, but the second primer does not hybridize to any E. dispar polynucleotide sequence if contacted with any E. dispar polynucleotide sequence at a temperature of at least about 50° C. in 5 mM MgCl2, 100 mM Tris, 10 mM NaOH, 0.019% ProClin300, 0.010% Tween-20, 1.96% Trehalose, 0.6 mg/ml BSA. In some embodiments, the first primer comprises a polynucleotide having at least about 90% identity to SEQ ID NO: 1 (GTACAAAATGGCCAATTCATTCAATG) or its complement. In some embodiments, the first primer consists essentially of SEQ ID NO: 1 (GTACAAAATGGCCAATTCATTCAATG) or its complement. In some embodiments, the second primer comprises a polynucleotide having at least about 90% identity to SEQ ID NO: 2 (ACTACCAACTGATTGATAGATCAG) or its complement. In some embodiments, the second primer comprises a polynucleotide having the sequence of SEQ ID NO: 2 (ACTACCAACTGATTGATAGATCAG) or its complement. In some embodiments, the probe comprises a polynucleotide having at least about 90% identity to SEQ ID NO: 3 (ATTGTCGTGGCATCCTAACTCA) or its complement. In some embodiments, the probe comprises a polynucleotide having the sequence of SEQ ID NO: 3 (ATTGTCGTGGCATCCTAACTCA) or its complement. In some embodiments, the amplicon comprises a polynucleotide having at least about 95% identity to SEQ ID NO: 7 (GTACAAAATGGCCAATTCATTCAATGAATTGAGAAATGACATTCTAAGTGAG TTAGGATGCCACGACAATTGTAGAACACACAGTGTTTAACAAGTAACCAATG AGAATTTCTGATCTATCAATCAGTTGGTAGT). In some embodiments, the amplicon comprises a polynucleotide having the sequence of SEQ ID NO: 7 (GTACAAAATGGCCAATTCATTCAATGAATTGAGAAATGACATTCTAAGTGAG TTAGGATGCCACGACAATTGTAGAACACACAGTGTTTAACAAGTAACCAATG AGAATTTCTGATCTATCAATCAGTTGGTAGT). In some embodiments, the sample comprises E. histolytica and E. dispar. In some embodiments, the sample comprises fecal material of a human. In some embodiments, the sample comprises fixed material. In some embodiments, the sample is non-fixed. In some embodiments, a 95% limit of detection for E. histolytica comprises no more than about 17 E. histolytica genomes per milliliter. In some embodiments, if used under standard amplification conditions, the primers and probes do not cross-react with any of the following organisms, if present in the sample: Abiotrophia defectiva, Acinetobacter baumannii, Acinetobacter Iwoffii, Aeromonas hydrophila, Alcaligenes faecalis subsp. faecalis, Anaerococcus tetradius, Arcobacter butzleri, Arcobacter cryaerophilus, Bacillus cereus, Bacteroides caccae, Bacteroides merdae, Bacteroides stercoris, Bifidobacterium adolescentis, Bifidobacterium longum, Camplylobacter coli, Campylobacter concisus, Campylobacter curvus, Campylobacter fetus subsp. fetus, Campylobacter fetus subsp. venerealis, Campylobacter gracilis, Campylobacter hominis, Camplylobacter jejuni, Campylobacter lari, Campylobacter rectus, Campylobacter upsaliensis, Candida albicans, Candida catenulate, Cedecea davisae, Chlamydia trachomatis, Citrobacter amalonaticus, Citrobacter fruendii, Citrobacter koseri, Citrobacter sedlakii, Clostridium difficile 17858, Clostridium difficile 43598, Clostridium difficile CCUG 8864-9689, Clostridium difficile 43255, Clostridium difficile BAA-1805, Clostridium difficile 43593, Clostridium perfringens, Collinsella aerofaciens, Corynebacterium genitalium, Desulfovibrio piger, Edwardsiella tarda, Eggerthella lenta, Enterobacter aerogenes, Enterobacter cloacae, Enterococcus casseliflavus, Enterococcus cecorum, Enterococcus dispar, Enterococus faecalis, Enterococcus gallinarum, Enterococcus hirae, Enterococcus raffinosus, Escherichia coli, Escherichia fergusonii, Escherichia hermannii, Escherichia vulneris, Fusobacterium varium, Gardnerella vaginalis, Gemella morbillorum, Hafnia alvei, Helicobacter fennelliae, Helicobacter pylori, Klebsiella oxytoca, Klebsiella pneumonia, Lactobacillus acidophilus, Lactobacillus reuteri, Lactococcus lactis, Leminorella grimontii, Listeria grayi, Listeria innocua, Listeria monocytogenes, Morganella morganii, Peptoniphilus asaccharolyticus, Peptostreptococcus anaerobius, Plesiomonas shigelloides, Porphyromonas asaccharolytica, Prevotella melaninogenica, Proteus mirabilis, Proteus penneri, Proteus vulgaris, Providencia alcalifaciens, Providencia rettgeri, Providencia stuartii, Pseudomonas aeruginosa, Pseudomonas fluorescens, Ruminococcus bromii, Salmonella typhimurium, Salmonella enteriditis, Serratia liquefaciens, Serratia marcescens, Shigella sonnei, Shigella flexneri, Staphylococcus aureus, Staphylococcus epidermidis, Stenotrophomonas maltophilia, Streptococcus agalactiae, Streptococcus dysgalactiae, Streptococcus intermedius, Streptococcus uberis, Trabulsiella guamensis, Veillonella parvula, Vibrio cholera, Vibrio parahaemolyticus, Yersinia bercovieri, Yersinia enterocolitica, Yersinia rohdei, Adenovirus type 2, Adenovirus type 14, Adenovirus type 40, Adenovirus type 41, Coxsackie A9, Coxsackie B1, HHV-5, Cytomegalovirus, Enterovirus type 69, Human Papillomavirus Type 16, Human Papillomavirus Type 18, Herpes Simplex Virus I, Herpes Simplex Virus II, Norovirus I, Norovirus II, Rotavirus, Blastocystis hominis, Encephalitozoon intestinalis, Encephalitozoon helium, Encephalitozoon cuniculi, Pentatrichomonas hominis, Entamoeba barrette, Entamoeba dispar, Entamoeba gigivalis, Entamoeba invadens, Entamoeba moshkovskii, Entamobea ranarum, Citrobacter fruendii (rpt), Enterobacter cloacae (rpt), Cryptosporidium parvum, Giardia lamblia, or Cryptosporidium meleagridis. In some embodiments, if used under standard amplification conditions, the primers and probes do not cross-react with any of the following organisms, if present in the sample: Entamoeba coli, Entamoeba dispar, Entamoeba polecki, Entamoeba muris, Entamoeba nuttalli, Entamoeba hartmanni, and Entamoeba bovis. In some embodiments, if used under standard amplification conditions, the primers and probes produce fewer than 1 in 1600 false positives for samples that do not comprise E. histolytica. In some embodiments, E. dispar, if present, does not inhibit production of the amplicon if the E. histolytica polynucleotide sequence is present in the sample. In some embodiments, E. dispar, if present, does not inhibit determining the presence or absence of E. histolytica.

In some embodiments, a method of determining the presence or absence of an E. histolytica nucleic acid sequence in a sample. The method can comprise performing a nucleic acid amplification reaction on the sample, the nucleic acid amplification comprising a first oligonucleotide primer and a second oligonucleotide primer, in which the first oligonucleotide primer has a length of 15-75 nucleotides and hybridizes under standard conditions to SEQ ID NO:10 or its complement, if present, but does not hybridize under standard conditions to SEQ ID NO: 11 or its complement, if present, and in which the second oligonucleotide primer has a length of 15-75 nucleotides and hybridizes under standard conditions to a SEQ ID NO:10 or its complement, if present, and wherein the second oligonucleotide primer hybridizes under standard conditions to SEQ ID NO: 11 or its complement, if present. The method can comprise detecting a signal, if present, from a detectably labeled probe that hybridizes to an amplicon of the first and second oligonucleotide primers under standard hybridization conditions if the amplicon is present, in which the signal indicates the presence or absence of the amplicon, and in which the amplicon has a length of 75-350 nucleotides. Optionally, the first oligonucleotide primer comprises at least 10 consecutive nucleotides of SEQ ID NO: 1, and wherein the first oligonucleotide primer has at least 80% identity to a target sequence of SEQ ID NO: 10 or its complement. Optionally, the second oligonucleotide primer comprises at least 10 consecutive nucleotides of SEQ ID NO: 2, and wherein the second oligonucleotide primer has at least 80% identity to a target sequence of SEQ ID NO: 10 or its complement. Optionally, the first oligonucleotide primer comprises at least 12 consecutive nucleotides of SEQ ID NO: 1. Optionally, the first oligonucleotide primer comprises at least 15 consecutive nucleotides of SEQ ID NO: 1. Optionally, the first oligonucleotide primer comprises at least 20 consecutive nucleotides of SEQ ID NO: 1. Optionally, the first oligonucleotide primer has at least 85% identity to a target sequence of SEQ ID NO: 10 or its complement. Optionally, the first oligonucleotide primer has at least 90% identity to a target sequence of SEQ ID NO: 10 or its complement. Optionally, the first oligonucleotide primer has at least 95% identity to a target sequence of SEQ ID NO: 10 or its complement. Optionally, the first oligonucleotide primer has 100% identity to a target sequence of SEQ ID NO: 10 or its complement. Optionally, the second oligonucleotide primer comprises at least 12 consecutive nucleotides of SEQ ID NO: 2. Optionally, the second oligonucleotide primer comprises at least 15 consecutive nucleotides of SEQ ID NO: 2. Optionally, the second oligonucleotide primer comprises at least 20 consecutive nucleotides of SEQ ID NO: 2. Optionally, the second oligonucleotide primer has at least 85% identity to a target sequence of SEQ ID NO: 10 or its complement. Optionally, the second oligonucleotide primer has at least 90% identity to a target sequence of SEQ ID NO: 10 or its complement. Optionally, the second oligonucleotide primer has at least 95% identity to a target sequence of SEQ ID NO: 10 or its complement. Optionally, the second oligonucleotide primer has 100% identity to a target sequence of SEQ ID NO: 10 or its complement. Optionally, the probe comprises at least 10 consecutive nucleotides of SEQ ID NO: 3, and wherein the probe has at least 80% identity to a target sequence of SEQ ID NO: 10 or its complement. Optionally, the probe comprises at least 12 consecutive nucleotides of SEQ ID NO: 3. Optionally, the probe comprises at least 15 consecutive nucleotides of SEQ ID NO: 3. Optionally, the probe comprises at least 20 consecutive nucleotides of SEQ ID NO: 3. Optionally, the probe has at least 85% identity to a target sequence of SEQ ID NO: 10 or its complement. Optionally, the probe has at least 90% identity to a target sequence of SEQ ID NO: 10 or its complement. Optionally, the probe has at least 95% identity to a target sequence of SEQ ID NO: 10 or its complement. Optionally, the probe has 100% identity to a target sequence of SEQ ID NO: 10 or its complement. Optionally, the first oligonucleotide primer is about 20-50 nucleotides long. Optionally, the first oligonucleotide primer is about 23-45 nucleotides long. Optionally, the second oligonucleotide primer is about 20-50 nucleotides long. Optionally, the second oligonucleotide primer is about 23-45 nucleotides long. Optionally, the detectably labeled probe is about 15-75 nucleotides long. Optionally, the detectably labeled probe is about 20-45 nucleotides long. Optionally, the probe is capable of hybridizing to SEQ ID NO:10 and to SEQ ID NO: 11 under standard hybridization conditions. Optionally, the probe is capable of hybridizing to SEQ ID NO:10 but not to SEQ ID NO: 11 under standard hybridization conditions. Optionally, the probe comprises a fluorophore or a quencher. Optionally, the amplicon has a length of 100-150 nucleotides. Optionally, the amplicon comprises SEQ ID NO: 7. In some embodiments, a kit comprising any of the first oligonucleotide primer, the second oligonucleotide primer, and the detectably labeled probe as described above is provided. In some embodiments, E. dispar, if present, does not inhibit production of the amplicon if the E. histolytica polynucleotide sequence is present in the sample. In some embodiments, E. dispar, if present, does not inhibit determining the presence or absence of E. histolytica.

### BRIEF DESCRIPTION OF THE DRAWINGS

FIG. 1 is a diagram showing primers and probes as used in some of the embodiments disclosed herein.

FIG. 2A is an alignment showing E. dispar and E. histolytica nucleic acids sequences.

FIG. 2B is an annotated diagram of an E. histolytica gene encoding small subunit ribosomal RNA (GenBank: AB608092.1) (SEQ ID NO: 10)

FIG. 3 is a graph showing quantitative PCR signal detection using previously-known primers and probes, for which the presence of E. dispar depresses amplification signal and can cause false negatives.

FIG. 4 is a graph showing quantitative PCR signal detection using primers and probes in embodiments as described herein, for which the presence of E. dispar does not interfere with amplification signal.

### DETAILED DESCRIPTION

Detection of E. histolytica, and quantification of relative levels of E. histolytica can be useful in guiding clinical decisions. Quantitative nucleic acid amplification, for example quantitative assays involving nucleic acid amplification, such as polymerase chain reaction (qPCR) can be highly sensitive, and useful for quantification of nucleic acid levels, and thus can be used to infer relative quantities of E. histolytica based on quantification of nucleic acid. However, it has been appreciated herein that the presence of E. dispar can interfere with the specificity and efficiency of some qPCR reagents for detecting E. histolytica, and can cause cross-reactivity, signal suppression, or even false negatives. Accordingly, some embodiments herein provide methods and reagents for detecting and quantifying E. hisotolytica nucleic acids, without substantial interference from the presence of E. dispar. Some embodiments herein provide methods of detecting E. hisotolytica nucleic acids by qPCR. Some embodiments herein provide reagents and/or kits for detecting E. hisotolytica without substantial interference from the presence of E. dispar.

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. Use of “or” means “and/or” unless stated otherwise. The term “and/or” means that the terms before and after can be taken together or separately. For illustration purposes, but not as a limitation, “X and/or Y” can mean “X” or “Y” or “X and Y”.

Whenever a range of values is provided herein, the range is meant to include the starting value and the ending value and any value or value range there between unless otherwise specifically stated. For example, “from 0.2 to 0.5” means 0.2, 0.3, 0.4, 0.5; ranges there between such as 0.2-0.3, 0.3-0.4, 0.2-0.4; increments there between such as 0.25, 0.35, 0.225, 0.335, 0.49; increment ranges there between such as 0.26-0.39; and the like.

The section headings used herein are for organizational purposes only and are not to be construed as limiting the subject matter described in any way. All literature and similar materials cited in this application including, but not limited to, patents, patent applications, articles, books, treatises, and internet web pages, regardless of the format of such literature and similar materials, are expressly incorporated by reference in their entirety for any purpose. In the event that one or more of the incorporated literature and similar materials defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Various embodiments of this disclosure describe compositions, and kits, and methods of using the same, for use in detecting and/or distinguishing or identifying Entamoeba nucleic acids. Accordingly, some embodiments provide nucleic acid sequences for use in nucleic acid detection assays, e.g., in amplification assays. A person skilled in the art will appreciate that for any nucleic acid sequence, the reverse compliment can be readily obtained, and that a disclosure of a nucleic acid sequence also provides a disclosure of the reverse compliment of that sequence. A person skilled in the art will appreciate that for any DNA sequence disclosed herein, a corresponding RNA sequence can be readily obtained, and that for any RNA sequence, a corresponding DNA can readily be obtained, for example by reverse transcription. A person skilled in the art will appreciate that subsequences of the nucleic sequences disclosed herein can be readily obtained. As used herein, “upstream” refers one or more locations 5′ of a position on a nucleic acid sequence, and “downstream” refers to one or more locations 3′ of a position on a nucleic acid sequence.

The nucleic acids provided herein can be in various forms. For example, in some embodiments, the nucleic acids are dissolved (either alone or in combination with various other nucleic acids) in solution, for example buffer. In some embodiments, nucleic acids are provided, either alone or in combination with other isolated nucleic acids, as a salt. In some embodiments, nucleic acids are provided in a lyophilized form that can be reconstituted. For example, in some embodiments, the isolated nucleic acids disclosed herein can be provided in a lyophilized pellet alone, or in a lyophilized pellet with other isolated nucleic acids. In some embodiments, nucleic acids are provided affixed to a solid substance, such as a bead, a membrane, or the like. In some embodiments, nucleic acids are provided in a host cell, for example a cell line carrying a plasmid, or a cell line carrying a stably integrated sequence. In some embodiments, nucleic acids are isolated from a host cell, for example one or more Entamoeba cells. In some embodiments, nucleic acids are synthesized, for example chemically or in a cell-free system.

### Nucleic Acid Amplification

In some embodiments, nucleic acid amplification can include qualitative nucleic acid amplification, e.g. to determine whether a nucleic acid sequence is present or absent in a sample, for example, an E. histolytica-specific or E. dispar-specific nucleic acid sequence. In some embodiments, nucleic acid amplification can include quantitative nucleic acids amplification, e.g. to measure the relative or absolute amount of nucleic acid present in a sample. In some embodiments, nucleic acid amplification can include quantitative and qualitative nucleic acid amplification, e.g. to determine whether a nucleic acid sequence is present in a sample, and if present, to measure the relative or absolute amount of nucleic acid sequence present in the sample. In some embodiments, the method of amplification includes a multiplex assay for identifying the presence of two or more parasitic organisms from a sample, such as a human stool sample, for example at least two or more of E. histolytica, E. dispar, Giardia lamblia, Cryptosporidium parvum, Cryptosporidium hominis, and the like.

Methods of nucleic acid amplification can include, but are not limited to: polymerase chain reaction (PCR), strand displacement amplification (SDA), for example multiple displacement amplification (MDA), loop-mediated isothermal amplification (LAMP), ligase chain reaction (LCR), immuno-amplification, and a variety of transcription-based amplification procedures, including transcription-mediated amplification (TMA), nucleic acid sequence based amplification (NASBA), self-sustained sequence replication (3SR), and rolling circle amplification. See, e.g., Mullis, “Process for Amplifying, Detecting, and/or Cloning Nucleic Acid Sequences,” U.S. Pat. No. 4,683,195; Walker, “Strand Displacement Amplification,” U.S. Pat. No. 5,455,166; Dean et al, “Multiple displacement amplification,” U.S. Pat. No. 6,977,148; Notomi et al., “Process for Synthesizing Nucleic Acid,” U.S. Pat. No. 6,410,278; Landegren et al. U.S. Pat. No. 4,988,617 “Method of detecting a nucleotide change in nucleic acids”; Birkenmeyer, “Amplification of Target Nucleic Acids Using Gap Filling Ligase Chain Reaction,” U.S. Pat. No. 5,427,930; Cashman, “Blocked-Polymerase Polynucleotide Immunoassay Method and Kit,” U.S. Pat. No. 5,849,478; Kacian et al., “Nucleic Acid Sequence Amplification Methods,” U.S. Pat. No. 5,399,491; Malek et al., “Enhanced Nucleic Acid Amplification Process,” U.S. Pat. No. 5,130,238; Lizardi et al., BioTechnology, 6:1197 (1988); Lizardi et al., U.S. Pat. No. 5,854,033 “Rolling circle replication reporter systems.” In some embodiments, two or more of the listed nucleic acid amplification methods are performed, for example sequentially. In some embodiments, a target RNA sequence is amplified. In some embodiments, the target RNA sequence is reverse-transcribed, and the reverse transcript includes a DNA that is amplified using a nucleic acid amplification method described herein.

In some embodiments, the nucleic acid amplification is quantitative. Quantitative nucleic acid amplification can include detection of the amount of amplicon produced. The detection can be performed continuously or periodically. For example, detection can be performed at a certain point, e.g., at the end of every Nth cycle or fraction thereof, where N is one of 1, 2, 3, 4, 5, 6, 7, 8, 9, 10, 11, 12, 13, 14, 15, 16, 17, 18, 19, 20, 21, 21, 23, 24, 25, 26, 27, 28, 29, 30, 21, 32, 32, 34, 35, 36, 37, 38, 39, 40, 41, 42, 43, 44, 45, 46, 47, 48, 49, 50, 51, 52, 53, 54, 55, 56, 58, 59, 60, 61, 62, 63, 64, 65, 66, 67, 68, 69, 70, 75, 80, 85, 95, 100 or the like. In some embodiments, detection can include measuring fluorescence, for example the intensity of electromagnetic radiation at the emission wavelength of a fluorophore tethered to a probe as described herein, or a wavelength range including the emission wavelength of the fluorophore tethered to the probe. As noted herein, exemplary probes include molecular beacons, SCORPIONS™ probes (Sigma), TAQMAN™ probes (Life Technologies), and the like. In some embodiments, detection can include detecting FRET. In some embodiments, detection can include detecting intensity of a non-specific detectable marker that binds to dsDNA, but does not bind to ssDNA. Examples of such non-specific dyes include intercalating agents such as SYBR Green I (Molecular Probes), PicoGreen (Molecular Probes), and the like.

As used herein, “substantial” amplification, and modifications of these root words (e.g. “substantially amplify,” “amplify substantially,” and the like), refers to amplification that produces exponential yields of an amplicon or amplicons under standard amplification conditions. For example, PCR-derived forms of amplification and LAMP can produce discrete, double stranded amplicons, for which each strand can serve as a template in successive rounds of amplification, thus permitting exponential amplification. It is contemplated herein that a template can be substantially amplified and detected by polynucleotide that have less than 100% complementarity to the template, for example primers and/or probes having degenerate nucleotides, inosines, or the like at one or more positions. On the other hand, if a forward primer anneals to a target non-specifically, or anneals to a region that is not flanked by a reverse primer binding site on the opposite strand, there can be low-level amplification of by extension of the forward primer in the 3′ direction to produce a new single strand, but the inability of this new single strand to serve as a template for successive amplification can result in non-exponential (for example linear), insubstantial amplification.

The skilled artisan will appreciate that the compositions disclosed herein can be used in various types of nucleic acid amplification reactions, as disclosed herein. In some embodiments, the compositions disclosed herein can be used in polymerase chain reaction (PCR). For a review of PCR technology, including amplification conditions, applied to clinical microbiology, see DNA Methods in Clinical Microbiology, Singleton P., published by Dordrecht; Boston: Kluwer Academic, (2000) Molecular Cloning to Genetic Engineering White, B. A. Ed. in Methods in Molecular Biology 67: Humana Press, Totowa (1997) and “PCR Methods and Applications”, from 1991 to 1995 (Cold Spring Harbor Laboratory Press), each of which is hereby incorporated by reference in its entirity. As used herein “standard amplification conditions” refer to 5 mM MgCl₂, 100 mM Tris, 10 mM NaOH, 0.019% ProClin300, 0.010% Tween-20, 1.96% Trehalose, 0.6 mg/ml BSA with a denaturation temperature of 97° C., and an annealing temperature of 62° C. While “standard amplification conditions” are described herein for reference purposes, it is contemplated that oligonucleotides in conjunction with some embodiments herein can readily be used under other “amplification conditions,” including but not limited to, modifications and variations of such “standard amplification conditions.” Non-limiting examples of “amplification conditions” include the conditions disclosed in the references cited herein, such as, for example, 5 mM MgCl₂, 100 mM Tris, 10 mM NaOH, 0.019% ProClin300, 0.010% Tween-20, 1.96% Trehalose, 0.6 mg/ml BSA with an annealing temperature of 72° C.; 5 mM MgCl₂; 100 mM Tris, 10 mM NaOH, 0.019% ProClin300, 0.010% Tween-20, 1.96% Trehalose, 0.6 mg/ml BSA with an annealing temperature of 62° C.; 5 mM MgCl₂, 100 mM Tris, 10 mM NaOH, 0.019% ProClin300, 0.010% Tween-20, 1.96% Trehalose, 0.6 mg/ml BSA with an annealing temperature of 60° C.; 5 mM MgCl₂; 5 mM MgCl₂, 100 mM Tris, 10 mM NaOH, 0.019% ProClin300, 0.010% Tween-20, 1.96% Trehalose, 0.6 mg/ml BSA with an annealing temperature of 55° C.; 50 mM KCl, 10 mM Tris-HCl (pH 9.0), 0.1% Triton X-100, 2.5 mM MgCl2, with an annealing temperature of 72° C.; or 4 mM MgCl2, 100 mM Tris, pH 8.3, 10 mM KCl, 5 mM (NH₄)2SO₄, 0.15 mg BSA, 4% Trehalose, with an annealing temperature of 62° C.; 4 mM MgCl2, 100 mM Tris, pH 8.3, 10 mM KCl, 5 mM (NH₄)2SO₄, 0.15 mg BSA, 4% Trehalose, with an annealing temperature of 60° C.; or 50 mM KCl, 10 mM Tris-HCl (pH 9.0), 0.1% Triton X-100, 2.5 mM MgCl2, with an annealing temperature of 55° C., or the like. In some embodiments, an annealing temperatures as described herein is modified, for example to at least about 50° C., for example 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., or 75° C.

In some embodiments, at least one polymerase is provided. The polymerase can be used for quantitative PCR. Different nucleic acid polymerases are available for use, including but not limited to the FASTSTART™ Taq DNA polymerase (Roche), the KlenTaq 1 (AB peptides Inc.), the HOTGOLDSTAR™ DNA polymerase (Eurogentec), the KAPATAQ™ HotStart DNA polymerase or the KAPA2G™ Fast HotStart DNA polymerase (Kapa Biosystemss), and the PHUSION™ Hot Start (Finnzymes).

### Thermal Cycling

Thermal cycling conditions can vary in time as well as in temperature for each of the different steps, depending on the thermal cycler used as well as other variables that could modify the amplification's performance. In some embodiments, a 2-step protocol is performed, in which the protocol combines the annealing and elongation steps at a common temperature, optimal for both the annealing of the primers and probes as well as for the extension step. In some embodiments, a 3-step protocol is performed, in which a denaturation step, an annealing step, and an elongation step are performed.

In some embodiments, the compositions disclosed herein can be used in connection with devices for real-time amplification reactions, e.g., the BD MAX® (Becton Dickinson and Co., Franklin Lakes, N.J.), the VIPER® (Becton Dickinson and Co., Franklin Lakes, N.J.), the VIPER LT® (Becton Dickinson and Co., Franklin Lakes, N.J.), the SMARTCYLCER® (Cepheid, Sunnyvale, Calif.), ABI PRISM 7700® (Applied Biosystems, Foster City, Calif.), ROTOR-GENE™ (Corbett Research, Sydney, Australia), LIGHTCYCLER® (Roche Diagnostics Corp, Indianapolis, Ind.), ICYCLER® (BioRad Laboratories, Hercules, Calif.), IMX4000® (Stratagene, La Jolla, Calif.), CFX96™ Real-Time PCR System (Bio-Rad Laboratories Inc), and the like.

### Isothermal Amplification

In some embodiments, the compositions disclosed herein can be used in methods comprising isothermal amplification of nucleic acids. Isothermal amplification conditions can vary in time as well as temperature, depending on variables such as the method, enzyme, template, and primer or primers used. Examples of amplification methods that can be performed under isothermal conditions include, but are not limited to, some versions of LAMP, SDA, and the like.

Isothermal amplification can include an optional denaturation step, followed by an isothermal incubation in which nucleic acid is amplified. In some embodiments, an isothermal incubation is performed without an initial denaturing step. In some embodiments, the isothermal incubation is performed at least about 25° C., for example about 25° C., 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75° C., including ranges between any of the listed values. In some embodiments, the isothermal incubation is performed at about 37° C. In some embodiments, the isothermal incubation is performed at about 64° C. In some embodiments, the isothermal incubation is performed for 180 minutes or less, for example about 180, 165, 150, 135, 120, 105, 90, 75, 60, 45, 30, or 15 minutes, including ranges between any two of the listed values.

### Oligonucleotides

In some embodiments, oligonucleotides are provided, for example primers and/or probes. As used herein, the terms “primer” and “probe” include, but are not limited to oligonucleotides. Preferably, the oligonucleotide primers and/or probes disclosed herein can be between 8 and 45 nucleotides in length. For example, the primers and or probes can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or more nucleotides in length. Primers and/or probes can be provided in any suitable form, included bound to a solid support, liquid, and lyophilized, for example. The primer and probe sequences disclosed herein can be modified to contain additional nucleotides at the 5′ or the 3′ terminus, or both. The skilled artisan will appreciate, however, that additional bases to the 3′ terminus of amplification primers (not necessarily probes) are generally complementary to the template sequence. The primer and probe sequences disclosed herein can also be modified to remove nucleotides at the 5′ or the 3′ terminus.

Oligonucleotide primers and probes can bind to their targets at an annealing temperature, which is a temperature less than the melting temperature (Tₘ). As used herein, “Tₘ” and “melting temperature” are interchangeable terms which refer to the temperature at which 50% of a population of double-stranded polynucleotide molecules becomes dissociated into single strands. Formulae for calculating the Tₘ of polynucleotides are well known in the art. For example, the Tm may be calculated by the following equation: Tm=69.3+0.41 x.(G+C)%-6-50/L, wherein L is the length of the probe in nucleotides. The Tm of a hybrid polynucleotide may also be estimated using a formula adopted from hybridization assays in 1 M salt, and commonly used for calculating Tm for PCR primers: [(number of A+T)×2° C.+(number of G+C)×4° C.]. See, e.g., C. R. Newton et al. PCR, 2nd Ed., Springer-Verlag (New York: 1997), p. 24. Other more sophisticated computations exist in the art, which take structural as well as sequence characteristics into account for the calculation of Tₘ. The melting temperature of an oligonucleotide can depend on complementarity between the oligonucleotide primer or probe and the binding sequence, and on salt conditions. In some embodiments, an oligonucleotide primer or probe provided herein has a Tₘ of less than about 90° C. in 50 mM KCl, 10 mM Tris-HCl buffer, for example about 89° C., 88, 87, 86, 85, 84, 83, 82, 81, 80 79, 78, 77, 76, 75, 74, 73, 72, 71, 70, 69, 68, 67, 66, 65, 64, 63, 62, 61, 60, 59, 58, 57, 56, 55, 54, 53, 52, 50, 49, 48, 47, 46, 45, 44, 43, 42, 41, 40, 39° C., or less, including ranges between any two of the listed values. In some embodiments, an oligonucleotide primer or probe provided herein has a Tₘ of less than about 90° C. in 5 mM MgCl₂, 100 mM Tris, 10 mM NaOH, 0.019% ProClin300, 0.010% Tween-20, 1.96% Trehalose, 0.6 mg/ml BSA, for example about 89° C., 88, 87, 86, 85, 84, 83, 82, 81, 80 79, 78, 77, 76, 75, 74, 73, 72, 71, 70, 69, 68, 67, 66, 65, 64, 63, 62, 61, 60, 59, 58, 57, 56, 55, 54, 53, 52, 50, 49, 48, 47, 46, 45, 44, 43, 42, 41, 40, 39° C., or less, including ranges between any two of the listed values. As discussed in further detail below, in some embodiments, the primers disclosed herein are provided as an amplification primer set, e.g., comprising a forward primer and a reverse primer. Preferably, the forward and reverse primers have Tm's that do not differ by more than 10° C., e.g., that differ by less than 10° C., less than 9° C., less than 8° C., less than 7° C., less than 6° C., less than 5° C., less than 4° C., less than 3° C., less than 2° C., or less than 1° C.

The primer and probe sequences may be modified by having nucleotide substitutions (relative to the target nucleic acid sequence) within the oligonucleotide sequence, provided that the oligonucleotide contains enough complementarity to hybridize specifically to the target nucleic acid sequence. In this manner, at least 1, 2, 3, 4, or up to about 5 nucleotides can be substituted. As used herein, the term “complementary” refers to sequence complementarity between regions of two polynucleotide strands or between two regions of the same polynucleotide strand. A first region of a polynucleotide is complementary to a second region of the same or a different polynucleotide if, when the two regions are arranged in an antiparallel fashion, at least one nucleotide of the first region is capable of base pairing with a base of the second region. Therefore, it is not required for two complementary polynucleotides to base pair at every nucleotide position. “Fully complementary” refers to a first polynucleotide that is 100% or “fully” complementary to a second polynucleotide and thus forms a base pair at every nucleotide position. “Partially complementary” also refers to a first polynucleotide that is not 100% complementary (e.g., 90%, or 80% or 70% complementary) and contains mismatched nucleotides at one or more nucleotide positions. In some embodiments, an oligonucleotide includes a universal base.

As used herein, the term “hybridization” is used in reference to the pairing of complementary (including partially complementary) polynucleotide strands. Hybridization and the strength of hybridization (i.e., the strength of the association between polynucleotide strands) is impacted by many factors well known in the art including the degree of complementarity between the polynucleotides, stringency of the conditions involved affected by such conditions as the concentration of salts, the melting temperature of the formed hybrid, the presence of other components (e.g., the presence or absence of polyethylene glycol), the molarity of the hybridizing strands and the G:C content of the polynucleotide strands. In some embodiments, the primers are designed such that the Tm of one primer in the set is within 2° C. of the Tₘ of the other primer in the set. An extensive guide to the hybridization of nucleic acids is found in Tijssen (1993) Laboratory Techniques in Biochemistry and Molecular Biology-Hybridization with Nucleic Acid Probes, Part I, Chapter 2 (Elsevier, New York); and Ausubel et al, eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.). As discussed further herein, the term “specific hybridization” or “specifically hybridizes” refers to the hybridization of a polynucleotide, e.g., an oligonucleotide primer or probe or the like to a target sequence, such as a sequence to be quantified in a sample, a positive control target nucleic acid sequence, or the like, and not to unrelated sequences, under conditions typically used for nucleic acid amplification.

In some embodiments, the primers and/or probes include oligonucleotides that hybridize to a target nucleic acid sequence over the entire length of the oligonucleotide sequence. Such sequences can be referred to as “fully complementary” with respect to each other. Where an oligonucleotide is referred to as “substantially complementary” with respect to a nucleic acid sequence herein, the two sequences can be fully complementary, or they may form mismatches upon hybridization, but retain the ability to hybridize under stringent conditions or standard PCR conditions as discussed below. As used herein, the term “substantially complementary” refers to the complementarity between two nucleic acids, e.g., the complementary region of the oligonucleotide and the target sequence. The complementarity need not be perfect; there may be any number of base pair mismatches that between the two nucleic acids. However, if the number of mismatches is so great that no hybridization can occur under even the least stringent of hybridization conditions, the sequence is not a substantially complementary sequence. When two sequences are referred to as “substantially complementary” herein, it is meant that the sequences are sufficiently complementary to the each other to hybridize under the selected reaction conditions. The relationship of nucleic acid complementarity and stringency of hybridization sufficient to achieve specificity is well known in the art and described further below in reference to sequence identity, melting temperature and hybridization conditions. Therefore, substantially complementary sequences can be used in any of the detection methods disclosed herein. Such probes can be, for example, perfectly complementary or can contain from 1 to many mismatches so long as the hybridization conditions are sufficient to allow, for example discrimination between a target sequence and a non-target sequence. Accordingly, substantially complementary sequences can refer to sequences ranging in percent identity from 100%, 99, 98, 97, 96, 95, 94, 93, 92, 91, 90, 89, 88, 87, 86, 85, 84, 83, 82, 81, 80, 75, 70% or less, or any number in between, compared to the reference sequence. For example, the oligonucleotides disclosed herein can contain 1, 2, 3, 4, 5, or more mismatches and/or degenerate bases (e.g. “variant oligonucleotides”), as compared to the target sequence to which the oligonucleotide hybridizes, with the proviso that the oligonucleotides are capable of specifically hybridizing to the target sequence under, for example, standard nucleic acid amplification conditions.

The primers described herein can be prepared using techniques known in the art, including, but not limited to, cloning and digestion of the appropriate sequences and direct chemical synthesis. Chemical synthesis methods that can be used to make the primers of the described herein, include, but are not limited to, the phosphotriester method described by Narang et al. (1979) Methods in Enzymology 68:90, the phosphodiester method disclosed by Brown et al. (1979) Methods in Enzymology 68:109, the diethylphosphoramidate method disclosed by Beaucage et al. (1981) Tetrahedron Letters 22:1859, and the solid support method described in U.S. Pat. No. 4,458,066. The use of an automated oligonucleotide synthesizer to prepare synthetic oligonucleotide primers described herein is also contemplated herein. Additionally, if desired, the primers can be labeled using techniques known in the art and described below.

### Primer Sets

In some embodiments, a set of amplification primers is provided. The set of amplification primers can include one or more, e.g., 1, 2, 3, 4, 5, 6, 7, 8, 9, 10, 11, 12, 13, 14, 15, or more primer pairs. As used herein, the term “primer pair” can refer to two amplification primers that individually hybridize to opposite strands of a target nucleic acid sequence (e.g., a sequence of E. histolytica, a sequence of E. dispar, or a sequence found in both E. histolytic and E. dispar), in which each primer can be extended at its 3′ end to form a target amplification product, for example in PCR. The target amplification product can include an amplicon. A primer pair can include a forward primer and a reverse primer. The skilled artisan will appreciate that the terms “forward primer” and “reverse primer” are frequently used for convenience in identifying each primer in a primer pair, for example with reference to a which strand is identified as the “+” strand or “top” strand of a target nucleic acid sequence, but that no further limitation should be inferred from “forward” or “reverse,” unless stated otherwise.

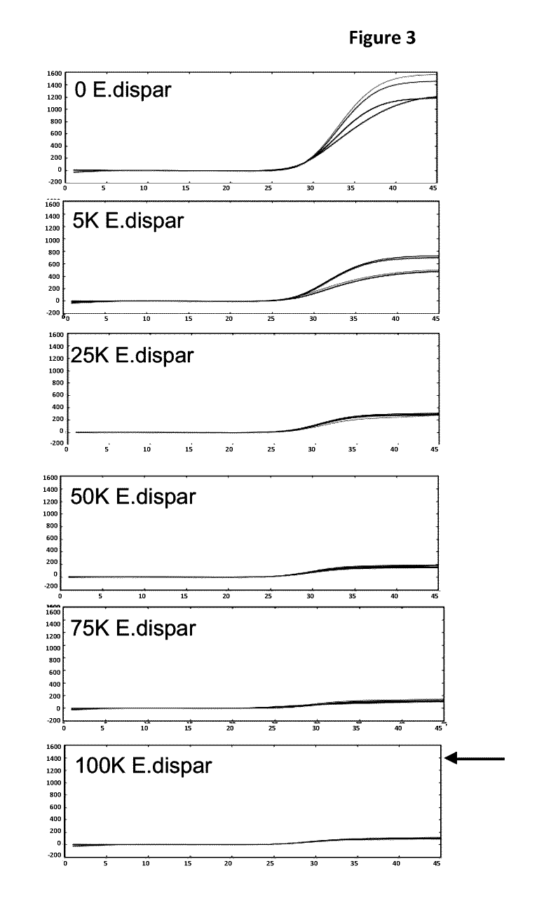

In some embodiments, the primer set includes amplification primers that will anneal to, and amplify, a sequence of E. histolytica under standard amplification conditions, but will not anneal to a sequence of E. dispar, or will anneal to a sequence of E. dispar, but not substantially amplify this sequence of E. dispar under the same or similar amplification conditions. Accordingly, in some embodiments, the primer set is used to detect the presence of E. histolytica, but not E. dispar. Due to the high degree of homology between E. histolytica and E. dispar, an alternative approach for quantitative amplification of E. histolytica sequences would be to select a primer set that amplifies a polynucleotide sequence found in both E. histolytica and E. dispar (e.g., a homologous sequence), and then use a probe that hybridizes only to the polynucleotide sequence of E. histolytica to detect amplification of E. histolytica product (see Verweij, et al., Clin. Microbiol. 42: 1220-23, 2004). Unexpectedly, it has been discovered herein that undertaking such an approach can result in reduction of the expected amplification signal from E. histolytica, especially as the dose of E. dispar target nucleic acid sequence increases. As E. histolytica and E. dispar may both infect the same individual, it is contemplated that previous approaches (e.g. of Verweij, et al., Clin. Microbiol. 42: 1220-23, 2004) could result in false negatives. As shown in Example 1 and FIG. 3, when a primer set that amplifies both E. histolytica and E. dispar nucleic acid sequences was used, a known copy number of E. histolytica target nucleic acid sequence became nearly undetectable in the presence of a high copy number of E. dispar target nucleic acid sequences. Without being bound by any one theory, it is contemplated that homo- and hetero duplex formation between amplification products of E. histolytica and E. dispar can block available E. histolytica probe binding sequences as the proportion of E. dispar amplicons increases in the reaction. Performing quantitative nucleic acid amplification of E. histolytica according to some embodiments herein can minimize or eliminate interfering effects of E. dispar nucleic acids. Thus, in some embodiments, depression of E. histolytica signal by E. dispar can be minimized. In some embodiments, depression of E. histolytica signal by E. dispar can be effectively eliminated. In some embodiments, primers are designed to substantially amplify a E. histolytica target nucleic acid sequence under standard amplification conditions, without substantially amplifying any E. dispar nucleic acid sequences.

In some embodiments, the primers of the primer set will individually hybridize to opposite strands of a target nucleic acid of E. histolytica under standard amplification conditions, so as to define a target amplification product. In some embodiments, when extended at their respective 3′ ends, the primers will produce a target amplification product. Accordingly, in some embodiments, when extended, the primers will substantially amplify an E. histolytica target nucleic acid sequence. In some embodiments, neither primer of the primer pair will hybridize to a strand of E. dispar nucleic acid under standard amplification conditions, and thus will not substantially amplify any sequence of E. dispar. In some embodiments, only one primer of the primer pair will hybridize to a strand of E. dispar nucleic acid under standard amplification conditions, while the other primer will not hybridize to any E. dispar nucleic acid under these conditions, so that the primer set will fail to substantially amplify any E. dispar sequence. In some embodiments, each primer of the primer pair will hybridize to E. dispar nucleic acid under standard amplification conditions, but these primers will not hybridize in an orientation that will form an amplification product when each primer is extended at its 3′ end (e.g. the primers may hybridize to the same strand, or hybridize too far apart to form an amplification product when extended, or hybridize in an orientation so that when extended at its 3′ end, at least one primer extends “away” from the other primer). Accordingly, in some embodiments, the primers of the primer pair will not substantially amplify any nucleic acid sequence of E. dispar.

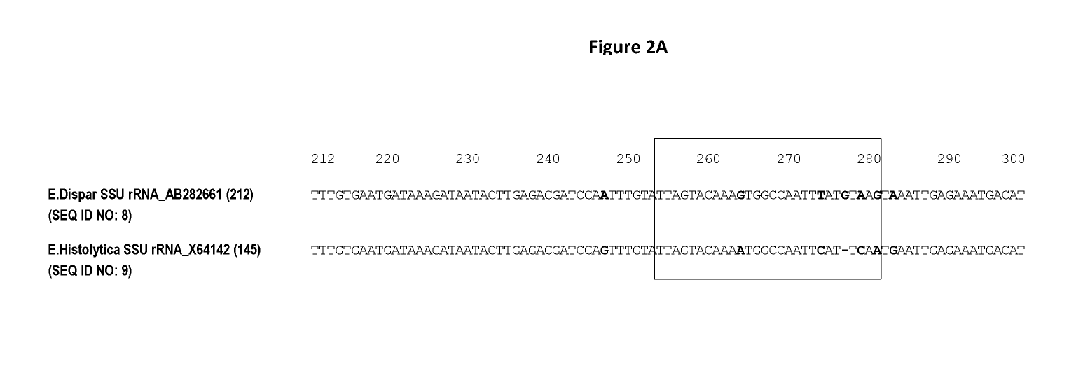

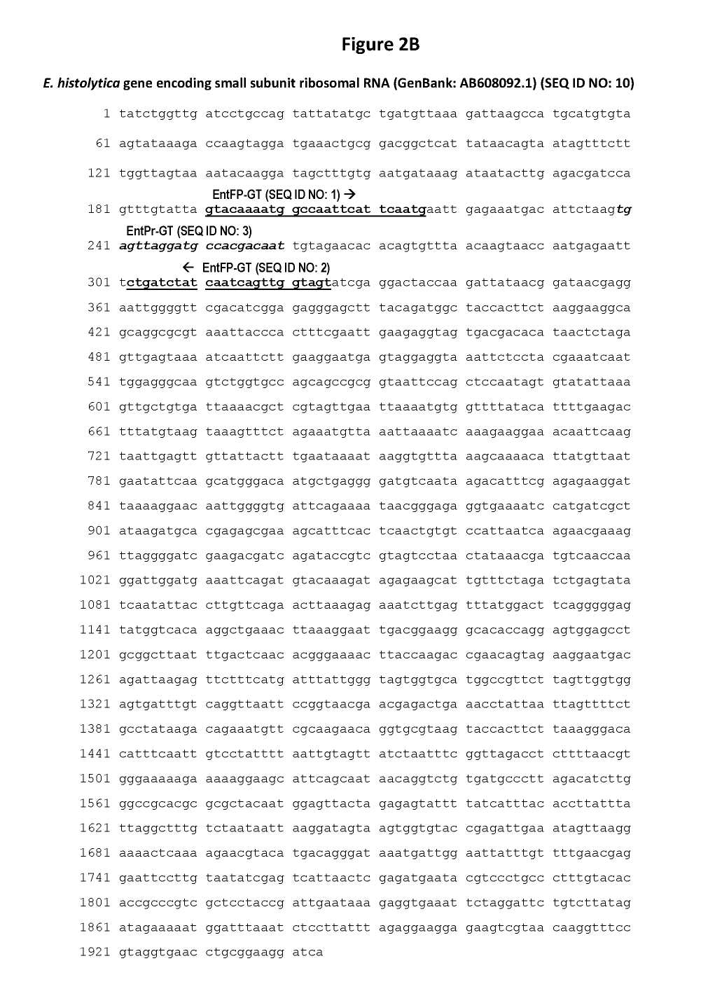

In some embodiments, in designing primer sets that reliably amplify sequences of E. histolytica but not E. dispar, it can be useful to select primers that amplify a conserved region of E. histolytica, so as to minimize false negatives due to strain-to-strain variation among E. histolytica, but do not amplify a conserved region of E. dispar, so as to minimize false positives that could otherwise be caused by the presence of E. dispar. For example, a highly conserved sequence with ancestral differences between E. dispar and E. histolytica can be a useful region from which to select a target nucleic acid (e.g. a “template”) for a target amplification sequence. In some embodiments, the target amplification sequence includes an rDNA gene or portion thereof. In some embodiments, a gene product (for example, an rRNA or portion thereof) is reverse-transcribed, and used as a target nucleic acid sequence for qualitative and/or quantitative nucleic acid amplification. While small ribosomal subunit genes are highly conserved, there are some apparently ancestral differences between the sequences of the small ribosomal subunit gene of E. histolytica and E. dispar, as shown in FIG. 2A. The skilled artisan will appreciate that these differences can be used to design primer sets that can produce a target amplification sequence of E. histolytica, but not E. dispar. In some embodiments, the primer pair amplifies a polynucleotide sequence that includes at least a portion of the gene encoding the E. histolytica small subunit ribosomal RNA (GenBank Accession No: AB608092.1)(SEQ ID NO: 10). An annotated diagram of the E. histolytica small subunit ribosomal RNA gene is illustrated in FIG. 2B. In some embodiments, the target sequence includes a polynucleotide having SEQ ID NO: 7 (e.g. positions 191-325 of SEQ ID NO: 10) (GTACAAAATGGCCAATTCATTCAATGAATTGAGAAATGACATTCTAAGTGAG TTAGGATGCCACGACAATTGTAGAACACACAGTGTTTAACAAGTAACCAATG AGAATTTCTGATCTATCAATCAGTTGGTAGT). In some embodiments the target amplification product includes at least about 30 continuous nucleotides of SEQ ID NO: 7, for example at least about 30, 35, 40, 45, 50, 55, 60, 65, 70, 75, 80, 85, 90, 95, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, or 135 continuous nucleotides of SEQ ID NO: 7, including ranges between any two of the listed values. In some embodiments, the target amplification sequence includes at least about 30-135 continuous nucleotides of SEQ ID NO: 7, for example about 30-100, 30-110, 30-115, 30-120, 30-125, 30-130, 30-135, 40-100, 40-110, 40-115, 40-120, 40-125, 40-130, 40-135, 50-100, 50-110, 50-115, 50-120, 50-125, 50-130, 50-135, 60-100, 60-110, 60-115, 60-120, 60-125, 60-130, 60-135, 70-100, 70-110, 70-115, 70-120, 70-125, 70-130, 70-135, 80-100, 80-110, 80-115, 80-120, 80-125, 80-130, 80-135, 90-100, 90-110, 90-115, 90-120, 90-125, 90-130, 90-135, 100-110, 100-115, 100-120, 100-125, 100-130, 100-135, 110-115, 110-120, 110-125, 110-130, 110-135, 115-120, 115-125, 115-130, 115-135, 120-125, 120-130, 120-135, 125-130, 125-135, or 130-135 continuous nucleotides of SEQ ID NO: 7. In some embodiments, the target amplification produce has at least 70% nt-nt identity to SEQ ID NO: 7, for example at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.2% nt-nt identity, including ranges between any two of the listed values. In some embodiments, the target amplification sequence includes a polynucleotide having the sequence of SEQ ID NO: 7, and at least one additional polynucleotide upstream and/or downstream of a 5′ end or 3′ end of SEQ ID NO: 7 (e.g. positions 191-325 of SEQ ID NO: 10). In some embodiment, the target amplification sequence includes at least about 1 nucleotide upstream of the 5′ end of SEQ ID NO: 7 as shown in SEQ ID NO: 10 (see FIG. 2B), for example at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, or 200 nucleotides upstream. In some embodiment, the target amplification includes at least about 1 nucleotide downstream of the 3′ end of SEQ ID NO: 7 as shown in SEQ ID NO: 10 (see FIG. 2B), for example at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, or 200 nucleotides upstream. In some embodiments, the target amplification sequence includes nucleotides both upstream and downstream of the ends of SEQ ID NO: 7 (as shown in SEQ ID NO: 10), as described herein. In some embodiments, the pimer pair does not amplify, under standard amplification conditions, the E. dispar small subunit rRNA. By way of example, a sequence of the E. dispar small subunit rRNA can be found Genbank accession umber AB282661, which is provided herein as SEQ ID NO: 11 (TGGATATAAATACAAAAGAGAAGTAAGAATAAAGAATCCTTCCTTTTAAAAA GGAAGAAGAATAAAATATCTGGTTGATCCTGCCAGTATTATATGCTGATGTTA AAGATTAAGCCATGCATGTGTAAGTATAAAGACCAAGTAGGATGAAACTGCG GACGGCTCATTATAACAGTAATAGTTTCTTTGGTTAGTAAAGTACAAGGATAG CTTTGTGAATGATAAAGATAATACTTGAGACGATCCAATTTGTATTAGTACAA AGTGGCCAATTTATGTAAGTAAATTGAGAAATGACATTCTAAGTGAGTTAGG ATGCCACGACAATTGTAGAACACACAGTGTTTAACAAGTAACCAATGAGAAT TTCTGATCTATCAATCAGTTGGTAGTATCGAGGACTACCAAGATTATAACGGA TAACGAGGAATTGGGGTTCGACATCGGAGAGGGAGCTTTACAGATGGCTACC ACTTCTAAGGAAGGCAGCAGGCGCGTAAATTACCCACTTTCGAATTGAAGAG GTAGTGACGACACATAACTCTAGAGTTGAGTAAAATCAATTCTTGAAGGAAT GAGTAGGAGGTAAATTCTCCTACGAAATCAATTGGAGGGCAAGTCTGGTGCC AGCAGCCGCGGTAATTCCAGCTCCAATAGTGTATATTAAAGTTGCTGTGATTA AAACGCTCGTAGTTGAATTAAAATGTGATTTTATACATTTTGAAGACTTTACA TTAAGTGAAGTTTCTAGAAATGTTAAATTAAAATCAAAGAAGGAGACAATTC AAGTAATTGAGTTGTCATTACTTTGAATAAAATAAGGTGTTTAAAGCAAAACA TTATGTTAATGAATATTCAAGCATGGGACAATGCTGAGGAGATGTCAATTAG ACATTTCGAGAGAAGGATTAAAAGGAACAATTGGGGTGATTCAGAAAATAAC GGGAGAGGTGAAAATCCATGATCGCTATAAGATGCACGAGAGCGAAAGCATT TCACTCAACTGGGTCCATTAATCAAGAACGAAAGTTAGGGGATCGAAGACGA TCAGATACCGTCGTAGTCCTAACTATAAACGATGTCAACCAAGGATTGGATG AAATTCAGATGTACAAAGATGAAGAAACATTGTTTCTAAATCCAAGTATATC AATACTACCTTGTTCAGAACTTAAAGAGAAATCTTGAGTTTATGGACTTCAGG GGGAGTATGGTCACAAGGCTGAAACTTAAAGGAATTGACGGAAGGGCACACC AGGAGTGGAGCCTGCGGCTTAATTTGACTCAACACGGGAAAACTTACCAAGA CC GAACAGTAGAAGGAATGACAGATTAAGAGTTCTTTCATGATTTATTGGGTA GTGGTGCATGGCCGTTCTTAGTTGGTGGAGTGATTTGTCAGGTTAATTCCGGT AACGAACGAGACTGAAACCTATTAATTAGTTTTCTGCCTATAAGACAGAAAT GTTCGCAAGAACAGGTGCGTAAGTACCACTTCTTAAAGGGACACATTTCAATT GTCCTATTTTAATTGTTAGTTATCTAATTTCGATTAGAACTCTTTTAACGTGGG AAAAAGAAAAAGGAAGCATTCAGCAATAACAGGTCTGTGATGCCCTTAGACA TCTTGGGCCGCACGCGCGCTACAATGGAGTTACTAGAGAGCATTTTATCATTT ACACCTTATTTATTAGGCTATGTCTAATAGGTAGGGATAGTAAGTGGTGTACC GAGATTGAAATAGTTAAGGAAAACTCAAAAGAACGTACATGACAGGGATAA ATGATTGGAATTATTTGTTTTGAACGAGGAATTCCTTGTAATATCGAGTCATT AACTCGAGATGAATACGTCCCTGCCCTTTGTACACACCGCCCGTCGCTCCTAC CGATTGAATAAAGAGGTGAAATTCTAGGATTCTGTCTTATAGATAGAAAAAT GGATTTAAATCTCCTTATTTAGAGGAAGGAGAAGTCGTAACAAGGTTTCCGTA GGTGAACCTGCGGAAGGATCATTAAAAGAAAAGAAATAATCTTTTAAAATAA AACAAGAAATTTATAGAATAAGATAATCTACAAAGAAAATAATAAAAGTAAG AATAAAAGGAATTAGAATATAAGAAGAAAGAAAAAGTATAATAAAATATTA CTTTGGATAGTTTAGTTTCCTGTGCGATGAAGAACGCAATGAATTGCGATAAG TGATAGGAACAATAAAATGTGAATATCCAAACTTTGAATGCTTGAAAGTATA CTTATGAACTTCAAGGTATATATGATATTCAATATCCAAAATAAAAGAGTATA TTAAAAGCAAATATTAGTAGAAGTGAGAAGTAGCTAGTGGGTAAAAGAGAG AAGAAGTAAAGAGCTTTAACCAGATATCTATAAGTGAGTTAATAAATAAAGA TTTGAGTATCGTAAGAG).

In some embodiments, the primer set includes a first primer that comprises SEQ ID NO: 1 (GTACAAAATGGCCAATTCATTCAATG). In some embodiments the primer set includes a second primer that comprises SEQ ID NO: 2 (ACTACCAACTGATTGATAGATCAG). As shown in FIG. 2A, a primer that comprises SEQ ID NO: 1 can have high (up to 100%) identity to a sequence encoding the E. histolytica small subunit rRNA, while having lower identity to a homologous region of the E. dispar genome. Accordingly, in some embodiments, a primer comprising SEQ ID NO: 1 hybridizes to E. histolytica genomic DNA, but does not hybridize to E. dispar genomic DNA under standard amplification conditions. A primer that comprises SEQ ID NO: 2 can hybridize to genomic DNA in the region of the small subunit rRNA gene of both E. histolytica and E. dispar. Accordingly, in some embodiments, a primer set that includes a first primer comprising SEQ ID NO: 1, and a second primer comprising SEQ ID NO: 2, can amplify a target sequence of E. histolytica nucleic acid, but not E. dispar nucleic acids. In some embodiments, the nucleic acid includes genomic DNA. In some embodiments, the nucleic acid includes nucleic acid reverse-transcribed from a gene product, for example an mRNA or rRNA.

In some embodiments, the first primer comprises at least about 10 consecutive nucleotides of SEQ ID NO:1, for example at least about 10, 11, 12, 13, 14, 15, 16, 17, 18, 19, 20, 21, 22, 23, 24, 25, or 26 consecutive nucleotides of SEQ ID NO: 1 or its complement. In some embodiments, the first primer comprises a polynucleotide sequence that is at least about 38% identical of SEQ ID NO: 1, for example at least about 38%, 42, 46, 50, 53, 57, 61, 65, 69, 73, 76, 80, 84, 88, 92, or 96% identical to SEQ ID NO: 1. In some embodiments, the first primer comprises SEQ ID NO: 1, and at least 1 additional nucleotide 5′ of the 5′ terminus of SEQ ID NO: 1, for example about 1, 2, 3, 4, 5, 6, 7, 8, 9, 10, 11, 12, 13, 14, 15, 16, 17, 18, 19, or 20 nucleotides 5′ of the 5′ terminus of SEQ ID NO: 1. In some embodiments, one or more of the nucleotides 5′ of SEQ ID NO: 1 are complementary to the template strand of SEQ ID NO: 10 as shown in FIG. 2B.

A number of Alternatives are contemplated for primers in accordance with some embodiments herein:

In accordance with Alternative 1, the first primer has a length of 15-50 nucleotides and comprises at least about 10 consecutive nucleotides of SEQ ID NO:1, for example at least about 10, 11, 12, 13, 14, 15, 16, 17, 18, 19, 20, 21, 22, 23, 24, 25, or 26 consecutive nucleotides of SEQ ID NO: 1 or its complement, including ranges between any two of the listed values, for example 10-15, 10-20, 15-20, 10-26, 15-26, or 20-26 consecutive nucleotides. The first primer can hybridize to a target sequence of SEQ ID NO: 10, and have at least about 80% identity to the target sequence.

In accordance with Alternative 2, the first primer has a length of 15-50 nucleotides and comprises at least about 10 consecutive nucleotides of SEQ ID NO:1, for example at least about 10, 11, 12, 13, 14, 15, 16, 17, 18, 19, 20, 21, 22, 23, 24, 25, or 26 consecutive nucleotides of SEQ ID NO: 1 or its complement, including ranges between any two of the listed values, for example 10-15, 10-20, 15-20, 10-26, 15-26, or 20-26 consecutive nucleotides. The first primer can hybridize to a target sequence of SEQ ID NO: 10, and have at least about 85% identity to the target sequence.

In accordance with Alternative 3, the first primer has a length of 15-50 nucleotides and comprises at least about 10 consecutive nucleotides of SEQ ID NO:1, for example at least about 10, 11, 12, 13, 14, 15, 16, 17, 18, 19, 20, 21, 22, 23, 24, 25, or 26 consecutive nucleotides of SEQ ID NO: 1 or its complement, including ranges between any two of the listed values, for example 10-15, 10-20, 15-20, 10-26, 15-26, or 20-26 consecutive nucleotides. The first primer can hybridize to a target sequence of SEQ ID NO: 10, and have at least about 90% identity to the target sequence.

In accordance with Alternative 4, the first primer has a length of 15-50 nucleotides and comprises at least about 10 consecutive nucleotides of SEQ ID NO:1, for example at least about 10, 11, 12, 13, 14, 15, 16, 17, 18, 19, 20, 21, 22, 23, 24, 25, or 26 consecutive nucleotides of SEQ ID NO: 1 or its complement, including ranges between any two of the listed values, for example 10-15, 10-20, 15-20, 10-26, 15-26, or 20-26 consecutive nucleotides. The first primer can hybridize to a target sequence of SEQ ID NO: 10, and have at least about 95% identity to the target sequence. Optionally, the first primer can have 100% identity to the target sequence.

In accordance with Alternative 5, the first primer of any of Alternatives 1-4 can be paired with the second primer. The second primer can primer have a length of 15-50 nucleotides and comprise at least about 10 consecutive nucleotides of SEQ ID NO:2, for example at least about 10, 11, 12, 13, 14, 15, 16, 17, 18, 19, 20, 21, 22, 23, 24, 25, or 26 consecutive nucleotides of SEQ ID NO: 2 or its complement, including ranges between any two of the listed values, for example 10-15, 10-20, 15-20, 10-26, 15-26, or 20-26 consecutive nucleotides. The second primer can hybridize to a target sequence of SEQ ID NO: 10, and have at least about 80% identity to the target sequence.

In accordance with Alternative 6, the first primer of any of Alternatives 1-4 can be paired with the second primer. The second primer can have a length of 15-50 nucleotides and comprise at least about 10 consecutive nucleotides of SEQ ID NO:2, for example at least about 10, 11, 12, 13, 14, 15, 16, 17, 18, 19, 20, 21, 22, 23, 24, 25, or 26 consecutive nucleotides of SEQ ID NO: 2 or its complement, including ranges between any two of the listed values, for example 10-15, 10-20, 15-20, 10-26, 15-26, or 20-26 consecutive nucleotides. The second primer can hybridize to a target sequence of SEQ ID NO: 10, and have at least about 85% identity to the target sequence.

In accordance with Alternative 7, the first primer of any of Alternatives 1-4 can be paired with the second primer. The second primer can have a length of 15-50 nucleotides and comprise at least about 10 consecutive nucleotides of SEQ ID NO:2, for example at least about 10, 11, 12, 13, 14, 15, 16, 17, 18, 19, 20, 21, 22, 23, 24, 25, or 26 consecutive nucleotides of SEQ ID NO: 2 or its complement, including ranges between any two of the listed values, for example 10-15, 10-20, 15-20, 10-26, 15-26, or 20-26 consecutive nucleotides. The second primer can hybridize to a target sequence of SEQ ID NO: 10, and have at least about 90% identity to the target sequence.

In accordance with Alternative 8, the first primer of any of Alternatives 1-4 can be paired with the second primer. The second primer have a length of 15-40 nucleotides and can comprise at least about 10 consecutive nucleotides of SEQ ID NO:2, for example at least about 10, 11, 12, 13, 14, 15, 16, 17, 18, 19, 20, 21, 22, 23, 24, 25, or 26 consecutive nucleotides of SEQ ID NO: 2 or its complement, including ranges between any two of the listed values, for example 10-15, 10-20, 15-20, 10-26, 15-26, or 20-26 consecutive nucleotides. The second primer can hybridize to a target sequence of SEQ ID NO: 10, and have at least about 95% identity to the target sequence. Optionally, the second primer can have 100% identity to the target sequence.

In some embodiments, the first primer is designed in accordance with the alignment shown in FIG. 2A, so that the first primer anneals to a sequence of E. histolytica, but not to the homologous sequence of E. dispar under standard amplification conditions. In some embodiments, the first primer anneals to a sequence of E. histolytica, but not to the homologous sequence of E. dispar under typical PCR conditions, e.g., at 50° C. in 50 mM KCl, 10 mM Tris-HCl buffer (pH 8.0), or, e.g., 62° C. in 5 mM MgCl₂, 100 mM Tris, 10 mM NaOH, 0.019% ProClin300, 0.010% Tween-20, 1.96% Trehalose, 0.6 mg/ml BSA. In some embodiments, at least the 3′-most nucleotide of the first primer is complementary to an E. histolytica nucleotide at a conserved position that differs in E. dispar, so that the first primer, when hybridized, can typically extend from its 3′ end in E. histolytica, but typically not in E. dispar. In some embodiments, the second primer comprises at least about 10 consecutive nucleotides of SEQ ID NO: 2, for example about 10, 11, 12, 13, 14, 15, 16, 17, 18, 19, 20, 21, 22, 23, or 24, consecutive nucleotides of SEQ ID NO: 2. In some embodiments, the second primer comprises a polynucleotide sequence that is at least about 41% identical of SEQ ID NO: 2, for example at least about 41, 45, 50, 54, 58, 62, 66, 70, 75, 79, 83, 87, 91, or 95% identical to SEQ ID NO: 2.

### Probes

In some embodiments, sequence-specific probes are provided. Probes include, but are not limited to oligonucleotides as described herein. In some embodiments, the sequence-specific probes disclosed herein specifically hybridize to a target nucleic acid sequence. In some embodiments, the sequence-specific probe can hybridize to a sequence that is found in both E. histolytica and E. dispar. In some embodiments, the sequence-specific probe can hybridize to a sequence that is found in E. histolytica, but not in E. dispar. In some embodiments, the sequence-specific probe specifically hybridizes to, and is fully or substantially complementary to a nucleotide sequence flanked by the binding sites of a pair of amplification primers disclosed herein. In some embodiments, the sequence-specific probe specifically hybridizes to, and is fully or substantially complementary a target amplification sequence of a primer set that amplifies E. histolytica, but not E. dispar, nucleic acids under standard amplification conditions. In some embodiments, the sequence-specific probe comprises the polynucleotide of SEQ ID NO: 3 (ATTGTCGTGGCATCCTAACTCA). In some embodiments, the sequence-specific probe comprises at least about 5 consecutive nucleotides of SEQ ID NO: 3, for example about 5, 6, 7, 8, 9, 10, 11, 12, 13, 14, 15, 16, 17, 18, 19, 20, 21, or 22 nucleotides of SEQ ID NO: 3. In some embodiments, the sequence-specific probe comprises a sequence that is at least about 22% identical so SEQ ID NO: 3, for example at least about 22%, 27, 31, 36, 40, 45, 54, 59, 63, 68, 72, 77, 81, 86, 90, or 95% identical so SEQ ID NO: 3. In some embodiments, the sequence-specific probe overlaps with the binding site of an amplification primer disclosed herein.

A number Alternatives are contemplated for probes in accordance with some embodiments herein.

In accordance with Alternative 9, the probe can have a length of 15-75 nucleotides and comprise at least 10 nucleotides of SEQ ID NO: 3, SEQ ID NO:1, for example at least about 10, 11, 12, 13, 14, 15, 16, 17, 18, 19, 20, 21, 22, 23, 24, 25, or 26 consecutive nucleotides of SEQ ID NO: 3 or its complement, including ranges between any two of the listed values, for example 10-15, 10-20, 15-20, 10-26, 15-26, or 20-26 consecutive nucleotides. The probe can hybridize to a target sequence of SEQ ID NO: 10, and have at least about 80% identity to the target sequence. The probe can be used in conjunction with any of the primer pairs of Alternatives 5-8. Optionally, the probe can also hybridize to SEQ ID NO: 11.

In accordance with Alternative 10, the probe can have a length of 15-75 nucleotides and comprises at least 10 nucleotides of SEQ ID NO: 3, SEQ ID NO:1, for example at least about 10, 11, 12, 13, 14, 15, 16, 17, 18, 19, 20, 21, 22, 23, 24, 25, or 26 consecutive nucleotides of SEQ ID NO: 3 or its complement, including ranges between any two of the listed values, for example 10-15, 10-20, 15-20, 10-26, 15-26, or 20-26 consecutive nucleotides. The probe can hybridize to a target sequence of SEQ ID NO: 10, and have at least about 85% identity to the target sequence. The probe can be used in conjunction with any of the primer pairs of Alternatives 5-8. Optionally, the probe can also hybridize to SEQ ID NO: 11.

In accordance with Alternative 11, the probe can have a length of 15-75 nucleotides and comprise at least 10 nucleotides of SEQ ID NO: 3, SEQ ID NO:1, for example at least about 10, 11, 12, 13, 14, 15, 16, 17, 18, 19, 20, 21, 22, 23, 24, 25, or 26 consecutive nucleotides of SEQ ID NO: 3 or its complement, including ranges between any two of the listed values, for example 10-15, 10-20, 15-20, 10-26, 15-26, or 20-26 consecutive nucleotides. The probe can hybridize to a target sequence of SEQ ID NO: 10, and have at least about 90% identity to the target sequence. The probe can be used in conjunction with any of the primer pairs of Alternatives 5-8. Optionally, the probe can also hybridize to SEQ ID NO: 11.

In accordance with Alternative 12, the probe can have a length of 15-75 nucleotides and comprise at least 10 nucleotides of SEQ ID NO: 3, SEQ ID NO:1, for example at least about 10, 11, 12, 13, 14, 15, 16, 17, 18, 19, 20, 21, 22, 23, 24, 25, or 26 consecutive nucleotides of SEQ ID NO: 3 or its complement, including ranges between any two of the listed values, for example 10-15, 10-20, 15-20, 10-26, 15-26, or 20-26 consecutive nucleotides. The probe can hybridize to a target sequence of SEQ ID NO: 10, and have at least about 95% identity to the target sequence. The probe can be used in conjunction with any of the primer pairs of Alternatives 5-8. Optionally, the probe can also hybridize to SEQ ID NO: 11.

In accordance with Alternative 13, the probe can have a length of 15-75 nucleotides and comprise at least 10 nucleotides of SEQ ID NO: 3, SEQ ID NO:1, for example at least about 10, 11, 12, 13, 14, 15, 16, 17, 18, 19, 20, 21, 22, 23, 24, 25, or 26 consecutive nucleotides of SEQ ID NO: 3 or its complement, including ranges between any two of the listed values, for example 10-15, 10-20, 15-20, 10-26, 15-26, or 20-26 consecutive nucleotides. The probe can hybridize to a target sequence of SEQ ID NO: 10, and have 100% identity to the target sequence. The probe can be used in conjunction with any of the primer pairs of Alternatives 5-8. Optionally, the probe can also hybridize to SEQ ID NO: 11.

Different types of detectable moieties have been described for the detection of amplification products. One class of detectable moieties is intercalating agents, which bind non-specifically to double-stranded nucleic acid. Intercalating agents have a relatively low fluorescence when unbound, and a relatively high fluorescence upon binding to double-stranded nucleic acids. As such, intercalating agents can be used to monitor the accumulation of double strained nucleic acids during a nucleic acid amplification reaction. Examples of such non-specific dyes include intercalating agents such as SYBR Green I (Molecular Probes), PicoGreen (Molecular Probes), TOTO, YOYO, propidium iodide, ethidium bromide, and the like. Other types of detectable moities employ derivatives of sequence-specific nucleic acid probes. For example, oligonucleotide probes can be labeled with one or more dyes, such that upon hybridization to a template nucleic acid, a detectable change in fluorescence is generated. While non-specific dyes may be desirable for some applications, sequence-specific probes can provide more accurate measurements of amplification. One configuration of sequence-specific probe can include one end of the probe tethered to a fluorophore, and the other end of the probe tethered to a quencher. When the probe is unhybridized, it can maintain a stem-loop configuration, in which the fluorophore is quenched by the quencher, thus preventing the fluorophore from fluorescing. When the probe is hybridized to a template nucleic sequence, it is linearized, distancing the fluorophore from the quencher, and thus permitting the fluorophore to fluoresce. Another configuration of sequence-specific probe can include a first probe tethered to a first fluorophore of a FRET pair, and a second probe tethered to a second fluorophore of a FRET pair. The first probe and second probe can be configured to hybridize to sequences of an amplicon that are within sufficient proximity to permit energy transfer by FRET when the first probe and second probe are hybridized to the same amplicon.

In some embodiments, the sequence specific probe comprises an oligonucleotide as disclosed herein conjugated to a fluorophore. In some embodiments, the probe is conjugated to two or more flurophores. Examples of fluorophores include: xanthene dyes, e.g., fluorescein and rhodamine dyes, such as fluorescein isothiocyanate (FITC), 2-[ethylamino)-3-(ethylimino)-2-7-dimethyl-3H-xanthen-9-yl]benzoic acid ethyl ester monohydrochloride (R6G)(emits a response radiation in the wavelength that ranges from about 500 to 560 nm), 1,1,3,3,3′,3′-Hexamethylindodicarbocyanine iodide (HIDC) (emits a response radiation in the wavelength that ranged from about 600 to 660 nm), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3 (emits a response radiation in the wavelength that ranges from about 540 to 580 nm), Cy5 (emits a response radiation in the wavelength that ranges from about 640 to 680 nm), etc; BODIPY dyes and quinoline dyes. Specific fluorophores of interest include: Pyrene, Coumarin, Diethylaminocoumarin, FAM, Fluorescein Chlorotriazinyl, Fluorescein, R110, Eosin, JOE, R6G, HIDC, Tetramethylrhodamine, TAMRA, Lissamine, ROX, Napthofluorescein, Texas Red, Napthofluorescein, Cy3, and Cy5, CalFluorOrange, and the like.

In some embodiments, the probe is conjugated to a quencher. A quencher can absorb electromagnetic radiation and dissipate it as heat, thus remaining dark. Example quenchers include Dabcyl, NFQ's, such as BHQ-1 or BHQ-2 (Biosearch), IOWA BLACK FQ (IDT), and IOWA BLACK RQ (IDT). In some embodiments, the quencher is selected to pair with a fluorphore so as to absorb electromagnetic radiation emitted by the fluorophore. Flourophore/quencher pairs useful in the compositions and methods disclosed herein are well-known in the art, and can be found, e.g., described in S. Marras, “Selection of Fluorophore and Quencher Pairs for Fluorescent Nucleic Acid Hybridization Probes” available at the world wide web site molecular-beacons.org/download/marras,mmb06%28335%293.pdf. In some embodiments, a flourophore/quencher pair includes CalFluor Orange and BHQ-1.

In some embodiments, a fluorophore is attached to a first end of the probe, and a quencher is attached to a second end of the probe. Attachment can include covalent bonding, and can optionally include at least one linker molecule positioned between the probe and the fluorophore or quencher. In some embodiments, a fluorophore is attached to a 5′ end of a probe, and a quencher is attached to a 3′ end of a probe. In some embodiments, a fluorphore is attached to a 3′ end of a probe, and a quencher is attached to a 5′ end of a probe. Examples of probes that can be used in quantitative nucleic acid amplification include molecular beacons, SCORPIONS™ probes (Sigma) and TAQMAN™ probes (Life Technologies).

It has been shown that primers and probes in accordance with embodiments herein detect E. histolytica if present, but do not cross-react when any of a number of other pathogens are present in the sample (see, e.g., Examples 4 and 6). As used herein “cross-react” refers to yielding a detectable signal from a template of the indicated organism (e.g. a non-E. hisotolytica organism as listed below). As shown, for example in Example 6, the presence of the organisms listed in Table 4 does not result in a detectable signal for amplification using primers and probes in accordance with some embodiments herein. In some embodiments, cross-reacting can further include depression of the E. histolytica signal when a template from the indicated organism is present. As shown, for example, in Examples 4 and 6, the presence of Cryptosporidium parvum, Giardia lamblia, or Entamoeba dispar (even at high-titer) neither yield a detectable signal, nor substantially suppresses the detectable signal from E. histolytica for primers and probes in accordance with some embodiments herein. In some embodiments, the primers and probes do not cross-react with any of the following organisms: Abiotrophia defectiva, Acinetobacter baumannii, Acinetobacter Iwoffii, Aeromonas hydrophila, Alcaligenes faecalis subsp. faecalis, Anaerococcus tetradius, Arcobacter butzleri, Arcobacter cryaerophilus, Bacillus cereus, Bacteroides caccae, Bacteroides merdae, Bacteroides stercoris, Bifidobacterium adolescentis, Bifidobacterium longum, Camplylobacter coli, Campylobacter concisus, Campylobacter curvus, Campylobacter fetus subsp. fetus, Campylobacter fetus subsp. venerealis, Campylobacter gracilis, Campylobacter hominis, Camplylobacter jejuni, Campylobacter lari, Campylobacter rectus, Campylobacter upsaliensis, Candida albicans, Candida catenulate, Cedecea davisae, Chlamydia trachomatis, Citrobacter amalonaticus, Citrobacter fruendii, Citrobacter koseri, Citrobacter sedlakii, Clostridium difficile 17858, Clostridium difficile 43598, Clostridium difficile CCUG 8864-9689, Clostridium difficile 43255, Clostridium difficile BAA-1805, Clostridium difficile 43593, Clostridium perfringens, Collinsella aerofaciens, Corynebacterium genitalium, Desulfovibrio piger, Edwardsiella tarda, Eggerthella lenta, Enterobacter aerogenes, Enterobacter cloacae, Enterococcus casseliflavus, Enterococcus cecorum, Enterococcus dispar, Enterococus faecalis, Enterococcus gallinarum, Enterococcus hirae, Enterococcus raffinosus, Escherichia coli, Escherichia fergusonii, Escherichia hermannii, Escherichia vulneris, Fusobacterium varium, Gardnerella vaginalis, Gemella morbillorum, Hafnia alvei, Helicobacter fennelliae, Helicobacter pylori, Klebsiella oxytoca, Klebsiella pneumonia, Lactobacillus acidophilus, Lactobacillus reuteri, Lactococcus lactis, Leminorella grimontii, Listeria grayi, Listeria innocua, Listeria monocytogenes, Morganella morganii, Peptoniphilus asaccharolyticus, Peptostreptococcus anaerobius, Plesiomonas shigelloides, Porphyromonas asaccharolytica, Prevotella melaninogenica, Proteus mirabilis, Proteus penneri, Proteus vulgaris, Providencia alcalifaciens, Providencia rettgeri, Providencia stuartii, Pseudomonas aeruginosa, Pseudomonas fluorescens, Ruminococcus bromii, Salmonella typhimurium, Salmonella enteriditis, Serratia liquefaciens, Serratia marcescens, Shigella sonnei, Shigella flexneri, Staphylococcus aureus, Staphylococcus epidermidis, Stenotrophomonas maltophilia, Streptococcus agalactiae, Streptococcus dysgalactiae, Streptococcus intermedius, Streptococcus uberis, Trabulsiella guamensis, Veillonella parvula, Vibrio cholera, Vibrio parahaemolyticus, Yersinia bercovieri, Yersinia enterocolitica, Yersinia rohdei, Adenovirus type 2, Adenovirus type 14, Adenovirus type 40, Adenovirus type 41, Coxsackie A9, Coxsackie B1, HHV-5, Cytomegalovirus, Enterovirus type 69, Human Papillomavirus Type 16, Human Papillomavirus Type 18, Herpes Simplex Virus I, Herpes Simplex Virus II, Norovirus Norovirus II, Rotavirus, Blastocystis hominis, Encephalitozoon intestinalis, Encephalitozoon helium, Encephalitozoon cuniculi, Pentatrichomonas hominis, Entamoeba barrette, Entamoeba gigivalis, Entamoeba invadens, Entamoeba moshkovskii, Entamobea ranarum, Citrobacter fruendii (rpt), Enterobacter cloacae (rpt), Cryptosporidium parvum, Giardia lamblia, or Cryptosporidium meleagridis.

It is noted that while a number of the above-listed organisms are typically found in human stool, several listed organisms are not. As such, it is contemplated herein that probes, primers, and methods of detection in accordance with some embodiments herein are robust in the presence of additional pathogens.

Furthermore, as shown in Examples 5 and 8, primer and probe sets in accordance with embodiments herein showed provided robust results for both fixed and unfixed sample types, and provided results consistent with those of commercial ELISA kits for the detection of E. histolytica. As such, it is contemplated that primer and probe sets in accordance with embodiments herein provide robust results across a variety of sample types (e.g. fixed and unpreserved or non-fixed samples), and consistent with other methods of determining the presence of absence of E. histolytica.

As shown in Example 7, the 95% limit of detection (LoD) for some primers and probes in accordance with embodiments herein is about 17 E. histolytica organisms per milliliter of sample. As used herein, the “95% LoD,” or unless stated otherwise, “LoD,” refers to the concentration that yields a positive result 95% of the time. Accordingly, in some embodiments, the primers and probes will produce a positive signal (e.g. a Ct score below the cutoff) if E. histolytica is present in the amplification reaction in a quantity that is at least the 95% limit of detection (LoD), but will not produce a positive signal if only one or more of the above-listed non-E. histolytica organisms are present. In some embodiments, the LoD of E. hisolytica is about 17 E. histolytica organisms (or quantity of template sequence corresponding to 17 E. histolyica organisms) per milliliter of sample. In some embodiments, the LoD is no more than about 50 organisms per militliter of reaction, for example no more than about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 E. histolytica organisms (or genomes thereof) per mililiter. It is noted that the in some embodiments, the LoD is comparable for both fixed and non-fixed samples (see Example 8 and Tables 6-7). As such, it is understood that primer and probes in accordance with some embodiments herein yield comparable E. histolytica detection properties, for example comparable LoD values, for both fixed and non-fixed samples.

As shown in Example 4, the amount of E. histolytica detected was not substantially altered by a high titer of Cryptosporidium parvum, Giardia lamblia, and Entamoeba dispar, nor was there substantial cross-reactivity with these organisms. Accordingly, in some embodiments, the LoD of E. histolytica organisms is not substantially altered by a high titer presence of another pathogenic organism in the sample. In some embodiments, the detection of E. histolytica organisms (measured, for example by Ct score) is not substantially altered by a high titer presence of another pathogenic organism in the sample. In some embodiments a high titer comprises a quantity of at least 1×10⁶ organisms/mL of sample, for example about 1×10⁶ organisms/mL, 1×10⁶, 2×10⁶, 3×10⁶, 4×10⁶, 5×10⁶, 6×10⁶, 7×10⁶, 8×10⁶, 9×10⁶, 1×10⁷, 1.5×10⁷, 2×10⁷, 3×10⁷, 4×10⁷, 5×10⁷, 6×10⁷, 7×10⁷, 8×10⁷, 9×10⁷, 1×10⁸, 1×10⁹, or 1×10¹⁰ organisms/mL of sample. In some embodiments a high titer comprises a quantity of at least about 1.5×10⁷ organisms/mL of sample.

### Kits

Some embodiments include kits. The kits can include at least one primer pair as described herein. In some embodiments, the primer pair can amplify an E. histolytica target sequence under standard amplification conditions, but cannot amplify an E. dispar target sequence under standard amplification conditions, as described herein. The kits can include a probe as described herein. In some embodiments, the probe is specific to a nucleic acid sequence that occurs in both E. histolytica and E. dispar as described herein. In some embodiments, the primer set includes a forward primer comprising an oligonucleotide having the sequence of SEQ ID NO: 1, or a variant thereof, a reverse primer comprising an oligonucleotide having the sequence of SEQ ID NO: 2, or a variant thereof, and a probe comprising an oligonucleotide having the sequence of SEQ ID NO: 3, or a variant thereof. In some embodiments, the probe comprises a fluorophore/quencher pair as described herein. In some embodiments, the kits include samples, for example positive controls that contain E. histolytica or E. histolytica DNA as described herein. The kits can further include negative controls, for example that contain only E. dispar, or E. dispar DNA. The kits can further include packaging and/or instructions.

In some embodiments, the kits further include reagents for a multiplex assay for detecting at least one other parasitic organism from a human stool sample, for example at least one of Giardia lamblia, Cryptosporidium parvum, Cryptosporidium hominis, and the like.

### Master Mix

In some embodiments, a master mix is provided. A master mix can include at least two reagents for an assay that are provided in relative concentrations that are proportional to the relative concentrations of the reagents in a quantitative nucleic acid amplification assay Thus, a single a single quantity of master mix can be added to a reaction to provide appropriate relative concentrations of two or more reagents. In some embodiments, a master mix can include at least two of: polymerase, buffer, salts, for example magnesium, nucleotide triphosphates, a primer set, and water. In some embodiments, a master mix can be provided at a higher concentration than will be used in a reaction. In some embodiments, a master mix is provided in a lyophilized form, and reconstituted at a higher concentration that will be used in the reaction. In some embodiments a master mix includes reagents at a concentration of at least about 2× of the reaction concentration, for example 2×, 2.5×, 3×, 4×, 5×, 6×, 7×, 8×, 9×, 10×, 15×, 20×, 25×, 40×, 50×, 100×, 200×, 250×, or 500×.

### Samples

Samples as provided herein include substances that may or may not contain Entamoeba nucleic acids. In some embodiments, the sample includes fecal matter from a human, or a portion or derivative thereof. In some embodiments, the sample includes a biopsy, for example tissue from a human that is possibly infected with Entamoeba, such as gastrointestinal, liver, lung, or central nervous system tissue. In some embodiments, the sample includes a cell culture, for example a culture derived from human fecal matter. In some embodiments, the sample has been processed, for example to isolate nucleic acids from other substances, or to remove non-nucleic acid substances from the sample (for example to remove lipids, proteins, cellular debris, and the like). In some embodiments, the sample has been treated with protease. It has been shown that primers and probes in accordance with embodiments herein achieve comparable detection properties for fixed and unpreserved samples (see, e.g., Example 8 and Tables 6-7). In some embodiments, the sample is fixed, for example in a quantity of fixative such as formalin. In some embodiments the sample is unpreserved (e.g. “non-fixed”).

In some embodiments, it is unknown whether the sample contains E. histolytica and/or E dispar nucleic acids. In some embodiments, it is known that the sample includes at least one of E. histolytica or E. dispar, but it is unknown which one sample includes, or whether the sample includes both. In some embodiments, the sample contains both E. hisotlytica and E. dispar.

In some embodiments, the sample includes a positive control, for example spiking the sample with nucleic acids of E. histolytica, E. dispar, or a combination of nucleic acids from E. histolytica, or E. dispar. In some embodiments, the sample is spiked with at least 1000 (“1K”) copies of E. dispar target amplification sequence, for example at least about 1K copies, 2K, 3K, 4K, 5K, 6K, 7K, 8K, 9K, 10K, 20K, 30K, 40K, 50K, 60K, 70K, 80K, 90K, 100K, 150K, 200K, 250K, 300K, 350K, 400K, 450K, 500K, 550K, 600K, 650K, 700K, 750K, 800K, 850K, 900K, 1000K, 1100K, 1200K, 1300K, 1400K, 1500K, 1600K, 1700K 1800K, 1900K, or 2000K copies. In some embodiments, the sample is spiked with at least 100 copies of E. histolytica target amplification sequence, for example at least about 100, 200, 300, 400, 500, 600, 700, 800, 900, 1K copies, 2K, 3K, 4K, 5K, 6K, 7K, 8K, 9K, 10K, 20K, 30K, 40K, 50K, 60K, 70K, 80K, 90K, 100K, 150K, 200K, 250K, 300K, 350K, 400K, 450K, 500K, 550K, 600K, 650K, 700K, 750K, 800K, 850K, 900K, 1000K, 1100K, 1200K, 1300K, 1400K, 1500K, 1600K, 1700K 1800K, 1900K, or 2000K copies. In some embodiments, the sample is spiked with E. histolytica and E. dispar target nucleic acids.

In some embodiments, the sample includes nucleic acids isolated from one or more of the above. Nucleic acids can be isolated using standard techniques, well-known to one skilled in the art.

### Additional Embodiments

In accordance with some embodiments, primer and probe sets, and methods of using the same are provided for the detection of E. histolyica. In some embodiments, the primers and probe sets and methods do not detect non-pathogenic E. dispar. In some embodiments, the primers and probe sets and methods produce robust results, that are not inhibited or interfered with in the case of a simulated mixed E. histolytica and E. dispar infection. In some embodiments, the primers and probe sets and methods detect Entamoeba histolytica from human clinical specimens identified by traditional microscopic methods (which at the time of the application represent the current standard of care). In some embodiments, the primers and probe sets and methods produce results that agree with a commercially available FDA-cleared ELISA assay for the appropriate specimen type using clinical specimens. In some embodiments, the primers and probe sets and methods do not cross-react with other organisms likely to be found in stool or a variety of other pathogens. In some embodiments, the primers and probe sets and methods do react with different Entamoeba histolytica isolates. In some embodiments, the primers and probe sets and methods are sensitive to detect down to, and below, 17 organisms per mL in the sample buffer tube (or a quantity of template sequence corresponding to 17 organisms).

### Example 1

### Amplification in the Presence of E. histolytica and E. dispar Plasmid Sequences

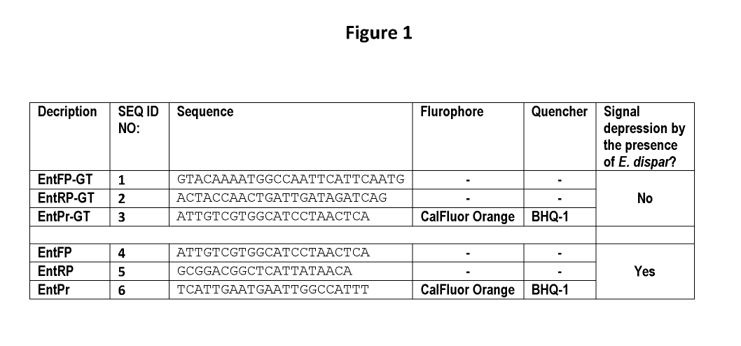

A previously-described primer set and probe combination (see Verweij et al., J. Clin. Microbiol. 42: 1220-23, 2004), which included a forward primer of SEQ ID NO: 4 (ATTGTCGTGGCATCCTAACTCA), a reverse primer of SEQ ID NO: 5 (GCGGACGGCTCATTATAACA), and a probe of SEQ ID NO: 6 (TCATTGAATGAATTGGCCATTT), which comprised a CalFluor Orange fluorphore and BHQ-1 quencher (see FIG. 1) were used in a quantitative PCR reaction on a BD MAX™ system. It is noted that the primer set of SEQ ID NO: 4 and SEQ ID NO: 5 amplify rDNA sequences of both E. histolytica and E. dispar. The probe of SEQ ID NO: 6 has 100% percent homology to the target amplification sequence (defined by the primer set of SEQ ID NOs: 4 and 5) in E. histolytica, but not E. dispar.

Reactions were provided with template plasmid that contained target rDNA gene sequence from E. histolytica, and/or E. dispar. Low-level cross-reactivity was observed between E. histolytica and E. dispar target DNA sequence. Furthermore, when plasmid containing E. dispar target nucleic acid was spiked into the PCR reaction at a higher proportion than plasmid containing E. histolytica nucleic acid, the specific signal from E. histolytica was drastically reduced (see FIG. 3). While quantitative PCR reactions with E. histolytica template produced detectable signal, the presence of 5,000 (“5K”), 25,000 (“25K”), 50,000 (“50K”), 75,000 (75K), and 100,000 (“100K”) copies of E. dispar template plasmid, in addition to a constant level of E. histolytica template, decreased the amount of detectable signal in a dose-dependent manner (FIG. 3). As summarized in FIG. 1, the primer/probe combination of this example resulted in E. histolytica signal depression.

Without being limited by any one theory, it is contemplated that the use of primers that amplify both E. histolytica and E. dispar DNA, and reliance on an E. histolytica-sequence-specific probe resulted in both cross-reactivity, and signal suppression in the presence of E. dispar, possibly due to homo- and hetero-duplex formation between amplification products of E. histolytica and E. dispar that blocks the availability of E. histolytica probe binding sites.

### Example 2

### Detection of E. histolytica in the Presence of E. histolytica and E. dispar Plasmid Sequences

A primer-probe set according to embodiments herein was used in a quantitative PCR amplification reaction performed on the BD MAX™ platform. The PCR mixture was heated to 97° C. for 10 minutes to activate the DNA Polymerase. Two-step thermal cycling was then carried out for 45 cycles with a 15 second denaturation step at 97° C. followed by an annealing/extension step for 64.5 seconds at 62° C. The primer set included a forward primer of SEQ ID NO: 1, a reverse primer of SEQ ID NO: 2, and a probe of SEQ ID NO: 3, which comprised a CalFluor Orange fluorphore and BHQ-1 quencher (see FIG. 1). It is noted that the primer of SEQ ID NO: 1 will anneal to E. histolytica, but not E. dispar target nucleic acids sequence of the small ribosomal subunit gene under standard amplification conditions (see FIG. 2), while the primer of SEQ ID NO: 2 will anneal to target nucleic acids sequence on either of the E. histolytica and E. dispar small ribosomal subunit gene. Accordingly, the primer set of SEQ ID NO: 1 and SEQ ID NO: 2 will substantially amplify E. histolytica, but not E. dispar target amplification sequence. The probe of SEQ ID NO: 3 has 100% complementarity to either of E. histolytica or E. dispar small ribosomal subunit gene DNA sequence.

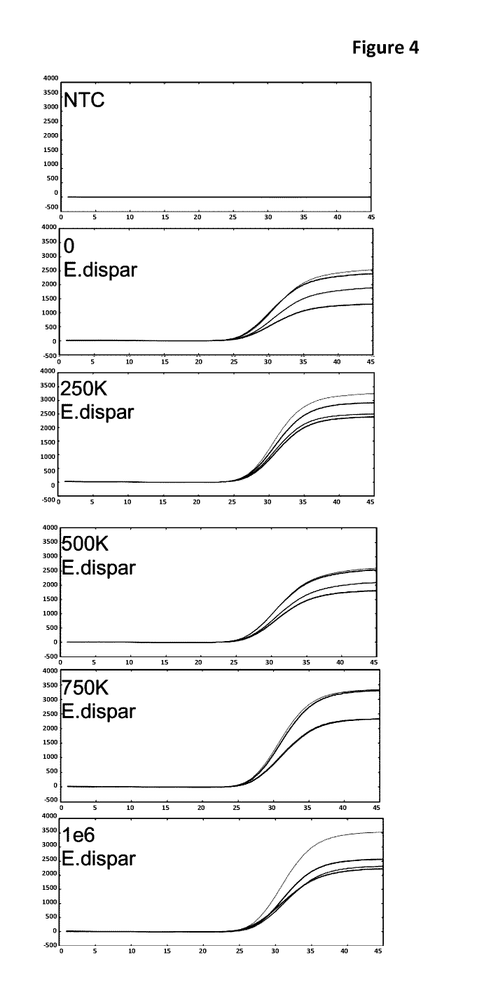

As in Example 1, reactions were provided with plasmid that contained target rDNA gene sequence template from E. histolytica, and/or E. dispar. Unlike Example 1, cross-reactivity was not seen with E. dispar template. Moreover, the presence of E. dispar template did not depress the amplification signal (see FIG. 4). In the presence of a constant amount of E. histolytica template, the presence of 0, 250,000 (“250K”), 500,000 (“500K”), 750,000 (“750K”), and 1,000,000 (“1e6”) copies of E. dispar template-containing plasmid did not decrease the amplification signal from E. histolytica (FIG. 4). A negative control that contained no template (“NTC”) was performed, and as expected, no signal was detected. As summarized in FIG. 1, the primer/probe combination of this example did not cause any identifiable E. histolytica signal depression.

Thus, even in the presence of a high copy number of E. dispar template, the primer set and probe as in Example 2 produced robust, and consistent levels of E. histolytica signal. Without being bound to any one theory, it is contemplated that a primer set designed to amplify a sequence specific to E. histolytica, but not E. dispar can permit the detection of E. histolytica-specific signal without interference from E. dispar sequences.

### Examples 3-13

### Detection of E. histolytica Sequences

The following methods were used in Examples 3-13.

Stool specimens were collected from patients and transported to the laboratory unpreserved in a clean container (unpreserved) or fixed (10% formalin).

DNA extraction from the stool specimens was performed as follows: Specimens were vortexed. A 10 μL loop was inserted in each specimen to the depth of the loop and then expressed using a swirling motion into BD MAX™ Sample Buffer Tubes (SBT) containing Sample Buffer [50 mM Tris-HCl (pH 7.0), 1% Triton X-100, 1 mM EDTA (pH 8.0), 20 mM H₃BO₃, 20 mM Na₃C₆H₅O₇. 2H₂O]. The SBTs were closed with a septum cap and then heated on the BD Prewarm Heater to approximately 110° C. for 20 minutes to facilitate lysis of organisms. The SBTs were cooled to room temperature by the BD Prewarm Heater, vortexed briefly, and then transferred to the BD MAX™ System. A 500 μl volume of sample buffer was extracted per sample for 10 minutes at 75° C. using 12 units of proteinase K, 0.12% trehalose, and 104 copies of an internal control DNA in the presence of 0.5 μg/μl PAMAM-coupled magnetic beads on the BD MAX™ System. The beads, with the bound nucleic acids, were washed with 500 μl of wash buffer [12.5 mM Tris (pH 6.8), 0.03% ProClin 300, 0.1% Tween-20]. Nucleic acids were then eluted by heating the beads for 3 minutes at 80° C. in 12.5 μl of elution buffer [20 mM NaOH]. Eluted nucleic acids were neutralized by the addition of 22.5 μl of neutralization buffer [7.78 mM MgCl2, 155.6 mM Tris (pH 8.0), 4.44 mM NaOH, 0.03% ProClin300, 0.016% Tween-20].

A PCR master mix was prepared as follows: Neutralized nucleic acids (35 μl) were used to rehydrate dried down master mix. The final concentration of components in the PCR master mix after rehydration with is as follows: 5 mM MgCl₂, 100 mM Tris, 10 mM NaOH, 0.019% ProClin300, 0.010% Tween-20, 1.96% Trehalose, 0.5 mM dNTPs (each), 0.6 mg/ml BSA, 0.04 U/μl Hot Gold Star DNA Polymerase. The master mix also included PCR primers and TaqMan® dual-labeled hydrolysis probes. Primers and probes for Entamoeba histolytica were included at 900 nM for forward and reverse primers and 550 nM for the probe. The primer set and probe set for the detection of E. histolytica included a forward primer having the nucleic acid sequence of SEQ ID NO: 1, a reverse primer having a nucleic acid sequence of SEQ ID NO: 2, and a probe having the nucleic acid sequence of SEQ ID NO: 3. The probe for Entamoeba histolytica was labeled with Cal Fluor Orange 560 and Black Hole Quencher-1. Primers and probes for the internal control were included at 300 nM each. The internal control probe was labeled with Quasar 705 and Black Hole Quencher-3. Primers and probes for Cryptosporidium parvum/hominis and Giardia lamblia were included at 200 nM for forward and reverse primers and 550 nM for probes. The probe for Cryptosporidium parvum/hominis was labeled with CalFluor Red 610 and Black Hole Quencher-2. The probe for Giardia lamblia was labeled with FAM and Black Hole Quencher-1.

After rehydration, the BD MAX™ System dispenses approximately 12 μl of PCR-ready solution into the BD MAX™ Microfluidic Cartridge. Microvalves in the BD MAX™ Microfluidic Cartridge are sealed by the system prior to initiating PCR to contain the amplification mixture thus preventing evaporation and contamination. The PCR mixture was heated to 97° C. for 10 minutes to activate the DNA Polymerase. Two-step thermal cycling was then carried out for 45 cycles with a 15 second denaturation step at 97° C. followed by an annealing/extension step for 64.5 seconds at 62° C. The BD MAX™ System monitors fluorescent signals at each cycle and interprets the data at the end of the program to report the final results. Result calls were based on a Ct.Score algorithm that includes an initial static endpoint threshold for each target channel and a secondary dynamic QC threshold that changes inversely with Ct. Endpoint fluorescence must exceed both thresholds and a final Ct must be <42 to be considered positive. Additional checks for excessively variable PCR curves were used to exclude reactions that had insufficient volume in the PCR chamber. Amplification failure of the internal control causes the system to return unresolved results for each target channel that fails to meet the Ct.Score thresholds for positivity.

### Example 3

Two lots of Entamoeba histolytica trophozoites were detected by the BD MAX™ assay as described. The BD MAX™ assay does not detect low (2,550 trophozoites per ml in specimen) or high (1.5e6 trophozoites per ml in specimen) titer Entamoeba dispar. The results are shown in Table 1.

TABLE 1

| | Trophs/ml | | Ct. | Assay |
|---|---|---|---|---|
| Input (trophozoites) | in Specimen | ymaxEP | Score | Result |
| E. histolytica lot 2 | 2550 | 6692.03 | 25 | Positive |
| E. dispar High | 1.50E+06 | 1.77 | NA | Negative |
| E. histolytica lot 1 | 2550 | 7866.65 | 23.58 | Positive |
| E. dispar Low | 2550 | 10.36 | NA | Negative |
| E. histolytica lot 2 | 2550 | 7228.88 | 25.54 | Positive |
| E. dispar High | 1.50E+06 | 1.49 | NA | Negative |
| E. histolytica lot 1 | 2550 | 6942.94 | 23.44 | Positive |
| E. dispar Low | 2550 | 2.08 | NA | Negative |
| E. histolytica lot 2 | 2550 | 7173.94 | 25.64 | Positive |
| E. dispar High | 1.50E+06 | 1.37 | NA | Negative |
| E. histolytica lot 1 | 2550 | 7107.91 | 24.01 | Positive |
| E. dispar Low | 2550 | 1.67 | NA | Negative |
| E. histolytica lot 2 | 2550 | 5881.98 | 25.68 | Positive |
| E. dispar High | 1.50E+06 | 0.71 | NA | Negative |
| E. histolytica lot 1 | 2550 | 6185.44 | 23.47 | Positive |
| E. dispar Low | 2550 | 0.5 | NA | Negative |
| E. histolytica lot 2 | 2550 | 6732.16 | 26.33 | Positive |
| E. dispar High | 1.50E+06 | 0.66 | NA | Negative |
| E. histolytica lot 1 | 2550 | 7168.81 | 23.75 | Positive |
| E. dispar Low | 2550 | 0.72 | NA | Negative |
| E. histolytica lot 2 | 2550 | 6774.47 | 26.05 | Positive |
| E. dispar High | 1.50E+06 | 1.16 | NA | Negative |
| E. histolytica lot 1 | 2550 | 8523.08 | 24.45 | Positive |
| E. dispar Low | 2550 | 0.81 | NA | Negative |

### Example 4

The BD MAX™ detected E. histolytica near the limit of detection (LoD) in simulated multiple infection specimens containing high titer Cryptosporidium parvum, Giardia lamblia, and Entamoeba dispar.

TABLE 2

| | Trophs/ml | | Ct. | Assay |
|---|---|---|---|---|
| Input (trophozoites) | in Specimen | ymaxEP | Score | Result |
| E. histolytica | 450 | 7902.56 | 24.05 | Positive |
| E. histolytica | 450 | 7855.85 | 24.72 | Positive |
| E. histolytica | 450 | 7864.77 | 23.49 | Positive |
| E. histolytica | 450 | 7381.68 | 24.16 | Positive |
| E. histolytica | 450 | 7257.07 | 25.46 | Positive |
| E. histolytica | 450 | 7823.11 | 24.74 | Positive |
| E. histolytica | 450 | 4966.57 | 24.81 | Positive |
| E. histolytica | 450 | 2444.99 | 24.33 | Positive |
| E. histolytica | 450 | 5637.2 | 24.97 | Positive |
| E. histolytica | 450 | 4913.83 | 24.23 | Positive |
| E. histolytica | 450 | 2584.9 | 25.31 | Positive |
| E. histolytica | 450 | 6631.45 | 25.33 | Positive |
| E. histolytica + | 450 (E. | 3402.82 | 25.78 | Positive |
| C. parvum, G. lamblia, | histolytica) | | | |
| E. dispar | 1.5e7 (Cp, Gl, Ed) | | | |
| E. histolytica + | 450 (E. | 2253.37 | 25.99 | Positive |
| C. parvum, G. lamblia, | histolytica) | | | |
| E. dispar | 1.5e7 (Cp, Gl, Ed) | | | |
| E. histolytica + | 450 (E. | 4681.89 | 23.49 | Positive |
| C. parvum, G. lamblia, | histolytica) | | | |
| E. dispar | 1.5e7 (Cp, Gl, Ed) | | | |
| E. histolytica + | 450 (E. | 4351.27 | 24.95 | Positive |
| C. parvum, G. lamblia, | histolytica) | | | |
| E. dispar | 1.5e7 (Cp, Gl, Ed) | | | |
| E. histolytica + | 450 (E. | 8041.45 | 24.41 | Positive |
| C. parvum, G. lamblia, | histolytica) | | | |
| E. dispar | 1.5e7 (Cp, Gl, Ed) | | | |
| E. histolytica + | 450 (E. | 6941.23 | 25.11 | Positive |
| C. parvum, G. lamblia, | histolytica) | | | |
| E. dispar | 1.5e7 (Cp, Gl, Ed) | | | |

### Example 5

The BD MAX™ system was used to detect the form of the Entamoeba histolytica organism shed in true human clinical specimens detected by traditional methods representing both unpreserved and 10% formalin fixed specimen types. For comparison, a commercially-available ELISA (TechLab E. histolytica II) was performed on the same samples.

The results of the BD MAX™ assay closely agree with a commercially available ELISA result (TechLab E. histolytica II) in unpreserved specimens for which the ELISA is cleared. It is noted that TechLab ELISA assay is not cleared for fixed specimens and therefore, the negative result for the fixed specimens is in-line with the properties of the TechLab ELISA assay.

TABLE 3

| Clinical | | | | | |
|---|---|---|---|---|---|
| Specimen | | | Ct. | BD MAX | TechLab |
| ID | Type | ymaxEP | Score | Result | EIA Result |
| EH16 | Unpreserved | 3584.6 | 29.49 | Positive | Positive |
| EH17 | Unpreserved | 3522.11 | 34.21 | Positive | Positive |
| EH18 | Unpreserved | 3868 | 31.51 | Positive | Positive |
| EH19 | Unpreserved | 1912.34 | 32.3 | Positive | Positive |
| EH20 | Unpreserved | 3893.49 | 31.95 | Positive | Positive |
| EH21 | Unpreserved | 245.52 | NA | UNR | Positive |
| EH22 | Unpreserved | 3053.63 | 33.71 | Positive | Positive |
| EH23 | Unpreserved | 3835.47 | 27.65 | Positive | Positive |
| EH24 | Unpreserved | 4079.21 | 31.79 | Positive | Positive |
| EH25 | Unpreserved | 3965.72 | 30.79 | Positive | Positive |
| +CTRL | N/A | 4279.29 | 26.11 | Positive | Positive |
| (spike) | | | | | |
| NEG CTRL | N/A | 1.25 | NA | Negative | Negative |
| EH01 | 10% Formalin | 4850 | 27.19 | Positive | Negative |
| EH02 | 10% Formalin | 5462.54 | 23.44 | Positive | Negative |
| EH03 | 10% Formalin | 4773.22 | 23.69 | Positive | Negative |
| EH04 | 10% Formalin | 4998.17 | 25.81 | Positive | Negative |
| EH05 | 10% Formalin | 5338.4 | 23.96 | Positive | Negative |
| EH06 | 10% Formalin | 5233.45 | 25.48 | Positive | Negative |
| EH07 | 10% Formalin | 5199.61 | 28.69 | Positive | Negative |
| EH08 | 10% Formalin | 5595.57 | 24.32 | Positive | Negative |
| EH09 | 10% Formalin | 5000.83 | 28.98 | Positive | Negative |
| EH10 | 10% Formalin | 2839.61 | 29.24 | Positive | Negative |
| EH11 | 10% Formalin | 5951.38 | 22.89 | Positive | Negative |
| EH12 | 10% Formalin | 4592.81 | 28.92 | Positive | Negative |
| EH13 | 10% Formalin | 5071.64 | 29.26 | Positive | Negative |
| EH14 | 10% Formalin | 5038.97 | 24.5 | Positive | Negative |
| EH15 | 10% Formalin | 4545.11 | 29.06 | Positive | Negative |

### Example 6

To determine whether the BD MAX™ assay cross-reacts with other organisms E. histolytica sequences were detected in the presence of template from other organisms, including organisms likely to be found in stool, and well as exemplary organisms that were not likely to be found in stool. Challenge organisms were spiked into an SBT without stool matrix. Each organism was tested in triplicate.

The results are shown in Table 4. The BD MAX™ assay does not cross-react with other organisms likely (or unlikely) to be found in stool.

TABLE 4

| | | Enty | Ent | | | | BD |
|---|---|---|---|---|---|---|---|
| | Sample | max | Ct. | Stock | Titer in | Spike | MAX |
| Organism | ID | EP | Score | Titer | SBT | Vol. | Result |
| Abiotrophia | 1 | 2.79 | NA | ≧1 × 10⁸ | ≧1 × 10⁶ | 15 μL | Neg. |
| defectiva | | | | CFU | CFU | | |
| Abiotrophia | 1 | 3.07 | NA | ≧1 × 10⁸ | ≧1 × 10⁶ | 15 μL | Neg. |
| defectiva | | | | CFU | CFU | | |
| Abiotrophia | 1 | 2.7 | NA | ≧1 × 10⁸ | ≧1 × 10⁶ | 15 μL | Neg. |
| defectiva | | | | CFU | CFU | | |
| Acinetobacter | 2 | 16.97 | NA | ≧1 × 10⁸ | ≧1 × 10⁶ | 15 μL | Neg. |
| baumannii | | | | CFU | CFU | | |
| Acinetobacter | 2 | 7.38 | NA | ≧1 × 10⁸ | ≧1 × 10⁶ | 15 μL | Neg. |
| baumannii | | | | CFU | CFU | | |
| Acinetobacter | 2 | 6.18 | NA | ≧1 × 10⁸ | ≧1 × 10⁶ | 15 μL | Neg. |
| baumannii | | | | CFU | CFU | | |
| Acinetobacter | 3 | 3.21 | NA | ≧1 × 10⁸ | ≧1 × 10⁶ | 15 μL | Neg. |
| Iwoffii | | | | CFU | CFU | | |
| Acinetobacter | 3 | 2.28 | NA | ≧1 × 10⁸ | ≧1 × 10⁶ | 15 μL | Neg. |
| Iwoffii | | | | CFU | CFU | | |
| Acinetobacter | 3 | 3.28 | NA | ≧1 × 10⁸ | ≧1 × 10⁶ | 15 μL | Neg. |
| Iwoffii | | | | CFU | CFU | | |
| Aeromonas | 4 | 3.79 | NA | ≧1 × 10⁸ | ≧1 × 10⁶ | 15 μL | Neg. |
| hydrophila | | | | CFU | CFU | | |
| Aeromonas | 4 | 3.47 | NA | ≧1 × 10⁸ | ≧1 × 10⁶ | 15 μL | Neg. |
| hydrophila | | | | CFU | CFU | | |
| Aeromonas | 4 | 3.35 | NA | ≧1 × 10⁸ | ≧1 × 10⁶ | 15 μL | Neg. |
| hydrophila | | | | CFU | CFU | | |
| Alcaligenes | 5 | 1.59 | NA | ≧1 × 10⁸ | ≧1 × 10⁶ | 15 μL | Neg. |
| faecalis subsp. | | | | CFU | CFU | | |
| faecalis | | | | | | | |
| Alcaligenes | 5 | 5.64 | NA | ≧1 × 10⁸ | ≧1 × 10⁶ | 15 μL | Neg. |
| faecalis subsp. | | | | CFU | CFU | | |
| faecalis | | | | | | | |
| Alcaligenes | 5 | 1.3 | NA | ≧1 × 10⁸ | ≧1 × 10⁶ | 15 μL | Neg. |
| faecalis subsp. | | | | CFU | CFU | | |
| faecalis | | | | | | | |
| Anaerococcus | 6 | 1 | NA | ≧1 × 10⁸ | ≧1 × 10⁶ | 15 μL | Neg. |
| tetradius | | | | CFU | CFU | | |
| Anaerococcus | 6 | 5.98 | NA | ≧1 × 10⁸ | ≧1 × 10⁶ | 15 μL | Neg. |
| tetradius | | | | CFU | CFU | | |
| Anaerococcus | 6 | 2.79 | NA | ≧1 × 10⁸ | ≧1 × 10⁶ | 15 μL | Neg. |
| tetradius | | | | CFU | CFU | | |
| Arcobacter | 7 | 4.02 | NA | ≧1 × 10⁸ | ≧1 × 10⁶ | 15 μL | Neg. |
| butzleri | | | | CFU | CFU | | |
| Arcobacter | 7 | 3.68 | NA | ≧1 × 10⁸ | ≧1 × 10⁶ | 15 μL | Neg. |
| butzleri | | | | CFU | CFU | | |
| Arcobacter | 7 | 4.43 | NA | ≧1 × 10⁸ | ≧1 × 10⁶ | 15 μL | Neg. |
| butzleri | | | | CFU | CFU | | |
| Arcobacter | 8 | 2.96 | NA | ≧1 × 10⁸ | ≧1 × 10⁶ | 15 μL | Neg. |
| cryaerophilus | | | | CFU | CFU | | |
| Arcobacter | 8 | 2.49 | NA | ≧1 × 10⁸ | ≧1 × 10⁶ | 15 μL | Neg. |
| cryaerophilus | | | | CFU | CFU | | |
| Arcobacter | 8 | 3.87 | NA | ≧1 × 10⁸ | ≧1 × 10⁶ | 15 μL | Neg. |
| cryaerophilus | | | | CFU | CFU | | |
| Bacillus cereus | 9 | 0.76 | NA | ≧1 × 10⁸ | ≧1 × 10⁶ | 15 μL | Neg. |
| | | | | CFU | CFU | | |
| Bacillus cereus | 9 | 0.66 | NA | ≧1 × 10⁸ | ≧1 × 10⁶ | 15 μL | Neg. |
| | | | | CFU | CFU | | |
| Bacillus cereus | 9 | 0.62 | NA | ≧1 × 10⁸ | ≧1 × 10⁶ | 15 μL | Neg. |
| | | | | CFU | CFU | | |
| Bacteroides caccae | 10 | 0.69 | NA | ≧1 × 10⁸ | ≧1 × 10⁶ | 15 μL | Neg. |
| | | | | CFU | CFU | | |
| Bacteroides caccae | 10 | 1.71 | NA | ≧1 × 10⁸ | ≧1 × 10⁶ | 15 μL | Neg. |
| | | | | CFU | CFU | | |
| Bacteroides caccae | 10 | 1.66 | NA | ≧1 × 10⁸ | ≧1 × 10⁶ | 15 μL | Neg. |
| | | | | CFU | CFU | | |
| Bacteroides | 11 | 0.87 | NA | ≧1 × 10⁸ | ≧1 × 10⁶ | 15 μL | Neg. |
| merdae | | | | CFU | CFU | | |
| Bacteroides | 11 | 0.6 | NA | ≧1 × 10⁸ | ≧1 × 10⁶ | 15 μL | Neg. |
| merdae | | | | CFU | CFU | | |
| Bacteroides | 11 | 0.8 | NA | ≧1 × 10⁸ | ≧1 × 10⁶ | 15 μL | Neg. |
| merdae | | | | CFU | CFU | | |
| Bacteroides | 12 | 2.9 | NA | ≧1 × 10⁸ | ≧1 × 10⁶ | 15 μL | Neg. |
| stercoris | | | | CFU | CFU | | |
| Bacteroides | 12 | 1.1 | NA | ≧1 × 10⁸ | ≧1 × 10⁶ | 15 μL | Neg. |
| stercoris | | | | CFU | CFU | | |
| Bacteroides | 12 | 1.14 | NA | ≧1 × 10⁸ | ≧1 × 10⁶ | 15 μL | Neg. |
| stercoris | | | | CFU | CFU | | |
| Bifidobacterium | 13 | 0.14 | NA | ≧1 × 10⁸ | ≧1 × 10⁶ | 15 μL | Neg. |
| adolescentis | | | | CFU | CFU | | |
| Bifidobacterium | 13 | 4.27 | NA | ≧1 × 10⁸ | ≧1 × 10⁶ | 15 μL | Neg. |
| adolescentis | | | | CFU | CFU | | |
| Bifidobacterium | 13 | 0.68 | NA | ≧1 × 10⁸ | ≧1 × 10⁶ | 15 μL | Neg. |
| adolescentis | | | | CFU | CFU | | |
| Bifidobacterium | 14 | 0.88 | NA | ≧1 × 10⁸ | ≧1 × 10⁶ | 15 μL | Neg. |
| longum | | | | CFU | CFU | | |
| Bifidobacterium | 14 | 3.16 | NA | ≧1 × 10⁸ | ≧1 × 10⁶ | 15 μL | Neg. |
| longum | | | | CFU | CFU | | |
| Bifidobacterium | 14 | 2.24 | NA | ≧1 × 10⁸ | ≧1 × 10⁶ | 15 μL | Neg. |
| longum | | | | CFU | CFU | | |
| Camplylobacter | 15 | 0.59 | NA | ≧1 × 10⁸ | ≧1 × 10⁶ | 15 μL | Neg. |
| coli | | | | CFU | CFU | | |
| Camplylobacter | 15 | 1.43 | NA | ≧1 × 10⁸ | ≧1 × 10⁶ | 15 μL | Neg. |
| coli | | | | CFU | CFU | | |
| Camplylobacter | 15 | 1.55 | NA | ≧1 × 10⁸ | ≧1 × 10⁶ | 15 μL | Neg. |
| coli | | | | CFU | CFU | | |
| Campylobacter | 16 | 0.57 | NA | ≧1 × 10⁸ | ≧1 × 10⁶ | 15 μL | Neg. |
| concisus | | | | CFU | CFU | | |
| Campylobacter | 16 | 0.26 | NA | ≧1 × 10⁸ | ≧1 × 10⁶ | 15 μL | Neg. |
| concisus | | | | CFU | CFU | | |
| Campylobacter | 16 | 0.83 | NA | ≧1 × 10⁸ | ≧1 × 10⁶ | 15 μL | Neg. |
| concisus | | | | CFU | CFU | | |
| Campylobacter | 17 | 0.33 | NA | ≧1 × 10⁸ | ≧1 × 10⁶ | 15 μL | Neg. |
| curvus | | | | CFU | CFU | | |
| Campylobacter | 17 | 2.34 | NA | ≧1 × 10⁸ | ≧1 × 10⁶ | 15 μL | Neg. |
| curvus | | | | CFU | CFU | | |
| Campylobacter | 17 | 1.46 | NA | ≧1 × 10⁸ | ≧1 × 10⁶ | 15 μL | Neg. |
| curvus | | | | CFU | CFU | | |
| Campylobacter | 18 | 0.65 | NA | ≧1 × 10⁸ | ≧1 × 10⁶ | 15 μL | Neg. |
| fetus subsp. fetus | | | | CFU | CFU | | |
| Campylobacter | 18 | 0.57 | NA | ≧1 × 10⁸ | ≧1 × 10⁶ | 15 μL | Neg. |
| fetus subsp. fetus | | | | CFU | CFU | | |
| Campylobacter | 18 | 3.49 | NA | ≧1 × 10⁸ | ≧1 × 10⁶ | 15 μL | Neg. |
| fetus subsp. fetus | | | | CFU | CFU | | |
| Campylobacter | 19 | 0.66 | NA | ≧1 × 10⁸ | ≧1 × 10⁶ | 15 μL | Neg. |
| fetus subsp. | | | | CFU | CFU | | |
| venerealis | | | | | | | |
| Campylobacter | 19 | 0.09 | NA | ≧1 × 10⁸ | ≧1 × 10⁶ | 15 μL | Neg. |
| fetus subsp. | | | | CFU | CFU | | |
| venerealis | | | | | | | |
| Campylobacter | 19 | 0.51 | NA | ≧1 × 10⁸ | ≧1 × 10⁶ | 15 μL | Neg. |
| fetus subsp. | | | | CFU | CFU | | |
| venerealis | | | | | | | |
| Campylobacter | 20 | 0.85 | NA | ≧1 × 10⁸ | ≧1 × 10⁶ | 15 μL | Neg. |
| gracilis | | | | CFU | CFU | | |
| Campylobacter | 20 | 0.31 | NA | ≧1 × 10⁸ | ≧1 × 10⁶ | 15 μL | Neg. |
| gracilis | | | | CFU | CFU | | |
| Campylobacter | 20 | 0.61 | NA | ≧1 × 10⁸ | ≧1 × 10⁶ | 15 μL | Neg. |
| gracilis | | | | CFU | CFU | | |
| Campylobacter | 21 | 0.64 | NA | ≧1 × 10⁸ | ≧1 × 10⁶ | 15 μL | Neg. |
| hominis | | | | CFU | CFU | | |
| Campylobacter | 21 | 0.81 | NA | ≧1 × 10⁸ | ≧1 × 10⁶ | 15 μL | Neg. |
| hominis | | | | CFU | CFU | | |
| Campylobacter | 21 | 0.96 | NA | ≧1 × 10⁸ | ≧1 × 10⁶ | 15 μL | Neg. |
| hominis | | | | CFU | CFU | | |
| Camplylobacter | 22 | 0.08 | NA | ≧1 × 10⁸ | ≧1 × 10⁶ | 15 μL | Neg. |
| jejuni | | | | CFU | CFU | | |
| Camplylobacter | 22 | 0.27 | NA | ≧1 × 10⁸ | ≧1 × 10⁶ | 15 μL | Neg. |
| jejuni | | | | CFU | CFU | | |
| Camplylobacter | 22 | 0.45 | NA | ≧1 × 10⁸ | ≧1 × 10⁶ | 15 μL | Neg. |
| jejuni | | | | CFU | CFU | | |
| Campylobacter lari | 23 | 1.23 | NA | ≧1 × 10⁸ | ≧1 × 10⁶ | 15 μL | Neg. |
| | | | | CFU | CFU | | |
| Campylobacter lari | 23 | 0.17 | NA | ≧1 × 10⁸ | ≧1 × 10⁶ | 15 μL | Neg. |
| | | | | CFU | CFU | | |
| Campylobacter lari | 23 | 0.99 | NA | ≧1 × 10⁸ | ≧1 × 10⁶ | 15 μL | Neg. |
| | | | | CFU | CFU | | |
| Campylobacter | 24 | 0.84 | NA | ≧1 × 10⁸ | ≧1 × 10⁶ | 15 μL | Neg. |
| rectus | | | | CFU | CFU | | |
| Campylobacter | 24 | 0.54 | NA | ≧1 × 10⁸ | ≧1 × 10⁶ | 15 μL | Neg. |
| rectus | | | | CFU | CFU | | |
| Campylobacter | 24 | 1.33 | NA | ≧1 × 10⁸ | ≧1 × 10⁶ | 15 μL | Neg. |
| rectus | | | | CFU | CFU | | |
| Campylobacter | 25 | 3.08 | NA | ≧1 × 10⁸ | ≧1 × 10⁶ | 15 μL | Neg. |
| upsaliensis | | | | CFU | CFU | | |
| Campylobacter | 25 | 9.74 | NA | ≧1 × 10⁸ | ≧1 × 10⁶ | 15 μL | Neg. |
| upsaliensis | | | | CFU | CFU | | |
| Campylobacter | 25 | 0.75 | NA | ≧1 × 10⁸ | ≧1 × 10⁶ | 15 μL | Neg. |
| upsaliensis | | | | CFU | CFU | | |
| Candida albicans | 26 | 2.87 | NA | ≧1 × 10⁸ | ≧1 × 10⁶ | 15 μL | Neg. |
| | | | | CFU | CFU | | |
| Candida albicans | 26 | 0.61 | NA | ≧1 × 10⁸ | ≧1 × 10⁶ | 15 μL | Neg. |
| | | | | CFU | CFU | | |
| Candida albicans | 26 | 1.25 | NA | ≧1 × 10⁸ | ≧1 × 10⁶ | 15 μL | Neg. |
| | | | | CFU | CFU | | |
| Candida catenulate | 27 | 5.42 | NA | ≧1 × 10⁸ | ≧1 × 10⁶ | 15 μL | Neg. |
| | | | | CFU | CFU | | |
| Candida catenulate | 27 | 3.55 | NA | ≧1 × 10⁸ | ≧1 × 10⁶ | 15 μL | Neg. |
| | | | | CFU | CFU | | |
| Candida catenulate | 27 | 6.89 | NA | ≧1 × 10⁸ | ≧1 × 10⁶ | 15 μL | Neg. |
| | | | | CFU | CFU | | |
| Cedecea davisae | 28 | 1.38 | NA | ≧1 × 10⁸ | ≧1 × 10⁶ | 15 μL | Neg. |
| | | | | CFU | CFU | | |
| Cedecea davisae | 28 | 1.67 | NA | ≧1 × 10⁸ | ≧1 × 10⁶ | 15 μL | Neg. |
| | | | | CFU | CFU | | |
| Cedecea davisae | 28 | 4.01 | NA | ≧1 × 10⁸ | ≧1 × 10⁶ | 15 μL | Neg. |
| | | | | CFU | CFU | | |
| Chlamydia | 29 | 0.42 | NA | ≧1 × 10⁸ | ≧1 × 10⁶ | 15 μL | Neg. |
| trachomatis | | | | CFU | CFU | | |
| Chlamydia | 29 | 0.54 | NA | ≧1 × 10⁸ | ≧1 × 10⁶ | 15 μL | Neg. |
| trachomatis | | | | CFU | CFU | | |
| Chlamydia | 29 | 2.02 | NA | ≧1 × 10⁸ | ≧1 × 10⁶ | 15 μL | Neg. |
| trachomatis | | | | CFU | CFU | | |
| Citrobacter | 30 | 0.87 | NA | ≧1 × 10⁸ | ≧1 × 10⁶ | 15 μL | Neg. |
| amalonaticus | | | | CFU | CFU | | |
| Citrobacter | 30 | 0.26 | NA | ≧1 × 10⁸ | ≧1 × 10⁶ | 15 μL | Neg. |
| amalonaticus | | | | CFU | CFU | | |
| Citrobacter | 30 | 5.82 | NA | ≧1 × 10⁸ | ≧1 × 10⁶ | 15 μL | Neg. |
| amalonaticus | | | | CFU | CFU | | |
| Citrobacter | 31 | 0.14 | NA | ≧1 × 10⁸ | ≧1 × 10⁶ | 15 μL | Neg. |
| fruendii | | | | CFU | CFU | | |
| Citrobacter | 31 | 4.48 | NA | ≧1 × 10⁸ | ≧1 × 10⁶ | 15 μL | Neg. |
| fruendii | | | | CFU | CFU | | |
| Citrobacter | 31 | 0.34 | NA | ≧1 × 10⁸ | ≧1 × 10⁶ | 15 μL | Neg. |
| fruendii | | | | CFU | CFU | | |
| Citrobacter koseri | 32 | 0.21 | NA | ≧1 × 10⁸ | ≧1 × 10⁶ | 15 μL | Neg. |
| | | | | CFU | CFU | | |
| Citrobacter koseri | 32 | 0.39 | NA | ≧1 × 10⁸ | ≧1 × 10⁶ | 15 μL | Neg. |
| | | | | CFU | CFU | | |
| Citrobacter koseri | 32 | 0.74 | NA | ≧1 × 10⁸ | ≧1 × 10⁶ | 15 μL | Neg. |
| | | | | CFU | CFU | | |
| Citrobacter | 33 | 0.93 | NA | ≧1 × 10⁸ | ≧1 × 10⁶ | 15 μL | Neg. |
| sedlakii | | | | CFU | CFU | | |
| Citrobacter | 33 | 0.58 | NA | ≧1 × 10⁸ | ≧1 × 10⁶ | 15 μL | Neg. |
| sedlakii | | | | CFU | CFU | | |
| Citrobacter | 33 | 0.16 | NA | ≧1 × 10⁸ | ≧1 × 10⁶ | 15 μL | Neg. |
| sedlakii | | | | CFU | CFU | | |
| Clostridium | 34 | 0.47 | NA | ≧1 × 10⁸ | ≧1 × 10⁶ | 15 μL | Neg. |
| difficile 17858 | | | | CFU | CFU | | |
| Clostridium | 34 | 0.42 | NA | ≧1 × 10⁸ | ≧1 × 10⁶ | 15 μL | Neg. |
| difficile 17858 | | | | CFU | CFU | | |
| Clostridium | 34 | 2.58 | NA | ≧1 × 10⁸ | ≧1 × 10⁶ | 15 μL | Neg. |
| difficile 17858 | | | | CFU | CFU | | |
| Clostridium | 35 | 2.7 | NA | ≧1 × 10⁸ | ≧1 × 10⁶ | 15 μL | Neg. |
| difficile 43598 | | | | CFU | CFU | | |
| Clostridium | 35 | 0.65 | NA | ≧1 × 10⁸ | ≧1 × 10⁶ | 15 μL | Neg. |
| difficile 43598 | | | | CFU | CFU | | |
| Clostridium | 35 | 1.95 | NA | ≧1 × 10⁸ | ≧1 × 10⁶ | 15 μL | Neg. |
| difficile 43598 | | | | CFU | CFU | | |
| Clostridium | 36 | 1.24 | NA | ≧1 × 10⁸ | ≧1 × 10⁶ | 15 μL | Neg. |
| difficile CCUG | | | | CFU | CFU | | |
| 8864-9689 | | | | | | | |
| Clostridium | 36 | 0.66 | NA | ≧1 × 10⁸ | ≧1 × 10⁶ | 15 μL | Neg. |
| difficile CCUG | | | | CFU | CFU | | |
| 8864-9689 | | | | | | | |
| Clostridium | 36 | 4.21 | NA | ≧1 × 10⁸ | ≧1 × 10⁶ | 15 μL | Neg. |
| difficile CCUG | | | | CFU | CFU | | |
| 8864-9689 | | | | | | | |
| Clostridium | 37 | 1.99 | NA | ≧1 × 10⁸ | ≧1 × 10⁶ | 15 μL | Neg. |
| difficile 43255 | | | | CFU | CFU | | |
| Clostridium | 37 | 1.3 | NA | ≧1 × 10⁸ | ≧1 × 10⁶ | 15 μL | Neg. |
| difficile 43255 | | | | CFU | CFU | | |
| Clostridium | 37 | 0.64 | NA | ≧1 × 10⁸ | ≧1 × 10⁶ | 15 μL | Neg. |
| difficile 43255 | | | | CFU | CFU | | |
| Clostridium | 38 | 4.9 | NA | ≧1 × 10⁸ | ≧1 × 10⁶ | 15 μL | Neg. |
| difficile BAA- | | | | CFU | CFU | | |
| 1805 | | | | | | | |
| Clostridium | 38 | 0.99 | NA | ≧1 × 10⁸ | ≧1 × 10⁶ | 15 μL | Neg. |
| difficile BAA- | | | | CFU | CFU | | |
| 1805 | | | | | | | |
| Clostridium | 38 | 4.86 | NA | ≧1 × 10⁸ | ≧1 × 10⁶ | 15 μL | Neg. |
| difficile BAA- | | | | CFU | CFU | | |
| 1805 | | | | | | | |
| Clostridium | 39 | 2.78 | NA | ≧1 × 10⁸ | ≧1 × 10⁶ | 15 μL | Neg. |
| difficile 43593 | | | | CFU | CFU | | |
| Clostridium | 39 | 0.69 | NA | ≧1 × 10⁸ | ≧1 × 10⁶ | 15 μL | Neg. |
| difficile 43593 | | | | CFU | CFU | | |
| Clostridium | 39 | 16.21 | NA | ≧1 × 10⁸ | ≧1 × 10⁶ | 15 μL | Neg. |
| difficile 43593 | | | | CFU | CFU | | |
| Clostridium | 40 | 0.47 | NA | ≧1 × 10⁸ | ≧1 × 10⁶ | 15 μL | Neg. |
| perfringens | | | | CFU | CFU | | |
| Clostridium | 40 | 1.22 | NA | ≧1 × 10⁸ | ≧1 × 10⁶ | 15 μL | Neg. |
| perfringens | | | | CFU | CFU | | |
| Clostridium | 40 | 3.69 | NA | ≧1 × 10⁸ | ≧1 × 10⁶ | 15 μL | Neg. |
| perfringens | | | | CFU | CFU | | |
| Collinsella | 41 | 7.7 | NA | ≧1 × 10⁸ | ≧1 × 10⁶ | 15 μL | Neg. |
| aerofaciens | | | | CFU | CFU | | |
| Collinsella | 41 | 11.81 | NA | ≧1 × 10⁸ | ≧1 × 10⁶ | 15 μL | Neg. |
| aerofaciens | | | | CFU | CFU | | |
| Collinsella | 41 | 1.07 | NA | ≧1 × 10⁸ | ≧1 × 10⁶ | 15 μL | Neg. |
| aerofaciens | | | | CFU | CFU | | |
| Corynebacterium | 42 | 8.67 | NA | ≧1 × 10⁸ | ≧1 × 10⁶ | 15 μL | Neg. |
| genitalium | | | | CFU | CFU | | |
| Corynebacterium | 42 | 7.3 | NA | ≧1 × 10⁸ | ≧1 × 10⁶ | 15 μL | Neg. |
| genitalium | | | | CFU | CFU | | |
| Corynebacterium | 42 | 5.65 | NA | ≧1 × 10⁸ | ≧1 × 10⁶ | 15 μL | Neg. |
| genitalium | | | | CFU | CFU | | |
| Desulfovibrio | 43 | 9 | NA | ≧1 × 10⁸ | ≧1 × 10⁶ | 15 μL | Neg. |
| piger | | | | CFU | CFU | | |
| Desulfovibrio | 43 | 5.08 | NA | ≧1 × 10⁸ | ≧1 × 10⁶ | 15 μL | Neg. |
| piger | | | | CFU | CFU | | |
| Desulfovibrio | 43 | 7.91 | NA | ≧1 × 10⁸ | ≧1 × 10⁶ | 15 μL | Neg. |
| piger | | | | CFU | CFU | | |
| Edwardsiella tarda | 44 | 5.89 | NA | ≧1 × 10⁸ | ≧1 × 10⁶ | 15 μL | Neg. |
| | | | | CFU | CFU | | |
| Edwardsiella tarda | 44 | 9.79 | NA | ≧1 × 10⁸ | ≧1 × 10⁶ | 15 μL | Neg. |
| | | | | CFU | CFU | | |
| Edwardsiella tarda | 44 | 4.66 | NA | ≧1 × 10⁸ | ≧1 × 10⁶ | 15 μL | Neg. |
| | | | | CFU | CFU | | |
| Eggerthella lenta | 45 | 9.33 | NA | ≧1 × 10⁸ | ≧1 × 10⁶ | 15 μL | Neg. |
| | | | | CFU | CFU | | |
| Eggerthella lenta | 45 | 9.61 | NA | ≧1 × 10⁸ | ≧1 × 10⁶ | 15 μL | Neg. |
| | | | | CFU | CFU | | |
| Eggerthella lenta | 45 | 12.4 | NA | ≧1 × 10⁸ | ≧1 × 10⁶ | 15 μL | Neg. |
| | | | | CFU | CFU | | |
| Enterobacter | 46 | 7.62 | NA | ≧1 × 10⁸ | ≧1 × 10⁶ | 15 μL | Neg. |
| aerogenes | | | | CFU | CFU | | |
| Enterobacter | 46 | 7.57 | NA | ≧1 × 10⁸ | ≧1 × 10⁶ | 15 μL | Neg. |
| aerogenes | | | | CFU | CFU | | |
| Enterobacter | 46 | 8.36 | NA | ≧1 × 10⁸ | ≧1 × 10⁶ | 15 μL | Neg. |
| aerogenes | | | | CFU | CFU | | |
| Enterobacter | 47 | 7.2 | NA | ≧1 × 10⁸ | ≧1 × 10⁶ | 15 μL | Neg. |
| cloacae | | | | CFU | CFU | | |
| Enterobacter | 47 | 9.51 | NA | ≧1 × 10⁸ | ≧1 × 10⁶ | 15 μL | Neg. |
| cloacae | | | | CFU | CFU | | |
| Enterobacter | 47 | 9.35 | NA | ≧1 × 10⁸ | ≧1 × 10⁶ | 15 μL | Neg. |
| cloacae | | | | CFU | CFU | | |
| Enterococcus | 48 | 9.7 | NA | ≧1 × 10⁸ | ≧1 × 10⁶ | 15 μL | Neg. |
| casseliflavus | | | | CFU | CFU | | |
| Enterococcus | 48 | 7.86 | NA | ≧1 × 10⁸ | ≧1 × 10⁶ | 15 μL | Neg. |
| casseliflavus | | | | CFU | CFU | | |
| Enterococcus | 48 | 8.66 | NA | ≧1 × 10⁸ | ≧1 × 10⁶ | 15 μL | Neg. |
| casseliflavus | | | | CFU | CFU | | |
| Enterococcus | 49 | 11.59 | NA | ≧1 × 10⁸ | ≧1 × 10⁶ | 15 μL | Neg. |
| cecorum | | | | CFU | CFU | | |
| Enterococcus | 49 | 10.93 | NA | ≧1 × 10⁸ | ≧1 × 10⁶ | 15 μL | Neg. |
| cecorum | | | | CFU | CFU | | |
| Enterococcus | 49 | 9.84 | NA | ≧1 × 10⁸ | ≧1 × 10⁶ | 15 μL | Neg. |
| cecorum | | | | CFU | CFU | | |
| Enterococcus | 50 | 6.68 | NA | ≧1 × 10⁸ | ≧1 × 10⁶ | 15 μL | Neg. |
| dispar | | | | CFU | CFU | | |
| Enterococcus | 50 | 7.06 | NA | ≧1 × 10⁸ | ≧1 × 10⁶ | 15 μL | Neg. |
| dispar | | | | CFU | CFU | | |
| Enterococcus | 50 | 6.2 | NA | ≧1 × 10⁸ | ≧1 × 10⁶ | 15 μL | Neg. |
| dispar | | | | CFU | CFU | | |
| Enterococus | 51 | 8.56 | NA | ≧1 × 10⁸ | ≧1 × 10⁶ | 15 μL | Neg. |
| faecalis | | | | CFU | CFU | | |
| Enterococus | 51 | 8.93 | NA | ≧1 × 10⁸ | ≧1 × 10⁶ | 15 μL | Neg. |
| faecalis | | | | CFU | CFU | | |
| Enterococus | 51 | 14.31 | NA | ≧1 × 10⁸ | ≧1 × 10⁶ | 15 μL | Neg. |
| faecalis | | | | CFU | CFU | | |
| Lactococcus lactis | 52 | 10.5 | NA | ≧1 × 10⁸ | ≧1 × 10⁶ | 15 μL | Neg. |
| | | | | CFU | CFU | | |
| Lactococcus lactis | 52 | 7.42 | NA | ≧1 × 10⁸ | ≧1 × 10⁶ | 15 μL | Neg. |
| | | | | CFU | CFU | | |
| Lactococcus lactis | 52 | 8.9 | NA | ≧1 × 10⁸ | ≧1 × 10⁶ | 15 μL | Neg. |
| | | | | CFU | CFU | | |
| Enterococcus | 53 | 9.14 | NA | ≧1 × 10⁸ | ≧1 × 10⁶ | 15 μL | Neg. |
| gallinarum | | | | CFU | CFU | | |
| Enterococcus | 53 | 8.76 | NA | ≧1 × 10⁸ | ≧1 × 10⁶ | 15 μL | Neg. |
| gallinarum | | | | CFU | CFU | | |
| Enterococcus | 53 | 9.47 | NA | ≧1 × 10⁸ | ≧1 × 10⁶ | 15 μL | Neg. |
| gallinarum | | | | CFU | CFU | | |
| Enterococcus hirae | 54 | 10.15 | NA | ≧1 × 10⁸ | ≧1 × 10⁶ | 15 μL | Neg. |
| | | | | CFU | CFU | | |
| Enterococcus hirae | 54 | 8.15 | NA | ≧1 × 10⁸ | ≧1 × 10⁶ | 15 μL | Neg. |
| | | | | CFU | CFU | | |
| Enterococcus hirae | 54 | 9.57 | NA | ≧1 × 10⁸ | ≧1 × 10⁶ | 15 μL | Neg. |
| | | | | CFU | CFU | | |
| Enterococcus | 55 | 9.96 | NA | ≧1 × 10⁸ | ≧1 × 10⁶ | 15 μL | Neg. |
| raffinosus | | | | CFU | CFU | | |
| Enterococcus | 55 | 3.15 | NA | ≧1 × 10⁸ | ≧1 × 10⁶ | 15 μL | Neg. |
| raffinosus | | | | CFU | CFU | | |
| Enterococcus | 55 | 9.48 | NA | ≧1 × 10⁸ | ≧1 × 10⁶ | 15 μL | Neg. |
| raffinosus | | | | CFU | CFU | | |
| Escherichia coli | 56 | 11.62 | NA | ≧1 × 10⁸ | ≧1 × 10⁶ | 15 μL | Neg. |
| 25922 | | | | CFU | CFU | | |
| Escherichia coli | 56 | 7.95 | NA | ≧1 × 10⁸ | ≧1 × 10⁶ | 15 μL | Neg. |
| 25922 | | | | CFU | CFU | | |
| Escherichia coli | 56 | 3.17 | NA | ≧1 × 10⁸ | ≧1 × 10⁶ | 15 μL | Neg. |
| 25922 | | | | CFU | CFU | | |
| E. coli O157 stx 1 | 57 | 6.46 | NA | ≧1 × 10⁸ | ≧1 × 10⁶ | 15 μL | Neg. |
| | | | | CFU | CFU | | |
| E. coli O157 stx 1 | 57 | 20.75 | NA | ≧1 × 10⁸ | ≧1 × 10⁶ | 15 μL | Neg. |
| | | | | CFU | CFU | | |
| E. coli O157 stx 1 | 57 | 4.51 | NA | ≧1 × 10⁸ | ≧1 × 10⁶ | 15 μL | Neg. |
| | | | | CFU | CFU | | |
| E. coli O157 stx 2 | 58 | 3.98 | NA | ≧1 × 10⁸ | ≧1 × 10⁶ | 15 μL | Neg. |
| | | | | CFU | CFU | | |
| E. coli O157 stx 2 | 58 | 6.2 | NA | ≧1 × 10⁸ | ≧1 × 10⁶ | 15 μL | Neg. |
| | | | | CFU | CFU | | |
| E. coli O157 stx 2 | 58 | 2.28 | NA | ≧1 × 10⁸ | ≧1 × 10⁶ | 15 μL | Neg. |
| | | | | CFU | CFU | | |
| Escherichia coli | 59 | 5.07 | NA | ≧1 × 10⁸ | ≧1 × 10⁶ | 15 μL | Neg. |
| 12014 | | | | CFU | CFU | | |
| Escherichia coli | 59 | 6.36 | NA | ≧1 × 10⁸ | ≧1 × 10⁶ | 15 μL | Neg. |
| 12014 | | | | CFU | CFU | | |
| Escherichia coli | 59 | 6.87 | NA | ≧1 × 10⁸ | ≧1 × 10⁶ | 15 μL | Neg. |
| 12014 | | | | CFU | CFU | | |
| Escherichia coli | 60 | 4.89 | NA | ≧1 × 10⁸ | ≧1 × 10⁶ | 15 μL | Neg. |
| 8739 | | | | CFU | CFU | | |
| Escherichia coli | 60 | 6.63 | NA | ≧1 × 10⁸ | ≧1 × 10⁶ | 15 μL | Neg. |
| 8739 | | | | CFU | CFU | | |
| Escherichia coli | 60 | 3.97 | NA | ≧1 × 10⁸ | ≧1 × 10⁶ | 15 μL | Neg. |
| 8739 | | | | CFU | CFU | | |
| Escherichia coli | 61 | 14.9 | NA | ≧1 × 10⁸ | ≧1 × 10⁶ | 15 μL | Neg. |
| 10536 | | | | CFU | CFU | | |
| Escherichia coli | 61 | 16.81 | NA | ≧1 × 10⁸ | ≧1 × 10⁶ | 15 μL | Neg. |
| 10536 | | | | CFU | CFU | | |
| Escherichia coli | 61 | 6.76 | NA | ≧1 × 10⁸ | ≧1 × 10⁶ | 15 μL | Neg. |
| 10536 | | | | CFU | CFU | | |
| Escherichia coli | 62 | 5.42 | NA | ≧1 × 10⁸ | ≧1 × 10⁶ | 15 μL | Neg. |
| 33605 | | | | CFU | CFU | | |
| Escherichia coli | 62 | 19.56 | NA | ≧1 × 10⁸ | ≧1 × 10⁶ | 15 μL | Neg. |
| 33605 | | | | CFU | CFU | | |
| Escherichia coli | 62 | 2.2 | NA | ≧1 × 10⁸ | ≧1 × 10⁶ | 15 μL | Neg. |
| 33605 | | | | CFU | CFU | | |
| Escherichia | 63 | 1.84 | NA | ≧1 × 10⁸ | ≧1 × 10⁶ | 15 μL | Neg. |
| fergusonii | | | | CFU | CFU | | |
| Escherichia | 63 | 5.88 | NA | ≧1 × 10⁸ | ≧1 × 10⁶ | 15 μL | Neg. |
| fergusonii | | | | CFU | CFU | | |
| Escherichia | 63 | 11.36 | NA | ≧1 × 10⁸ | ≧1 × 10⁶ | 15 μL | Neg. |
| fergusonii | | | | CFU | CFU | | |
| Escherichia | 64 | 17.27 | NA | ≧1 × 10⁸ | ≧1 × 10⁶ | 15 μL | Neg. |
| hermannii | | | | CFU | CFU | | |
| Escherichia | 64 | 4.04 | NA | ≧1 × 10⁸ | ≧1 × 10⁶ | 15 μL | Neg. |
| hermannii | | | | CFU | CFU | | |
| Escherichia | 64 | 2.54 | NA | ≧1 × 10⁸ | ≧1 × 10⁶ | 15 μL | Neg. |
| hermannii | | | | CFU | CFU | | |
| Escherichia | 65 | 6.71 | NA | ≧1 × 10⁸ | ≧1 × 10⁶ | 15 μL | Neg. |
| vulneris | | | | CFU | CFU | | |
| Escherichia | 65 | 11.1 | NA | ≧1 × 10⁸ | ≧1 × 10⁶ | 15 μL | Neg. |
| vulneris | | | | CFU | CFU | | |
| Escherichia | 65 | 6.4 | NA | ≧1 × 10⁸ | ≧1 × 10⁶ | 15 μL | Neg. |
| vulneris | | | | CFU | CFU | | |
| Fusobacterium | 66 | 5.59 | NA | ≧1 × 10⁸ | ≧1 × 10⁶ | 15 μL | Neg. |
| varium | | | | CFU | CFU | | |
| Fusobacterium | 66 | 2.6 | NA | ≧1 × 10⁸ | ≧1 × 10⁶ | 15 μL | Neg. |
| varium | | | | CFU | CFU | | |
| Fusobacterium | 66 | 4.26 | NA | ≧1 × 10⁸ | ≧1 × 10⁶ | 15 μL | Neg. |
| varium | | | | CFU | CFU | | |
| Gardnerella | 67 | 3.45 | NA | ≧1 × 10⁸ | ≧1 × 10⁶ | 15 μL | Neg. |
| vaginalis | | | | CFU | CFU | | |
| Gardnerella | 67 | 5.99 | NA | ≧1 × 10⁸ | ≧1 × 10⁶ | 15 μL | Neg. |
| vaginalis | | | | CFU | CFU | | |
| Gardnerella | 67 | 7.12 | NA | ≧1 × 10⁸ | ≧1 × 10⁶ | 15 μL | Neg. |
| vaginalis | | | | CFU | CFU | | |
| Gemella | 68 | 4.48 | NA | ≧1 × 10⁸ | ≧1 × 10⁶ | 15 μL | Neg. |
| morbillorum | | | | CFU | CFU | | |
| Gemella | 68 | 8.52 | NA | ≧1 × 10⁸ | ≧1 × 10⁶ | 15 μL | Neg. |
| morbillorum | | | | CFU | CFU | | |
| Gemella | 68 | 5.28 | NA | ≧1 × 10⁸ | ≧1 × 10⁶ | 15 μL | Neg. |
| morbillorum | | | | CFU | CFU | | |
| Hafnia alvei | 69 | 2.4 | NA | ≧1 × 10⁸ | ≧1 × 10⁶ | 15 μL | Neg. |
| | | | | CFU | CFU | | |
| Hafnia alvei | 69 | 3.16 | NA | ≧1 × 10⁸ | ≧1 × 10⁶ | 15 μL | Neg. |
| | | | | CFU | CFU | | |
| Hafnia alvei | 69 | 3.92 | NA | ≧1 × 10⁸ | ≧1 × 10⁶ | 15 μL | Neg. |
| | | | | CFU | CFU | | |
| Helicobacter | 70 | 7.61 | NA | ≧1 × 10⁸ | ≧1 × 10⁶ | 15 μL | Neg. |
| fennelliae | | | | CFU | CFU | | |
| Helicobacter | 70 | 10.55 | NA | ≧1 × 10⁸ | ≧1 × 10⁶ | 15 μL | Neg. |
| fennelliae | | | | CFU | CFU | | |
| Helicobacter | 70 | 2.61 | NA | ≧1 × 10⁸ | ≧1 × 10⁶ | 15 μL | Neg. |
| fennelliae | | | | CFU | CFU | | |
| Helicobacter | 71 | 4.81 | NA | ≧1 × 10⁸ | ≧1 × 10⁶ | 15 μL | Neg. |
| pylori | | | | CFU | CFU | | |
| Helicobacter | 71 | 2.46 | NA | ≧1 × 10⁸ | ≧1 × 10⁶ | 15 μL | Neg. |
| pylori | | | | CFU | CFU | | |
| Helicobacter | 71 | 2.11 | NA | ≧1 × 10⁸ | ≧1 × 10⁶ | 15 μL | Neg. |
| pylori | | | | CFU | CFU | | |
| Klebsiella oxytoca | 72 | 11.75 | NA | ≧1 × 10⁸ | ≧1 × 10⁶ | 15 μL | Neg. |
| | | | | CFU | CFU | | |
| Klebsiella oxytoca | 72 | 3.74 | NA | ≧1 × 10⁸ | ≧1 × 10⁶ | 15 μL | Neg. |
| | | | | CFU | CFU | | |
| Klebsiella oxytoca | 72 | 4.13 | NA | ≧1 × 10⁸ | ≧1 × 10⁶ | 15 μL | Neg. |
| | | | | CFU | CFU | | |
| Klebsiella | 73 | 1.9 | NA | ≧1 × 10⁸ | ≧1 × 10⁶ | 15 μL | Neg. |
| pneumoniae | | | | CFU | CFU | | |
| Klebsiella | 73 | 2.45 | NA | ≧1 × 10⁸ | ≧1 × 10⁶ | 15 μL | Neg. |
| pneumoniae | | | | CFU | CFU | | |
| Klebsiella | 73 | 6.13 | NA | ≧1 × 10⁸ | ≧1 × 10⁶ | 15 μL | Neg. |
| pneumoniae | | | | CFU | CFU | | |
| Lactobacillus | 74 | 1.1 | NA | ≧1 × 10⁸ | ≧1 × 10⁶ | 15 μL | Neg. |
| acidophilus | | | | CFU | CFU | | |
| Lactobacillus | 74 | 0.44 | NA | ≧1 × 10⁸ | ≧1 × 10⁶ | 15 μL | Neg. |
| acidophilus | | | | CFU | CFU | | |
| Lactobacillus | 74 | 0.54 | NA | ≧1 × 10⁸ | ≧1 × 10⁶ | 15 μL | Neg. |
| acidophilus | | | | CFU | CFU | | |
| Lactobacillus | 75 | 6.71 | NA | ≧1 × 10⁸ | ≧1 × 10⁶ | 15 μL | Neg. |
| reuteri | | | | CFU | CFU | | |
| Lactobacillus | 75 | 3.93 | NA | ≧1 × 10⁸ | ≧1 × 10⁶ | 15 μL | Neg. |
| reuteri | | | | CFU | CFU | | |
| Lactobacillus | 75 | 0.16 | NA | ≧1 × 10⁸ | ≧1 × 10⁶ | 15 μL | Neg. |
| reuteri | | | | CFU | CFU | | |
| Lactococcus lactis | 76 | 0.21 | NA | ≧1 × 10⁸ | ≧1 × 10⁶ | 15 μL | Neg. |
| | | | | CFU | CFU | | |
| Lactococcus lactis | 76 | 2.62 | NA | ≧1 × 10⁸ | ≧1 × 10⁶ | 15 μL | Neg. |
| | | | | CFU | CFU | | |
| Lactococcus lactis | 76 | 0.91 | NA | ≧1 × 10⁸ | ≧1 × 10⁶ | 15 μL | Neg. |
| | | | | CFU | CFU | | |
| Leminorella | 77 | 1.03 | NA | ≧1 × 10⁸ | ≧1 × 10⁶ | 15 μL | Neg. |
| grimontii | | | | CFU | CFU | | |
| Leminorella | 77 | 0.7 | NA | ≧1 × 10⁸ | ≧1 × 10⁶ | 15 μL | Neg. |
| grimontii | | | | CFU | CFU | | |
| Leminorella | 77 | 1.32 | NA | ≧1 × 10⁸ | ≧1 × 10⁶ | 15 μL | Neg. |
| grimontii | | | | CFU | CFU | | |
| Listeria grayi | 78 | 1.27 | NA | ≧1 × 10⁸ | ≧1 × 10⁶ | 15 μL | Neg. |
| | | | | CFU | CFU | | |
| Listeria grayi | 78 | 0.99 | NA | ≧1 × 10⁸ | ≧1 × 10⁶ | 15 μL | Neg. |
| | | | | CFU | CFU | | |
| Listeria grayi | 78 | 7.31 | NA | ≧1 × 10⁸ | ≧1 × 10⁶ | 15 μL | Neg. |
| | | | | CFU | CFU | | |
| Listeria innocua | 79 | 0.35 | NA | ≧1 × 10⁸ | ≧1 × 10⁶ | 15 μL | Neg. |
| | | | | CFU | CFU | | |
| Listeria innocua | 79 | 1.56 | NA | ≧1 × 10⁸ | ≧1 × 10⁶ | 15 μL | Neg. |
| | | | | CFU | CFU | | |
| Listeria innocua | 79 | 11.49 | NA | ≧1 × 10⁸ | ≧1 × 10⁶ | 15 μL | Neg. |
| | | | | CFU | CFU | | |
| Listeria | 80 | 3.4 | NA | ≧1 × 10⁸ | ≧1 × 10⁶ | 15 μL | Neg. |
| monocytogenes | | | | CFU | CFU | | |
| Listeria | 80 | 0.26 | NA | ≧1 × 10⁸ | ≧1 × 10⁶ | 15 μL | Neg. |
| monocytogenes | | | | CFU | CFU | | |
| Listeria | 80 | 1.74 | NA | ≧1 × 10⁸ | ≧1 × 10⁶ | 15 μL | Neg. |
| monocytogenes | | | | CFU | CFU | | |
| Morganella | 81 | 0.92 | NA | ≧1 × 10⁸ | ≧1 × 10⁶ | 15 μL | Neg. |
| morganii | | | | CFU | CFU | | |
| Morganella | 81 | 6.18 | NA | ≧1 × 10⁸ | ≧1 × 10⁶ | 15 μL | Neg. |
| morganii | | | | CFU | CFU | | |
| Morganella | 81 | 4.62 | NA | ≧1 × 10⁸ | ≧1 × 10⁶ | 15 μL | Neg. |
| morganii | | | | CFU | CFU | | |
| Peptoniphilus | 82 | 1.05 | NA | ≧1 × 10⁸ | ≧1 × 10⁶ | 15 μL | Neg. |
| asaccharolyticus | | | | CFU | CFU | | |
| Peptoniphilus | 82 | 0.81 | NA | ≧1 × 10⁸ | ≧1 × 10⁶ | 15 μL | Neg. |
| asaccharolyticus | | | | CFU | CFU | | |
| Peptoniphilus | 82 | 6.99 | NA | ≧1 × 10⁸ | ≧1 × 10⁶ | 15 μL | Neg. |
| asaccharolyticus | | | | CFU | CFU | | |
| Peptostreptococcus | 83 | 5.98 | NA | ≧1 × 10⁸ | ≧1 × 10⁶ | 15 μL | Neg. |
| anaerobius | | | | CFU | CFU | | |
| Peptostreptococcus | 83 | 0.27 | NA | ≧1 × 10⁸ | ≧1 × 10⁶ | 15 μL | Neg. |
| anaerobius | | | | CFU | CFU | | |
| Peptostreptococcus | 83 | 0.88 | NA | ≧1 × 10⁸ | ≧1 × 10⁶ | 15 μL | Neg. |
| anaerobius | | | | CFU | CFU | | |
| Plesiomonas | 84 | 3.18 | NA | ≧1 × 10⁸ | ≧1 × 10⁶ | 15 μL | Neg. |
| shigelloides | | | | CFU | CFU | | |
| Plesiomonas | 84 | 0.89 | NA | ≧1 × 10⁸ | ≧1 × 10⁶ | 15 μL | Neg. |
| shigelloides | | | | CFU | CFU | | |
| Plesiomonas | 84 | 1.17 | NA | ≧1 × 10⁸ | ≧1 × 10⁶ | 15 μL | Neg. |
| shigelloides | | | | CFU | CFU | | |
| Porphyromonas | 85 | 3.82 | NA | ≧1 × 10⁸ | ≧1 × 10⁶ | 15 μL | Neg. |
| asaccharolytica | | | | CFU | CFU | | |
| Porphyromonas | 85 | 3.18 | NA | ≧1 × 10⁸ | ≧1 × 10⁶ | 15 μL | Neg. |
| asaccharolytica | | | | CFU | CFU | | |
| Porphyromonas | 85 | 0.75 | NA | ≧1 × 10⁸ | ≧1 × 10⁶ | 15 μL | Neg. |
| asaccharolytica | | | | CFU | CFU | | |
| Prevotella | 86 | 6.94 | NA | ≧1 × 10⁸ | ≧1 × 10⁶ | 15 μL | Neg. |
| melaninogenica | | | | CFU | CFU | | |
| Prevotella | 86 | 6.87 | NA | ≧1 × 10⁸ | ≧1 × 10⁶ | 15 μL | Neg. |
| melaninogenica | | | | CFU | CFU | | |
| Prevotella | 86 | 4.62 | NA | ≧1 × 10⁸ | ≧1 × 10⁶ | 15 μL | Neg. |
| melaninogenica | | | | CFU | CFU | | |
| Proteus mirabilis | 87 | 8.08 | NA | ≧1 × 10⁸ | ≧1 × 10⁶ | 15 μL | Neg. |
| | | | | CFU | CFU | | |
| Proteus mirabilis | 87 | 15.63 | NA | ≧1 × 10⁸ | ≧1 × 10⁶ | 15 μL | Neg. |
| | | | | CFU | CFU | | |
| Proteus mirabilis | 87 | 14.58 | NA | ≧1 × 10⁸ | ≧1 × 10⁶ | 15 μL | Neg. |
| | | | | CFU | CFU | | |
| Proteus penneri | 88 | 10 | NA | ≧1 × 10⁸ | ≧1 × 10⁶ | 15 μL | Neg. |
| | | | | CFU | CFU | | |
| Proteus penneri | 88 | 4.92 | NA | ≧1 × 10⁸ | ≧1 × 10⁶ | 15 μL | Neg. |
| | | | | CFU | CFU | | |
| Proteus penneri | 88 | 10.77 | NA | ≧1 × 10⁸ | ≧1 × 10⁶ | 15 μL | Neg. |
| | | | | CFU | CFU | | |
| Proteus vulgaris | 89 | 5.41 | NA | ≧1 × 10⁸ | ≧1 × 10⁶ | 15 μL | Neg. |
| | | | | CFU | CFU | | |
| Proteus vulgaris | 89 | 5.81 | NA | ≧1 × 10⁸ | ≧1 × 10⁶ | 15 μL | Neg. |
| | | | | CFU | CFU | | |
| Proteus vulgaris | 89 | 0.87 | NA | ≧1 × 10⁸ | ≧1 × 10⁶ | 15 μL | Neg. |
| | | | | CFU | CFU | | |
| Providencia | 90 | 9.11 | NA | ≧1 × 10⁸ | ≧1 × 10⁶ | 15 μL | Neg. |
| alcalifaciens | | | | CFU | CFU | | |
| Providencia | 90 | 4.68 | NA | ≧1 × 10⁸ | ≧1 × 10⁶ | 15 μL | Neg. |
| alcalifaciens | | | | CFU | CFU | | |
| Providencia | 90 | 1.49 | NA | ≧1 × 10⁸ | ≧1 × 10⁶ | 15 μL | Neg. |
| alcalifaciens | | | | CFU | CFU | | |
| Providencia | 91 | 5.33 | NA | ≧1 × 10⁸ | ≧1 × 10⁶ | 15 μL | Neg. |
| rettgeri | | | | CFU | CFU | | |
| Providencia | 91 | 0.11 | NA | ≧1 × 10⁸ | ≧1 × 10⁶ | 15 μL | Neg. |
| rettgeri | | | | CFU | CFU | | |
| Providencia | 91 | 0.67 | NA | ≧1 × 10⁸ | ≧1 × 10⁶ | 15 μL | Neg. |
| rettgeri | | | | CFU | CFU | | |
| Providencia | 92 | 3.35 | NA | ≧1 × 10⁸ | ≧1 × 10⁶ | 15 μL | Neg. |
| stuartii | | | | CFU | CFU | | |
| Providencia | 92 | 1.05 | NA | ≧1 × 10⁸ | ≧1 × 10⁶ | 15 μL | Neg. |
| stuartii | | | | CFU | CFU | | |
| Providencia | 92 | 1.55 | NA | ≧1 × 10⁸ | ≧1 × 10⁶ | 15 μL | Neg. |
| stuartii | | | | CFU | CFU | | |
| Pseudomonas | 93 | 4.61 | NA | ≧1 × 10⁸ | ≧1 × 10⁶ | 15 μL | Neg. |
| aeruginosa | | | | CFU | CFU | | |
| Pseudomonas | 93 | 4.31 | NA | ≧1 × 10⁸ | ≧1 × 10⁶ | 15 μL | Neg. |
| aeruginosa | | | | CFU | CFU | | |
| Pseudomonas | 93 | 2.15 | NA | ≧1 × 10⁸ | ≧1 × 10⁶ | 15 μL | Neg. |
| aeruginosa | | | | CFU | CFU | | |
| Pseudomonas | 94 | 2.03 | NA | ≧1 × 10⁸ | ≧1 × 10⁶ | 15 μL | Neg. |
| fluorescens | | | | CFU | CFU | | |
| Pseudomonas | 94 | 2.32 | NA | ≧1 × 10⁸ | ≧1 × 10⁶ | 15 μL | Neg. |
| fluorescens | | | | CFU | CFU | | |
| Pseudomonas | 94 | 5.55 | NA | ≧1 × 10⁸ | ≧1 × 10⁶ | 15 μL | Neg. |
| fluorescens | | | | CFU | CFU | | |
| Ruminococcus | 95 | 1.25 | NA | ≧1 × 10⁸ | ≧1 × 10⁶ | 15 μL | Neg. |
| bromii | | | | CFU | CFU | | |
| Ruminococcus | 95 | 0.73 | NA | ≧1 × 10⁸ | ≧1 × 10⁶ | 15 μL | Neg. |
| bromii | | | | CFU | CFU | | |
| Ruminococcus | 95 | 0.73 | NA | ≧1 × 10⁸ | ≧1 × 10⁶ | 15 μL | Neg. |
| bromii | | | | CFU | CFU | | |
| Salmonella | 96 | 0.77 | NA | ≧1 × 10⁸ | ≧1 × 10⁶ | 15 μL | Neg. |
| typhimurium | | | | CFU | CFU | | |
| Salmonella | 96 | 3.81 | NA | ≧1 × 10⁸ | ≧1 × 10⁶ | 15 μL | Neg. |
| typhimurium | | | | CFU | CFU | | |
| Salmonella | 96 | 2.84 | NA | ≧1 × 10⁸ | ≧1 × 10⁶ | 15 μL | Neg. |
| typhimurium | | | | CFU | CFU | | |
| Salmonella | 97 | 1.28 | NA | ≧1 × 10⁸ | ≧1 × 10⁶ | 15 μL | Neg. |
| enteriditis | | | | CFU | CFU | | |
| Salmonella | 97 | 1.36 | NA | ≧1 × 10⁸ | ≧1 × 10⁶ | 15 μL | Neg. |
| enteriditis | | | | CFU | CFU | | |
| Salmonella | 97 | 1.13 | NA | ≧1 × 10⁸ | ≧1 × 10⁶ | 15 μL | Neg. |
| enteriditis | | | | CFU | CFU | | |
| Serratia | 98 | 2.19 | NA | ≧1 × 10⁸ | ≧1 × 10⁶ | 15 μL | Neg. |
| liquefaciens | | | | CFU | CFU | | |
| Serratia | 98 | 1.69 | NA | ≧1 × 10⁸ | ≧1 × 10⁶ | 15 μL | Neg. |
| liquefaciens | | | | CFU | CFU | | |
| Serratia | 98 | 0.91 | NA | ≧1 × 10⁸ | ≧1 × 10⁶ | 15 μL | Neg. |
| liquefaciens | | | | CFU | CFU | | |
| Serratia | 99 | 1.79 | NA | ≧1 × 10⁸ | ≧1 × 10⁶ | 15 μL | Neg. |
| marcescens | | | | CFU | CFU | | |
| Serratia | 99 | 4.78 | NA | ≧1 × 10⁸ | ≧1 × 10⁶ | 15 μL | Neg. |
| marcescens | | | | CFU | CFU | | |
| Serratia | 99 | 0.77 | NA | ≧1 × 10⁸ | ≧1 × 10⁶ | 15 μL | Neg. |
| marcescens | | | | CFU | CFU | | |
| Shigella sonnei | 100 | 2.33 | NA | ≧1 × 10⁸ | ≧1 × 10⁶ | 15 μL | Neg. |
| | | | | CFU | CFU | | |
| Shigella sonnei | 100 | 1.2 | NA | ≧1 × 10⁸ | ≧1 × 10⁶ | 15 μL | Neg. |
| | | | | CFU | CFU | | |
| Shigella sonnei | 100 | 3.12 | NA | ≧1 × 10⁸ | ≧1 × 10⁶ | 15 μL | Neg. |
| | | | | CFU | CFU | | |
| Shigella flexneri | 101 | 2.58 | NA | ≧1 × 10⁸ | ≧1 × 10⁶ | 15 μL | Neg. |
| | | | | CFU | CFU | | |
| Shigella flexneri | 101 | 2.02 | NA | ≧1 × 10⁸ | ≧1 × 10⁶ | 15 μL | Neg. |
| | | | | CFU | CFU | | |
| Shigella flexneri | 101 | 2.09 | NA | ≧1 × 10⁸ | ≧1 × 10⁶ | 15 μL | Neg. |
| | | | | CFU | CFU | | |
| Staphylococcus | 102 | 2.78 | NA | ≧1 × 10⁸ | ≧1 × 10⁶ | 15 μL | Neg. |
| aureus | | | | CFU | CFU | | |
| Staphylococcus | 102 | 2.48 | NA | ≧1 × 10⁸ | ≧1 × 10⁶ | 15 μL | Neg. |
| aureus | | | | CFU | CFU | | |
| Staphylococcus | 102 | 2.91 | NA | ≧1 × 10⁸ | ≧1 × 10⁶ | 15 μL | Neg. |
| aureus | | | | CFU | CFU | | |
| Staphylococcus | 103 | 2.89 | NA | ≧1 × 10⁸ | ≧1 × 10⁶ | 15 μL | Neg. |
| epidermidis | | | | CFU | CFU | | |
| Staphylococcus | 103 | 2.92 | NA | ≧1 × 10⁸ | ≧1 × 10⁶ | 15 μL | Neg. |
| epidermidis | | | | CFU | CFU | | |
| Staphylococcus | 103 | 2.73 | NA | ≧1 × 10⁸ | ≧1 × 10⁶ | 15 μL | Neg. |
| epidermidis | | | | CFU | CFU | | |
| Stenotrophomonas | 104 | 3.86 | NA | ≧1 × 10⁸ | ≧1 × 10⁶ | 15 μL | Neg. |
| maltophilia | | | | CFU | CFU | | |
| Stenotrophomonas | 104 | 2.05 | NA | ≧1 × 10⁸ | ≧1 × 10⁶ | 15 μL | Neg. |
| maltophilia | | | | CFU | CFU | | |
| Stenotrophomonas | 104 | 2.62 | NA | ≧1 × 10⁸ | ≧1 × 10⁶ | 15 μL | Neg. |
| maltophilia | | | | CFU | CFU | | |
| Streptococcus | 105 | 5.5 | NA | ≧1 × 10⁸ | ≧1 × 10⁶ | 15 μL | Neg. |
| agalactiae | | | | CFU | CFU | | |
| Streptococcus | 105 | 2.98 | NA | ≧1 × 10⁸ | ≧1 × 10⁶ | 15 μL | Neg. |
| agalactiae | | | | CFU | CFU | | |
| Streptococcus | 105 | 8.92 | NA | ≧1 × 10⁸ | ≧1 × 10⁶ | 15 μL | Neg. |
| agalactiae | | | | CFU | CFU | | |
| Streptococcus | 106 | 6.85 | NA | ≧1 × 10⁸ | ≧1 × 10⁶ | 15 μL | Neg. |
| dysgalactiae | | | | CFU | CFU | | |
| Streptococcus | 106 | 1.45 | NA | ≧1 × 10⁸ | ≧1 × 10⁶ | 15 μL | Neg. |
| dysgalactiae | | | | CFU | CFU | | |
| Streptococcus | 106 | 4.53 | NA | ≧1 × 10⁸ | ≧1 × 10⁶ | 15 μL | Neg. |
| dysgalactiae | | | | CFU | CFU | | |
| Streptococcus | 107 | 5.55 | NA | ≧1 × 10⁸ | ≧1 × 10⁶ | 15 μL | Neg. |
| intermedius | | | | CFU | CFU | | |
| Streptococcus | 107 | 1.27 | NA | ≧1 × 10⁸ | ≧1 × 10⁶ | 15 μL | Neg. |
| intermedius | | | | CFU | CFU | | |
| Streptococcus | 107 | 1.56 | NA | ≧1 × 10⁸ | ≧1 × 10⁶ | 15 μL | Neg. |
| intermedius | | | | CFU | CFU | | |
| Streptococcus | 108 | 5.03 | NA | ≧1 × 10⁸ | ≧1 × 10⁶ | 15 μL | Neg. |
| uberis | | | | CFU | CFU | | |
| Streptococcus | 108 | 6.12 | NA | ≧1 × 10⁸ | ≧1 × 10⁶ | 15 μL | Neg. |
| uberis | | | | CFU | CFU | | |
| Streptococcus | 108 | 6.26 | NA | ≧1 × 10⁸ | ≧1 × 10⁶ | 15 μL | Neg. |
| uberis | | | | CFU | CFU | | |
| Trabulsiella | 109 | 8.68 | NA | ≧1 × 10⁸ | ≧1 × 10⁶ | 15 μL | Neg. |
| guamensis | | | | CFU | CFU | | |
| Trabulsiella | 109 | 9.48 | NA | ≧1 × 10⁸ | ≧1 × 10⁶ | 15 μL | Neg. |
| guamensis | | | | CFU | CFU | | |
| Trabulsiella | 109 | 8.58 | NA | ≧1 × 10⁸ | ≧1 × 10⁶ | 15 μL | Neg. |
| guamensis | | | | CFU | CFU | | |
| Veillonella parvula | 110 | 9.54 | NA | ≧1 × 10⁸ | ≧1 × 10⁶ | 15 μL | Neg. |
| | | | | CFU | CFU | | |
| Veillonella parvula | 110 | 17.28 | NA | ≧1 × 10⁸ | ≧1 × 10⁶ | 15 μL | Neg. |
| | | | | CFU | CFU | | |
| Veillonella parvula | 110 | 0.73 | NA | ≧1 × 10⁸ | ≧1 × 10⁶ | 15 μL | Neg. |
| | | | | CFU | CFU | | |
| Vibrio cholerae | 111 | 0.95 | NA | ≧1 × 10⁸ | ≧1 × 10⁶ | 15 μL | Neg. |
| | | | | CFU | CFU | | |
| Vibrio cholerae | 111 | 8.9 | NA | ≧1 × 10⁸ | ≧1 × 10⁶ | 15 μL | Neg. |
| | | | | CFU | CFU | | |
| Vibrio cholerae | 111 | 8.13 | NA | ≧1 × 10⁸ | ≧1 × 10⁶ | 15 μL | Neg. |
| | | | | CFU | CFU | | |
| Vibrio | 112 | 0.85 | NA | ≧1 × 10⁸ | ≧1 × 10⁶ | 15 μL | Neg. |
| parahaemolyticus | | | | CFU | CFU | | |
| Vibrio | 112 | 14.44 | NA | ≧1 × 10⁸ | ≧1 × 10⁶ | 15 μL | Neg. |
| parahaemolyticus | | | | CFU | CFU | | |
| Vibrio | 112 | 7.87 | NA | ≧1 × 10⁸ | ≧1 × 10⁶ | 15 μL | Neg. |
| parahaemolyticus | | | | CFU | CFU | | |
| Yersinia bercovieri | 113 | 6.08 | NA | ≧1 × 10⁸ | ≧1 × 10⁶ | 15 μL | Neg. |
| | | | | CFU | CFU | | |
| Yersinia bercovieri | 113 | 1.91 | NA | ≧1 × 10⁸ | ≧1 × 10⁶ | 15 μL | Neg. |
| | | | | CFU | CFU | | |
| Yersinia bercovieri | 113 | 10.32 | NA | ≧1 × 10⁸ | ≧1 × 10⁶ | 15 μL | Neg. |
| | | | | CFU | CFU | | |
| Yersinia | 114 | 5.34 | NA | ≧1 × 10⁸ | ≧1 × 10⁶ | 15 μL | Neg. |
| enterocolitica | | | | CFU | CFU | | |
| Yersinia | 114 | 5.3 | NA | ≧1 × 10⁸ | ≧1 × 10⁶ | 15 μL | Neg. |
| enterocolitica | | | | CFU | CFU | | |
| Yersinia | 114 | 2.24 | NA | ≧1 × 10⁸ | ≧1 × 10⁶ | 15 μL | Neg. |
| enterocolitica | | | | CFU | CFU | | |
| Yersinia rohdei | 115 | 11.14 | NA | ≧1 × 10⁸ | ≧1 × 10⁶ | 15 μL | Neg. |
| | | | | CFU | CFU | | |
| Yersinia rohdei | 115 | 1.79 | NA | ≧1 × 10⁸ | ≧1 × 10⁶ | 15 μL | Neg. |
| | | | | CFU | CFU | | |
| Yersinia rohdei | 115 | 1.82 | NA | ≧1 × 10⁸ | ≧1 × 10⁶ | 15 μL | Neg. |
| | | | | CFU | CFU | | |
| Adenovirus type 2 | 116 | 9.51 | NA | 8.9 × 10⁷ | ≧1 × 10⁴ TCID₅₀/mL | 150 μL | Neg. |
| | | | | TCID₅₀ | | | |
| Adenovirus type 2 | 116 | 9.74 | NA | 8.9 × 10⁷ | ≧1 × 10⁴ TCID₅₀/mL | 150 μL | Neg. |
| | | | | TCID₅₀ | | | |
| Adenovirus type 2 | 116 | 5.56 | NA | 8.9 × 10⁷ | ≧1 × 10⁴ TCID₅₀/mL | 150 μL | Neg. |
| | | | | TCID₅₀ | | | |
| Adenovirus type | 117 | 1.99 | NA | 8.9 × 10⁸ | ≧1 × 10⁴ TCID₅₀/mL | 150 μL | Neg. |
| 14 | | | | TCID₅₀ | | | |
| Adenovirus type | 117 | 19.31 | NA | 8.9 × 10⁸ | ≧1 × 10⁴ TCID₅₀/mL | 150 μL | Neg. |
| 14 | | | | TCID₅₀ | | | |
| Adenovirus type | 117 | 9.2 | NA | 8.9 × 10⁸ | ≧1 × 10⁴ TCID₅₀/mL | 150 μL | Neg. |
| 14 | | | | TCID₅₀ | | | |
| Adenovirus type | 118 | 2.22 | NA | 1.8 × 10⁸ | ≧1 × 10⁴ TCID₅₀/mL | 150 μL | Neg. |
| 40 | | | | TCID₅₀ | | | |
| Adenovirus type | 118 | 36.79 | NA | 1.8 × 10⁸ | ≧1 × 10⁴ TCID₅₀/mL | 150 μL | Neg. |
| 40 | | | | TCID₅₀ | | | |
| Adenovirus type | 118 | 1.77 | NA | 1.8 × 10⁸ | ≧1 × 10⁴ TCID₅₀/mL | 150 μL | Neg. |
| 40 | | | | TCID₅₀ | | | |
| Adenovirus type | 119 | 5.89 | NA | 2.8 × 10⁷ | ≧1 × 10⁴ TCID₅₀/mL | 150 μL | Neg. |
| 41 | | | | TCID₅₀ | | | |
| Adenovirus type | 119 | 8.03 | NA | 2.8 × 10⁷ | ≧1 × 10⁴ TCID₅₀/mL | 150 μL | Neg. |
| 41 | | | | TCID₅₀ | | | |
| Adenovirus type | 119 | 1.79 | NA | 2.8 × 10⁷ | ≧1 × 10⁴ TCID₅₀/mL | 150 μL | Neg. |
| 41 | | | | TCID₅₀ | | | |
| Coxsackie A9 | 120 | 2.84 | NA | 1.6 × 10⁵ | ≧1 × 10⁴ TCID₅₀/mL | 150 μL | Neg. |
| | | | | TCID₅₀ | | | |
| Coxsackie A9 | 120 | 5.39 | NA | 1.6 × 10⁵ | ≧1 × 10⁴ TCID₅₀/mL | 150 μL | Neg. |
| | | | | TCID₅₀ | | | |
| Coxsackie A9 | 120 | 9.33 | NA | 1.6 × 10⁵ | ≧1 × 10⁴ TCID₅₀/mL | 150 μL | Neg. |
| | | | | TCID₅₀ | | | |
| Coxsackie B1 | 121 | 0.08 | NA | 8.9 × 10⁷ | ≧1 × 10⁴ TCID₅₀/mL | 150 μL | Neg. |
| | | | | TCID₅₀ | | | |
| Coxsackie B1 | 121 | 0.43 | NA | 8.9 × 10⁷ | ≧1 × 10⁴ TCID₅₀/mL | 150 μL | Neg. |
| | | | | TCID₅₀ | | | |
| Coxsackie B1 | 121 | 0.5 | NA | 8.9 × 10⁷ | ≧1 × 10⁴ TCID₅₀/mL | 150 μL | Neg. |
| | | | | TCID₅₀ | | | |
| HHV-5 | 122 | 1.01 | NA | 8.9 × 10⁵ | ≧1 × 10⁴ TCID₅₀/mL | 150 μL | Neg. |
| Cytomegalovirus | | | | TCID₅₀ | | | |
| HHV-5 | 122 | 2.56 | NA | 8.9 × 10⁵ | ≧1 × 10⁴ TCID₅₀/mL | 150 μL | Neg. |
| Cytomegalovirus | | | | TCID₅₀ | | | |
| HHV-5 | 122 | 1.72 | NA | 8.9 × 10⁵ | ≧1 × 10⁴ TCID₅₀/mL | 150 μL | Neg. |
| Cytomegalovirus | | | | TCID₅₀ | | | |
| Enterovirus type | 123 | 1.18 | NA | 1.6 × 10⁵ | ≧1 × 10⁴ TCID₅₀/mL | 150 μL | Neg. |
| 69 | | | | TCID₅₀ | | | |
| Enterovirus type | 123 | 0.19 | NA | 1.6 × 10⁵ | ≧1 × 10⁴ TCID₅₀/mL | 150 μL | Neg. |
| 69 | | | | TCID₅₀ | | | |
| Enterovirus type | 123 | 2.71 | NA | 1.6 × 10⁵ | ≧1 × 10⁴ TCID₅₀/mL | 150 μL | Neg. |
| 69 | | | | TCID₅₀ | | | |
| Human | 124 | 1.16 | NA | HPV | Highest | 150 μL | Neg. |
| Papillomavirus | | | | plasmid in | Spike | | |
| Type 16 | | | | E. coli- | | | |
| | | | | Unknown | | | |
| Human | 124 | 0.52 | NA | HPV | Highest | 150 μL | Neg. |
| Papillomavirus | | | | plasmid in | Spike | | |
| Type 16 | | | | E. coli- | | | |
| | | | | Unknown | | | |
| Human | 124 | 2.88 | NA | HPV | Highest | 150 μL | Neg. |
| Papillomavirus | | | | plasmid in | Spike | | |
| Type 16 | | | | E. coli- | | | |
| | | | | Unknown | | | |
| Human | 125 | 3.11 | NA | HPV | Highest | 150 μL | Neg. |
| Papillomavirus | | | | plasmid in | Spike | | |
| Type 18 | | | | E. coli- | | | |
| | | | | Unknown | | | |
| Human | 125 | 2.06 | NA | HPV | Highest | 150 μL | Neg. |
| Papillomavirus | | | | plasmid in | Spike | | |
| Type 18 | | | | E. coli- | | | |
| | | | | Unknown | | | |
| Human | 125 | 1.8 | NA | HPV | Highest | 150 μL | Neg. |
| Papillomavirus | | | | plasmid in | Spike | | |
| Type 18 | | | | E. coli- | | | |
| | | | | Unknown | | | |
| Herpes Simplex | 126 | 1.2 | NA | 8.9 × 10⁸ | ≧1 × 10⁴ TCID₅₀/mL | 150 μL | Neg. |
| Virus I | | | | TCID₅₀ | | | |
| Herpes Simplex | 126 | 0.79 | NA | 8.9 × 10⁸ | ≧1 × 10⁴ TCID₅₀/mL | 150 μL | Neg. |
| Virus I | | | | TCID₅₀ | | | |
| Herpes Simplex | 126 | 1.28 | NA | 8.9 × 10⁸ | ≧1 × 10⁴ TCID₅₀/mL | 150 μL | Neg. |
| Virus I | | | | TCID₅₀ | | | |
| Herpes Simplex | 127 | 0.58 | NA | 2.8 × 10⁶ | ≧1 × 10⁴ TCID₅₀/mL | 150 μL | Neg. |
| Virus II | | | | TCID₅₀ | | | |
| Herpes Simplex | 127 | 2.55 | NA | 2.8 × 10⁶ | ≧1 × 10⁴ TCID₅₀/mL | 150 μL | Neg. |
| Virus II | | | | TCID₅₀ | | | |
| Herpes Simplex | 127 | 4.46 | NA | 2.8 × 10⁶ | ≧1 × 10⁴ TCID₅₀/mL | 150 μL | Neg. |
| Virus II | | | | TCID₅₀ | | | |
| Norovirus I | 128 | 2.59 | NA | 8.5 × 10⁷ | ≧1 × 10⁴ TCID₅₀/mL | 150 μL | Neg. |
| | | | | TCID₅₀ | | | |
| Norovirus I | 128 | 1.65 | NA | 8.5 × 10⁷ | ≧1 × 10⁴ TCID₅₀/mL | 150 μL | Neg. |
| | | | | TCID₅₀ | | | |
| Norovirus I | 128 | 1.12 | NA | 8.5 × 10⁷ | ≧1 × 10⁴ TCID₅₀/mL | 150 μL | Neg. |
| | | | | TCID₅₀ | | | |
| Norovirus II | 129 | 0.94 | NA | 8.5 × 10⁷ | ≧1 × 10⁴ TCID₅₀/mL | 150 μL | Neg. |
| | | | | TCID₅₁ | | | |
| Norovirus II | 129 | 3.59 | NA | 8.5 × 10⁷ | ≧1 × 10⁴ TCID₅₀/mL | 150 μL | Neg. |
| | | | | TCID₅₁ | | | |
| Norovirus II | 129 | 0.22 | NA | 8.5 × 10⁷ | ≧1 × 10⁴ TCID₅₀/mL | 150 μL | Neg. |
| | | | | TCID₅₁ | | | |
| Rotavirus | 130 | 13.75 | NA | High Titer | Highest | 150 μL | Neg. |
| | | | | Qualitative | Spike | | |
| Rotavirus | 130 | 23.44 | NA | High Titer | Highest | 150 μL | Neg. |
| | | | | Qualitative | Spike | | |
| Rotavirus | 130 | 42.69 | NA | High Titer | Highest | 150 μL | Neg. |
| | | | | Qualitative | Spike | | |
| Blastocystis | 131 | 3.38 | NA | 4.20 × 10⁶ cells/mL | ≧1 × 10⁵ cells/mL | 150 μL | Neg. |
| hominis | | | | | | | |
| Blastocystis | 131 | 6.17 | NA | 4.20 × 10⁶ cells/mL | ≧1 × 10⁵ cells/mL | 150 μL | Neg. |
| hominis | | | | | | | |
| Blastocystis | 131 | 1.81 | NA | 4.20 × 10⁶ cells/mL | ≧1 × 10⁵ cells/mL | 150 μL | Neg. |
| hominis | | | | | | | |
| Encephalitozoon | 132 | 1.49 | NA | 4.28 × 10⁷ cells/mL | ≧1 × 10⁵ cells/mL | 15 μL | Neg. |
| intestinalis | | | | | | | |
| Encephalitozoon | 132 | 2.52 | NA | 4.28 × 10⁷ cells/mL | ≧1 × 10⁵ cells/mL | 15 μL | Neg. |
| intestinalis | | | | | | | |
| Encephalitozoon | 132 | 1.39 | NA | 4.28 × 10⁷ cells/mL | ≧1 × 10⁵ cells/mL | 15 μL | Neg. |
| intestinalis | | | | | | | |
| Encephalitozoon | 133 | 5.3 | NA | 2.00 × 10⁷ cells/mL | ≧1 × 10⁵ cells/mL | 15 μL | Neg. |
| hellum | | | | | | | |
| Encephalitozoon | 133 | 0.74 | NA | 2.00 × 10⁷ cells/mL | ≧1 × 10⁵ cells/mL | 15 μL | Neg. |
| hellum | | | | | | | |
| Encephalitozoon | 133 | 1.41 | NA | 2.00 × 10⁷ cells/mL | ≧1 × 10⁵ cells/mL | 15 μL | Neg. |
| hellum | | | | | | | |
| Encephalitozoon | 134 | 7.64 | NA | 3.40 × 10⁷ cells/mL | ≧1 × 10⁵ cells/mL | 15 μL | Neg. |
| cuniculi | | | | | | | |
| Encephalitozoon | 134 | 2.25 | NA | 3.40 × 10⁷ cells/mL | ≧1 × 10⁵ cells/mL | 15 μL | Neg. |
| cuniculi | | | | | | | |
| Encephalitozoon | 134 | 1.23 | NA | 3.40 × 10⁷ cells/mL | ≧1 × 10⁵ cells/mL | 15 μL | Neg. |
| cuniculi | | | | | | | |
| Pentatrichomonas | 135 | 3.61 | NA | 8.50 × 10⁶ cells/mL | ≧1 × 10⁵ cells/mL | 150 μL | Neg. |
| hominis | | | | | | | |
| Pentatrichomonas | 135 | 27.13 | NA | 8.50 × 10⁶ cells/mL | ≧1 × 10⁵ cells/mL | 150 μL | Neg. |
| hominis | | | | | | | |
| Pentatrichomonas | 135 | 15.04 | NA | 8.50 × 10⁶ cells/mL | ≧1 × 10⁵ cells/mL | 150 μL | Neg. |
| hominis | | | | | | | |
| Entamoeba barretti | 136 | 1.63 | NA | Unknown | Highest | 150 μL | Neg. |
| | | | | Titer | Spike | | |
| Entamoeba barretti | 136 | 5.63 | NA | Unknown | Highest | 150 μL | Neg. |
| | | | | Titer | Spike | | |
| Entamoeba barretti | 136 | 1.02 | NA | Unknown | Highest | 150 μL | Neg. |
| | | | | Titer | Spike | | |
| Entamoeba dispar | 137 | 1.82 | NA | ~1.50 × 10⁵ cells/mL | Highest | 150 μL | Neg. |
| | | | | | Spike | | |
| Entamoeba dispar | 137 | 2.43 | NA | ~1.50 × 10⁵ cells/mL | Highest | 150 μL | Neg. |
| | | | | | Spike | | |
| Entamoeba dispar | 137 | 32.69 | NA | ~1.50 × 10⁵ cells/mL | Highest | 150 μL | Neg. |
| | | | | | Spike | | |
| Entamoeba | 138 | 5.88 | NA | ~1.00 × 10⁵ cells/mL | Highest | 150 μL | Neg. |
| gigivalis | | | | | Spike | | |
| Entamoeba | 138 | 8.41 | NA | ~1.00 × 10⁵ cells/mL | Highest | 150 μL | Neg. |
| gigivalis | | | | | Spike | | |
| Entamoeba | 138 | 4.77 | NA | ~1.00 × 10⁵ cells/mL | Highest | 150 μL | Neg. |
| gigivalis | | | | | Spike | | |
| Entamoeba | 139 | 3.15 | NA | 2.90 × 10⁶ cells/mL | ≧1 × 10⁵ cells/mL | 150 μL | Neg. |
| invadens | | | | | | | |
| Entamoeba | 139 | 4.52 | NA | 2.90 × 10⁶ cells/mL | ≧1 × 10⁵ cells/mL | 150 μL | Neg. |
| invadens | | | | | | | |
| Entamoeba | 139 | 1.15 | NA | 2.90 × 10⁶ cells/mL | ≧1 × 10⁵ cells/mL | 150 μL | Neg. |
| invadens | | | | | | | |
| Entamoeba | 140 | 1.51 | NA | 2.20 × 10⁵ cells/mL | ≧1 × 10⁴ cells/mL | 150 μL | Neg. |
| moshkovskii | | | | | | | |
| Entamoeba | 140 | 8.7 | NA | 2.20 × 10⁵ cells/mL | ≧1 × 10⁴ cells/mL | 150 μL | Neg. |
| moshkovskii | | | | | | | |
| Entamoeba | 140 | 4 | NA | 2.20 × 10⁵ cells/mL | ≧1 × 10⁴ cells/mL | 150 μL | Neg. |
| moshkovskii | | | | | | | |
| Entamobea | 141 | 13.14 | NA | 7.33 × 10⁵ cells/mL | ≧1 × 10⁴ cells/mL | 150 μL | Neg. |
| ranarum | | | | | | | |
| Entamobea | 141 | 12.42 | NA | 7.33 × 10⁵ cells/mL | ≧1 × 10⁴ cells/mL | 150 μL | Neg. |
| ranarum | | | | | | | |
| Entamobea | 141 | 9.68 | NA | 7.33 × 10⁵ cells/mL | ≧1 × 10⁴ cells/mL | 150 μL | Neg. |
| ranarum | | | | | | | |
| Citrobacter | 31 | 2.11 | NA | ≧1 × 10⁸ | ≧1 × 10⁶ | 15 μL | Neg. |
| fruendii (rpt) | | | | CFU | CFU | | |
| Enterobacter | 47 | 0.85 | NA | ≧1 × 10⁸ | ≧1 × 10⁶ | 15 μL | Neg. |
| cloacae (rpt) | | | | CFU | CFU | | |
| Cryptosporidium | 143 | 3.85 | NA | 6.25 × 10⁶ cells/mL | ≧1 × 10⁵ cells/mL | 150 μL | Neg. |
| parvum | | | | | | | |
| Cryptosporidium | 143 | 2.83 | NA | 6.25 × 10⁶ cells/mL | ≧1 × 10⁵ cells/mL | 150 μL | Neg. |
| parvum | | | | | | | |
| Cryptosporidium | 143 | 6.22 | NA | 6.25 × 10⁶ cells/mL | ≧1 × 10⁵ cells/mL | 150 μL | Neg. |
| parvum | | | | | | | |
| Entamoeba | 144 | 4691.2 | 15.5 | 1.10 × 10⁶ cells/mL | ≧1 × 10⁵ cells/mL | 150 μL | Pos. |
| histolytica | | | | | | | |
| Entamoeba | 144 | 4740.92 | 14.61 | 1.10 × 10⁶ cells/mL | ≧1 × 10⁵ cells/mL | 150 μL | Pos. |
| histolytica | | | | | | | |
| Entamoeba | 144 | 4941.93 | 14.48 | 1.10 × 10⁶ cells/mL | ≧1 × 10⁵ cells/mL | 150 μL | Pos. |
| histolytica | | | | | | | |
| Giardia lamblia | 145 | 9.81 | NA | 6.25 × 10⁶ cells/mL | ≧1 × 10⁵ cells/mL | 150 μL | Neg. |
| Giardia lamblia | 145 | 10.92 | NA | 6.25 × 10⁶ cells/mL | ≧1 × 10⁵ cells/mL | 150 μL | Neg. |
| Giardia lamblia | 145 | 6.22 | NA | 6.25 × 10⁶ cells/mL | ≧1 × 10⁵ cells/mL | 150 μL | Neg. |
| Cryptosporidium | 142 | 3.04 | NA | 3.0 × 10⁷ cells/mL | ≧1 × 10⁵ cells/mL | 15 μL | Neg. |
| meleagridis | | | | | | | |
| Cryptosporidium | 142 | 3.33 | NA | 3.0 × 10⁷ cells/mL | ≧1 × 10⁵ cells/mL | 15 μL | Neg. |
| meleagridis | | | | | | | |
| Cryptosporidium | 142 | 0.88 | NA | 3.0 × 10⁷ cells/mL | ≧1 × 10⁵ cells/mL | 15 μL | Neg. |
| meleagridis | | | | | | | |
| Cryptosporidium | 142 | 3.75 | NA | 3.0 × 10⁷ cells/mL | ≧1 × 10⁵ cells/mL | 15 μL | Neg. |
| meleagridis | | | | | | | |
| Cryptosporidium | 142 | 1.78 | NA | 3.0 × 10⁷ cells/mL | ≧1 × 10⁵ cells/mL | 15 μL | Neg. |
| meleagridis | | | | | | | |
| Cryptosporidium | 142 | 4.15 | NA | 3.0 × 10⁷ cells/mL | ≧1 × 10⁵ cells/mL | 15 μL | Neg. |
| meleagridis | | | | | | | |
| Cryptosporidium | 142 | 9.89 | NA | 3.0 × 10⁷ cells/mL | ≧1 × 10⁵ cells/mL | 15 μL | Neg. |
| meleagridis | | | | | | | |
| Cryptosporidium | 142 | 2.49 | NA | 3.0 × 10⁷ cells/mL | ≧1 × 10⁵ cells/mL | 15 μL | Neg. |
| meleagridis | | | | | | | |
| Cryptosporidium | 142 | 8.82 | NA | 3.0 × 10⁷ cells/mL | ≧1 × 10⁵ cells/mL | 15 μL | Neg. |
| meleagridis | | | | | | | |
| Cryptosporidium | 142 | 1.64 | NA | 3.0 × 10⁷ cells/mL | ≧1 × 10⁵ cells/mL | 15 μL | Neg. |
| meleagridis | | | | | | | |
| Cryptosporidium | 142 | 1.24 | NA | 3.0 × 10⁷ cells/mL | ≧1 × 10⁵ cells/mL | 15 μL | Neg. |
| meleagridis | | | | | | | |
| Cryptosporidium | 142 | 1.66 | NA | 3.0 × 10⁷ cells/mL | ≧1 × 10⁵ cells/mL | 15 μL | Neg. |
| meleagridis | | | | | | | |
| Cryptosporidium | 142 | 1.79 | NA | 3.0 × 10⁷ cells/mL | ≧1 × 10⁵ cells/mL | 15 μL | Neg. |
| meleagridis | | | | | | | |
| Cryptosporidium | 142 | 2.05 | NA | 3.0 × 10⁷ cells/mL | ≧1 × 10⁵ cells/mL | 15 μL | Neg. |
| meleagridis | | | | | | | |
| Cryptosporidium | 142 | 1.18 | NA | 3.0 × 10⁷ cells/mL | ≧1 × 10⁵ cells/mL | 15 μL | Neg. |
| meleagridis | | | | | | | |
| Cryptosporidium | 142 | 2.82 | NA | 3.0 × 10⁷ cells/mL | ≧1 × 10⁵ cells/mL | 15 μL | Neg. |
| meleagridis | | | | | | | |
| Cryptosporidium | 142 | 1.83 | NA | 3.0 × 10⁷ cells/mL | ≧1 × 10⁵ cells/mL | 15 μL | Neg. |
| meleagridis | | | | | | | |
| Cryptosporidium | 142 | 0.34 | NA | 3.0 × 10⁷ cells/mL | ≧1 × 10⁵ cells/mL | 15 μL | Neg. |
| meleagridis | | | | | | | |
| Cryptosporidium | 142 | 3.25 | NA | 3.0 × 10⁷ cells/mL | ≧1 × 10⁵ cells/mL | 15 μL | Neg. |
| meleagridis | | | | | | | |
| Cryptosporidium | 142 | 0.42 | NA | 3.0 × 10⁷ cells/mL | ≧1 × 10⁵ cells/mL | 15 μL | Neg. |
| meleagridis | | | | | | | |
| Cryptosporidium | 142 | 0.14 | NA | 3.0 × 10⁷ cells/mL | ≧1 × 10⁵ cells/mL | 15 μL | Neg. |
| meleagridis | | | | | | | |
| Cryptosporidium | 142 | 1.49 | NA | 3.0 × 10⁷ cells/mL | ≧1 × 10⁵ cells/mL | 15 μL | Neg. |
| meleagridis | | | | | | | |
| Cryptosporidium | 142 | 0.58 | NA | 3.0 × 10⁷ cells/mL | ≧1 × 10⁵ cells/mL | 15 μL | Neg. |
| meleagridis | | | | | | | |
| Cryptosporidium | 142 | 1.55 | NA | 3.0 × 10⁷ cells/mL | ≧1 × 10⁵ cells/mL | 15 μL | Neg. |
| meleagridis | | | | | | | |

### Example 7

The BD MAX™ assay was tested with multiple different isolates of Entamoeba histolytica at the assay LOD in the presence of 10 μL of unpreserved stool matrix per test. 24 replicates per isolate were tested. The BD MAX™ assay detected a variety of different Entamoeba histolytica isolates. The results are shown in Table 5.

TABLE 5

| | | | Conc | | Ct. | BD MAX |
|---|---|---|---|---|---|---|
| Isolate | ATCC# | Type | in SBT | ymaxEP | Score | Result |
| HB-301: | 30190 | Cultured | 13 | 3692.29 | 28.8 | Positive |
| NIH | | Isolate | org/mL | 4259.8 | 30.12 | Positive |
| | | | | 4658.63 | 28.35 | Positive |
| | | | | 4445.23 | 29.16 | Positive |
| | | | | 3623.57 | 31.24 | Positive |
| | | | | 4248.16 | 30.84 | Positive |
| | | | | 4993.12 | 27.91 | Positive |
| | | | | 4143.38 | 30.82 | Positive |
| | | | | 4718.04 | 29.98 | Positive |
| | | | | 4322.62 | 31.11 | Positive |
| | | | | 4827.17 | 30.9 | Positive |
| | | | | 4938.83 | 29.19 | Positive |
| | | | | 2513.48 | 29.57 | Positive |
| | | | | 2953.5 | 32.62 | Positive |
| | | | | 3036.11 | 30.18 | Positive |
| | | | | 3657.4 | 31.59 | Positive |
| | | | | 2761.39 | 29.94 | Positive |
| | | | | 2886.84 | 30.98 | Positive |
| | | | | 3633.27 | 29.05 | Positive |
| | | | | 3160.31 | 31.27 | Positive |
| | | | | 332.57 | 35.89 | Positive |
| | | | | 3020.09 | 32.98 | Positive |
| | | | | 3448.17 | 28.43 | Positive |
| | | | | 2856.23 | 31.77 | Positive |
| H-458: | 30889 | Cultured | 13 | 4208.86 | 31.92 | Positive |
| CDC | | Isolate | org/mL | 3852.52 | 29.38 | Positive |
| | | | | 4600.73 | 28.83 | Positive |
| | | | | 4093.43 | 28.42 | Positive |
| | | | | 3440.67 | 28.84 | Positive |
| | | | | 3705.75 | 29.69 | Positive |
| | | | | 3676.13 | 28.74 | Positive |
| | | | | 3725.05 | 30.58 | Positive |
| | | | | 4989.86 | 28.27 | Positive |
| | | | | 4775.94 | 29.92 | Positive |
| | | | | 4591.33 | 29.98 | Positive |
| | | | | 4110.95 | 32.51 | Positive |
| | | | | 4566.19 | 31.58 | Positive |
| | | | | 4467.29 | 29.24 | Positive |
| | | | | 4157.47 | 31.47 | Positive |
| | | | | 4042.66 | 30.3 | Positive |
| | | | | 4053.97 | 32.32 | Positive |
| | | | | 5247.62 | 28.93 | Positive |
| | | | | 5267.41 | 27.97 | Positive |
| | | | | 4754.97 | 28.65 | Positive |
| | | | | 4415.17 | 29.59 | Positive |
| | | | | 4797.29 | 28.48 | Positive |
| | | | | 4915.8 | 31.86 | Positive |
| | | | | 3839.02 | 28.51 | Positive |
| DKB | 50007 | Cultured | 13 | 890.79 | 37.16 | Positive |
| | | Isolate | org/mL | 1312.98 | 36.43 | Positive |
| | | | | 2225.24 | 35.43 | Positive |
| | | | | 2.99 | NA | Negative |
| | | | | 26.41 | NA | Negative |
| | | | | 2201.85 | 33.34 | Positive |
| | | | | 1179.67 | 36.23 | Positive |
| | | | | 1510.96 | 36.22 | Positive |
| | | | | 3368.77 | 32.84 | Positive |
| | | | | 3177.34 | 35.02 | Positive |
| | | | | 743.44 | 40.11 | Positive |
| | | | | 1865.96 | 35.09 | Positive |
| | | | | 2163.25 | 35.18 | Positive |
| | | | | 1050.86 | 36.33 | Positive |
| | | | | 1655 | 35.52 | Positive |
| | | | | 1791.46 | 35.21 | Positive |
| | | | | 1941.9 | 35.09 | Positive |
| | | | | 7.77 | NA | Negative |
| | | | | 1070.97 | 37.1 | Positive |
| | | | | 17.03 | NA | Negative |
| | | | | 1.91 | NA | Negative |
| | | | | 0.32 | NA | Negative |
| | | | | 2789.16 | 36.18 | Positive |
| | | | | 2537.6 | 36.42 | Positive |
| 200: | 30458 | Frozen | 13 | 3766.93 | 27.82 | Positive |
| NIH | | Isolate | org/mL | 4708.97 | 27.44 | Positive |
| | | | | 5143.31 | 27.74 | Positive |
| | | | | 4078.25 | 27.7 | Positive |
| | | | | 4641.32 | 26.09 | Positive |
| | | | | 4880.21 | 27.54 | Positive |
| | | | | 4477.49 | 27.97 | Positive |
| | | | | 4558.3 | 27.66 | Positive |
| | | | | 5068.43 | 26.51 | Positive |
| | | | | 5213.9 | 27.68 | Positive |
| | | | | 5122.26 | 27.46 | Positive |
| | | | | 5252.53 | 27.4 | Positive |
| | | | | 3436.73 | 27.8 | Positive |
| | | | | 3654.58 | 27.66 | Positive |
| | | | | 4203.64 | 28.36 | Positive |
| | | | | 3587.22 | 27.78 | Positive |
| | | | | 3374.32 | 27.81 | Positive |
| | | | | 3649.92 | 27.34 | Positive |
| | | | | 3285.35 | 27.83 | Positive |
| | | | | 3358.72 | 27.56 | Positive |
| | | | | 3556.5 | 27.81 | Positive |
| | | | | 3893.07 | 27.91 | Positive |
| | | | | 3909.42 | 27.84 | Positive |
| | | | | 3860.65 | 27.68 | Positive |
| HM-1: | 30459 | Frozen | 13 | 3741.63 | 21.85 | Positive |
| IMSS | | Isolate | org/mL | 4025.64 | 21.8 | Positive |
| | | | | 4224.04 | 21.73 | Positive |
| | | | | 4408.48 | 21.9 | Positive |
| | | | | 4033.02 | 21.86 | Positive |
| | | | | 4035.99 | 21.79 | Positive |
| | | | | 4606.63 | 21.8 | Positive |
| | | | | 3689.37 | 21.76 | Positive |
| | | | | 4306.67 | 21.78 | Positive |
| | | | | 4485.92 | 21.63 | Positive |
| | | | | 4236.68 | 21.72 | Positive |
| | | | | 4025.16 | 21.66 | Positive |
| | | | | 4731.57 | 21.61 | Positive |
| | | | | 3861.55 | 21.67 | Positive |
| | | | | 4472.74 | 21.9 | Positive |
| | | | | 4661.65 | 21.77 | Positive |
| | | | | 5151.08 | 21.82 | Positive |
| | | | | 3788.4 | 21.65 | Positive |
| | | | | 5016.46 | 21.81 | Positive |
| | | | | 4821.04 | 23.1 | Positive |
| | | | | 5269.22 | 21.62 | Positive |
| | | | | 4122.29 | 22.18 | Positive |
| | | | | 4838.41 | 21.77 | Positive |
| | | | | 4535.04 | 21.85 | Positive |
| IP: | PRA-357 | Frozen | 13 | 4558.59 | 27.03 | Positive |
| 1182: 2 | | Isolate | org/mL | 4282.77 | 26.7 | Positive |
| | | | | 4403.06 | 26.71 | Positive |
| | | | | 4519.79 | 26.6 | Positive |
| | | | | 4464.05 | 27.17 | Positive |
| | | | | 4514.94 | 26.94 | Positive |
| | | | | 4740.94 | 26.89 | Positive |
| | | | | 4092.67 | 26.65 | Positive |
| | | | | 5027.85 | 26.96 | Positive |
| | | | | 5064.84 | 26.46 | Positive |
| | | | | 5109.14 | 26.79 | Positive |
| | | | | 4958.97 | 26.86 | Positive |
| | | | | 4407 | 27.6 | Positive |
| | | | | 4535.17 | 27.23 | Positive |
| | | | | 4571.14 | 27.15 | Positive |
| | | | | 4196.92 | 26.6 | Positive |
| | | | | 4401.92 | 26.75 | Positive |
| | | | | 4685.94 | 26.89 | Positive |
| | | | | 1735.56 | 35.5 | Positive |
| | | | | 3911.61 | 26.87 | Positive |
| | | | | 4558.06 | 27.33 | Positive |
| | | | | 4681.03 | 26.91 | Positive |
| | | | | 4736.92 | 27.56 | Positive |
| | | | | 5206.35 | 26.91 | Positive |
| HM-3: | 30890 | Cultured | 13 | 4547.02 | 32.7 | Positive |
| IMSS | | Isolate | org/mL | 846.46 | 38.73 | Positive |
| | | | | 4040.67 | 35.61 | Positive |
| | | | | 3912.84 | 35.34 | Positive |
| | | | | 3868.84 | 34.78 | Positive |
| | | | | 2494.87 | 36.65 | Positive |
| | | | | 3765.99 | 33.92 | Positive |
| | | | | 3399.28 | 35.09 | Positive |
| | | | | 4459.57 | 32.73 | Positive |
| | | | | 3083.3 | 35.02 | Positive |
| | | | | 3188.32 | 33.77 | Positive |
| | | | | 4784.16 | 31.72 | Positive |
| | | | | 2379.38 | 35.63 | Positive |
| | | | | 1.7 | NA | Negative |
| | | | | 3686.26 | 33.6 | Positive |
| | | | | 2440.99 | 35.4 | Positive |
| | | | | 4049.49 | 29.89 | Positive |
| | | | | 525.31 | 41.28 | Positive |
| | | | | 3221.64 | 35.16 | Positive |
| | | | | 1822.64 | 36.33 | Positive |
| | | | | 2621.45 | 35.09 | Positive |
| | | | | 2244.92 | 34.96 | Positive |
| | | | | 1.4 | NA | Negative |
| | | | | 3558.48 | 34.14 | Positive |

### Example 8

The 95% LoD for each specimen type was determined by linear dilution of Entamoeba histolytica trophozoites in sample buffer with 104 of the appropriate stool matrix. A minimum of 36 replicates per test level were performed. The LoD is approximately 17 organisms/ml in the sample buffer tube. The results are shown in Table 6 (unpreserved samples) and Table 7 (samples fixed in 10% formalin).

TABLE 6
(unpreserved samples)

| Orgs/mL | | Ct. | BD MAX |
|---|---|---|---|
| in SBT | ymaxEP | Score | Result |
| 0 | 8.09 | NA | Negative |
| 0 | 1.06 | NA | Negative |
| 0 | 3.26 | NA | Negative |
| 0 | 4.88 | NA | Negative |
| 0 | 23.2 | NA | Negative |
| 0 | 1.33 | NA | Negative |
| 0 | 28.37 | NA | Negative |
| 0 | 1.4 | NA | Negative |
| 0 | 0.53 | NA | Negative |
| 0 | 2.24 | NA | Negative |
| 0 | 9.38 | NA | Negative |
| 0 | 0.21 | NA | Negative |
| 0 | 7.35 | NA | Negative |
| 0 | 4.24 | NA | Negative |
| 0 | 37.2 | NA | Negative |
| 0 | 35.49 | NA | Negative |
| 0 | 21.54 | NA | Negative |
| 0 | 7.19 | NA | Negative |
| 0 | 54.92 | NA | Negative |
| 0 | 2.83 | NA | Negative |
| 0 | 10.48 | NA | UNR |
| 0 | 8.33 | NA | Negative |
| 0 | 9.47 | NA | Negative |
| 0 | 2.64 | NA | Negative |
| 0 | 6.22 | NA | Negative |
| 0 | 2.65 | NA | Negative |
| 0 | 1.73 | NA | Negative |
| 0 | 7.28 | NA | Negative |
| 0 | 13.06 | NA | Negative |
| 0 | 0.52 | NA | Negative |
| 0 | 24.9 | NA | Negative |
| 0 | 3.8 | NA | Negative |
| 0 | 10.57 | NA | Negative |
| 0 | 0.81 | NA | Negative |
| 0 | 18.32 | NA | Negative |
| 0 | 5.25 | NA | Negative |
| 1.5 | 3491.96 | 28.83 | Positive |
| 1.5 | 2.31 | NA | Negative |
| 1.5 | 10.87 | NA | Negative |
| 1.5 | 3.16 | NA | Negative |
| 1.5 | 21.73 | NA | Negative |
| 1.5 | 0.16 | NA | Negative |
| 1.5 | 45.07 | NA | Negative |
| 1.5 | 6.79 | NA | Negative |
| 1.5 | 1.67 | NA | Negative |
| 1.5 | 4715.47 | 29.13 | Positive |
| 1.5 | 7.24 | NA | Negative |
| 1.5 | 5.99 | NA | Negative |
| 1.5 | 3095.48 | 33.29 | Positive |
| 1.5 | 3482.41 | 34.47 | Positive |
| 1.5 | 4300.99 | 34.14 | Positive |
| 1.5 | 19.73 | NA | Negative |
| 1.5 | 24.09 | NA | Negative |
| 1.5 | 4456.34 | 34.2 | Positive |
| 1.5 | 54.38 | NA | Negative |
| 1.5 | 3.01 | NA | Negative |
| 1.5 | 8.39 | NA | Negative |
| 1.5 | 7.88 | NA | Negative |
| 1.5 | 1.75 | NA | Negative |
| 1.5 | 7.07 | NA | Negative |
| 1.5 | 33.9 | NA | Negative |
| 1.5 | 2.56 | NA | Negative |
| 1.5 | 8.51 | NA | Negative |
| 1.5 | 2.04 | NA | Negative |
| 1.5 | 17.23 | NA | Negative |
| 1.5 | 1.06 | NA | Negative |
| 1.5 | 67.26 | NA | Negative |
| 1.5 | 5.14 | NA | Negative |
| 1.5 | 8.24 | NA | Negative |
| 1.5 | 0.4 | NA | Negative |
| 1.5 | 10.67 | NA | Negative |
| 1.5 | 3.11 | NA | Negative |
| 3 | 3567.46 | 31.39 | Positive |
| 3 | 0.64 | NA | Negative |
| 3 | 3462.65 | 32.5 | Positive |
| 3 | 3.23 | NA | Negative |
| 3 | 4948.93 | 28.59 | Positive |
| 3 | 2.4 | NA | Negative |
| 3 | 3143.06 | 32.1 | Positive |
| 3 | 2.41 | NA | Negative |
| 3 | 12.22 | NA | Negative |
| 3 | 0.57 | NA | Negative |
| 3 | 12.03 | NA | Negative |
| 3 | 3948.89 | 31.95 | Positive |
| 3 | 13.89 | NA | Negative |
| 3 | 3678.22 | 33.52 | Positive |
| 3 | 23.9 | NA | Negative |
| 3 | 34.43 | NA | Negative |
| 3 | 50.52 | NA | Negative |
| 3 | 7.36 | NA | Negative |
| 3 | 50.44 | NA | Negative |
| 3 | 36.33 | NA | Negative |
| 3 | 24.59 | NA | Negative |
| 3 | 4797.13 | 31.82 | Positive |
| 3 | 7.94 | NA | Negative |
| 3 | 5156.56 | 27.71 | Positive |
| 3 | 13.05 | NA | Negative |
| 3 | 4059.67 | 30.97 | Positive |
| 3 | 4.88 | NA | Negative |
| 3 | 4.07 | NA | Negative |
| 3 | 5148.21 | 32.48 | Positive |
| 3 | 5848.21 | 27.65 | Positive |
| 3 | 23.72 | NA | Negative |
| 3 | 3971.89 | 26.78 | Positive |
| 3 | 13.25 | NA | Negative |
| 3 | 4462.91 | 29.75 | Positive |
| 3 | 5246.72 | 27.84 | Positive |
| 3 | 5685.43 | 30.5 | Positive |
| 6 | 3904.47 | 29.53 | Positive |
| 6 | 4222.69 | 26.97 | Positive |
| 6 | 16.53 | NA | Negative |
| 6 | 31.52 | NA | Negative |
| 6 | 6.81 | NA | Negative |
| 6 | 0.59 | NA | Negative |
| 6 | 8.42 | NA | Negative |
| 6 | 3240.6 | 31.37 | Positive |
| 6 | 4490.59 | 28.01 | Positive |
| 6 | 4083.43 | 33.06 | Positive |
| 6 | 7.7 | NA | Negative |
| 6 | 2.8 | NA | Negative |
| 6 | 3524.76 | 33.5 | Positive |
| 6 | 4533.07 | 27.87 | Positive |
| 6 | 20.68 | NA | Negative |
| 6 | 8.97 | NA | Negative |
| 6 | 2756.57 | 37.21 | Positive |
| 6 | 4436.58 | 32.45 | Positive |
| 6 | 2664.51 | 34.96 | Positive |
| 6 | 33.21 | NA | Negative |
| 6 | 6077.66 | 28.47 | Positive |
| 6 | 4.8 | NA | Negative |
| 6 | 24.36 | NA | Negative |
| 6 | 4964.91 | 28.27 | Positive |
| 6 | 4318 | 25.96 | Positive |
| 6 | 2354.93 | 34.63 | Positive |
| 6 | 3905.86 | 30.62 | Positive |
| 6 | 4.91 | NA | Negative |
| 6 | 4549.72 | 28.82 | Positive |
| 6 | 1.85 | NA | Negative |
| 6 | 3215.39 | 32.45 | Positive |
| 6 | 49.2 | NA | Negative |
| 6 | 3309.9 | 33.41 | Positive |
| 6 | 2.33 | NA | Negative |
| 6 | 15.14 | NA | Negative |
| 6 | 6286.84 | 31.04 | Positive |
| 12 | 1.82 | NA | Negative |
| 12 | 3898.36 | 28.51 | Positive |
| 12 | 2577.56 | 32.74 | Positive |
| 12 | 3947.57 | 31.65 | Positive |
| 12 | 4232.4 | 29.27 | Positive |
| 12 | 4056.49 | 30.95 | Positive |
| 12 | 3445.95 | 28.9 | Positive |
| 12 | 3205.26 | 29.07 | Positive |
| 12 | 4172.74 | 31.9 | Positive |
| 12 | 4480.14 | 26.57 | Positive |
| 12 | 3929.88 | 30.82 | Positive |
| 12 | 3.12 | NA | Negative |
| 12 | 4313.96 | 26.69 | Positive |
| 12 | 1185.52 | 36.43 | Positive |
| 12 | 5597.67 | 29.16 | Positive |
| 12 | 4376.16 | 27.52 | Positive |
| 12 | 5636.53 | 27.57 | Positive |
| 12 | 3860.33 | 34.42 | Positive |
| 12 | 2750.81 | 34.32 | Positive |
| 12 | 3349.49 | 28.92 | Positive |
| 12 | 5062.72 | 27.54 | Positive |
| 12 | 4569.68 | 31.42 | Positive |
| 12 | 3937.91 | 33.31 | Positive |
| 12 | 4276.11 | 30.54 | Positive |
| 12 | 3854.83 | 25.66 | Positive |
| 12 | 4152.17 | 28.01 | Positive |
| 12 | 4215.86 | 27.49 | Positive |
| 12 | 3870.53 | 32.41 | Positive |
| 12 | 2043.52 | 36.35 | Positive |
| 12 | 5068.25 | 25.19 | Positive |
| 12 | 3218 | 31.61 | Positive |
| 12 | 1152.03 | 32.64 | Positive |
| 12 | 3687.64 | 29.77 | Positive |
| 12 | 3955.63 | 30.17 | Positive |
| 12 | 5469.84 | 25.48 | Positive |
| 12 | 4896.75 | 31.41 | Positive |
| 24 | 3386.13 | 28.93 | Positive |
| 24 | 4359.59 | 30.68 | Positive |
| 24 | 4561.76 | 26.86 | Positive |
| 24 | 5393.38 | 25.78 | Positive |
| 24 | 4074.4 | 26.36 | Positive |
| 24 | 5100.02 | 25.65 | Positive |
| 24 | 3279.79 | 27.87 | Positive |
| 24 | 3909.66 | 26.05 | Positive |
| 24 | 4640.2 | 24.72 | Positive |
| 24 | 4026.76 | 31.43 | Positive |
| 24 | 4491.63 | 27.01 | Positive |
| 24 | 4446.85 | 27.16 | Positive |
| 24 | 3212.29 | 32.23 | Positive |
| 24 | 3943.76 | 30.58 | Positive |
| 24 | 5016.95 | 30.6 | Positive |
| 24 | 4219.82 | 31.99 | Positive |
| 24 | 52.57 | NA | Negative |
| 24 | 4963.69 | 29.01 | Positive |
| 24 | 2642.13 | 33.08 | Positive |
| 24 | 3991.36 | 28.02 | Positive |
| 24 | 4739.55 | 26.39 | Positive |
| 24 | 5324.49 | 26.34 | Positive |
| 24 | 4533.39 | 33.16 | Positive |
| 24 | 5013.46 | 28.74 | Positive |
| 24 | 3740.59 | 26.8 | Positive |
| 24 | 4114.65 | 24.72 | Positive |
| 24 | 5059.97 | 28.01 | Positive |
| 24 | 4404.69 | 29.08 | Positive |
| 24 | 4268.5 | 26.44 | Positive |
| 24 | 4405.85 | 29.75 | Positive |
| 24 | 3104.97 | 27 | Positive |
| 24 | 3671.19 | 25.34 | Positive |
| 24 | 3847.73 | 26.56 | Positive |
| 24 | 4490.29 | 25.81 | Positive |
| 24 | 5898.68 | 26.59 | Positive |
| 24 | 5421.98 | 28.57 | Positive |

TABLE 7
(Samples fixed in 10% formalin)

| Orgs/mL | | Ct. | BD MAX |
|---|---|---|---|
| in SBT | ymaxEP | Score | Result |
| 0 | 15.1 | NA | Negative |
| 0 | 7.52 | NA | Negative |
| 0 | 26.78 | NA | Negative |
| 0 | 11.24 | NA | Negative |
| 0 | 15.03 | NA | Negative |
| 0 | 7.83 | NA | Negative |
| 0 | 20.63 | NA | Negative |
| 0 | 2.11 | NA | Negative |
| 0 | 21.41 | NA | Negative |
| 0 | 16.82 | NA | Negative |
| 0 | 61.39 | NA | Negative |
| 0 | 4.4 | NA | Negative |
| 0 | 27.86 | NA | Negative |
| 0 | 5.56 | NA | Negative |
| 0 | 3.4 | NA | Negative |
| 0 | 2.05 | NA | Negative |
| 0 | 14.6 | NA | Negative |
| 0 | 7.94 | NA | Negative |
| 0 | 44.54 | NA | Negative |
| 0 | 2.04 | NA | Negative |
| 0 | 3.06 | NA | Negative |
| 0 | 13.37 | NA | Negative |
| 0 | 22.34 | NA | Negative |
| 0 | 5.02 | NA | Negative |
| 0 | 22.35 | NA | Negative |
| 0 | 5.09 | NA | Negative |
| 0 | 6.65 | NA | Negative |
| 0 | 4.91 | NA | Negative |
| 0 | 14.62 | NA | Negative |
| 0 | 7.49 | NA | Negative |
| 0 | 4.98 | NA | Negative |
| 0 | 7.55 | NA | Negative |
| 0 | 9.14 | NA | Negative |
| 0 | 3.71 | NA | Negative |
| 0 | 95.74 | NA | Negative |
| 0 | 5.05 | NA | Negative |
| 1.5 | 2593.13 | 33.67 | Positive |
| 1.5 | 5.04 | NA | Negative |
| 1.5 | 40.45 | NA | Negative |
| 1.5 | 14.97 | NA | Negative |
| 1.5 | 3763.41 | 29.38 | Positive |
| 1.5 | 9.24 | NA | Negative |
| 1.5 | 2788.37 | 30.25 | Positive |
| 1.5 | 11.37 | NA | Negative |
| 1.5 | 4.45 | NA | Negative |
| 1.5 | 10.18 | NA | Negative |
| 1.5 | 32.84 | NA | Negative |
| 1.5 | 4376.92 | 31.41 | Positive |
| 1.5 | 2837.85 | 30.61 | Positive |
| 1.5 | 7.01 | NA | Negative |
| 1.5 | 28.18 | NA | Negative |
| 1.5 | 19.62 | NA | Negative |
| 1.5 | 3.05 | NA | Negative |
| 1.5 | 4116.38 | 34.29 | Positive |
| 1.5 | 26.08 | NA | Negative |
| 1.5 | 2954.88 | 31.9 | Positive |
| 1.5 | 14.31 | NA | Negative |
| 1.5 | 7.44 | NA | Negative |
| 1.5 | 16 | NA | Negative |
| 1.5 | 2307.69 | 32.03 | Positive |
| 1.5 | 16.65 | NA | Negative |
| 1.5 | 5.7 | NA | Negative |
| 1.5 | 32.94 | NA | Negative |
| 1.5 | 26.7 | NA | Negative |
| 1.5 | 12.55 | NA | Negative |
| 1.5 | 3.14 | NA | Negative |
| 1.5 | 55.11 | NA | Negative |
| 1.5 | 2985.7 | 33.85 | Positive |
| 1.5 | 3257.04 | 35.16 | Positive |
| 1.5 | 4.76 | NA | Negative |
| 1.5 | 18.07 | NA | Negative |
| 1.5 | 21.08 | NA | Negative |
| 3 | 3153.69 | 32.16 | Positive |
| 3 | 3694.7 | 33.31 | Positive |
| 3 | 2278.04 | 34.18 | Positive |
| 3 | 3876.12 | 29.43 | Positive |
| 3 | 27.92 | NA | Negative |
| 3 | 5065.1 | 29.76 | Positive |
| 3 | 23.23 | NA | Negative |
| 3 | 11.5 | NA | Negative |
| 3 | 10.1 | NA | Negative |
| 3 | 12.91 | NA | Negative |
| 3 | 44.64 | NA | Negative |
| 3 | 3501.07 | 33.35 | Positive |
| 3 | 29.05 | NA | Negative |
| 3 | 8.96 | NA | Negative |
| 3 | 2.24 | NA | Negative |
| 3 | 2390.42 | 32.18 | Positive |
| 3 | 4.19 | NA | Negative |
| 3 | 4616.76 | 31.14 | Positive |
| 3 | 31.35 | NA | Negative |
| 3 | 9.45 | NA | Negative |
| 3 | 2.9 | NA | Negative |
| 3 | 4153.36 | 32.83 | Positive |
| 3 | 33.42 | NA | Negative |
| 3 | 21.34 | NA | Negative |
| 3 | 3384.98 | 32.33 | Positive |
| 3 | 1.43 | NA | Negative |
| 3 | 6.79 | NA | Negative |
| 3 | 3324.58 | 32.66 | Positive |
| 3 | 3294.33 | 31.54 | Positive |
| 3 | 13.62 | NA | Negative |
| 3 | 3.85 | NA | Negative |
| 3 | 3590.8 | 33.27 | Positive |
| 3 | 2.18 | NA | Negative |
| 3 | 4876.76 | 30.19 | Positive |
| 3 | 68.35 | NA | Negative |
| 3 | 24.04 | NA | Negative |
| 6 | 10.62 | NA | Negative |
| 6 | 3579.07 | 34.67 | Positive |
| 6 | 20.58 | NA | Negative |
| 6 | 4019.37 | 28.65 | Positive |
| 6 | 4506.32 | 27.55 | Positive |
| 6 | 4583.14 | 30.85 | Positive |
| 6 | 3318.77 | 31.31 | Positive |
| 6 | 3132.25 | 28.12 | Positive |
| 6 | 4535.77 | 26.08 | Positive |
| 6 | 5.31 | NA | Negative |
| 6 | 3849.97 | 30.09 | Positive |
| 6 | 36.99 | NA | Negative |
| 6 | 3080.65 | 31.44 | Positive |
| 6 | 6.35 | NA | Negative |
| 6 | 2186.43 | 30.14 | Positive |
| 6 | 4122.31 | 29.24 | Positive |
| 6 | 4615.67 | 28.06 | Positive |
| 6 | 4671.13 | 32.32 | Positive |
| 6 | 3.47 | NA | Negative |
| 6 | 3341.52 | 29.83 | Positive |
| 6 | 16.04 | NA | Negative |
| 6 | 4549.83 | 32.4 | Positive |
| 6 | 5203.16 | 28.67 | Positive |
| 6 | 3739.9 | 30.76 | Positive |
| 6 | 4.01 | NA | Negative |
| 6 | 4856.83 | 33.09 | Positive |
| 6 | 3898.6 | 31.38 | Positive |
| 6 | 3887.04 | 29.64 | Positive |
| 6 | 5.94 | NA | Negative |
| 6 | 4947.47 | 27.78 | Positive |
| 6 | 3198.17 | 30.84 | Positive |
| 6 | 2987.71 | 31.81 | Positive |
| 6 | 7.67 | NA | Negative |
| 6 | 3910.92 | 33.44 | Positive |
| 6 | 8.25 | NA | Negative |
| 6 | 24.5 | NA | Negative |
| 12 | 3621.08 | 29.5 | Positive |
| 12 | 3719.92 | 27.32 | Positive |
| 12 | 2277.49 | 32.02 | Positive |
| 12 | 3896.73 | 29.38 | Positive |
| 12 | 3731.83 | 30.55 | Positive |
| 12 | 3593.57 | 32.18 | Positive |
| 12 | 3000.19 | 31.1 | Positive |
| 12 | 3227.4 | 30.55 | Positive |
| 12 | 3637.22 | 32.33 | Positive |
| 12 | 4641.37 | 29.24 | Positive |
| 12 | 3335.53 | 32.74 | Positive |
| 12 | 3266.99 | 31.75 | Positive |
| 12 | 3393.99 | 29 | Positive |
| 12 | 26.89 | NA | Negative |
| 12 | 3387.11 | 31.86 | Positive |
| 12 | 3685.94 | 29.31 | Positive |
| 12 | 4538.31 | 31.05 | Positive |
| 12 | 3873.71 | 31.47 | Positive |
| 12 | 1216.2 | 35.98 | Positive |
| 12 | 14.66 | NA | Negative |
| 12 | 3660.51 | 29.7 | Positive |
| 12 | 3925.71 | 29.NA | Positive |
| 12 | 3891.51 | 30.67 | Positive |
| 12 | 33.19 | NA | Negative |
| 12 | 3308.03 | 32.32 | Positive |
| 12 | 3753.85 | 29.96 | Positive |
| 12 | 3798.92 | 29.85 | Positive |
| 12 | 3863.38 | 32.11 | Positive |
| 12 | 4.91 | NA | Negative |
| 12 | 5013.19 | 27.47 | Positive |
| 12 | 3495.4 | 27.87 | Positive |
| 12 | 3673.51 | 29.83 | Positive |
| 12 | 3532.78 | 31.31 | Positive |
| 12 | 4185.77 | 28.92 | Positive |
| 12 | 5206.82 | 28.89 | Positive |
| 12 | 4941.56 | 29.81 | Positive |
| 24 | 3474.34 | 27.41 | Positive |
| 24 | 3701.02 | 28.45 | Positive |
| 24 | 3292.8 | 27.48 | Positive |
| 24 | 2971.68 | 32.76 | Positive |
| 24 | 4027.63 | 27.64 | Positive |
| 24 | 4598.32 | 26.39 | Positive |
| 24 | 2909.48 | 30.82 | Positive |
| 24 | 3795.26 | 27.74 | Positive |
| 24 | 4331.92 | 27.15 | Positive |
| 24 | 4939.44 | 26.47 | Positive |
| 24 | 3931.84 | 30.56 | Positive |
| 24 | 4517.49 | 29.47 | Positive |
| 24 | 3195.57 | 27.8 | Positive |
| 24 | 3213.45 | 31.64 | Positive |
| 24 | 3810.56 | 30.76 | Positive |
| 24 | 3794.57 | 28.82 | Positive |
| 24 | 3823.73 | 27.67 | Positive |
| 24 | 3995.24 | 29.56 | Positive |
| 24 | 2261.02 | 33.99 | Positive |
| 24 | 2942.54 | 29.82 | Positive |
| 24 | 3945.75 | 26.62 | Positive |
| 24 | 4596.59 | 28.05 | Positive |
| 24 | 3531.07 | 31.65 | Positive |
| 24 | 3676.28 | 30.64 | Positive |
| 24 | 3102.46 | 30.05 | Positive |
| 24 | 4364.4 | 27.12 | Positive |
| 24 | 4463.09 | 29.61 | Positive |
| 24 | 4207.13 | 28.5 | Positive |
| 24 | 4370.07 | 28.92 | Positive |
| 24 | 4883.31 | 26.95 | Positive |
| 24 | 3342.28 | 28.06 | Positive |
| 24 | 3213.18 | 28.92 | Positive |
| 24 | 3638.74 | 29.5 | Positive |
| 24 | 4580.64 | 28.87 | Positive |
| 24 | 4936.54 | 30.09 | Positive |
| 24 | 5545.29 | 28.4 | Positive |

### Example 9

### Detection of E. histolytica in Mixed Infections

E. histolytica detection was validated in samples comprising mixtures of two or more organisms, which can simulate multiple infection specimens. A low level of one target (Low Target) was spiked into unpreserved stool with high levels (High Level) of other organisms.

As shown in Table 8, the BD MAX™ assay detected E. histolytica near the LoD in simulated multiple infection specimens containing high titer Cryptosporidium parvum, Giardia lamblia, and Entamoeba dispar. E. dispar was included in High Level mixes to confirm that presence of E. dispar does not block amplification of E. histolytica.

TABLE 8

| | | | E. | BD MAX ™ |
|---|---|---|---|---|
| | High Level | E. | histolytica | Entamoeba |
| Low Target | 1e5 | histolytica | Ct. | histolytica |
| 2X LoD | organisms/mL | ymaxEP | Score | Result |
| Giardia (14.8 orgs/mL) | Crypto, E. hist | 4828.31 | 13.6 | Positive |
| Crypto (320 orgs/mL) | Giardia, E. hist | 6089.62 | 12.49 | Positive |
| E. hist (34 orgs/mL) | Giardia, Crypto | 5478.54 | 24.19 | Positive |
| E. hist (34 orgs/mL) | Giardia, Crypto, | 5030.86 | 24.97 | Positive |
| | E. dispar | | | |
| Giardia (14.8 orgs/mL) | Crypto, E. hist | 5044.41 | 13.6 | Positive |
| Crypto (320 orgs/mL) | Giardia, E. hist | 5943.12 | 13.42 | Positive |
| E. hist (34 orgs/mL) | Giardia, Crypto | 5416.67 | 24.05 | Positive |
| E. hist (34 orgs/mL) | Giardia, Crypto, | 5117.91 | 23.79 | Positive |
| | E. dispar | | | |
| Giardia (14.8 orgs/mL) | Crypto, E. hist | 6676.09 | 12.41 | Positive |
| Crypto (320 orgs/mL) | Giardia, E. hist | 6516.32 | 12.27 | Positive |
| E. hist (34 orgs/mL) | Giardia, Crypto | 5676.63 | 22.77 | Positive |
| E. hist (34 orgs/mL) | Giardia, Crypto, | 5359.65 | 24.02 | Positive |
| | E. dispar | | | |
| Giardia (14.8 orgs/mL) | Crypto, E. hist | 5495.68 | 13.58 | Positive |
| Crypto (320 orgs/mL) | Giardia, E. hist | 6387.42 | 12.31 | Positive |
| E. hist (34 orgs/mL) | Giardia, Crypto | 6064.2 | 24.56 | Positive |
| E. hist (34 orgs/mL) | Giardia, Crypto, | 5696.13 | 26.12 | Positive |
| | E. dispar | | | |
| Giardia (14.8 orgs/mL) | Crypto, E. hist | 5033.92 | 14.39 | IND |
| Crypto (320 orgs/mL) | Giardia, E. hist | 6903.24 | 12.6 | Positive |
| E. hist (34 orgs/mL) | Giardia, Crypto | 5631.31 | 22.57 | Positive |
| E. hist (34 orgs/mL) | Giardia, Crypto, | 5465.78 | 24.82 | Positive |
| | E. dispar | | | |
| Giardia (14.8 orgs/mL) | Crypto, E. hist | 6008.94 | 13.46 | Positive |
| Crypto (320 orgs/mL) | Giardia, E. hist | 7223.09 | 12.37 | Positive |
| E. hist (34 orgs/mL) | Giardia, Crypto | 6434 | 24.19 | Positive |
| E. hist (34 orgs/mL) | Giardia, Crypto, | 5328.59 | 25.26 | Positive |
| | E. dispar | | | |
| Giardia (14.8 orgs/mL) | Crypto, E. hist | 4477.14 | 17.61 | Positive |
| Crypto (320 orgs/mL) | Giardia, E. hist | 5084 | 16.84 | Positive |
| E. hist (34 orgs/mL) | Giardia, Crypto | 4251.15 | 28.77 | Positive |
| E. hist (34 orgs/mL) | Giardia, Crypto, | 3772.95 | 29.51 | Positive |
| | E. dispar | | | |
| Giardia (14.8 orgs/mL) | Crypto, E. hist | 4757.42 | 16.02 | Positive |
| Crypto (320 orgs/mL) | Giardia, E. hist | 5054.86 | 15.36 | Positive |
| E. hist (34 orgs/mL) | Giardia, Crypto | 4645.74 | 28.32 | Positive |
| E. hist (34 orgs/mL) | Giardia, Crypto, | 3533.73 | 30.24 | Positive |
| | E. dispar | | | |
| Giardia (14.8 orgs/mL) | Crypto, E. hist | 5811.4 | 15.81 | Positive |
| Crypto (320 orgs/mL) | Giardia, E. hist | 5439.11 | 16.9 | Positive |
| E. hist (34 orgs/mL) | Giardia, Crypto | 5751.51 | 27.71 | Positive |
| E. hist (34 orgs/mL) | Giardia, Crypto, | 3627.36 | 32.99 | Positive |
| | E. dispar | | | |
| Giardia (14.8 orgs/mL) | Crypto, E. hist | 5751.85 | 17.9 | Positive |
| Crypto (320 orgs/mL) | Giardia, E. hist | 6723.57 | 17.28 | Positive |
| E. hist (34 orgs/mL) | Giardia, Crypto | 5849.82 | 27.19 | Positive |
| E. hist (34 orgs/mL) | Giardia, Crypto, | 5061.58 | 30.36 | Positive |
| | E. dispar | | | |
| Giardia (14.8 orgs/mL) | Crypto, E. hist | 6473.61 | 17.22 | Positive |
| Crypto (320 orgs/mL) | Giardia, E. hist | 7477.47 | 17.13 | Positive |
| E. hist (34 orgs/mL) | Giardia, Crypto | 5327.13 | 28.24 | Positive |
| E. hist (34 orgs/mL) | Giardia, Crypto, | 4489.42 | 30.43 | Positive |
| | E. dispar | | | |
| Giardia (14.8 orgs/mL) | Crypto, E. hist | 8090 | 17.11 | Positive |
| Crypto (320 orgs/mL) | Giardia, E. hist | 6631.47 | 16.16 | Positive |
| E. hist (34 orgs/mL) | Giardia, Crypto | 5981.26 | 27.13 | Positive |
| E. hist (34 orgs/mL) | Giardia, Crypto, | 5430.95 | 30.13 | Positive |
| | E. dispar | | | |
| Giardia (14.8 orgs/mL) | Crypto, E. hist | 5022.12 | 12.73 | Positive |
| Crypto (320 orgs/mL) | Giardia, E. hist | 5027.84 | 12.69 | Positive |
| E. hist (34 orgs/mL) | Giardia, Crypto | 4204.53 | 23.16 | Positive |
| E. hist (34 orgs/mL) | Giardia, Crypto, | 4585.06 | 22.89 | Positive |
| | E. dispar | | | |
| Giardia (14.8 orgs/mL) | Crypto, E. hist | 4390.51 | 13.49 | Positive |
| Crypto (320 orgs/mL) | Giardia, E. hist | 5386.32 | 12.33 | Positive |
| E. hist (34 orgs/mL) | Giardia, Crypto | 2968.36 | 23.97 | Positive |
| E. hist (34 orgs/mL) | Giardia, Crypto, | 3867.53 | 24.04 | Positive |
| | E. dispar | | | |
| Giardia (14.8 orgs/mL) | Crypto, E. hist | 5022.29 | 12.44 | Positive |
| Crypto (320 orgs/mL) | Giardia, E. hist | 5639.25 | 12.24 | Positive |
| E. hist (34 orgs/mL) | Giardia, Crypto | 4489.11 | 24.18 | Positive |
| E. hist (34 orgs/mL) | Giardia, Crypto, | 4661.22 | 24.43 | Positive |
| | E. dispar | | | |
| Giardia (14.8 orgs/mL) | Crypto, E. hist | 5832.61 | 13.61 | Positive |
| Crypto (320 orgs/mL) | Giardia, E. hist | 6164.83 | 13.87 | Positive |
| E. hist (34 orgs/mL) | Giardia, Crypto | 5297.19 | 24.22 | Positive |
| E. hist (34 orgs/mL) | Giardia, Crypto, | 6082.09 | 23.92 | Positive |
| | E. dispar | | | |
| Giardia (14.8 orgs/mL) | Crypto, E. hist | 6067.02 | 13.59 | Positive |
| Crypto (320 orgs/mL) | Giardia, E. hist | 5287.48 | 12.78 | Positive |
| E. hist (34 orgs/mL) | Giardia, Crypto | 4913.7 | 23.74 | Positive |
| E. hist (34 orgs/mL) | Giardia, Crypto, | 5697.28 | 23.65 | Positive |
| | E. dispar | | | |
| Giardia (14.8 orgs/mL) | Crypto, E. hist | 5484.9 | 13.62 | Positive |
| Crypto (320 orgs/mL) | Giardia, E. hist | 5370.17 | 13.59 | Positive |
| E. hist (34 orgs/mL) | Giardia, Crypto | 3754.33 | 28.22 | Positive |
| E. hist (34 orgs/mL) | Giardia, Crypto, | 5239.2 | 24.87 | Positive |
| | E. dispar | | | |
| Giardia (14.8 orgs/mL) | Crypto, E. hist | 5605.26 | 15.98 | Positive |
| Crypto (320 orgs/mL) | Giardia, E. hist | 5264.29 | 17.72 | Positive |
| E. hist (34 orgs/mL) | Giardia, Crypto | 4254.99 | 28.8 | Positive |
| E. hist (34 orgs/mL) | Giardia, Crypto, | 3565.21 | 32.04 | Positive |
| | E. dispar | | | |
| Giardia (14.8 orgs/mL) | Crypto, E. hist | 4356.19 | 18.15 | Positive |
| Crypto (320 orgs/mL) | Giardia, E. hist | 4987.91 | 16.89 | Positive |
| E. hist (34 orgs/mL) | Giardia, Crypto | 4404.36 | 26.14 | Positive |
| E. hist (34 orgs/mL) | Giardia, Crypto, | 2850.61 | 33.56 | Positive |
| | E. dispar | | | |
| Giardia (14.8 orgs/mL) | Crypto, E. hist | 5061.4 | 15.43 | Positive |
| Crypto (320 orgs/mL) | Giardia, E. hist | 5565.34 | 16.43 | Positive |
| E. hist (34 orgs/mL) | Giardia, Crypto | 5059.12 | 28.52 | Positive |
| E. hist (34 orgs/mL) | Giardia, Crypto, | 3525.34 | 33.36 | Positive |
| | E. dispar | | | |
| Giardia (14.8 orgs/mL) | Crypto, E. hist | 7281.34 | 15.65 | Positive |
| Crypto (320 orgs/mL) | Giardia, E. hist | 4989.58 | 18.9 | Positive |
| E. hist (34 orgs/mL) | Giardia, Crypto | 4563.95 | 27.35 | Positive |
| E. hist (34 orgs/mL) | Giardia, Crypto, | 4122.36 | 31.2 | Positive |
| | E. dispar | | | |
| Giardia (14.8 orgs/mL) | Crypto, E. hist | 5660.56 | 16.28 | Positive |
| Crypto (320 orgs/mL) | Giardia, E. hist | 5290.42 | 16.6 | Positive |
| E. hist (34 orgs/mL) | Giardia, Crypto | 4444.64 | 29.27 | Positive |
| E. hist (34 orgs/mL) | Giardia, Crypto, | 4499.42 | 30.73 | Positive |
| | E. dispar | | | |
| Giardia (14.8 orgs/mL) | Crypto, E. hist | 6979.62 | 16.22 | Positive |
| Crypto (320 orgs/mL) | Giardia, E. hist | 6492.92 | 17.24 | Positive |
| E. hist (34 orgs/mL) | Giardia, Crypto | 4439.37 | 28.88 | Positive |
| E. hist (34 orgs/mL) | Giardia, Crypto, | 4859.4 | 29.41 | Positive |
| | E. dispar | | | |

As shown in Table 8, the BD MAX™ assay detected the presence of E. histolytica at low levels, and at high levels. Moreover, the presence of high levels of E. dispar did not interfere with detection of E. histolytica. Accordingly, it is contemplated that methods of detecting E. histolytica in accordance with some embodiments herein are sensitive to very low levels of E. histolytica, and are not compromised by the presence of high levels of E. dispar.

### Example 10

### Validation of BD MAX™ Results by Sequencing

The BD MAX™ assay was compared to a validated alternate PCR and bi-directional sequencing approach. A clinical simulation study was performed using retrospective archived stool specimens representing both unpreserved and 10% formalin fixed stool types. The BD MAX™ assay was performed on the specimens. A validated alternate PCR and bi-directional sequencing assay was also performed on the specimens. Specimens were considered positive for the alternate PCR and bi-directional sequencing assay if their top BLAST hit was E. histolytica. Only specimens for which the alternate PCR/bi-directional sequencing results agreed with the original site reference method were included in performance calculations. The results are summarized in Tables 9.1, 9.2 and 9.3.

TABLE 9.1

| Entamoeba histolytica | | Confirmed Alternate PCR and Sequencing | | |
|---|---|---|---|---|
| Combined Specimen Type | | Positive | Negative | Total |
| BD MAX ™ | Positive | 7 | 0 | 7 |
| Enteric | Negative | 0 | 522 | 522 |
| Parasite | Total | 7 | 522 | 529 |
| Panel | | | | |

Positive Percent Agreement: 100% [95% CI: 64.57%-100%]
Negative Percent Agreement: 100% [95% CI: 99.27%-100%]

TABLE 9.2

| Entamoeba histolytica | | Confirmed Alternate PCR and Sequencing | | |
|---|---|---|---|---|
| Unpreserved Specimens | | Positive | Negative | Total |
| BD MAX ™ | Positive | 1 | 0 | 1 |
| Enteric | Negative | 0 | 301 | 301 |
| Parasite | Total | 1 | 301 | 302 |
| Panel | | | | |

Positive Percent Agreement: 100% [95% CI: 20.65%-100%]
Negative Percent Agreement: 100% [95% CI: 98.74%-100%]

TABLE 9.3

| Entamoeba histolytica | | Confirmed Alternate PCR and Sequencing | | |
|---|---|---|---|---|
| 10% Formalin-Fixed Specimens | | Positive | Negative | Total |
| BD MAX ™ | Positive | 6 | 0 | 6 |
| Enteric | Negative | 0 | 221 | 221 |
| Parasite | Total | 6 | 221 | 227 |
| Panel | | | | |

Positive Percent Agreement: 100% [95% CI: 60.97%-100%]
Negative Percent Agreement: 100% [95% CI: 98.29%-100%]

Both unpreserved specimens and 10% formalin-fixed specimens exhibited 100% concordance between the BDMAX™ assay and the alternate PCR and sequencing method. Furthermore, a number of specimens were found to contain non-pathogenic Entamoeba species which the BD MAX™ E. histolytica assay correctly called as “negative”. Accordingly, it is contemplated that methods of detecting E. histolytica nucleic acids in accordance with some embodiments herein provide highly accurate results, characterized by minimizing cross-reactivity with other organisms, and minimizing both false negatives and false positives.

The raw data for Tables 9.1-9.3 are shown in Tables 10.1-10.2.

TABLE 10.1

| | | | | | AltPCR | AltPCR | AltPCR |
|---|---|---|---|---|---|---|---|
| | | BD MAX | BD MAX | BD MAX | Final | Final | Final |
| | | Ent Ct. | Ent | E. histolytica | Call | Call | Call |
| Specimen ID | Type | Score | ymaxEP | Result | Ent | Giardia | Crypto |
| 12S0000574 | Fixed | 46 | 0.62 | Negative | NA | NA | NA |
| 13S0000190 | Fixed | 46 | 1.42 | Negative | NA | NA | NA |
| F25 | Fixed | 46 | 2.47 | Giardia | NA | Positive | NA |
| 12S0000734 | Unpreserved | 46 | 2.26 | Negative | Negative | NA | NA |
| 13S0000172 | Fixed | 46 | 0.38 | Negative | NA | NA | NA |
| 12S0000684 | Unpreserved | 46 | 2.23 | Negative | Negative | NA | NA |
| 6304 | Fixed | 46 | 1.67 | Crypto | NA | NA | Positive |
| 13S0000575 | Fixed | 46 | 2.07 | Negative | NA | NA | NA |
| 12S0000758 | Unpreserved | 46 | 2.5 | Negative | NA | NA | NA |
| 6419 | Fixed | 46 | 2.47 | Negative | NA | NA | Positive |
| EH22 | Unpreserved | 36.39 | 1379.22 | Entamoeba | NA | NA | NA |
| | | | | Histolytica | | | |
| 13S0000080 | Unpreserved | 46 | 0.58 | Negative | NA | NA | NA |
| Leiden 75 | Unpreserved | 46 | 4.52 | Giardia | Negative | Positive | NA |
| Leiden 83 | Unpreserved | 46 | 2.57 | Giardia | Negative | Positive | NA |
| F43 | Fixed | 46 | 3.71 | Giardia | NA | Positive | NA |
| 12S0000769 | Unpreserved | 46 | 2.73 | Negative | Negative | NA | NA |
| DLS13-05810-01-01 | Fixed | 46 | 16.1 | Giardia | NA | Positive | NA |
| 12S0000739 | Unpreserved | 46 | 1.86 | Negative | NA | NA | NA |
| 13S0000130 | Fixed | 46 | 2.91 | Negative | Negative | NA | NA |
| 12S0000565 | Fixed | 46 | 0.48 | Negative | NA | NA | NA |
| 13S0000141 | Fixed | 46 | 3.67 | Negative | Negative | NA | NA |
| 13S0000561 | Fixed | 46 | 2.14 | Negative | NA | NA | NA |
| 11995 | Fixed | 46 | 0.63 | Crypto | NA | NA | Positive |
| 181 | Fixed | 46 | 3.3 | Giardia | Negative | Positive | Negative |
| 13S0000069 | Unpreserved | 46 | 3.96 | Negative | Negative | NA | NA |
| E20 | Fixed | 46 | 3.51 | Negative | Negative | NA | NA |
| 12S0000765 | Unpreserved | 46 | 4.47 | Negative | NA | NA | NA |
| Leiden 12 | Unpreserved | 46 | 2.57 | Crypto | NA | NA | Positive |
| 12S0000693 | Unpreserved | 46 | 60.46 | Negative | Negative | NA | NA |
| Leiden 78 | Unpreserved | 46 | 1.02 | Giardia | Negative | Positive | NA |
| EH17 | Unpreserved | 34.89 | 2249.05 | Entamoeba | NA | NA | NA |
| | | | | Histolytica | | | |
| CCF06 | Fixed | 46 | 1.82 | Giardia | NA | NA | Negative |
| EH19 | Unpreserved | 33.95 | 2976.38 | Entamoeba | NA | NA | NA |
| | | | | Histolytica | | | |
| EH20 | Unpreserved | 35.48 | 2753.42 | Entamoeba | NA | NA | NA |
| | | | | Histolytica | | | |
| 1438 | Fixed | 46 | 8.86 | Crypto | NA | NA | NA |
| 13S0000005 | Unpreserved | 46 | 2.81 | Negative | NA | NA | Negative |
| 13S0000159 | Fixed | 46 | 9.3 | Negative | NA | NA | NA |
| 12S0000722 | Unpreserved | 46 | 7.03 | Negative | NA | NA | NA |
| DLS13-05812-01-01 | Fixed | 46 | 0.68 | Giardia | Negative | Positive | NA |
| 12S0000706 | Unpreserved | 46 | 7.28 | Negative | NA | NA | NA |
| Leiden 64 | Unpreserved | 46 | 30.24 | Giardia | NA | Positive | NA |
| 13S0000559 | Fixed | 46 | 15.83 | Negative | NA | NA | NA |
| 12S0000673 | Unpreserved | 46 | 21 | Negative | NA | NA | NA |
| DLS13-05780-01-01 | Fixed | 46 | 5.1 | Giardia | NA | NA | NA |
| 12S0000788 | Unpreserved | 46 | 8.01 | Negative | Negative | NA | NA |
| 13S0000059 | Unpreserved | 46 | 7.52 | Negative | NA | NA | NA |
| DLS13-05960 | Unpreserved | 46 | 6.56 | Crypto | Negative | NA | Positive |
| 13S0000139 | Fixed | 46 | 11.4 | Negative | Negative | NA | Negative |
| 13S0000037 | Unpreserved | 46 | 0.36 | Negative | NA | NA | NA |
| CIN01005 | Fixed | 46 | 1.94 | Giardia | NA | NA | NA |
| 13S0000185 | Fixed | 46 | 15.96 | Negative | NA | NA | NA |
| 12S0000699 | Unpreserved | 46 | 20.76 | Negative | Negative | NA | NA |
| 55 | Fixed | 46 | 6.23 | Giardia | Negative | Positive | NA |
| CCF08 | Fixed | 46 | 38.38 | Giardia | NA | NA | NA |
| 13S0000564 | Fixed | 46 | 4.82 | Negative | NA | NA | NA |
| 13S0000131 | Fixed | 46 | 21.12 | Negative | Negative | NA | NA |
| 12S0000561 | Fixed | 46 | 1.43 | Negative | NA | NA | NA |
| EH23 | Unpreserved | 28.12 | 4057.88 | Entamoeba | Positive | NA | NA |
| | | | | Histolytica | | | |
| 564 | Fixed | 46 | 4.66 | Crypto | NA | NA | Positive |
| Leiden 26 | Unpreserved | 46 | 1.44 | Crypto | NA | NA | Positive |
| 12S0000771 | Unpreserved | 46 | 4.71 | Negative | NA | NA | NA |
| 12S0000729 | Unpreserved | 46 | 1.32 | Negative | Negative | NA | NA |
| Leiden 57 | Unpreserved | 46 | 2.32 | Giardia | NA | Positive | NA |
| 12997 | Fixed | 46 | 4.22 | Crypto | NA | NA | Positive |
| 13S0000070 | Unpreserved | 46 | 14.93 | Negative | NA | NA | NA |
| 13S0000171 | Fixed | 46 | 22.03 | Negative | NA | NA | NA |
| DLS13-06001 | Unpreserved | 46 | 0.94 | Negative | Negative | NA | NA |
| Leiden 39 | Unpreserved | 46 | 7.58 | Crypto | Negative | NA | Positive |
| DLS13-05967 | Unpreserved | 46 | 10.82 | Crypto | Negative | NA | Positive |
| EH18 | Unpreserved | 32.94 | 3130.69 | Entamoeba | NA | NA | NA |
| | | | | Histolytica | | | |
| 13S0000122 | Fixed | 46 | 0.8 | Negative | NA | NA | NA |
| 13S0000057 | Unpreserved | 46 | 24.3 | Negative | NA | NA | NA |
| 12S0000575 | Fixed | 46 | 16.63 | Negative | NA | NA | NA |
| 12S0000737 | Unpreserved | 46 | 20.8 | Negative | NA | NA | NA |
| 13S0000188 | Fixed | 46 | 3.77 | Negative | NA | NA | NA |
| 12S0000569 | Fixed | 46 | 21.08 | Negative | NA | NA | NA |
| 12S0000692 | Unpreserved | 46 | 49.89 | Negative | NA | NA | NA |
| 13S0000036 | Unpreserved | 46 | 32.29 | Negative | Negative | NA | NA |
| 13S0000566 | Fixed | 46 | 17.56 | Negative | NA | NA | NA |
| 13S0000109 | Unpreserved | 46 | 23.01 | Negative | NA | NA | NA |
| 13S0000090 | Unpreserved | 46 | 7.58 | Negative | NA | NA | NA |
| 36 | Fixed | 46 | 4.42 | Giardia | NA | Positive | NA |
| 12S0000697 | Unpreserved | 46 | 34.47 | Negative | NA | NA | NA |
| 12S0000572 | Fixed | 46 | 15.66 | Negative | NA | NA | NA |
| 13S0000106 | Unpreserved | 46 | 2.5 | Negative | NA | NA | NA |
| F35 | Fixed | 46 | 2.28 | Giardia | NA | Positive | NA |
| 12S0000735 | Unpreserved | 46 | 2.02 | Negative | NA | NA | NA |
| 13S0000177 | Fixed | 46 | 2.16 | Negative | NA | NA | NA |
| 12S0000707 | Unpreserved | 46 | 2.37 | Negative | NA | NA | NA |
| 12S0000708 | Unpreserved | 46 | 13.91 | Negative | Negative | NA | NA |
| DLS13-05974 | Unpreserved | 46 | 2.83 | Negative | NA | NA | NA |
| 13S0000008 | Unpreserved | 46 | 5.71 | Negative | NA | NA | NA |
| 13S0000011 | Unpreserved | 46 | 3.13 | Negative | NA | NA | NA |
| DLS13-05782-01-01 | Fixed | 46 | 1.36 | Giardia, | NA | NA | Positive |
| | | | | Crypto | | | |
| 12S0000670 | Unpreserved | 46 | 2.58 | Negative | NA | NA | NA |
| CIN01020 | Fixed | 46 | 2.86 | Giardia | NA | NA | NA |
| 13S0000156 | Fixed | 46 | 3.5 | Negative | NA | NA | NA |
| 13S0000039 | Unpreserved | 46 | 11.25 | Negative | NA | NA | NA |
| 13S0000035 | Unpreserved | 46 | 25.79 | Negative | Negative | NA | NA |
| 12S0000570 | Fixed | 46 | 18.56 | Negative | NA | NA | NA |
| 11334 | Fixed | 46 | 23.01 | Crypto | NA | NA | Positive |
| 13S0000569 | Fixed | 46 | 4.48 | Negative | NA | NA | NA |
| 13S0000065 | Unpreserved | 46 | 2.09 | Negative | NA | NA | NA |
| EH12 | Fixed | 30.93 | 3244.67 | Entamoeba | NA | NA | NA |
| | | | | Histolytica | | | |
| 12S0000777 | Unpreserved | 46 | 4.09 | Negative | NA | NA | NA |
| 13S0000116 | Unpreserved | 46 | 4.06 | Negative | NA | NA | NA |
| DLS13-05994 | Unpreserved | 46 | 1.53 | Crypto | NA | NA | Positive |
| 13S0000138 | Fixed | 46 | 1.32 | Negative | NA | NA | NA |
| Leiden 68 | Unpreserved | 46 | 1.09 | Giardia | NA | Positive | NA |
| Leiden 63 | Unpreserved | 46 | 1.64 | Giardia | NA | Positive | NA |
| 13S0000149 | Fixed | 46 | 9.42 | Negative | NA | NA | NA |
| 12S0000732 | Unpreserved | 46 | 8.91 | Negative | NA | NA | NA |
| DLS13-05976 | Unpreserved | 46 | 32.29 | Giardia, | Negative | Positive | Positive |
| | | | | Crypto | | | |
| 12S0000688 | Unpreserved | 46 | 20.19 | Negative | NA | NA | NA |
| EH08 | Fixed | 25.49 | 4426.18 | Entamoeba | Positive | NA | NA |
| | | | | Histolytica | | | |
| CIN01004 | Fixed | 46 | 1.77 | Giardia | NA | NA | NA |
| DLS13-05956 | Unpreserved | 46 | 18.49 | Crypto | NA | NA | Positive |
| Leiden 10 | Unpreserved | 46 | 22.73 | Crypto | NA | NA | Positive |
| Leiden 37 | Unpreserved | 46 | 3.65 | Crypto | Negative | NA | Positive |
| DLS13-05790-01-01 | Fixed | 46 | 5.62 | Crypto | NA | NA | Positive |
| DLS13-05996 | Unpreserved | 46 | 1.52 | Negative | NA | NA | NA |
| F33 | Fixed | 46 | 3.66 | Giardia | NA | Positive | NA |
| Leiden 96 | Unpreserved | 46 | 6.72 | Giardia | NA | Positive | NA |
| Leiden 99 | Unpreserved | 46 | 2.14 | Giardia | NA | Positive | NA |
| 13S0000183 | Fixed | 46 | 3.56 | Negative | NA | NA | NA |
| 12S0000503 | Fixed | 46 | 25.82 | Negative | NA | NA | NA |
| 13S0000092 | Unpreserved | 46 | 27.75 | Negative | NA | NA | NA |
| Leiden 84 | Unpreserved | 46 | 2.92 | Giardia | Negative | Positive | NA |
| 12S0000786 | Unpreserved | 46 | 1.75 | Negative | NA | NA | NA |
| DLS13-05817-01-01 | Fixed | 46 | 4.16 | Giardia, | Negative | Positive | Positive |
| | | | | Crypto | | | |
| Leiden 79 | Unpreserved | 46 | 10.49 | Giardia | NA | NA | NA |
| 13S0000111 | Unpreserved | 46 | 5.26 | Negative | NA | NA | NA |
| CCF11 | Fixed | 46 | 3.61 | Giardia | NA | NA | NA |
| 124 | Fixed | 46 | 3.64 | Giardia | NA | Positive | NA |
| 13S0000563 | Fixed | 46 | 2.05 | Negative | NA | NA | NA |
| 12S0000678 | Unpreserved | 46 | 8.8 | Negative | NA | NA | NA |
| 12S0000698 | Unpreserved | 46 | 2.76 | Negative | NA | NA | NA |
| 77 | Fixed | 46 | 2.32 | Giardia | Negative | Positive | NA |
| 13S0000084 | Unpreserved | 46 | 0.51 | Negative | NA | NA | NA |
| 12S0000746 | Unpreserved | 46 | 14.43 | Negative | NA | NA | NA |
| CIN01040 | Fixed | 46 | 2.92 | Giardia | NA | Positive | NA |
| 13S0000117 | Unpreserved | 46 | 1.03 | Negative | NA | NA | NA |
| Leiden 32 | Unpreserved | 46 | 6.42 | Crypto | NA | NA | Positive |
| 13S0000112 | Unpreserved | 46 | 2.19 | Negative | NA | NA | NA |
| 12S0000711 | Unpreserved | 46 | 2.05 | Negative | NA | NA | NA |
| DLS13-05982 | Unpreserved | 46 | 6.28 | Negative | Negative | NA | NA |
| 13S0000032 | Unpreserved | 46 | 5.8 | Negative | NA | NA | NA |
| 13S0000565 | Fixed | 46 | 0.54 | Negative | NA | NA | NA |
| DLS13-05787-01-01 | Fixed | 46 | 13.03 | Crypto | NA | NA | Positive |
| 12S0000760 | Unpreserved | 46 | 1.75 | Negative | NA | NA | NA |
| Leiden 98 | Unpreserved | 46 | 0.99 | Giardia | NA | Positive | NA |
| 13S0000087 | Unpreserved | 46 | 0.98 | Negative | NA | NA | NA |
| F41 | Fixed | 46 | 2.97 | Giardia | NA | Positive | NA |
| 6289 | Fixed | 46 | 3.55 | Crypto | NA | NA | Positive |
| DLS13-05947 | Unpreserved | 46 | 15.37 | Crypto | Negative | NA | Positive |
| E18 | Fixed | 46 | 2.05 | Negative | Negative | NA | NA |
| 13S0000568 | Fixed | 46 | 2.96 | Negative | NA | NA | NA |
| F37 | Fixed | 46 | 1.77 | Giardia | NA | Positive | NA |
| Leiden 36 | Unpreserved | 46 | 7.32 | Crypto | Negative | NA | Positive |
| 13S0000158 | Fixed | 46 | 1.21 | Negative | NA | NA | NA |
| DLS13-05973 | Unpreserved | 46 | 23.69 | Negative | NA | NA | NA |
| 13S0000053 | Unpreserved | 46 | 1.93 | Negative | NA | NA | NA |
| Leiden 91 | Unpreserved | 46 | 0.13 | Giardia | NA | Positive | NA |
| DLS13-05966 | Unpreserved | 46 | 13.22 | Crypto | Negative | NA | Positive |
| 12S0000778 | Unpreserved | 46 | 10.25 | Negative | NA | NA | NA |
| 13S0000151 | Fixed | 46 | 2.11 | Negative | NA | NA | NA |
| 13S0000147 | Fixed | 46 | 6.24 | Negative | NA | NA | NA |
| 13S0000179 | Fixed | 46 | 1.09 | Negative | NA | NA | NA |
| 13S0000006 | Unpreserved | 46 | 11.64 | Negative | NA | NA | NA |
| 13S0000189 | Fixed | 46 | 8.69 | Negative | NA | NA | NA |
| 12S0000573 | Fixed | 46 | 11.49 | Negative | NA | NA | NA |
| DLS13-05980 | Unpreserved | 46 | 16.94 | Crypto | Negative | NA | Positive |
| 12S0000791 | Unpreserved | 46 | 20.37 | Negative | NA | NA | NA |
| Leiden 25 | Unpreserved | 46 | 16.23 | Crypto | Negative | NA | Positive |
| DLS13-05952 | Unpreserved | 46 | 17.92 | Crypto | Negative | NA | Positive |
| 13S0000019 | Unpreserved | 46 | 10.66 | Negative | NA | NA | NA |
| 91 | Fixed | 46 | 2.56 | Giardia | NA | Positive | NA |
| DLS13-05972 | Unpreserved | 46 | 6.19 | Negative | Negative | NA | NA |
| 13S0000043 | Unpreserved | 46 | 7 | Negative | NA | NA | NA |
| 12S0000714 | Unpreserved | 46 | 1.87 | Negative | NA | NA | NA |
| 12S0000675 | Unpreserved | 46 | 1.75 | Negative | NA | NA | NA |
| DLS13-05801-01-01 | Fixed | 46 | 1.94 | Crypto | NA | NA | Positive |
| 12S0000682 | Unpreserved | 46 | 23.33 | Negative | NA | NA | NA |
| DLS13-05802-01-01 | Fixed | 46 | 5.22 | Crypto | NA | NA | Positive |
| 113 | Fixed | 46 | 21.51 | Giardia | Negative | Positive | NA |
| Leiden 85 | Unpreserved | 46 | 63.6 | Giardia | NA | Positive | NA |
| EH14 | Fixed | 26.09 | 3348.86 | Entamoeba | Positive | NA | NA |
| | | | | Histolytica | | | |
| 13S0000096 | Unpreserved | 46 | 48.77 | Negative | NA | NA | NA |
| 12S0000733 | Unpreserved | 46 | 37 | Negative | Negative | NA | NA |
| 12S0000689 | Unpreserved | 46 | 0.68 | Negative | NA | NA | NA |
| CCF03 | Fixed | 46 | 5.54 | Giardia | NA | NA | NA |
| 13S0000049 | Unpreserved | 46 | 5.73 | Negative | NA | NA | NA |
| Leiden 8 | Unpreserved | 46 | 53.94 | Crypto | NA | NA | Positive |
| Leiden 29 | Unpreserved | 46 | 8.26 | Crypto | Negative | NA | Positive |
| 12S0000576 | Fixed | 46 | 35.02 | Negative | NA | NA | NA |
| Leiden 6 | Unpreserved | 46 | 17.46 | Crypto | Negative | NA | Positive |
| 13S0000007 | Unpreserved | 46 | 26.52 | Negative | NA | NA | NA |
| 12S0000717 | Unpreserved | 46 | 9.55 | Negative | NA | NA | NA |
| 13S0000001 | Unpreserved | 46 | 12.88 | Negative | NA | NA | NA |
| 13S0000134 | Fixed | 46 | 36.35 | Negative | NA | Negative | NA |
| 13S0000104 | Unpreserved | 46 | 3.96 | Negative | NA | NA | NA |
| 13S0000089 | Unpreserved | 46 | 12.21 | Negative | NA | NA | NA |
| 12S0000762 | Unpreserved | 46 | 7.7 | Negative | NA | NA | NA |
| 13S0000562 | Fixed | 46 | 4.05 | Negative | NA | NA | NA |
| 13S0000010 | Unpreserved | 46 | 17.88 | Negative | NA | NA | NA |
| 125 | Fixed | 46 | 6.4 | Giardia | NA | Positive | NA |
| 13S0000135 | Fixed | 46 | 5.82 | Negative | NA | NA | NA |
| 13S0000118 | Unpreserved | 46 | 9.18 | Negative | NA | NA | NA |
| 12S0000713 | Unpreserved | 46 | 38.22 | Negative | NA | NA | NA |
| 13S0000162 | Fixed | 46 | 26.34 | Negative | NA | NA | NA |
| DLS13-05970 | Unpreserved | 46 | 14.32 | Crypto | Negative | NA | Positive |
| 12S0000727 | Unpreserved | 46 | 3.79 | Negative | Negative | NA | NA |
| 39546 | Fixed | 46 | 5.5 | Giardia | Negative | Positive | NA |
| 13S0000083 | Unpreserved | 46 | 2.22 | Negative | NA | NA | NA |
| Leiden 49 | Unpreserved | 46 | 11.51 | Crypto | Negative | NA | Positive |
| EH09 | Fixed | 31.04 | 4213.82 | Entamoeba | NA | NA | NA |
| | | | | Histolytica | | | |
| F31 | Fixed | 46 | 7.62 | Giardia | Negative | Positive | NA |
| 12S0000751 | Unpreserved | 46 | 7.09 | Negative | Negative | NA | NA |
| 13S0000153 | Fixed | 46 | 9.34 | Negative | Negative | NA | NA |
| 13S0000166 | Fixed | 46 | 0.22 | Negative | Negative | NA | NA |
| 12S0000685 | Unpreserved | 46 | 17.56 | Negative | NA | NA | NA |
| CCF10 | Fixed | 46 | 4.23 | Giardia | NA | NA | NA |
| EH10 | Fixed | 32.34 | 2999.22 | Entamoeba | NA | NA | NA |
| | | | | Histolytica | | | |
| 13S0000098 | Unpreserved | 46 | 15.43 | Negative | NA | NA | NA |
| 81 | Fixed | 46 | 7.28 | Giardia | Negative | Positive | NA |
| 13S0000095 | Unpreserved | 46 | 22.84 | Negative | NA | NA | NA |
| Leiden 1 | Unpreserved | 46 | 4.94 | Crypto | Negative | NA | Positive |
| 13S0000173 | Fixed | 46 | 18.66 | Negative | Negative | NA | NA |
| DLS13-05784-01-01 | Fixed | 46 | 19.18 | Giardia | NA | Positive | NA |
| Leiden 51 | Unpreserved | 46 | 32.95 | Giardia | Negative | Positive | NA |
| 12S0000725 | Unpreserved | 46 | 52.6 | Negative | NA | NA | NA |
| 162 | Fixed | 46 | 38.96 | Giardia | Negative | Positive | NA |
| 12S0000781 | Unpreserved | 46 | 22.39 | Negative | NA | NA | NA |
| 13S0000143 | Fixed | 46 | 45.21 | Negative | NA | NA | NA |
| 13S0000003 | Unpreserved | 46 | 18.9 | Negative | NA | NA | NA |
| 12S0000710 | Unpreserved | 46 | 88.74 | Crypto | NA | NA | NA |
| Leiden 86 | Unpreserved | 46 | 6.46 | Giardia | Negative | Positive | NA |
| 13S0000027 | Unpreserved | 46 | 47.45 | Negative | NA | NA | NA |
| DLS13-05949 | Unpreserved | 46 | 5.75 | Negative | NA | NA | NA |
| 12S0000702 | Unpreserved | 46 | 7.58 | Negative | Negative | NA | NA |
| Leiden 82 | Unpreserved | 46 | 4.01 | Giardia | Negative | Positive | NA |
| 1247 | Fixed | 46 | 6.93 | Negative | NA | NA | Positive |
| 13S0000140 | Fixed | 46 | 9.5 | Negative | Negative | NA | NA |
| Leiden 59 | Unpreserved | 46 | 10.33 | Giardia | Negative | Positive | NA |
| 13S0000558 | Fixed | 46 | 2.85 | Negative | Negative | NA | NA |
| 13S0000157 | Fixed | 46 | 12.44 | Negative | Negative | NA | NA |
| 8174 | Fixed | 46 | 15.07 | Crypto | NA | NA | NA |
| F29 | Fixed | 46 | 30.36 | Giardia | Negative | Negative | NA |
| CIN01026 | Fixed | 46 | 3.99 | Giardia | Negative | Positive | NA |
| Leiden 58 | Unpreserved | 46 | 12.14 | Giardia | NA | Positive | NA |
| 13S0000165 | Fixed | 46 | 10.87 | Negative | Negative | NA | NA |
| 13S0000164 | Fixed | 46 | 3.14 | Negative | Negative | NA | NA |
| DLS13-05808-01-01 | Fixed | 46 | 8.14 | Crypto | Negative | NA | Positive |
| 12S0000716 | Unpreserved | 46 | 10.74 | Negative | Negative | NA | NA |
| 12S0000784 | Unpreserved | 46 | 3.05 | Negative | Negative | NA | NA |
| EH24 | Unpreserved | 31.84 | 2717.46 | Entamoeba | Negative | NA | NA |
| | | | | Histolytica | | | |
| 12S0000681 | Unpreserved | 46 | 13.88 | Negative | Negative | NA | NA |
| DLS13-05969 | Unpreserved | 46 | 24.35 | Crypto | Negative | NA | Positive |
| Leiden 62 | Unpreserved | 46 | 9.24 | Giardia | Negative | Positive | NA |
| 13S0000002 | Unpreserved | 46 | 1.57 | Negative | Negative | NA | NA |
| 12S0000770 | Unpreserved | 46 | 6.25 | Negative | Negative | NA | NA |
| DLS13-05789-01-01 | Fixed | 46 | 1.48 | Giardia | NA | NA | Positive |
| 13S0000560 | Fixed | 46 | 3.8 | Negative | Negative | NA | NA |
| 13S0000145 | Fixed | 46 | 5.11 | Negative | Negative | NA | NA |
| DLS13-05958 | Unpreserved | 46 | 1.49 | Crypto | Negative | NA | Positive |
| 12S0000563 | Fixed | 46 | 2.25 | Negative | Negative | NA | NA |
| 12S0000683 | Unpreserved | 46 | 2.48 | Negative | Negative | NA | NA |
| 13S0000168 | Fixed | 46 | 1.16 | Negative | NA | NA | NA |
| Leiden 46 | Unpreserved | 46 | 2.6 | Negative | NA | NA | Positive |
| E24 | Fixed | 46 | 2.28 | Negative | Negative | NA | NA |
| 13S0000101 | Unpreserved | 46 | 2.28 | Negative | NA | NA | NA |
| 13S0000155 | Fixed | 46 | 6.29 | Negative | NA | NA | NA |
| Leiden 71 | Unpreserved | 46 | 1.65 | Giardia | NA | Positive | NA |
| EH06 | Fixed | 27.49 | 3596.16 | Entamoeba | NA | NA | NA |
| | | | | Histolytica | | | |
| DLS13-05816-01-01 | Fixed | 46 | 2.68 | Giardia, | NA | NA | Positive |
| | | | | Crypto | | | |
| F27 | Fixed | 46 | 1.22 | Giardia | NA | Positive | NA |
| DLS13-05997 | Unpreserved | 46 | 2.71 | Negative | Negative | NA | NA |
| EH04 | Fixed | 30.16 | 3073.89 | Entamoeba | NA | NA | NA |
| | | | | Histolytica | | | |
| 12S0000726 | Unpreserved | 46 | 2.46 | Negative | NA | NA | NA |
| DLS13-05963 | Unpreserved | 46 | 2.55 | Negative | Negative | NA | NA |
| DLS13-05793-01-01 | Fixed | 46 | 0.47 | Crypto | NA | NA | Positive |
| 13S0000126 | Fixed | 46 | 2.6 | Negative | NA | NA | NA |
| EH25 | Unpreserved | 33.04 | 2731.07 | Entamoeba | NA | NA | NA |
| | | | | Histolytica | | | |
| 40015 | Fixed | 46 | 0.24 | Negative | NA | NA | Positive |
| 13S0000181 | Fixed | 46 | 4.58 | Negative | Negative | NA | NA |
| 58 | Fixed | 46 | 6.16 | Giardia | Negative | Positive | NA |
| CCF02 | Fixed | 46 | 3.45 | Giardia | NA | Positive | NA |
| 12S0000568 | Fixed | 46 | 5.8 | Negative | NA | NA | NA |
| 96 | Fixed | 46 | 2.94 | Giardia | NA | Positive | NA |
| Leiden 43 | Unpreserved | 46 | 11.23 | Crypto | NA | NA | Positive |
| 13S0000123 | Fixed | 46 | 18.72 | Negative | NA | NA | NA |
| 12S0000705 | Unpreserved | 46 | 6.71 | Negative | NA | NA | NA |
| DLS13-05794-01-01 | Fixed | 46 | 1.34 | Crypto | NA | NA | Positive |
| 13S0000136 | Fixed | 46 | 7.28 | Negative | NA | NA | NA |
| Leiden 97 | Unpreserved | 46 | 1.83 | Giardia | Negative | Positive | NA |
| DLS13-05798-01-01 | Fixed | 46 | 20.49 | Crypto | NA | NA | Positive |
| 12S0000719 | Unpreserved | 46 | 0.23 | Negative | Negative | NA | NA |
| 12S0000668 | Unpreserved | 46 | 1.12 | Negative | NA | NA | NA |
| Leiden 80 | Unpreserved | 46 | 1.93 | Giardia | NA | NA | NA |
| 12S0000756 | Unpreserved | 46 | 0.55 | Negative | NA | NA | NA |
| 13S0000107 | Unpreserved | 46 | 0.63 | Negative | NA | NA | NA |
| 13S0000068 | Unpreserved | 46 | 1.3 | Negative | Negative | NA | NA |
| 12S0000749 | Unpreserved | 46 | 1.61 | Negative | NA | NA | NA |
| Leiden 16 | Unpreserved | 46 | 16.82 | Crypto | Negative | NA | Positive |
| Leiden 72 | Unpreserved | 46 | 1.56 | Giardia | NA | Positive | NA |
| 12S0000763 | Unpreserved | 46 | 1.78 | Negative | Negative | NA | NA |
| 12S0000709 | Unpreserved | 46 | 0.52 | Negative | Negative | NA | NA |
| 13S0000072 | Unpreserved | 46 | 0.92 | Negative | NA | NA | NA |
| 13S0000013 | Unpreserved | 46 | 4.63 | Negative | NA | NA | NA |
| CCF01 | Fixed | 46 | 4.11 | Giardia | NA | Positive | NA |
| DLS13-05953 | Unpreserved | 46 | 12.38 | Crypto | Negative | NA | Positive |
| 12S0000761 | Unpreserved | 46 | 0.27 | Negative | NA | NA | NA |
| Leiden 9 | Unpreserved | 46 | 22.34 | Negative | NA | NA | Positive |
| 13S0000054 | Unpreserved | 46 | 1.61 | Negative | NA | NA | NA |
| EH16 | Unpreserved | 30.2 | 3188.71 | Entamoeba | NA | NA | NA |
| | | | | Histolytica | | | |
| 13S0000081 | Unpreserved | 46 | 24.01 | Negative | NA | NA | NA |
| DLS13-05951 | Unpreserved | 46 | 11.67 | Crypto | Negative | NA | Positive |
| DLS13-05820-01-01 | Fixed | 46 | 9.97 | Giardia | Negative | Positive | NA |
| Leiden 5 | Unpreserved | 46 | 9.52 | Crypto | Negative | NA | Positive |
| EH07 | Fixed | 30.45 | 2634.49 | Entamoeba | NA | NA | NA |
| | | | | Histolytica | | | |
| 13S0000146 | Fixed | 46 | 0.59 | Negative | NA | NA | NA |
| CIN01012 | Fixed | 46 | 2.03 | Giardia | NA | Positive | NA |
| DLS13-05818-01-01 | Fixed | 46 | 0.67 | Giardia | Negative | Positive | NA |
| 13S0000169 | Fixed | 46 | 0.73 | Negative | Negative | NA | NA |
| 13S0000186 | Fixed | 46 | 1.8 | Negative | NA | NA | NA |
| 13S0000152 | Fixed | 46 | 5.8 | Negative | Negative | NA | NA |
| 12S0000669 | Unpreserved | 46 | 2.63 | Negative | NA | NA | NA |
| 12S0000566 | Fixed | 46 | 1.25 | Negative | NA | NA | NA |
| 14790 | Fixed | 46 | 20.38 | Crypto | NA | NA | Positive |
| Leiden 18 | Unpreserved | 46 | 28.25 | Crypto | Negative | NA | Positive |
| 13S0000094 | Unpreserved | 46 | 26.47 | Negative | Negative | NA | NA |
| 13S0000154 | Fixed | 46 | 1.94 | Negative | NA | NA | NA |
| 12S0000724 | Unpreserved | 46 | 4.83 | Negative | NA | NA | NA |
| 13S0000161 | Fixed | 46 | 1.42 | Negative | Negative | NA | NA |
| 12S0000562 | Fixed | 46 | 0.23 | Negative | Negative | NA | NA |
| 13S0000040 | Unpreserved | 46 | 0.86 | Negative | NA | NA | NA |
| 13S0000024 | Unpreserved | 46 | 2.28 | Negative | NA | NA | NA |
| 13S0000148 | Fixed | 46 | 14.85 | Negative | NA | NA | NA |
| DLS13-05983 | Unpreserved | 46 | 9.67 | Crypto | Negative | NA | Positive |
| 159 | Fixed | 46 | 51.22 | Giardia | Negative | Positive | NA |
| Leiden 74 | Unpreserved | 46 | 1.51 | Giardia | NA | Positive | NA |
| 13S0000031 | Unpreserved | 46 | 18.76 | Negative | NA | NA | NA |
| EH05 | Fixed | 24.52 | 4316.84 | Entamoeba | Positive | NA | NA |
| | | | | Histolytica | | | |
| 3645 | Fixed | 46 | 1.56 | Crypto | NA | NA | Positive |
| 13S0000119 | Unpreserved | 46 | 0.63 | Negative | NA | NA | NA |
| 13S0000021 | Unpreserved | 46 | 3.81 | Negative | Negative | NA | NA |
| 13S0000132 | Fixed | 46 | 3.39 | Negative | NA | NA | NA |
| 12S0000764 | Unpreserved | 46 | 2.81 | Negative | Negative | NA | NA |
| 13S0000064 | Unpreserved | 46 | 14.37 | Negative | NA | NA | NA |
| 183 | Fixed | 46 | 35.62 | Giardia | NA | Positive | NA |
| DLS13-05950 | Unpreserved | 46 | 28.06 | Crypto | Negative | NA | Positive |
| 13S0000557 | Fixed | 46 | 24.46 | Negative | NA | NA | NA |
| CIN01038 | Fixed | 46 | 0.33 | Giardia | NA | Positive | NA |
| 13S0000120 | Unpreserved | 46 | 20.14 | Negative | NA | NA | NA |
| EH01 | Fixed | 27.55 | 3452.35 | Entamoeba | Positive | NA | NA |
| | | | | Histolytica | | | |
| 12S0000679 | Unpreserved | 46 | 11.58 | Negative | NA | NA | NA |
| Leiden 73 | Unpreserved | 46 | 26.9 | Giardia | NA | Positive | NA |
| DLS13-05781-01-01 | Fixed | 46 | 1.5 | Giardia, | NA | Positive | NA |
| | | | | Crypto | | | |
| 13S0000150 | Fixed | 46 | 0.65 | Negative | NA | NA | NA |
| 13S0000115 | Unpreserved | 46 | 6.8 | Negative | NA | NA | NA |
| 13S0000142 | Fixed | 46 | 30.68 | Negative | NA | NA | NA |
| Leiden 70 | Unpreserved | 46 | 9.98 | Giardia | NA | Positive | NA |
| Leiden 69 | Unpreserved | 46 | 24.98 | Giardia | NA | Positive | NA |
| 12S0000704 | Unpreserved | 46 | 43.32 | Negative | NA | NA | NA |
| 129 | Fixed | 46 | 35.34 | Giardia | NA | Positive | NA |
| Leiden 61 | Unpreserved | 46 | 30.72 | Giardia | Negative | Positive | NA |
| EH03 | Fixed | 27.63 | 4138.66 | Entamoeba | NA | NA | NA |
| | | | | Histolytica | | | |
| 13S0000121 | Fixed | 46 | 8.94 | Negative | Negative | NA | NA |
| 7458 | Fixed | 46 | 21.52 | Negative | NA | NA | NA |
| 13S0000129 | Fixed | 46 | 13.08 | Negative | NA | NA | NA |
| 12S0000703 | Unpreserved | 46 | 15.65 | Negative | NA | NA | NA |
| 13S0000056 | Unpreserved | 46 | 9.93 | Negative | Negative | NA | NA |
| 12S0000766 | Unpreserved | 46 | 4.49 | Negative | NA | NA | NA |
| F23 | Fixed | 46 | 3.28 | Giardia | NA | Positive | NA |
| 13S0000103 | Unpreserved | 46 | 2.52 | Negative | NA | NA | NA |
| 12S0000723 | Unpreserved | 46 | 38.51 | Negative | NA | NA | NA |
| 130 | Fixed | 46 | 14.19 | Giardia | NA | Positive | NA |
| Leiden 52 | Unpreserved | 46 | 18.59 | Giardia | NA | Positive | NA |
| Leiden 60 | Unpreserved | 46 | 12.77 | Giardia | NA | Positive | NA |
| 13S0000038 | Unpreserved | 46 | 29.99 | Negative | NA | NA | NA |
| 13S0000100 | Unpreserved | 46 | 11.06 | Negative | NA | NA | NA |
| 13S0000102 | Unpreserved | 46 | 7.13 | Negative | NA | NA | NA |
| 13S0000576 | Fixed | 46 | 14.16 | Negative | NA | NA | NA |
| 12S0000567 | Fixed | 46 | 11.31 | Negative | NA | NA | NA |
| 12S0000672 | Unpreserved | 46 | 9.87 | Negative | NA | NA | NA |
| 12S0000757 | Unpreserved | 46 | 1.05 | Negative | NA | NA | NA |
| 12S0000742 | Unpreserved | 46 | 1.37 | Negative | NA | NA | NA |
| Leiden 17 | Unpreserved | 46 | 2.26 | Crypto | NA | NA | Positive |
| Leiden 15 | Unpreserved | 46 | 73.62 | Crypto | NA | NA | Positive |
| 13S0000009 | Unpreserved | 46 | 0.24 | Negative | NA | NA | NA |
| CIN01028 | Fixed | 46 | 3.36 | Giardia | NA | Positive | NA |
| 13S0000127 | Fixed | 46 | 0.85 | Negative | Negative | NA | NA |
| KH12-5156 | Unpreserved | 46 | 4.73 | Giardia | Negative | Positive | NA |
| 12S0000674 | Unpreserved | 46 | 1.41 | Negative | NA | NA | NA |
| 13S0000571 | Fixed | 46 | 2.72 | Negative | NA | NA | NA |
| 13S0000128 | Fixed | 46 | 1.01 | Negative | NA | NA | NA |
| 140 | Fixed | 46 | 1.12 | Giardia | Negative | Positive | NA |
| 12S0000738 | Unpreserved | 46 | 2.45 | Negative | NA | NA | NA |
| CIN01039 | Fixed | 46 | 1.62 | Giardia | NA | Positive | NA |
| KH12-4357 | Unpreserved | 46 | 2.14 | Giardia | NA | Positive | NA |
| Leiden 30 | Unpreserved | 46 | 1.76 | Crypto | Negative | NA | Positive |
| 13S0000114 | Unpreserved | 46 | 0.3 | Negative | NA | NA | NA |
| Leiden 55 | Unpreserved | 46 | 4.4 | Giardia | Negative | Positive | NA |
| 12S0000680 | Unpreserved | 46 | 2.94 | Negative | NA | NA | NA |
| E22 | Fixed | 46 | 3.31 | Negative | Negative | NA | NA |
| 39543 | Fixed | 46 | 0.36 | Giardia | NA | Positive | NA |
| 12S0000776 | Unpreserved | 46 | 12.99 | Negative | NA | NA | NA |
| KH12-6359 | Unpreserved | 46 | 0.2 | Giardia | NA | Positive | NA |
| 13S0000160 | Fixed | 46 | 56.76 | Negative | NA | NA | NA |
| DLS13-05945 | Unpreserved | 46 | 6.49 | Giardia, | NA | Positive | Positive |
| | | | | Crypto | | | |
| 11796 | Fixed | 46 | 11.69 | Crypto | NA | NA | Positive |
| DLS13-05978 | Unpreserved | 46 | 23.82 | Giardia | NA | Positive | NA |
| 13S0000187 | Fixed | 46 | 28.62 | Negative | NA | NA | NA |
| 13S0000063 | Unpreserved | 46 | 1.54 | Negative | Negative | NA | NA |
| 12S0000779 | Unpreserved | 46 | 6.07 | Negative | NA | NA | NA |
| DLS13-05800-01-01 | Fixed | 46 | 24.79 | Crypto | Negative | NA | Positive |
| 13S0000110 | Unpreserved | 46 | 4.48 | Negative | NA | NA | NA |
| 13S0000113 | Unpreserved | 46 | 7.12 | Negative | NA | NA | NA |
| CIN01027 | Fixed | 46 | 5.72 | Giardia | NA | NA | NA |
| 13S0000180 | Fixed | 46 | 4.41 | Negative | NA | NA | NA |
| 12S0000564 | Fixed | 46 | 2.75 | Negative | NA | NA | NA |
| CCF05 | Fixed | 46 | 4.06 | Giardia | NA | NA | NA |
| DLS13-05991 | Unpreserved | 46 | 3.96 | Negative | Negative | NA | NA |
| 12S0000667 | Unpreserved | 46 | 39.87 | Negative | NA | NA | NA |
| 13S0000182 | Fixed | 46 | 48.02 | Negative | NA | NA | NA |
| 12S0000790 | Unpreserved | 46 | 3.34 | Negative | NA | NA | NA |
| CIN01003 | Fixed | 46 | 12.21 | Giardia | NA | NA | NA |
| Leiden 19 | Unpreserved | 46 | 20.7 | Crypto | Negative | NA | Positive |
| DLS13-05792-01-01 | Fixed | 46 | 4.47 | Crypto | NA | NA | Positive |
| 13S0000099 | Unpreserved | 46 | 1.5 | Negative | NA | NA | NA |
| CIN01024 | Fixed | 46 | 3.55 | Giardia | NA | NA | NA |
| 13S0000062 | Unpreserved | 46 | 1.41 | Negative | Negative | NA | NA |
| EH13 | Fixed | 32.23 | 2934.41 | Entamoeba | NA | NA | NA |
| | | | | Histolytica | | | |
| 12S0000715 | Unpreserved | 46 | 8.69 | Negative | NA | NA | NA |
| DLS13-05961 | Unpreserved | 46 | 6.92 | Giardia, | Negative | Positive | Positive |
| | | | | Crypto | | | |
| DLS13-05981 | Unpreserved | 46 | 24.25 | Giardia, | Negative | Positive | Positive |
| | | | | Crypto | | | |
| CCF04 | Fixed | 46 | 2.56 | Giardia | NA | NA | NA |
| Leiden 22 | Unpreserved | 46 | 22.76 | Crypto | NA | NA | Positive |
| EH02 | Fixed | 24.05 | 4776.64 | Entamoeba | Positive | NA | NA |
| | | | | Histolytica | | | |
| 13S0000184 | Fixed | 46 | 5.29 | Negative | NA | NA | NA |
| Leiden 35 | Unpreserved | 46 | 5.77 | Negative | Negative | NA | Positive |
| DLS13-05807-01-01 | Fixed | 46 | 2.82 | Giardia | NA | NA | NA |
| 13S0000175 | Fixed | 46 | 6.57 | Negative | NA | NA | NA |
| 13S0000091 | Unpreserved | 46 | 4.62 | Negative | NA | NA | NA |
| 13S0000075 | Unpreserved | 46 | 12.08 | Negative | NA | NA | NA |
| DLS13-05795-01-01 | Fixed | 46 | 8.41 | Crypto | NA | NA | Positive |
| 12998 | Fixed | 46 | 15.49 | Crypto | Negative | NA | Positive |
| 13S0000174 | Fixed | 46 | 7.46 | Negative | NA | NA | NA |
| 13S0000055 | Unpreserved | 46 | 21.59 | Negative | Negative | NA | NA |
| 12S0000782 | Unpreserved | 46 | 8.4 | Negative | NA | NA | NA |
| 13S0000163 | Fixed | 46 | 12.96 | Negative | NA | NA | NA |
| 13S0000574 | Fixed | 46 | 15.3 | Negative | NA | NA | NA |
| 13S0000071 | Unpreserved | 46 | 7.56 | Negative | NA | NA | NA |
| Leiden 2 | Unpreserved | 46 | 19.52 | Crypto | Negative | Positive | Positive |
| 13S0000167 | Fixed | 46 | 16.57 | Negative | NA | NA | NA |
| 13S0000133 | Fixed | 46 | 3.83 | Negative | NA | NA | NA |
| 13S0000046 | Unpreserved | 46 | 29.37 | Negative | NA | NA | NA |
| 12S0000759 | Unpreserved | 46 | 4.19 | Negative | NA | NA | NA |
| Leiden 7 | Unpreserved | 46 | 17.57 | Crypto | Negative | NA | Positive |
| 13S0000041 | Unpreserved | 46 | 8.81 | Negative | NA | NA | NA |
| 12S0000671 | Unpreserved | 46 | 9.1 | Negative | Negative | NA | NA |
| 13S0000093 | Unpreserved | 46 | 2.36 | Negative | Negative | NA | NA |
| CIN01006 | Fixed | 46 | 1.33 | Giardia | NA | NA | NA |
| Leiden 33 | Unpreserved | 46 | 26.84 | Crypto | NA | NA | Positive |
| DLS13-05882 | Unpreserved | 46 | 17.62 | Negative | Negative | NA | NA |
| 13S0000144 | Fixed | 46 | 6.03 | Negative | Negative | NA | NA |
| DLS13-05893 | Unpreserved | 46 | 0.96 | Negative | Negative | NA | NA |
| 13S0000088 | Unpreserved | 46 | 7.76 | Negative | Negative | NA | NA |
| DLS13-05954 | Unpreserved | 46 | 9.72 | Crypto | Negative | NA | Positive |
| 20 | Fixed | 46 | 0.47 | Giardia | Negative | Positive | NA |
| 13S0000060 | Unpreserved | 46 | 2.91 | Negative | Negative | NA | NA |
| 64 | Fixed | 46 | 2.57 | Giardia | NA | Positive | NA |
| Leiden 67 | Unpreserved | 46 | 29.44 | Giardia | NA | Positive | NA |
| 13S0000066 | Unpreserved | 46 | 13.65 | Negative | NA | NA | NA |
| 13S0000178 | Fixed | 46 | 11.61 | Negative | Negative | NA | NA |
| 13S0000570 | Fixed | 46 | 23.22 | Negative | NA | NA | NA |
| 12S0000712 | Unpreserved | 46 | 26 | Negative | Negative | NA | NA |
| DLS13-05962 | Unpreserved | 46 | 8.49 | Crypto | NA | NA | Positive |
| 73 | Fixed | 46 | 10.18 | Giardia | NA | Positive | NA |
| EH15 | Fixed | 32.49 | 3811.37 | Entamoeba | NA | NA | NA |
| | | | | Histolytica | | | |
| Leiden 81 | Unpreserved | 46 | 0.37 | Giardia | NA | Positive | NA |
| Leiden 13 | Unpreserved | 46 | 4.25 | Crypto | NA | NA | Positive |
| 12S0000731 | Unpreserved | 46 | 7.83 | Negative | Negative | NA | NA |
| 12S0000686 | Unpreserved | 46 | 12.69 | Negative | NA | NA | NA |
| 12S0000676 | Unpreserved | 46 | 1.85 | Negative | NA | NA | NA |
| 13S0000067 | Unpreserved | 46 | 6.33 | Negative | NA | NA | NA |
| CIN01011 | Fixed | 46 | 1.44 | Giardia | NA | Positive | NA |
| Leiden 20 | Unpreserved | 46 | 12.19 | Negative | Negative | NA | NA |
| EH11 | Fixed | 24.65 | 4407.66 | Entamoeba | Positive | NA | NA |
| | | | | Histolytica | | | |
| Leiden 23 | Unpreserved | 46 | 1.37 | Crypto | NA | NA | Positive |
| 161 | Fixed | 46 | 0.43 | Giardia | NA | NA | NA |
| 13S0000058 | Unpreserved | 46 | 1.45 | Negative | NA | NA | NA |
| 13S0000077 | Unpreserved | 46 | 12.28 | Negative | NA | NA | NA |
| DLS13-05809-01-01 | Fixed | 46 | 3.4 | Crypto | NA | NA | Positive |
| DLS13-05788-01-01 | Fixed | 46 | 32.89 | Crypto | Negative | NA | Positive |
| DLS13-05987 | Unpreserved | 46 | 36.67 | Crypto | Negative | NA | Positive |
| Leiden 95 | Unpreserved | 46 | 20.81 | Giardia | Negative | Positive | NA |
| 68 | Fixed | 46 | 68.16 | Giardia | NA | Positive | NA |
| Leiden 53 | Unpreserved | 46 | 17.42 | Negative | NA | Positive | NA |
| 12S0000677 | Unpreserved | 46 | 37.95 | Crypto | NA | NA | NA |
| 12S0000721 | Unpreserved | 46 | 12.76 | Negative | NA | NA | NA |
| DLS13-05814-01-01 | Fixed | 46 | 26.94 | Giardia | NA | Positive | NA |
| Leiden 11 | Unpreserved | 46 | 20.87 | Crypto | NA | NA | Positive |
| Leiden 3 | Unpreserved | 46 | 14.15 | Crypto | NA | NA | Positive |
| 12S0000577 | Fixed | 46 | 0.9 | Negative | NA | NA | NA |
| 12S0000701 | Unpreserved | 46 | 9.16 | Negative | NA | NA | NA |
| 37 | Fixed | 46 | 0.75 | Giardia | NA | Positive | NA |
| 13S0000076 | Unpreserved | 46 | 7.89 | Negative | NA | NA | NA |
| DLS13-05968 | Unpreserved | 46 | 8.78 | Crypto | NA | NA | Positive |
| DLS13-05805-01-01 | Fixed | 46 | 9.6 | Crypto | NA | NA | Positive |
| 13S0000078 | Unpreserved | 46 | 6.16 | Negative | NA | NA | NA |
| Leiden 65 | Unpreserved | 46 | 4.28 | Giardia | Negative | Positive | NA |
| 13S0000573 | Fixed | 46 | 7.2 | Negative | NA | NA | NA |
| 13S0000097 | Unpreserved | 46 | 4.94 | Negative | NA | NA | NA |
| DLS13-05791-01-01 | Fixed | 46 | 10.14 | Crypto | NA | NA | Positive |
| 12S0000691 | Unpreserved | 46 | 7.04 | Negative | NA | NA | NA |
| 13S0000105 | Unpreserved | 46 | 3.83 | Negative | NA | NA | NA |
| 13S0000108 | Unpreserved | 46 | 1.46 | Negative | NA | NA | NA |
| 12S0000560 | Fixed | 46 | 3.5 | Negative | NA | NA | NA |
| 13S0000042 | Unpreserved | 46 | 6.1 | Negative | NA | NA | NA |
| 12S0000690 | Unpreserved | 46 | 18.57 | Negative | NA | NA | NA |
| Leiden 92 | Unpreserved | 46 | 53.63 | Giardia | NA | Positive | NA |
| DLS13-05804-01-01 | Fixed | 46 | 16.64 | Crypto | NA | NA | Positive |
| 11556 | Fixed | 46 | 42.2 | Crypto | NA | NA | Positive |
| 13S0000014 | Unpreserved | 46 | 48.81 | Negative | NA | NA | NA |
| EH21 | Unpreserved | 27.75 | 3533.77 | Entamoeba | NA | NA | NA |
| | | | | Histolytica | | | |
| CCF07 | Fixed | 46 | 0.64 | Giardia | NA | NA | NA |
| DLS13-05799-01-01 | Fixed | 46 | 4.03 | Giardia, | NA | Positive | Positive |
| | | | | Crypto | | | |
| CIN01010 | Fixed | 46 | 3.1 | Giardia | NA | Positive | NA |
| 13S0000137 | Fixed | 46 | 1.84 | Negative | Negative | NA | NA |
| 13S0000034 | Unpreserved | 46 | 15.23 | Negative | NA | NA | NA |
| 4369 | Fixed | 46 | 3.26 | Crypto | NA | NA | Positive |
| 12S0000787 | Unpreserved | 46 | 7.78 | Negative | Negative | NA | NA |
| DLS13-05977 | Unpreserved | 46 | 21.38 | Crypto | Negative | NA | Positive |
| 13S0000125 | Fixed | 46 | 5.03 | Negative | Negative | NA | NA |
| Leiden 28 | Unpreserved | 46 | 46.83 | Negative | NA | NA | Positive |
| 138 | Fixed | 46 | 0.67 | Giardia | NA | Positive | NA |
| CIN01013 | Fixed | 46 | 1.94 | Giardia | NA | Negative | NA |
| 13S0000124 | Fixed | 46 | 19.7 | Negative | NA | NA | NA |
| 13S0000170 | Fixed | 46 | 0.88 | Negative | NA | NA | NA |
| 13S0000176 | Fixed | 46 | 0.7 | Negative | NA | NA | NA |
| 13S0000052 | Unpreserved | 46 | 0.65 | Negative | Negative | NA | NA |
| 13S0000073 | Unpreserved | 46 | 13.93 | Negative | NA | NA | NA |
| 13S0000051 | Unpreserved | 46 | 0.77 | Negative | NA | NA | NA |
| 13S0000567 | Fixed | 46 | 4.26 | Negative | NA | NA | NA |
| DLS13-05986 | Unpreserved | 46 | 2.24 | Negative | NA | NA | Positive |
| Leiden 66 | Unpreserved | 46 | 1.32 | Giardia | NA | Positive | NA |
| DLS13-05959 | Unpreserved | 46 | 3.76 | Crypto | NA | NA | Positive |
| Leiden 4 | Unpreserved | 46 | 12.08 | Giardia | NA | Positive | NA |
| KH12-6358 | Unpreserved | 46 | 4.31 | Giardia | NA | Positive | NA |
| CCF12 | Fixed | 46 | 2.65 | Giardia | NA | NA | NA |
| F21 | Fixed | 46 | 3 | Giardia | NA | Positive | NA |
| DLS13-05796-01-01 | Fixed | 46 | 2.64 | Crypto | NA | NA | Positive |
| Leiden 21 | Unpreserved | 46 | 8.02 | Crypto | NA | NA | Positive |
| 13S0000572 | Fixed | 46 | 1.6 | Negative | NA | NA | NA |
| Leiden 34 | Unpreserved | 46 | 7.64 | Crypto | NA | NA | Positive |

TABLE 10.2

| | Original Site | | | | |
|---|---|---|---|---|---|
| | Reference | Alt. PCR Amp? | Alt. PCR Amp? | Alt. PCR Amp? | Seq other |
| Specimen ID | Method Result | Entamoeba | Giardia | Result Crypto | organisms |
| 12S0000574 | Negative | Negative | Negative | Negative | |
| 13S0000190 | Negative | Negative | Negative | Negative | |
| F25 | Giardia | Negative | Amp | Negative | |
| 12S0000734 | Negative | Amp | Negative | Negative | |
| 13S0000172 | Negative | Negative | Negative | Negative | |
| 12S0000684 | Negative | Amp | Negative | Negative | |
| 6304 | Crypto | Negative | Negative | Amp | |
| 13S0000575 | Negative | Negative | Negative | Negative | |
| 12S0000758 | Negative | Negative | Negative | Negative | |
| 6419 | Crypto | Negative | Negative | Amp | |
| EH22 | Entamoeba | Negative | Negative | Negative | |
| | Histolytica | | | | |
| 13S0000080 | Negative | Negative | Negative | Negative | |
| Leiden 75 | Giardia | Amp | Amp | Negative | |
| Leiden 83 | Giardia | Amp | Amp | Negative | |
| F43 | Giardia | Negative | Amp | Negative | |
| 12S0000769 | Negative | Amp | Negative | Negative | |
| DLS13- | Giardia | Negative | Amp | Negative | |
| 05810-01-01 | | | | | |
| 12S0000739 | Negative | Negative | Negative | Negative | |
| 13S0000130 | Negative | Amp | Negative | Negative | |
| 12S0000565 | Negative | Negative | Negative | Negative | |
| 13S0000141 | Negative | Amp | Negative | Negative | |
| 13S0000561 | Negative | Negative | Negative | Negative | |
| 11995 | Crypto | Negative | Negative | Amp | |
| 181 | Giardia | Amp | Amp | Amp | |
| 13S0000069 | Negative | Amp | Negative | Negative | |
| E20 | Entamoeba | Amp | Negative | Negative | Entamoeba |
| | Histolytica | | | | dispar genes |
| | | | | | for 18S rRNA, |
| | | | | | ITS1, 5.8S |
| | | | | | rRNA, ITS2, |
| | | | | | complete |
| | | | | | sequence |
| 12S0000765 | Negative | Negative | Negative | Negative | |
| Leiden 12 | Crypto | Negative | Negative | Amp | |
| 12S0000693 | Negative | Amp | Negative | Negative | |
| Leiden 78 | Giardia | Amp | Amp | Negative | |
| EH17 | Entamoeba | Negative | Negative | Negative | |
| | Histolytica | | | | |
| CCF06 | Giardia | Negative | Negative | Amp | |
| EH19 | Entamoeba | Negative | Negative | Negative | |
| | Histolytica | | | | |
| EH20 | Entamoeba | Negative | Negative | Negative | |
| | Histolytica | | | | |
| 1438 | Crypto | Negative | Negative | Negative | |
| 13S0000005 | Negative | Negative | Negative | Amp | |
| 13S0000159 | Negative | Negative | Negative | Negative | |
| 12S0000722 | Negative | Negative | Negative | Negative | |
| DLS13- | Giardia | Amp | Amp | Negative | |
| 05812-01-01 | | | | | |
| 12S0000706 | Negative | Negative | Negative | Negative | |
| Leiden 64 | Giardia | Negative | Amp | Negative | |
| 13S0000559 | Negative | Negative | Negative | Negative | |
| 12S0000673 | Negative | Negative | Negative | Negative | |
| DLS13- | Giardia | Negative | Negative | Negative | |
| 05780-01-01 | | | | | |
| 12S0000788 | Negative | Amp | Negative | Negative | |
| 13S0000059 | Negative | Negative | Negative | Negative | |
| DLS13- | Crypto | Amp | Negative | Amp | |
| 05960 | | | | | |
| 13S0000139 | Negative | Amp | Negative | Amp | |
| 13S0000037 | Negative | Negative | Negative | Negative | |
| CIN01005 | Giardia | Negative | Negative | Negative | |
| 13S0000185 | Negative | Negative | Negative | Negative | |
| 12S0000699 | Negative | Amp | Negative | Negative | |
| 55 | Giardia | Amp | Amp | Negative | |
| CCF08 | Giardia | Negative | Negative | Negative | |
| 13S0000564 | Negative | Negative | Negative | Negative | |
| 13S0000131 | Negative | Amp | Negative | Negative | |
| 12S0000561 | Negative | Negative | Negative | Negative | |
| EH23 | Entamoeba | Amp | Negative | Negative | Entamoeba |
| | Histolytica | | | | histolytica |
| | | | | | gene for small |
| | | | | | subunit |
| | | | | | ribosomal |
| | | | | | RNA, strain: |
| | | | | | BF-841 cl1 |
| 564 | Crypto | Negative | Negative | Amp | |
| Leiden 26 | Crypto | Negative | Negative | Amp | |
| 12S0000771 | Negative | Negative | Negative | Negative | |
| 12S0000729 | Negative | Amp | Negative | Negative | |
| Leiden 57 | Giardia | Negative | Amp | Negative | |
| 12997 | Crypto | Negative | Negative | Amp | |
| 13S0000070 | Negative | Negative | Negative | Negative | |
| 13S0000171 | Negative | Negative | Negative | Negative | |
| DLS13- | Crypto | Amp | Negative | Negative | |
| 06001 | | | | | |
| Leiden 39 | Crypto | Amp | Negative | Amp | |
| DLS13- | Crypto | Amp | Negative | Amp | |
| 05967 | | | | | |
| EH18 | Entamoeba | Negative | Negative | Negative | |
| | Histolytica | | | | |
| 13S0000122 | Negative | Negative | Negative | Negative | |
| 13S0000057 | Negative | Negative | Negative | Negative | |
| 12S0000575 | Negative | Negative | Negative | Negative | |
| 12S0000737 | Negative | Negative | Negative | Negative | |
| 13S0000188 | Negative | Negative | Negative | Negative | |
| 12S0000569 | Negative | Negative | Negative | Negative | |
| 12S0000692 | Negative | Negative | Negative | Negative | |
| 13S0000036 | Negative | Amp | Negative | Negative | |
| 13S0000566 | Negative | Negative | Negative | Negative | |
| 13S0000109 | Negative | Negative | Negative | Negative | |
| 13S0000090 | Negative | Negative | Negative | Negative | |
| 36 | Giardia | Negative | Amp | Negative | |
| 12S0000697 | Negative | Negative | Negative | Negative | |
| 12S0000572 | Negative | Negative | Negative | Negative | |
| 13S0000106 | Negative | Negative | Negative | Negative | |
| F35 | Giardia | Negative | Amp | Negative | |
| 12S0000735 | Negative | Negative | Negative | Negative | |
| 13S0000177 | Negative | Negative | Negative | Negative | |
| 12S0000707 | Negative | Negative | Negative | Negative | |
| 12S0000708 | Negative | Amp | Negative | Negative | |
| DLS13- | Giardia | Negative | Negative | Negative | |
| 05974 | | | | | |
| 13S0000008 | Negative | Negative | Negative | Negative | |
| 13S0000011 | Negative | Negative | Negative | Negative | |
| DLS13- | Crypto | Negative | Negative | Amp | |
| 05782-01-01 | | | | | |
| 12S0000670 | Negative | Negative | Negative | Negative | |
| CIN01020 | Giardia | Negative | Negative | Negative | |
| 13S0000156 | Negative | Negative | Negative | Negative | |
| 13S0000039 | Negative | Negative | Negative | Negative | |
| 13S0000035 | Negative | Amp | Negative | Negative | |
| 12S0000570 | Negative | Negative | Negative | Negative | |
| 11334 | Crypto | Negative | Negative | Amp | |
| 13S0000569 | Negative | Negative | Negative | Negative | |
| 13S0000065 | Negative | Negative | Negative | Negative | |
| EH12 | Entamoeba | Negative | Negative | Negative | |
| | Histolytica | | | | |
| 12S0000777 | Negative | Negative | Negative | Negative | |
| 13S0000116 | Negative | Negative | Negative | Negative | |
| DLS13- | Crypto | Negative | Negative | Amp | |
| 05994 | | | | | |
| 13S0000138 | Negative | Negative | Negative | Negative | |
| Leiden 68 | Giardia | Negative | Amp | Negative | |
| Leiden 63 | Giardia | Negative | Amp | Negative | |
| 13S0000149 | Negative | Negative | Negative | Negative | |
| 12S0000732 | Negative | Negative | Negative | Negative | |
| DLS13- | Giardia | Amp | Amp | Amp | |
| 05976 | | | | | |
| 12S0000688 | Negative | Negative | Negative | Negative | |
| EH08 | Entamoeba | Amp | Negative | Negative | Entamoeba |
| | Histolytica | | | | histolytica |
| | | | | | gene for small |
| | | | | | subunit |
| | | | | | ribosomal |
| | | | | | RNA, strain: |
| | | | | | BF-841 cl1 |
| CIN01004 | Giardia | Negative | Negative | Negative | |
| DLS13- | Crypto | Negative | Negative | Amp | |
| 05956 | | | | | |
| Leiden 10 | Crypto | Negative | Negative | Amp | |
| Leiden 37 | Crypto | Amp | Negative | Amp | |
| DLS13- | Giardia | Negative | Negative | Amp | |
| 05790-01-01 | | | | | |
| DLS13- | Entamoeba | Negative | Negative | Negative | |
| 05996 | Histolytica | | | | |
| F33 | Giardia | Negative | Amp | Negative | |
| Leiden 96 | Giardia | Negative | Amp | Negative | |
| Leiden 99 | Giardia | Negative | Amp | Negative | |
| 13S0000183 | Negative | Negative | Negative | Negative | |
| 12S0000503 | Negative | Negative | Negative | Negative | |
| 13S0000092 | Negative | Negative | Negative | Negative | |
| Leiden 84 | Giardia | Amp | Amp | Negative | |
| 12S0000786 | Negative | Negative | Negative | Negative | |
| DLS13- | Crypto | Amp | Amp | Amp | |
| 05817-01-01 | | | | | |
| Leiden 79 | Giardia | Negative | Negative | Negative | |
| 13S0000111 | Negative | Negative | Negative | Negative | |
| CCF11 | Giardia | Negative | Negative | Negative | |
| 124 | Giardia | Negative | Amp | Negative | |
| 13S0000563 | Negative | Negative | Negative | Negative | |
| 12S0000678 | Negative | Negative | Negative | Negative | |
| 12S0000698 | Negative | Negative | Negative | Negative | |
| 77 | Giardia | Amp | Amp | Negative | Entamoeba |
| | | | | | coli partial |
| | | | | | 18S rRNA |
| | | | | | gene, isolate |
| | | | | | EM049 |
| 13S0000084 | Negative | Negative | Negative | Negative | |
| 12S0000746 | Negative | Negative | Negative | Negative | |
| CIN01040 | Giardia | Negative | Amp | Negative | |
| 13S0000117 | Negative | Negative | Negative | Negative | |
| Leiden 32 | Crypto | Negative | Negative | Amp | |
| 13S0000112 | Negative | Negative | Negative | Negative | |
| 12S0000711 | Negative | Negative | Negative | Negative | |
| DLS13- | Entamoeba | Amp | Negative | Negative | |
| 05982 | Histolytica | | | | |
| 13S0000032 | Negative | Negative | Negative | Negative | |
| 13S0000565 | Negative | Negative | Negative | Negative | |
| DLS13- | Crypto | Negative | Negative | Amp | |
| 05787-01-01 | | | | | |
| 12S0000760 | Negative | Negative | Negative | Negative | |
| Leiden 98 | Giardia | Negative | Amp | Negative | |
| 13S0000087 | Negative | Negative | Negative | Negative | |
| F41 | Giardia | Negative | Amp | Negative | |
| 6289 | Crypto | Negative | Negative | Amp | |
| DLS13- | Crypto | Amp | Negative | Amp | |
| 05947 | | | | | |
| E18 | Entamoeba | Amp | Negative | Negative | Entamoeba |
| | Histolytica | | | | dispar genes |
| | | | | | for 18S rRNA, |
| | | | | | ITS1, 5.8S |
| | | | | | rRNA, ITS2, |
| | | | | | complete |
| | | | | | sequence |
| 13S0000568 | Negative | Negative | Negative | Negative | |
| F37 | Giardia | Negative | Amp | Negative | |
| Leiden 36 | Crypto | Amp | Negative | Amp | |
| 13S0000158 | Negative | Negative | Negative | Negative | |
| DLS13-05973 | Entamoeba | Negative | Negative | Negative | |
| | Histolytica | | | | |
| 13S0000053 | Negative | Negative | Negative | Negative | |
| Leiden 91 | Giardia | Negative | Amp | Negative | |
| DLS13- | Giardia | Amp | Negative | Amp | |
| 05966 | | | | | |
| 12S0000778 | Negative | Negative | Negative | Negative | |
| 13S0000151 | Negative | Negative | Negative | Negative | |
| 13S0000147 | Negative | Negative | Negative | Negative | |
| 13S0000179 | Negative | Negative | Negative | Negative | |
| 13S0000006 | Negative | Negative | Negative | Negative | |
| 13S0000189 | Negative | Negative | Negative | Negative | |
| 12S0000573 | Negative | Negative | Negative | Negative | |
| DLS13- | Crypto | Amp | Negative | Amp | |
| 05980 | | | | | |
| 12S0000791 | Negative | Negative | Negative | Negative | |
| Leiden 25 | Crypto | Amp | Negative | Amp | |
| DLS13- | Crypto | Amp | Negative | Amp | |
| 05952 | | | | | |
| 13S0000019 | Negative | Negative | Negative | Negative | |
| 91 | Giardia | Negative | Amp | Negative | |
| DLS13-05972 | Crypto | Amp | Negative | Negative | |
| 13S0000043 | Negative | Negative | Negative | Negative | |
| 12S0000714 | Negative | Negative | Negative | Negative | |
| 12S0000675 | Negative | Negative | Negative | Negative | |
| DLS13- | Crypto | Negative | Negative | Amp | |
| 05801-01-01 | | | | | |
| 12S0000682 | Negative | Negative | Negative | Negative | |
| DLS13- | Crypto | Negative | Negative | Amp | |
| 05802-01-01 | | | | | |
| 113 | Giardia | Amp | Amp | Negative | |
| Leiden 85 | Giardia | Negative | Amp | Negative | |
| EH14 | Entamoeba | Amp | Negative | Negative | Entamoeba |
| | Histolytica | | | | histolytica |
| | | | | | gene for small |
| | | | | | subunit |
| | | | | | ribosomal |
| | | | | | RNA, strain: |
| | | | | | BF-841 cl1 |
| 13S0000096 | Negative | Negative | Negative | Negative | |
| 12S0000733 | Negative | Amp | Negative | Negative | |
| 12S0000689 | Negative | Negative | Negative | Negative | |
| CCF03 | Giardia | Negative | Negative | Negative | |
| 13S0000049 | Negative | Negative | Negative | Negative | |
| Leiden 8 | Crypto | Negative | Negative | Amp | |
| Leiden 29 | Crypto | Amp | Negative | Amp | |
| 12S0000576 | Negative | Negative | Negative | Negative | |
| Leiden 6 | Crypto | Amp | Negative | Amp | |
| 13S0000007 | Negative | Negative | Negative | Negative | |
| 12S0000717 | Negative | Negative | Negative | Negative | |
| 13S0000001 | Negative | Negative | Negative | Negative | |
| 13S0000134 | Negative | Negative | Amp | Negative | |
| 13S0000104 | Negative | Negative | Negative | Negative | |
| 13S0000089 | Negative | Negative | Negative | Negative | |
| 12S0000762 | Negative | Negative | Negative | Negative | |
| 13S0000562 | Negative | Negative | Negative | Negative | |
| 13S0000010 | Negative | Negative | Negative | Negative | |
| 125 | Giardia | Negative | Amp | Negative | |
| 13S0000135 | Negative | Negative | Negative | Negative | |
| 13S0000118 | Negative | Negative | Negative | Negative | |
| 12S0000713 | Negative | Negative | Negative | Negative | |
| 13S0000162 | Negative | Negative | Negative | Negative | |
| DLS13- | Crypto | Amp | Negative | Amp | |
| 05970 | | | | | |
| 12S0000727 | Negative | Amp | Negative | Negative | |
| 39546 | Giardia | Amp | Amp | Negative | |
| 13S0000083 | Negative | Negative | Negative | Negative | |
| Leiden 49 | Crypto | Amp | Negative | Amp | |
| EH09 | Entamoeba | Negative | Negative | Negative | |
| | Histolytica | | | | |
| F31 | Giardia | Amp | Amp | Negative | |
| 12S0000751 | Negative | Amp | Negative | Negative | |
| 13S0000153 | Negative | Amp | Negative | Negative | |
| 13S0000166 | Negative | Amp | Negative | Negative | |
| 12S0000685 | Negative | Negative | Negative | Negative | |
| CCF10 | Giardia | Negative | Negative | Negative | |
| EH10 | Entamoeba | Negative | Negative | Negative | |
| | Histolytica | | | | |
| 13S0000098 | Negative | Negative | Negative | Negative | |
| 81 | Giardia | Amp | Amp | Negative | |
| 13S0000095 | Negative | Negative | Negative | Negative | |
| Leiden 1 | Crypto | Amp | Negative | Amp | |
| 13S0000173 | Negative | Amp | Negative | Negative | |
| DLS13- | Giardia | Negative | Amp | Negative | |
| 05784-01-01 | | | | | |
| Leiden 51 | Giardia | Amp | Amp | Negative | |
| 12S0000725 | Negative | Negative | Negative | Negative | |
| 162 | Giardia | Amp | Amp | Negative | Entamoeba |
| | | | | | coli partial |
| | | | | | 18S rRNA |
| | | | | | gene, isolate |
| | | | | | J65 |
| 12S0000781 | Negative | Negative | Negative | Negative | |
| 13S0000143 | Negative | Negative | Negative | Negative | |
| 13S0000003 | Negative | Negative | Negative | Negative | |
| 12S0000710 | Negative | Negative | Negative | Negative | |
| Leiden 86 | Giardia | Amp | Amp | Negative | |
| 13S0000027 | Negative | Negative | Negative | Negative | |
| DLS13- | Crypto | Negative | Negative | Negative | |
| 05949 | | | | | |
| 12S0000702 | Negative | Amp | Negative | Negative | |
| Leiden 82 | Giardia | Amp | Amp | Negative | |
| 1247 | Crypto | Negative | Negative | Amp | |
| 13S0000140 | Negative | Amp | Negative | Negative | |
| Leiden 59 | Giardia | Amp | Amp | Negative | Entamoeba |
| | | | | | dispar genes |
| | | | | | for 18S rRNA, |
| | | | | | ITS1, 5.8S |
| | | | | | rRNA, ITS2, |
| | | | | | complete |
| | | | | | sequence |
| 13S0000558 | Negative | Amp | Negative | Negative | |
| 13S0000157 | Negative | Amp | Negative | Negative | |
| 8174 | Crypto | Negative | Negative | Negative | |
| F29 | Giardia | Amp | Amp | Negative | |
| CIN01026 | Giardia | Amp | Amp | Negative | |
| Leiden 58 | Giardia | Negative | Amp | Negative | |
| 13S0000165 | Negative | Amp | Negative | Negative | |
| 13S0000164 | Negative | Amp | Negative | Negative | |
| DLS13- | Crypto | Amp | Negative | Amp | |
| 05808-01-01 | | | | | |
| 12S0000716 | Negative | Amp | Negative | Negative | |
| 12S0000784 | Negative | Amp | Negative | Negative | |
| EH24 | Entamoeba | Amp | Negative | Negative | |
| | Histolytica | | | | |
| 12S0000681 | Negative | Amp | Negative | Negative | |
| DLS13- | Crypto | Amp | Negative | Amp | |
| 05969 | | | | | |
| Leiden 62 | Giardia | Amp | Amp | Negative | |
| 13S0000002 | Negative | Amp | Negative | Negative | |
| 12S0000770 | Negative | Amp | Negative | Negative | |
| DLS13- | Giardia | Negative | Negative | Amp | |
| 05789-01-01 | | | | | |
| 13S0000560 | Negative | Amp | Negative | Negative | |
| 13S0000145 | Negative | Amp | Negative | Negative | |
| DLS13- | Crypto | Amp | Negative | Amp | |
| 05958 | | | | | |
| 12S0000563 | Negative | Amp | Negative | Negative | |
| 12S0000683 | Negative | Amp | Negative | Negative | |
| 13S0000168 | Negative | Negative | Negative | Negative | |
| Leiden 46 | Crypto | Negative | Negative | Amp | |
| E24 | Entamoeba | Amp | Negative | Negative | Entamoeba |
| | Histolytica | | | | dispar genes |
| | | | | | for 18S rRNA, |
| | | | | | ITS1, 5.8S |
| | | | | | rRNA, ITS2, |
| | | | | | complete |
| | | | | | sequence |
| 13S0000101 | Negative | Negative | Negative | Negative | |
| 13S0000155 | Negative | Negative | Negative | Negative | |
| Leiden 71 | Giardia | Negative | Amp | Negative | |
| EH06 | Entamoeba | Negative | Negative | Negative | |
| | Histolytica | | | | |
| DLS13- | Crypto | Negative | Negative | Amp | |
| 05816-01-01 | | | | | |
| F27 | Giardia | Negative | Amp | Negative | |
| DLS13- | Crypto | Amp | Negative | Negative | |
| 05997 | | | | | |
| EH04 | Entamoeba | Negative | Negative | Negative | |
| | Histolytica | | | | |
| 12S0000726 | Negative | Negative | Negative | Negative | |
| DLS13- | Entamoeba | Amp | Negative | Negative | |
| 05963 | Histolytica | | | | |
| DLS13- | Crypto | Negative | Negative | Amp | |
| 05793-01-01 | | | | | |
| 13S0000126 | Negative | Negative | Negative | Negative | |
| EH25 | Entamoeba | Negative | Negative | Negative | |
| | Histolytica | | | | |
| 40015 | Crypto | Negative | Negative | Amp | |
| 13S0000181 | Negative | Amp | Negative | Negative | |
| 58 | Giardia | Amp | Amp | Negative | Entamoeba |
| | | | | | hartmanni |
| | | | | | partial 18S |
| | | | | | rRNA gene, |
| | | | | | isolate |
| | | | | | EM061a |
| CCF02 | Giardia | Negative | Amp | Negative | |
| 12S0000568 | Negative | Negative | Negative | Negative | |
| 96 | Giardia | Negative | Amp | Negative | |
| Leiden 43 | Crypto | Negative | Negative | Amp | |
| 13S0000123 | Negative | Negative | Negative | Negative | |
| 12S0000705 | Negative | Negative | Negative | Negative | |
| DLS13- | Crypto | Negative | Negative | Amp | |
| 05794-01-01 | | | | | |
| 13S0000136 | Negative | Negative | Negative | Negative | |
| Leiden 97 | Giardia | Amp | Amp | Negative | |
| DLS13- | Crypto | Negative | Negative | Amp | |
| 05798-01-01 | | | | | |
| 12S0000719 | Negative | Amp | Negative | Negative | |
| 12S0000668 | Negative | Negative | Negative | Negative | |
| Leiden 80 | Giardia | Negative | Negative | Negative | |
| 12S0000756 | Negative | Negative | Negative | Negative | |
| 13S0000107 | Negative | Negative | Negative | Negative | |
| 13S0000068 | Negative | Amp | Negative | Negative | |
| 12S0000749 | Negative | Negative | Negative | Negative | |
| Leiden 16 | Crypto | Amp | Negative | Amp | |
| Leiden 72 | Giardia | Negative | Amp | Negative | |
| 12S0000763 | Negative | Amp | Negative | Negative | Entamoeba |
| | | | | | coli partial |
| | | | | | 18S rRNA |
| | | | | | gene, isolate |
| | | | | | J65 |
| 12S0000709 | Negative | Amp | Negative | Negative | |
| 13S0000072 | Negative | Negative | Negative | Negative | |
| 13S0000013 | Negative | Negative | Negative | Negative | |
| CCF01 | Giardia | Negative | Amp | Negative | |
| DLS13- | Crypto | Amp | Negative | Amp | |
| 05953 | | | | | |
| 12S0000761 | Negative | Negative | Negative | Negative | |
| Leiden 9 | Crypto | Negative | Negative | Amp | |
| 13S0000054 | Negative | Negative | Negative | Negative | |
| EH16 | Entamoeba | Negative | Negative | Negative | |
| | Histolytica | | | | |
| 13S0000081 | Negative | Negative | Negative | Negative | |
| DLS13- | Crypto | Amp | Negative | Amp | |
| 05951 | | | | | |
| DLS13- | Giardia | Amp | Amp | Negative | |
| 05820-01-01 | | | | | |
| Leiden 5 | Crypto | Amp | Negative | Amp | |
| EH07 | Entamoeba | Negative | Negative | Negative | |
| | Histolytica | | | | |
| 13S0000146 | Negative | Negative | Negative | Negative | |
| CIN01012 | Giardia | Negative | Amp | Negative | |
| DLS13- | Giardia | Amp | Amp | Negative | |
| 05818-01-01 | | | | | |
| 13S0000169 | Negative | Amp | Negative | Negative | |
| 13S0000186 | Negative | Negative | Negative | Negative | |
| 13S0000152 | Negative | Amp | Negative | Negative | |
| 12S0000669 | Negative | Negative | Negative | Negative | |
| 12S0000566 | Negative | Negative | Negative | Negative | |
| 14790 | Crypto | Negative | Negative | Amp | |
| Leiden 18 | Crypto | Amp | Negative | Amp | |
| 13S0000094 | Negative | Amp | Negative | Negative | |
| 13S0000154 | Negative | Negative | Negative | Negative | |
| 12S0000724 | Negative | Negative | Negative | Negative | |
| 13S0000161 | Negative | Amp | Negative | Negative | |
| 12S0000562 | Negative | Amp | Negative | Negative | |
| 13S0000040 | Negative | Negative | Negative | Negative | |
| 13S0000024 | Negative | Negative | Negative | Negative | |
| 13S0000148 | Negative | Negative | Negative | Negative | |
| DLS13- | Crypto | Amp | Negative | Amp | |
| 05983 | | | | | |
| 159 | Giardia | Amp | Amp | Negative | |
| Leiden 74 | Giardia | Negative | Amp | Negative | |
| 13S0000031 | Negative | Negative | Negative | Negative | |
| EH05 | Entamoeba | Amp | Negative | Negative | Entamoeba |
| | Histolytica | | | | histolytica |
| | | | | | gene for small |
| | | | | | subunit |
| | | | | | ribosomal |
| | | | | | RNA, strain: |
| | | | | | BF-841 cl1 |
| 3645 | Crypto | Negative | Negative | Amp | |
| 13S0000119 | Negative | Negative | Negative | Negative | |
| 13S0000021 | Negative | Amp | Negative | Negative | |
| 13S0000132 | Negative | Negative | Negative | Negative | |
| 12S0000764 | Negative | Amp | Negative | Negative | |
| 13S0000064 | Negative | Negative | Negative | Negative | |
| 183 | Giardia | Negative | Amp | Negative | |
| DLS13- | Crypto | Amp | Negative | Amp | |
| 05950 | | | | | |
| 13S0000557 | Negative | Negative | Negative | Negative | |
| CIN01038 | Giardia | Negative | Amp | Negative | |
| 13S0000120 | Negative | Negative | Negative | Negative | |
| EH01 | Entamoeba | Amp | Negative | Negative | Entamoeba |
| | Histolytica | | | | histolytica |
| | | | | | gene for small |
| | | | | | subunit |
| | | | | | ribosomal |
| | | | | | RNA, strain: |
| | | | | | BF-841 cl1 |
| 12S0000679 | Negative | Negative | Negative | Negative | |
| Leiden 73 | Giardia | Negative | Amp | Negative | |
| DLS13- | Crypto | Negative | Amp | Negative | |
| 05781-01-01 | | | | | |
| 13S0000150 | Negative | Negative | Negative | Negative | |
| 13S0000115 | Negative | Negative | Negative | Negative | |
| 13S0000142 | Negative | Negative | Negative | Negative | |
| Leiden 70 | Giardia | Negative | Amp | Negative | |
| Leiden 69 | Giardia | Negative | Amp | Negative | |
| 12S0000704 | Negative | Negative | Negative | Negative | |
| 129 | Giardia | Negative | Amp | Negative | |
| Leiden 61 | Giardia | Amp | Amp | Negative | |
| EH03 | Entamoeba | Negative | Negative | Negative | |
| | Histolytica | | | | |
| 13S0000121 | Negative | Amp | Negative | Negative | |
| 7458 | Crypto | Negative | Negative | Negative | |
| 13S0000129 | Negative | Negative | Negative | Negative | |
| 12S0000703 | Negative | Negative | Negative | Negative | |
| 13S0000056 | Negative | Amp | Negative | Negative | |
| 12S0000766 | Negative | Negative | Negative | Negative | |
| F23 | Giardia | Negative | Amp | Negative | |
| 13S0000103 | Negative | Negative | Negative | Negative | |
| 12S0000723 | Negative | Negative | Negative | Negative | |
| 130 | Giardia | Negative | Amp | Negative | |
| Leiden 52 | Giardia | Negative | Amp | Negative | |
| Leiden 60 | Giardia | Negative | Amp | Negative | |
| 13S0000038 | Negative | Negative | Negative | Negative | |
| 13S0000100 | Negative | Negative | Negative | Negative | |
| 13S0000102 | Negative | Negative | Negative | Negative | |
| 13S0000576 | Negative | Negative | Negative | Negative | |
| 12S0000567 | Negative | Negative | Negative | Negative | |
| 12S0000672 | Negative | Negative | Negative | Negative | |
| 12S0000757 | Negative | Negative | Negative | Negative | |
| 12S0000742 | Negative | Negative | Negative | Negative | |
| Leiden 17 | Crypto | Negative | Negative | Amp | |
| Leiden 15 | Crypto | Negative | Negative | Amp | |
| 13S0000009 | Negative | Negative | Negative | Negative | |
| CIN01028 | Giardia | Negative | Amp | Negative | |
| 13S0000127 | Negative | Amp | Negative | Negative | Entamoeba |
| | | | | | hartmanni |
| | | | | | partial 18S |
| | | | | | rRNA gene, |
| | | | | | isolate EM042 |
| KH12-5156 | Giardia | Amp | Amp | Negative | |
| 12S0000674 | Negative | Negative | Negative | Negative | |
| 13S0000571 | Negative | Negative | Negative | Negative | |
| 13S0000128 | Negative | Negative | Negative | Negative | |
| 140 | Giardia | Amp | Amp | Negative | Entamoeba |
| | | | | | coli strain |
| | | | | | IH: 96/135 |
| | | | | | 16S-like small |
| | | | | | subunit |
| | | | | | ribosomal |
| | | | | | RNA gene, |
| | | | | | complete |
| | | | | | sequence |
| 12S0000738 | Negative | Negative | Negative | Negative | |
| CIN01039 | Giardia | Negative | Amp | Negative | |
| KH12-4357 | Giardia | Negative | Amp | Negative | |
| Leiden 30 | Crypto | Amp | Negative | Amp | |
| 13S0000114 | Negative | Negative | Negative | Negative | |
| Leiden 55 | Giardia | Amp | Amp | Negative | |
| 12S0000680 | Negative | Negative | Negative | Negative | |
| E22 | Entamoeba | Amp | Negative | Negative | Entamoeba |
| | Histolytica | | | | dispar genes |
| | | | | | for 18S rRNA, |
| | | | | | ITS1, 5.8S |
| | | | | | rRNA, ITS2, |
| | | | | | complete |
| | | | | | sequence |
| 39543 | Giardia | Negative | Amp | Negative | |
| 12S0000776 | Negative | Negative | Negative | Negative | |
| KH12-6359 | Giardia | Negative | Amp | Negative | |
| 13S0000160 | Negative | Negative | Negative | Negative | |
| DLS13- | Giardia | Negative | Amp | Amp | |
| 05945 | | | | | |
| 11796 | Crypto | Negative | Negative | Amp | |
| DLS13- | Crypto | Negative | Amp | Negative | |
| 05978 | | | | | |
| 13S0000187 | Negative | Negative | Negative | Negative | |
| 13S0000063 | Negative | Amp | Negative | Negative | |
| 12S0000779 | Negative | Negative | Negative | Negative | |
| DLS13- | Crypto | Amp | Negative | Amp | |
| 05800-01-01 | | | | | |
| 13S0000110 | Negative | Negative | Negative | Negative | |
| 13S0000113 | Negative | Negative | Negative | Negative | |
| CIN01027 | Giardia | Negative | Negative | Negative | |
| 13S0000180 | Negative | Negative | Negative | Negative | |
| 12S0000564 | Negative | Negative | Negative | Negative | |
| CCF05 | Giardia | Negative | Negative | Negative | |
| DLS13- | Crypto | Amp | Negative | Negative | |
| 05991 | | | | | |
| 12S0000667 | Negative | Negative | Negative | Negative | |
| 13S0000182 | Negative | Negative | Negative | Negative | |
| 12S0000790 | Negative | Negative | Negative | Negative | |
| CIN01003 | Giardia | Negative | Negative | Negative | |
| Leiden 19 | Crypto | Amp | Negative | Amp | |
| DLS13- | Crypto | Negative | Negative | Amp | |
| 05792-01-01 | | | | | |
| 13S0000099 | Negative | Negative | Negative | Negative | |
| CIN01024 | Giardia | Negative | Negative | Negative | |
| 13S0000062 | Negative | Amp | Negative | Negative | |
| EH13 | Entamoeba | Negative | Negative | Negative | |
| | Histolytica | | | | |
| 12S0000715 | Negative | Negative | Negative | Negative | |
| DLS13- | Crypto | Amp | Amp | Amp | |
| 05961 | | | | | |
| DLS13- | Crypto | Amp | Amp | Amp | |
| 05981 | | | | | |
| CCF04 | Giardia | Negative | Negative | Negative | |
| Leiden 22 | Crypto | Negative | Negative | Amp | |
| EH02 | Entamoeba | Amp | Negative | Negative | Entamoeba |
| | Histolytica | | | | histolytica |
| | | | | | gene for small |
| | | | | | subunit |
| | | | | | ribosomal |
| | | | | | RNA, strain: |
| | | | | | BF-841 cl1 |
| 13S0000184 | Negative | Negative | Negative | Negative | |
| Leiden 35 | Crypto | Amp | Negative | Amp | |
| DLS13- | Giardia | Negative | Negative | Negative | |
| 05807-01-01 | | | | | |
| 13S0000175 | Negative | Negative | Negative | Negative | |
| 13S0000091 | Negative | Negative | Negative | Negative | |
| 13S0000075 | Negative | Negative | Negative | Negative | |
| DLS13- | Crypto | Negative | Negative | Amp | |
| 05795-01-01 | | | | | |
| 12998 | Crypto | Amp | Negative | Amp | |
| 13S0000174 | Negative | Negative | Negative | Negative | |
| 13S0000055 | Negative | Amp | Negative | Negative | |
| 12S0000782 | Negative | Negative | Negative | Negative | |
| 13S0000163 | Negative | Negative | Negative | Negative | |
| 13S0000574 | Negative | Negative | Negative | Negative | |
| 13S0000071 | Negative | Negative | Negative | Negative | |
| Leiden 2 | Crypto | Amp | Amp | Amp | |
| 13S0000167 | Negative | Negative | Negative | Negative | |
| 13S0000133 | Negative | Negative | Negative | Negative | |
| 13S0000046 | Negative | Negative | Negative | Negative | |
| 12S0000759 | Negative | Negative | Negative | Negative | |
| Leiden 7 | Crypto | Amp | Negative | Amp | |
| 13S0000041 | Negative | Negative | Negative | Negative | |
| 12S0000671 | Negative | Amp | Negative | Negative | |
| 13S0000093 | Negative | Amp | Negative | Negative | |
| CIN01006 | Giardia | Negative | Negative | Negative | |
| Leiden 33 | Crypto | Negative | Negative | Amp | |
| DLS13- | Crypto | Amp | Negative | Negative | |
| 05882 | | | | | |
| 13S0000144 | Negative | Amp | Negative | Negative | |
| DLS13- | Crypto | Amp | Negative | Negative | |
| 05893 | | | | | |
| 13S0000088 | Negative | Amp | Negative | Negative | |
| DLS13- | Crypto | Amp | Negative | Amp | |
| 05954 | | | | | |
| 20 | Giardia | Amp | Amp | Negative | |
| 13S0000060 | Negative | Amp | Negative | Negative | |
| 64 | Giardia | Negative | Amp | Negative | |
| Leiden 67 | Giardia | Negative | Amp | Negative | |
| 13S0000066 | Negative | Negative | Negative | Negative | |
| 13S0000178 | Negative | Amp | Negative | Negative | |
| 13S0000570 | Negative | Negative | Negative | Negative | |
| 12S0000712 | Negative | Amp | Negative | Negative | |
| DLS13- | Crypto | Negative | Negative | Amp | |
| 05962 | | | | | |
| 73 | Giardia | Negative | Amp | Negative | |
| EH15 | Entamoeba | Negative | Negative | Negative | |
| | Histolytica | | | | |
| Leiden 81 | Giardia | Negative | Amp | Negative | |
| Leiden 13 | Crypto | Negative | Negative | Amp | |
| 12S0000731 | Negative | Amp | Negative | Negative | |
| 12S0000686 | Negative | Negative | Negative | Negative | |
| 12S0000676 | Negative | Negative | Negative | Negative | |
| 13S0000067 | Negative | Negative | Negative | Negative | |
| CIN01011 | Giardia | Negative | Amp | Negative | |
| Leiden 20 | Crypto | Amp | Negative | Negative | |
| EH11 | Entamoeba | Amp | Negative | Negative | Entamoeba |
| | Histolytica | | | | histolytica |
| | | | | | gene for small |
| | | | | | subunit |
| | | | | | ribosomal |
| | | | | | RNA, strain: |
| | | | | | BF-841 cl1 |
| Leiden 23 | Crypto | Negative | Negative | Amp | |
| 161 | Giardia | Negative | Negative | Negative | |
| 13S0000058 | Negative | Negative | Negative | Negative | |
| 13S0000077 | Negative | Negative | Negative | Negative | |
| DLS13- | Crypto | Negative | Negative | Amp | |
| 05809-01-01 | | | | | |
| DLS13- | Crypto | Amp | Negative | Amp | |
| 05788-01-01 | | | | | |
| DLS13- | Crypto | Amp | Negative | Amp | |
| 05987 | | | | | |
| Leiden 95 | Giardia | Amp | Amp | Negative | |
| 68 | Giardia | Negative | Amp | Negative | |
| Leiden 53 | Giardia | Negative | Amp | Negative | |
| 12S0000677 | Negative | Negative | Negative | Negative | |
| 12S0000721 | Negative | Negative | Negative | Negative | |
| DLS13- | Crypto/Giardia | Negative | Amp | Negative | |
| 05814-01-01 | | | | | |
| Leiden 11 | Crypto | Negative | Negative | Amp | |
| Leiden 3 | Crypto | Negative | Negative | Amp | |
| 12S0000577 | Negative | Negative | Negative | Negative | |
| 12S0000701 | Negative | Negative | Negative | Negative | |
| 37 | Giardia | Negative | Amp | Negative | |
| 13S0000076 | Negative | Negative | Negative | Negative | |
| DLS13- | Crypto | Negative | Negative | Amp | |
| 05968 | | | | | |
| DLS13- | Crypto | Negative | Negative | Amp | |
| 05805-01-01 | | | | | |
| 13S0000078 | Negative | Negative | Negative | Negative | |
| Leiden 65 | Giardia | Amp | Amp | Negative | |
| 13S0000573 | Negative | Negative | Negative | Negative | |
| 13S0000097 | Negative | Negative | Negative | Negative | |
| DLS13- | Crypto | Negative | Negative | Amp | |
| 05791-01-01 | | | | | |
| 12S0000691 | Negative | Negative | Negative | Negative | |
| 13S0000105 | Negative | Negative | Negative | Negative | |
| 13S0000108 | Negative | Negative | Negative | Negative | |
| 12S0000560 | Negative | Negative | Negative | Negative | |
| 13S0000042 | Negative | Negative | Negative | Negative | |
| 12S0000690 | Negative | Negative | Negative | Negative | |
| Leiden 92 | Giardia | Negative | Amp | Negative | |
| DLS13- | Crypto | Negative | Negative | Amp | |
| 05804-01-01 | | | | | |
| 11556 | Crypto | Negative | Negative | Amp | |
| 13S0000014 | Negative | Negative | Negative | Negative | |
| EH21 | Entamoeba | Negative | Negative | Negative | |
| | Histolytica | | | | |
| CCF07 | Giardia | Negative | Negative | Negative | |
| DLS13- | Crypto | Negative | Amp | Amp | |
| 05799-01-01 | | | | | |
| CIN01010 | Giardia | Negative | Amp | Negative | |
| 13S0000137 | Negative | Amp | Negative | Negative | |
| 13S0000034 | Negative | Negative | Negative | Negative | |
| 4369 | Crypto | Negative | Negative | Amp | |
| 12S0000787 | Negative | Amp | Negative | Negative | |
| DLS13-05977 | Crypto | Amp | Negative | Amp | |
| 13S0000125 | Negative | Amp | Negative | Negative | |
| Leiden 28 | Crypto | Negative | Negative | Amp | |
| 138 | Giardia | Negative | Amp | Negative | |
| CIN01013 | Giardia | Negative | Amp | Negative | |
| 13S0000124 | Negative | Negative | Negative | Negative | |
| 13S0000170 | Negative | Negative | Negative | Negative | |
| 13S0000176 | Negative | Negative | Negative | Negative | |
| 13S0000052 | Negative | Amp | Negative | Negative | |
| 13S0000073 | Negative | Negative | Negative | Negative | |
| 13S0000051 | Negative | Negative | Negative | Negative | |
| 13S0000567 | Negative | Negative | Negative | Negative | |
| DLS13- | Crypto | Negative | Negative | Amp | |
| 05986 | | | | | |
| Leiden 66 | Giardia | Negative | Amp | Negative | |
| DLS13- | Crypto | Negative | Negative | Amp | |
| 05959 | | | | | |
| Leiden 4 | Crypto | Negative | Amp | Negative | |
| KH12-6358 | Giardia | Negative | Amp | Negative | |
| CCF12 | Giardia | Negative | Negative | Negative | |
| F21 | Giardia | Negative | Amp | Negative | |
| DLS13- | Crypto | Negative | Negative | Amp | |
| 05796-01-01 | | | | | |
| Leiden 21 | Crypto | Negative | Negative | Amp | |
| 13S0000572 | Negative | Negative | Negative | Negative | |
| Leiden 34 | Crypto | Negative | Negative | Amp | |

### Example 11

### Clinical Simulation

To further confirm the ability of the BD MAX™ assay to detect E. histolytica in samples positive for E. histolytica, a contrived clinical simulation was performed. Individual unpreserved and 10% formalin-fixed stool specimens screened as negative for Entamoeba histolytica were spiked with E. histolytica trophozoites near the assay LOD. Contrived specimens were tested by blinded operators with the BD MAX™ Enteric Parasite Panel. The results of this clinical simulation are shown in Table 11.

TABLE 11

| | | | | BD MAX ™ |
|---|---|---|---|---|
| | Known | Ent | Ent | E. histolytica |
| Stool Type | Input | ymaxEP | Ct. Score | Result |
| Unpreserved | 2X LoD | 3927.44 | 24.74 | Positive |
| Unpreserved | 2X LoD | 3625.32 | 24.76 | Positive |
| Unpreserved | 2X LoD | 3299.44 | 28.09 | Positive |
| Unpreserved | 2X LoD | 3071.72 | 26.02 | Positive |
| Unpreserved | 2X LoD | 3194.67 | 26.85 | Positive |
| Unpreserved | 2X LoD | 4241.9 | 24.56 | Positive |
| Unpreserved | 2X LoD | 3926.39 | 24.13 | Positive |
| Unpreserved | Negative | 2.78 | 46 | Negative |
| Unpreserved | Negative | 1.77 | 46 | Negative |
| Unpreserved | 2X LoD | 4560.95 | 24.09 | Positive |
| Unpreserved | 2X LoD | 4489.85 | 25.38 | Positive |
| Unpreserved | 2X LoD | 4052.13 | 27.56 | Positive |
| Unpreserved | Negative | 2.75 | 46 | Negative |
| Unpreserved | Negative | 6.41 | 46 | Negative |
| Unpreserved | Negative | 4.67 | 46 | Negative |
| Unpreserved | Negative | 7.13 | 46 | Negative |
| Unpreserved | Negative | 0.28 | 46 | Negative |
| Unpreserved | Negative | 5.35 | 46 | Negative |
| Unpreserved | Negative | 4.39 | 46 | Negative |
| Unpreserved | Negative | 1.39 | 46 | Negative |
| Unpreserved | Negative | 5.76 | 46 | Negative |
| Unpreserved | Negative | 0.85 | 46 | Negative |
| Unpreserved | 2X LoD | 3717.61 | 25.51 | Positive |
| Unpreserved | 2X LoD | 2409.1 | 33.38 | Positive |
| Unpreserved | Negative | 5.66 | 46 | Negative |
| Unpreserved | Negative | 1.82 | 46 | Negative |
| Unpreserved | 2X LoD | 3896.98 | 24.59 | Positive |
| Unpreserved | 2X LoD | 2842.28 | 27.26 | Positive |
| Unpreserved | Negative | 23.37 | 46 | Negative |
| Unpreserved | 2X LoD | 2881.86 | 33.79 | Positive |
| Unpreserved | Negative | 0.77 | 46 | Negative |
| Unpreserved | Negative | 25.47 | 46 | Negative |
| Unpreserved | 2X LoD | 3627.53 | 27.41 | Positive |
| Unpreserved | 2X LoD | 3549.91 | 28.78 | Positive |
| Unpreserved | 2X LoD | 2557.79 | 33.6 | Positive |
| Unpreserved | Negative | 3.43 | 46 | Negative |
| Unpreserved | Negative | 3.34 | 46 | Negative |
| Unpreserved | 2X LoD | 2914.65 | 28.07 | Positive |
| Unpreserved | Negative | 1.52 | 46 | Negative |
| Unpreserved | Negative | 6.69 | 46 | Negative |
| Unpreserved | 2X LoD | 3216.07 | 25.18 | Positive |
| Unpreserved | Negative | 0.59 | 46 | Negative |
| Unpreserved | Negative | 2.75 | 46 | Negative |
| Unpreserved | Negative | 4.93 | 46 | Negative |
| Unpreserved | 2X LoD | 4301.96 | 27.85 | Positive |
| Unpreserved | 2X LoD | 4383.23 | 28.45 | Positive |
| Unpreserved | 2X LoD | 2292.14 | 31.58 | Positive |
| Unpreserved | 2X LoD | 3490.01 | 30.58 | Positive |
| 10% Formalin | 2X LoD | 3905.11 | 27.33 | Positive |
| Fixed | | | | |
| 10% Formalin | 2X LoD | 3587.14 | 24.96 | Positive |
| Fixed | | | | |
| 10% Formalin | 2X LoD | 4042.64 | 28.14 | Positive |
| Fixed | | | | |
| 10% Formalin | 2X LoD | 4088.09 | 25.63 | Positive |
| Fixed | | | | |
| 10% Formalin | 2X LoD | 3217.87 | 26.04 | Positive |
| Fixed | | | | |
| 10% Formalin | 2X LoD | 3611.21 | 23.54 | Positive |
| Fixed | | | | |
| 10% Formalin | 2X LoD | 3407.33 | 26.3 | Positive |
| Fixed | | | | |
| 10% Formalin | 2X LoD | 3522.62 | 24.42 | Positive |
| Fixed | | | | |
| 10% Formalin | 2X LoD | 4156.1 | 25.21 | Positive |
| Fixed | | | | |
| 10% Formalin | 2X LoD | 4722.12 | 26.83 | Positive |
| Fixed | | | | |
| 10% Formalin | Negative | 0.55 | 46 | Negative |
| Fixed | | | | |
| 10% Formalin | Negative | 0.55 | 46 | Negative |
| Fixed | | | | |
| 10% Formalin | Negative | 12.92 | 46 | Negative |
| Fixed | | | | |
| 10% Formalin | Negative | 3.54 | 46 | Negative |
| Fixed | | | | |
| 10% Formalin | Negative | 5.95 | 46 | Negative |
| Fixed | | | | |
| 10% Formalin | 2X LoD | 3460.87 | 29.1 | Positive |
| Fixed | | | | |
| 10% Formalin | 2X LoD | 3207.24 | 24.99 | Positive |
| Fixed | | | | |
| 10% Formalin | Negative | 1.21 | 46 | Negative |
| Fixed | | | | |
| 10% Formalin | Negative | 2.57 | 46 | Negative |
| Fixed | | | | |
| 10% Formalin | 2X LoD | 4342.59 | 25.23 | Positive |
| Fixed | | | | |
| 10% Formalin | 2X LoD | 2809.65 | 27.47 | Positive |
| Fixed | | | | |
| 10% Formalin | Negative | 5.87 | 46 | Negative |
| Fixed | | | | |
| 10% Formalin | 2X LoD | 3467.59 | 24.63 | Positive |
| Fixed | | | | |
| 10% Formalin | 2X LoD | 3940.15 | 24.39 | Positive |
| Fixed | | | | |
| 10% Formalin | Negative | 1.73 | 46 | Negative |
| Fixed | | | | |
| 10% Formalin | Negative | 5.04 | 46 | Negative |
| Fixed | | | | |
| 10% Formalin | 2X LoD | 3898.36 | 26 | Positive |
| Fixed | | | | |
| 10% Formalin | Negative | 1.6 | 46 | Negative |
| Fixed | | | | |
| 10% Formalin | Negative | 2.28 | 46 | Negative |
| Fixed | | | | |
| 10% Formalin | Negative | 0.89 | 46 | Negative |
| Fixed | | | | |
| 10% Formalin | 2X LoD | 4136.67 | 27.6 | Positive |
| Fixed | | | | |
| 10% Formalin | Negative | 8.34 | 46 | Negative |
| Fixed | | | | |
| 10% Formalin | 2X LoD | 3609.34 | 27.12 | Positive |
| Fixed | | | | |
| 10% Formalin | 2X LoD | 4351.88 | 26.7 | Positive |
| Fixed | | | | |
| 10% Formalin | Negative | 1.37 | 46 | Negative |
| Fixed | | | | |
| 10% Formalin | Negative | 0.55 | 46 | Negative |
| Fixed | | | | |
| 10% Formalin | Negative | 0.86 | 46 | Negative |
| Fixed | | | | |
| 10% Formalin | Negative | 6.67 | 46 | Negative |
| Fixed | | | | |
| 10% Formalin | 2X LoD | 3951.5 | 29.41 | Positive |
| Fixed | | | | |
| 10% Formalin | Negative | 1.27 | 46 | Negative |
| Fixed | | | | |
| 10% Formalin | Negative | 13.13 | 46 | Negative |
| Fixed | | | | |
| 10% Formalin | 2X LoD | 2784.26 | 30.06 | Positive |
| Fixed | | | | |
| 10% Formalin | 2X LoD | 4015.27 | 26.76 | Positive |
| Fixed | | | | |
| 10% Formalin | Negative | 6.14 | 46 | Negative |
| Fixed | | | | |
| 10% Formalin | Negative | 2.61 | 46 | Negative |
| Fixed | | | | |
| 10% Formalin | 2X LoD | 2553.31 | 27.45 | Positive |
| Fixed | | | | |
| 10% Formalin | Negative | 0.74 | 46 | Negative |
| Fixed | | | | |
| 10% Formalin | Negative | 8.64 | 46 | Negative |
| Fixed | | | | |

100% of spiked specimens were positive and 100% of non-spiked specimens were negative. As such, it is contemplated that methods of detecting E. histolytica nucleic acids in accordance with some embodiments herein accurately detect E. histolytica, with minimal false negatives.

### Example 12

### Comparison of BD MAX™ Assay to Reference Methods

The results of the BD MAX™ assay were compared to various reference methods. A “final reference method (RM) result” (also referred to herein as a “composite RM”) was based on a combined input from a Trichrome Entamoeba spp. assay and an alternative PCR and sequencing approach. For E. histolytica, the composite reference method (RM) included 1) a microscopic examination of a trichrome staining of PVA fixed stool, in parallel with 2) an analytically validated alternate PCR and bi-directional sequencing. The study involved a total of five (5) US investigational Clinical Centers where specimens were collected as part of the routine patient care, enrolled in the trial and tested with the BD MAX™ Enteric Parasite Panel. Three specimen collection centers and additional specimen brokers sent specimens to investigational clinical centers for testing.

For prospective samples, the inclusion criteria were as follows: Specimens were obtained from pediatric or adult patients suspected of acute gastroenteritis or colitis for which target parasitic diagnostic tests have been ordered by a healthcare provider. A stool specimen was collected either unpreserved or 10% formalin-fixed. Only one specimen of each specimen type (fixed or unpreserved), collected from a single patient was allowed. The study required a sufficient volume of stool to be available for adequate reference method testing (depending on each clinical center standard procedure) and a minimum of 0.5 mL or 0.5 gram of stool to be available for BD MAX™ EPP testing.

For retrospective samples, the inclusion criteria were as follows: Unpreserved and fixed specimen for which the original results of the routine test method were available, for at least one (1) of the three (3) EPP targets. Each specimen had a known collection date. Each specimen was stored at −20° C. or colder if unpreserved or 2-8° C. if preserved in formalin throughout the entire storage period.

As summarized in Table 12.1, a “final RM result” was scored as positive if both Trichrome Entamoeba spp. assay and alternative PCR and sequencing were positive, and was scored as a negative if either or both of these methods was negative. As summarized in Table 12.2, a result was scored as a “true positive” if the BD MAX™ assay and final RM result were both positive, and a “true negative” if the BD MAX™ assay and final RM result were both negative. A result was scored as a “false positive” if the BD MAX™ assay was positive and final RM result was negative, and a “false negative” if the BD MAX™ assay was negative and the final RM result was positive (see Table 12.2).

Abbreviations used include: P=Positive; N=Negative; LB=Lower Bound; UB=Upper Bound; PPA=Positive Percent Agreement (Sensitivity); NPA=Negative Percent Agreement (Specificity).

TABLE 12.1

Segments

| Trichrome | Alt PCR and | Final RM |
|---|---|---|
| Entamoeba spp. | Sequencing | Result |
| Positive | Positive | Positive |
| Positive | Negative | Negative |
| Negative | Positive | Negative |
| Negative | Negative | Negative |

TABLE 12.2

Accuracy Results

| BD MAX ™ | Composite | | |
|---|---|---|---|
| EPP | RM | Interpretation | Final Status |
| Positive | Positive | Concordant | True Positive |
| Positive | Negative | Discrepant | False Positive |
| Negative | Positive | Discrepant | False Negative |
| Negative | Negative | Concordant | True Negative |

The overall performance results are summarized in Table 12.3. It is noted that for 1660 samples screened, there were 11 “true positives”, 1649 “true negatives”, 0 “false positives”, and 0 “false negatives”.

TABLE 12.3

| | |
|---|---|
| BD MAX | RM |

| E. histolytica | Postive | Negative | Total |
|---|---|---|---|
| Postive | 11 | 0 | 11 |
| Negative | 0 | 1649 | 1649 |
| Total | 11 | 1649 | 1660 |

PPA (95% CI (LB, UB)): 100% (74.1%, 100%)
NPA (95% CI (LB, UB)): 100% (99.8%, 100%)

As such, the results summarized in Table 12.3 showed a high degree concordance between the BD MAX™ assay and reference methods. There were no false positives or false negatives among 1660 samples. Accordingly, it is contemplated that methods of detecting E. histolytica nucleic acids in accordance with some embodiments herein accurately detect E. histolytica, with minimal false negatives and minimal false positives, for example fewer than one false negative in 1660, and fewer than one false negative in 1660.

The overall performance results were further analyzed for prospective and retrospective specimen origins, as described herein. These results of this analysis are summarized in Table 12.4.

TABLE 12.4

| Specimen | BD MAX ™ | RM | | |
|---|---|---|---|---|
| Origin | E. histolytica | Positive | Negative | Total |
| Prospective | Positive | 0 | 0 | 0 |
| | Negative | 0 | 1404 | 1404 |
| | Total | 0 | 1404 | 1404 |
| | PPA (95% CI (LB, UB)): No data for calculation | | | |
| | NPA (95% CI (LB, UB)): 100% (99.7%, 100%) | | | |
| Retrospective | Positive | 11 | 0 | 11 |
| | Negative | 0 | 245 | 245 |
| | Total | 11 | 245 | 256 |

PPA (95% CI (LB, UB)): 100% (74.1%, 100%)
NPA (95% CI (LB, UB)): 100% (98.5%, 100%)

As shown in Table 12.4, the BD Max™ assay yielded accurate results for both prospective and retrospective specimens. Accordingly, it is contemplated that methods of detecting E. histolytica nucleic acids in accordance with some embodiments herein accurately detect E. histolytica, with minimal false negatives.

The overall performance results were further analyzed for unpreserved specimens, and specimens fixed in 10% formalin. The results of this analysis are summarized in Table 12.5.

TABLE 12.5

| | | BD | | | |
|---|---|---|---|---|---|
| Specimen | Specimen | MAX ™ | RM | | |
| Type | Origin | EPP | P | N | Total |
| Formalin | Prospective | P | 0 | 0 | 0 |
| 10% | | N | 0 | 827 | 827 |
| | | Total | 0 | 827 | 827 |
| | | SENSITIVITY (95% CI (LB, UB)): No data for calculation | | | |
| | | SPECIFICITY (95% CI (LB, UB)): 100% (99.5%, 100%) | | | |
| | Retrospective | P | 0 | 0 | 0 |
| | | N | 0 | 54 | 54 |
| | | Total | 0 | 54 | 54 |
| | | PPA (95% CI (LB, UB)): No data for calculation | | | |
| | | NPA (95% CI (LB, UB)): 100% (93.4%, 100%) | | | |
| | Combined | P | 0 | 0 | 0 |
| | | N | 0 | 881 | 881 |
| | | Total | 0 | 881 | 881 |
| | | PPA (95% CI (LB, UB)): No data for calculation | | | |
| | | NPA (95% CI (LB, UB)): 100% (99.6%, 100%) | | | |
| Un- | Prospective | P | 0 | 0 | 0 |
| preserved | | N | 0 | 577 | 577 |
| | | Total | 0 | 577 | 577 |
| | | SENSITIVITY (95% CI (LB, UB)): No data for calculation | | | |
| | | SPECIFICITY (95% CI (LB, UB)): 100% (99.3%, 100%) | | | |
| | Retrospective | P | 11 | 0 | 11 |
| | | N | 0 | 191 | 191 |
| | | Total | 11 | 0 | 202 |
| | | PPA (95% CI (LB, UB)): 100% (74.1%, 100%) | | | |
| | | NPA (95% CI (LB, UB)): 100% (98.0%, 100%) | | | |
| | Combined | P | 11 | 0 | 11 |
| | | N | 0 | 768 | 768 |
| | | Total | 11 | 768 | 779 |

PPA (95% CI (LB, UB)): 100% (74.1%, 100%)
NPA (95% CI (LB, UB)): 100% (99.5%, 100%)

As shown in Table 12.5, the BD Max™ assay yielded accurate results for both formalin fixed and unpreserved specimens. Accordingly, it is contemplated that methods of detecting E. histolytica nucleic acids in accordance with some embodiments herein are suitable for a variety of sample formats, including, but not limited to unpreserved samples and, fixed samples. As such, methods in accordance with some embodiments herein can be suitable for screening samples at clinical sites, at off-site testing centers that may require fixing samples and/or a substantial lag time between sample collection and testing.

Particular sequence features (e.g. organisms, genes and/or portions thereof) identified by the analytically validated alternate PCR and bi-directional sequencing were consistent with the BD MAX™ assay result. As summarized in Table 12.6 below, samples that yielded non-E. histolytica sequencing results were identified as negative by the BD MAX™ assay, and samples that yielded sequence characteristic of E. histolytica sequencing results were identified as positive by the BD MAX™ assay.

TABLE 12.6

| Tri- | | Overall | MAX | | Final Call |
|---|---|---|---|---|---|
| chrome | | RM | Ct. | MAX | (TP/FP/TN/ |
| Result | Sequencing Result | Call | Score | Result | FN) |
| NEG | Entamoeba dispar genes for 18S rRNA, | NEG | NA | NEG | TN |
| | ITS1, 5.8S rRNA, ITS2, complete sequence | | | | |
| NEG | Entamoeba dispar genes for 18S rRNA, | NEG | NA | NEG | TN |
| | ITS1, 5.8S rRNA, ITS2, complete sequence | | | | |
| NEG | Entamoeba dispar genes for 18S rRNA, | NEG | NA | NEG | TN |
| | ITS1, 5.8S rRNA, ITS2, complete sequence | | | | |
| NEG | Entamoeba dispar genes for 18S rRNA, | NEG | NA | NEG | TN |
| | ITS1, 5.8S rRNA, ITS2, complete sequence | | | | |
| NEG | Entamoeba hartmanni partial 18S rRNA | NEG | NA | NEG | TN |
| | gene, isolate 08/1040 | | | | |
| NEG | Entamoeba dispar genes for 18S rRNA, | NEG | NA | NEG | TN |
| | ITS1, 5.8S rRNA, ITS2, complete sequence | | | | |
| NEG | Entamoeba dispar genes for 18S rRNA, | NEG | NA | NEG | TN |
| | ITS1, 5.8S rRNA, ITS2, complete sequence | | | | |
| NEG | Entamoeba coli partial 18S rRNA gene, | NEG | NA | NEG | TN |
| | isolate J65 | | | | |
| NEG | Entamoeba coli partial 18S rRNA gene, | NEG | NA | NEG | TN |
| | isolate J65 | | | | |
| NEG | Entamoeba dispar genes for 18S rRNA, | NEG | NA | NEG | TN |
| | ITS1, 5.8S rRNA, ITS2, complete sequence | | | | |
| NEG | Entamoeba coli partial 18S rRNA gene, | NEG | NA | NEG | TN |
| | isolate J65 | | | | |
| NEG | Entamoeba coli partial 18S rRNA gene, | NEG | NA | NEG | TN |
| | isolate J65 | | | | |
| NEG | Entamoeba coli partial 18S rRNA gene, | NEG | NA | NEG | TN |
| | isolate J65 | | | | |
| POS | Entamoeba dispar genes for 18S rRNA, | NEG | NA | NEG | TN |
| | ITS1, 5.8S rRNA, ITS2, complete sequence | | | | |
| NEG | Entamoeba sp. RL2 partial 18S rRNA gene, | NEG | NA | NEG | TN |
| | isolate Cow350 | | | | |
| NEG | Entamoeba gingivalis SrRNA gene | NEG | NA | NEG | TN |
| NEG | Entamoeba hartmanni partial 18S rRNA gene, | NEG | NA | NEG | TN |
| | isolate 08/1040 | | | | |
| NEG | Entamoeba coli partial 18S rRNA gene, | NEG | NA | NEG | TN |
| | isolate EM047 | | | | |
| NEG | Entamoeba coli partial 18S rRNA gene, | NEG | NA | NEG | TN |
| | isolate J65 | | | | |
| NEG | Entamoeba dispar genes for 18S rRNA, | NEG | NA | NEG | TN |
| | ITS1, 5.8S rRNA, ITS2, complete sequence | | | | |
| NEG | Entamoeba gingivalis SrRNA gene | NEG | NA | NEG | TN |
| NEG | Entamoeba coli partial 18S rRNA gene, | NEG | NA | NEG | TN |
| | isolate EM047 | | | | |
| POS | Entamoeba histolytica gene for small | POS | 22.7 | POS | TP |
| | subunit ribosomal RNA, strain: BF-841 cl1 | | | | |
| POS | Entamoeba histolytica gene for small | POS | 19.8 | POS | TP |
| | subunit ribosomal RNA, strain: BF-841 cl1 | | | | |
| POS | Entamoeba histolytica gene for small | POS | 31.4 | POS | TP |
| | subunit ribosomal RNA, strain: BF-841 cl1 | | | | |
| POS | Entamoeba histolytica gene for small | POS | 24.8 | POS | TP |
| | subunit ribosomal RNA, strain: BF-841 cl1 | | | | |
| POS | Entamoeba histolytica gene for small | POS | 23.7 | POS | TP |
| | subunit ribosomal RNA, strain: BF-841 cl1 | | | | |
| POS | Entamoeba histolytica gene for small | POS | 33.5 | POS | TP |
| | subunit ribosomal RNA, strain: BF-841 cl1 | | | | |
| POS | Entamoeba histolytica gene for small | POS | 24.9 | POS | TP |
| | subunit ribosomal RNA, strain: BF-841 cl1 | | | | |
| POS | Entamoeba histolytica gene for small | POS | 27.6 | POS | TP |
| | subunit ribosomal RNA, strain: BF-841 cl1 | | | | |
| POS | Entamoeba histolytica gene for small | POS | 27.1 | POS | TP |
| | subunit ribosomal RNA, strain: BF-841 cl1 | | | | |
| POS | Entamoeba histolytica gene for small | POS | 25.2 | POS | TP |
| | subunit ribosomal RNA, strain: BF-841 cl1 | | | | |
| NEG | Entamoeba hartmanni partial 18S rRNA | NEG | NA | NEG | TN |
| | gene, isolate EM042 | | | | |
| NEG | Entamoeba dispar genes for 18S rRNA, | NEG | NA | NEG | TN |
| | ITS1, 5.8S rRNA, ITS2, complete sequence | | | | |
| NEG | Entamoeba coli partial 18S rRNA gene, | NEG | NA | NEG | TN |
| | isolate J65 | | | | |
| NEG | Entamoeba coli partial 18S rRNA gene, | NEG | NA | NEG | TN |
| | isolate J65 | | | | |
| NEG | Entamoeba coli partial 18S rRNA gene, | NEG | NA | NEG | TN |
| | isolate EM049 | | | | |
| NEG | Entamoeba hartmanni partial 18S rRNA | NEG | NA | NEG | TN |
| | gene, isolate J92 | | | | |
| POS | Entamoeba histolytica gene for small subunit | POS | 30.9 | POS | TP |
| | ribosomal RNA, strain: BF-841 cl1 | | | | |
| NEG | Entamoeba hartmanni partial 18S rRNA gene, | NEG | NA | NEG | TN |
| | isolate EM061a | | | | |

As such, it is contemplated that methods of detecting E. histolytica nucleic acids in accordance with some embodiments herein are highly accurate, and yield results in line with particular sequence features of the samples examined.

Samples that did not meet the criteria for the study were excluded. By way of example, trichrome, sequencing, and BD MAX™ assay results for the samples excluded from the study are provided in Table 12.7.

TABLE 12.7

| Specimen | Trichrome | | Overall | MAX | MAX |
|---|---|---|---|---|---|
| Type | Result | Sequencing Result | RM Call | Ct. Score | Result |
| Formalin 10% | Non-Compliant | Entamoeba coli partial 18S | NA | NA | NEG |
| | | rRNA gene, isolate EM076 | | | |
| Formalin 10% | Non-Compliant | Entamoeba coli partial 18S | NA | NA | NEG |
| | | rRNA gene, isolate EM076 | | | |
| Formalin 10% | NEG | Entamoeba dispar genes for | NA | NA | NEG |
| | | 18S rRNA, ITS1, 5.8S rRNA, | | | |
| | | ITS2, complete sequence | | | |
| Formalin 10% | NEG | Entamoeba polecki partial | NA | NA | NEG |
| | | 18S rRNA gene, isolate UNE9 | | | |
| Formalin 10% | Non-Compliant | Entamoeba muris 16S rRNA | NA | NA | NEG |
| | | gene, isolated in Madrid, | | | |
| | | Spain | | | |
| Formalin 10% | Non-Compliant | Entamoeba dispar genes for | NA | NA | NEG |
| | | 18S rRNA, ITS1, 5.8S rRNA, | | | |
| | | ITS2, complete sequence | | | |
| Unpreserved | NEG | Entamoeba coli partial 18S | NA | NA | NEG |
| | | rRNA gene, isolate J65 | | | |
| Unpreserved | NEG | Entamoeba dispar genes for | NA | NA | NEG |
| | | 18S rRNA, ITS1, 5.8S rRNA, | | | |
| | | ITS2, complete sequence | | | |
| Unpreserved | NEG | Entamoeba dispar genes for | NA | NA | NEG |
| | | 18S rRNA, ITS1, 5.8S rRNA, | | | |
| | | ITS2, complete sequence | | | |
| Unpreserved | NEG | Entamoeba nuttalli genes for | NA | NA | NEG |
| | | 18S rRNA, ITS1, 5.8S rRNA, | | | |
| | | ITS2, complete sequence, | | | |
| | | strain: NMP9 | | | |
| Unpreserved | NEG | Entamoeba dispar genes for | NA | NA | NEG |
| | | 18S rRNA, ITS1, 5.8S rRNA, | | | |
| | | ITS2, complete sequence | | | |
| Unpreserved | NEG | Entamoeba dispar genes for | NA | NA | NEG |
| | | 18S rRNA, ITS1, 5.8S rRNA, | | | |
| | | ITS2, complete sequence | | | |
| Formalin 10% | NEG | Entamoeba dispar genes for | NA | NA | NEG |
| | | 18S rRNA, ITS1, 5.8S rRNA, | | | |
| | | ITS2, complete sequence | | | |
| Formalin 10% | NEG | Entamoeba coli partial 18S | NA | NA | NEG |
| | | rRNA gene, isolate EM049 | | | |
| Formalin 10% | NEG | Entamoeba dispar genes for | NA | NA | NEG |
| | | 18S rRNA, ITS1, 5.8S rRNA, | | | |
| | | ITS2, complete sequence | | | |
| Formalin 10% | Non-Compliant | Entamoeba coli partial 18S | NA | NA | NEG |
| | | rRNA gene, isolate EM047 | | | |
| Formalin 10% | Non-Compliant | Entamoeba coli partial 18S | NA | NA | NEG |
| | | rRNA gene, isolate EM077 | | | |
| Formalin 10% | Non-Compliant | Entamoeba coli partial 18S | NA | NA | NEG |
| | | rRNA gene, isolate J65 | | | |
| Formalin 10% | Non-Compliant | Entamoeba hartmanni | NA | NA | NEG |
| | | partial 18S rRNA gene, | | | |
| | | isolate EM061a | | | |
| Unpreserved | Non-Compliant | Entamoeba hartmanni | NA | NA | NEG |
| | | partial 18S rRNA gene, | | | |
| | | isolate EM061a | | | |
| Formalin 10% | Non-Compliant | Entamoeba hartmanni | NA | NA | NEG |
| | | partial 18S rRNA gene, | | | |
| | | isolate 09/1140 | | | |
| Unpreserved | Non-Compliant | Entamoeba bovis 18S rRNA | NA | NA | NEG |
| | | gene, isolate Sheep310 | | | |

It is noted that the sequencing results of the non-compliant samples shown in Table 12.7 indicated that the BD MAX™ assay is not cross-reactive with other Entamoeba sequences (such as Entamoeba coli, Entamoeba dispar, Entamoeba polecki, Entamoeba muris, Entamoeba nuttalli, Entamoeba hartmanni, and Entamoeba bovis). As such, it is contemplated that methods of detecting E. histolytica nucleic acids in accordance with some embodiments herein do not cross react with Entamoeba coli, Entamoeba dispar, Entamoeba polecki, Entamoeba muris, Entamoeba nuttalli, Entamoeba hartmanni, and/or Entamoeba bovis.

### Example 13

### Contrived Clinical Supplemental Study

It is noted that E. histolytica infection can be relatively rare, and consistent with this relative rarity, a number of the clinical studies produced many more negative results than positive results. So as to characterize additional positive results for the BD MAX™ assay, a contrived clinical supplemental study was designed and performed, in which a number of specimens were spiked with E. histolytica trophozites.

In particular, individual unpreserved and 10% formalin-fixed stool specimens screened as negative for Entamoeba histolytica were spiked with E. histolytica trophozoites at levels spanning the assay range. Contrived specimens were tested by blinded operators with the BD MAX™ Enteric Parasite Panel (EPP). The results of the contrived clinical study are shown in Table 13.

TABLE 13

| ID | Type | Input | Ent Ct. Score | MAX Ent Result |
|---|---|---|---|---|
| CF001 | Fixed | Negative | 46 | NEG |
| CF002 | Fixed | Negative | 46 | NEG |
| CF003 | Fixed | 2X LoD | 24.58 | POS |
| CF004 | Fixed | 50X LoD | 21.1 | POS |
| CF005 | Fixed | 2X LoD | 25.26 | POS |
| CF006 | Fixed | Negative | 46 | NEG |
| CF007 | Fixed | Negative | 46 | NEG |
| CF008 | Fixed | 10X LoD | 23.19 | POS |
| CF009 | Fixed | 100X LoD | 20.97 | POS |
| CF010 | Fixed | 2X LoD | 25.01 | POS |
| CF011 | Fixed | Negative | 46 | NEG |
| CF012 | Fixed | 2X LoD | 25.54 | POS |
| CF013 | Fixed | 2X LoD | 24.81 | POS |
| CF014 | Fixed | Negative | 46 | NEG |
| CF015 | Fixed | Negative | 46 | NEG |
| CF016 | Fixed | 4X LoD | 24.53 | POS |
| CF017 | Fixed | 2X LoD | 25.27 | POS |
| CF018 | Fixed | 50X LoD | 21.83 | POS |
| CF019 | Fixed | 4X LoD | 25.12 | POS |
| CF020 | Fixed | Negative | 46 | NEG |
| CF021 | Fixed | 2X LoD | 24.96 | POS |
| CF022 | Fixed | 100X LoD | 19.28 | POS |
| CF023 | Fixed | 2X LoD | 25.1 | POS |
| CF024 | Fixed | Negative | 46 | NEG |
| CF025 | Fixed | Negative | 46 | NEG |
| CF026 | Fixed | Negative | 46 | NEG |
| CF027 | Fixed | 10X LoD | 24.08 | POS |
| CF028 | Fixed | 50X LoD | 21.31 | POS |
| CF029 | Fixed | 2X LoD | 25.87 | POS |
| CF030 | Fixed | Negative | 46 | NEG |
| CF031 | Fixed | Negative | 46 | NEG |
| CF032 | Fixed | 2X LoD | 25.08 | POS |
| CF033 | Fixed | 50X LoD | 21.6 | POS |
| CF034 | Fixed | 4X LoD | 24.64 | POS |
| CF035 | Fixed | 100X LoD | 22.14 | POS |
| CF036 | Fixed | 4X LoD | 24.84 | POS |
| CF037 | Fixed | Negative | 46 | NEG |
| CF038 | Fixed | Negative | 46 | NEG |
| CF039 | Fixed | 4X LoD | 24.47 | POS |
| CF040 | Fixed | Negative | 46 | NEG |
| CF041 | Fixed | 2X LoD | 26.28 | POS |
| CF042 | Fixed | Negative | 46 | NEG |
| CF043 | Fixed | 2X LoD | 25.76 | POS |
| CF044 | Fixed | 4X LoD | 24.24 | POS |
| CF045 | Fixed | Negative | 46 | NEG |
| CF046 | Fixed | Negative | 46 | NEG |
| CF047 | Fixed | Negative | 46 | NEG |
| CF048 | Fixed | 4X LoD | 24.77 | POS |
| CF049 | Fixed | 2X LoD | 27.53 | POS |
| CF050 | Fixed | Negative | 46 | NEG |
| CF051 | Fixed | Negative | 46 | NEG |
| CF052 | Fixed | 10X LoD | 23.33 | POS |
| CF053 | Fixed | Negative | 46 | NEG |
| CF054 | Fixed | Negative | 46 | NEG |
| CF055 | Fixed | 10X LoD | 24.69 | POS |
| CF056 | Fixed | Negative | 46 | NEG |
| CF057 | Fixed | Negative | 46 | NEG |
| CF058 | Fixed | 2X LoD | 27.31 | POS |
| CF059 | Fixed | Negative | 46 | NEG |
| CF060 | Fixed | Negative | 46 | NEG |
| CF061 | Fixed | Negative | 46 | NEG |
| CF062 | Fixed | 2X LoD | 26.19 | POS |
| CF063 | Fixed | 50X LoD | 21.11 | POS |
| CF064 | Fixed | 2X LoD | 27.83 | POS |
| CF065 | Fixed | Negative | 46 | NEG |
| CF066 | Fixed | Negative | 46 | NEG |
| CF067 | Fixed | Negative | 46 | NEG |
| CF068 | Fixed | 2X LoD | 24.63 | POS |
| CF069 | Fixed | Negative | 46 | NEG |
| CF070 | Fixed | Negative | 46 | NEG |
| CF071 | Fixed | Negative | 46 | NEG |
| CF072 | Fixed | 2X LoD | 25.43 | POS |
| CF073 | Fixed | 100X LoD | 20.38 | POS |
| CF074 | Fixed | 10X LoD | 23.5 | POS |
| CF075 | Fixed | Negative | 46 | NEG |
| CF076 | Fixed | Negative | 46 | NEG |
| CF077 | Fixed | 2X LoD | 26.37 | POS |
| CF078 | Fixed | 10X LoD | 23.89 | POS |
| CF079 | Fixed | 100X LoD | 21.87 | POS |
| CF080 | Fixed | 2X LoD | 26.67 | POS |
| CF081 | Fixed | Negative | 46 | NEG |
| CF082 | Fixed | Negative | 46 | NEG |
| CF083 | Fixed | 2X LoD | 27.09 | POS |
| CF084 | Fixed | 100X LoD | 21.05 | POS |
| CF085 | Fixed | Negative | 46 | NEG |
| CF086 | Fixed | 2X LoD | 26.25 | POS |
| CF087 | Fixed | 50X LoD | 21.96 | POS |
| CF088 | Fixed | 2X LoD | 25.46 | POS |
| CF089 | Fixed | Negative | 46 | NEG |
| CF090 | Fixed | Negative | 46 | NEG |
| CF091 | Fixed | Negative | 46 | NEG |
| CF092 | Fixed | Negative | 46 | NEG |
| CF093 | Fixed | Negative | 46 | NEG |
| CF094 | Fixed | 2X LoD | 25.92 | POS |
| CF095 | Fixed | 2X LoD | 24.12 | POS |
| CF096 | Fixed | Negative | 46 | NEG |
| CF097 | Fixed | Negative | 46 | NEG |
| CF098 | Fixed | Negative | 46 | NEG |
| CF099 | Fixed | Negative | 46 | NEG |
| CF100 | Fixed | Negative | 46 | NEG |
| CU001 | Unpreserved | 2X LoD | 28.7 | POS |
| CU002 | Unpreserved | 50X LoD | 22.18 | POS |
| CU003 | Unpreserved | 2X LoD | 24.27 | POS |
| CU004 | Unpreserved | Negative | 46 | NEG |
| CU005 | Unpreserved | Negative | 46 | NEG |
| CU006 | Unpreserved | Negative | 46 | NEG |
| CU007 | Unpreserved | Negative | 46 | NEG |
| CU008 | Unpreserved | 2X LoD | 24.73 | POS |
| CU009 | Unpreserved | 100X LoD | 24.43 | POS |
| CU010 | Unpreserved | 2X LoD | 28 | POS |
| CU011 | Unpreserved | Negative | 46 | NEG |
| CU012 | Unpreserved | Negative | 46 | NEG |
| CU013 | Unpreserved | 2X LoD | 27.6 | POS |
| CU014 | Unpreserved | Negative | 46 | NEG |
| CU015 | Unpreserved | Negative | 46 | NEG |
| CU016 | Unpreserved | Negative | 46 | NEG |
| CU017 | Unpreserved | Negative | 46 | NEG |
| CU018 | Unpreserved | 4X LoD | 25.95 | POS |
| CU019 | Unpreserved | Negative | 46 | NEG |
| CU020 | Unpreserved | Negative | 46 | NEG |
| CU021 | Unpreserved | 2X LoD | 27 | POS |
| CU022 | Unpreserved | 100X LoD | 19.77 | POS |
| CU023 | Unpreserved | 2X LoD | 22.86 | POS |
| CU024 | Unpreserved | Negative | 46 | NEG |
| CU025 | Unpreserved | Negative | 46 | NEG |
| CU026 | Unpreserved | 4X LoD | 22.39 | POS |
| CU027 | Unpreserved | Negative | 46 | NEG |
| CU028 | Unpreserved | 10X LoD | 22.54 | POS |
| CU029 | Unpreserved | Negative | 46 | NEG |
| CU030 | Unpreserved | Negative | 46 | NEG |
| CU031 | Unpreserved | Negative | 46 | NEG |
| CU032 | Unpreserved | 10X LoD | 21.78 | POS |
| CU033 | Unpreserved | Negative | 46 | NEG |
| CU034 | Unpreserved | Negative | 46 | NEG |
| CU035 | Unpreserved | 2X LoD | 25.11 | POS |
| CU036 | Unpreserved | 4X LoD | 23.61 | POS |
| CU037 | Unpreserved | 100X LoD | 19.23 | POS |
| CU038 | Unpreserved | 2X LoD | 27.79 | POS |
| CU039 | Unpreserved | Negative | 46 | NEG |
| CU040 | Unpreserved | Negative | 46 | NEG |
| CU041 | Unpreserved | Negative | 46 | NEG |
| CU042 | Unpreserved | Negative | 46 | NEG |
| CU043 | Unpreserved | Negative | 46 | NEG |
| CU044 | Unpreserved | 2X LoD | 24.24 | POS |
| CU045 | Unpreserved | 100X LoD | 20.28 | POS |
| CU046 | Unpreserved | 4X LoD | 23.88 | POS |
| CU047 | Unpreserved | 2X LoD | 25.09 | POS |
| CU048 | Unpreserved | Negative | 46 | NEG |
| CU049 | Unpreserved | Negative | 46 | NEG |
| CU050 | Unpreserved | Negative | 46 | NEG |
| CU051 | Unpreserved | 4X LoD | 23.86 | POS |
| CU052 | Unpreserved | 50X LoD | 20.96 | POS |
| CU053 | Unpreserved | 4X LoD | 24.41 | POS |
| CU054 | Unpreserved | 10X LoD | 22.37 | POS |
| CU055 | Unpreserved | Negative | 46 | NEG |
| CU056 | Unpreserved | 2X LoD | 25.03 | POS |
| CU057 | Unpreserved | 50X LoD | 22 | POS |
| CU058 | Unpreserved | 2X LoD | 25.57 | POS |
| CU059 | Unpreserved | Negative | 46 | NEG |
| CU060 | Unpreserved | Negative | 46 | NEG |
| CU061 | Unpreserved | 2X LoD | 23.58 | POS |
| CU062 | Unpreserved | 100X LoD | 21.18 | POS |
| CU063 | Unpreserved | 2X LoD | 25.34 | POS |
| CU064 | Unpreserved | Negative | 46 | NEG |
| CU065 | Unpreserved | 2X LoD | 25.46 | POS |
| CU066 | Unpreserved | Negative | 46 | NEG |
| CU067 | Unpreserved | Negative | 46 | NEG |
| CU068 | Unpreserved | 2X LoD | 23.55 | POS |
| CU069 | Unpreserved | 50X LoD | 20.95 | POS |
| CU070 | Unpreserved | 2X LoD | 23.14 | POS |
| CU071 | Unpreserved | Negative | 46 | NEG |
| CU072 | Unpreserved | 10X LoD | 21.97 | POS |
| CU073 | Unpreserved | Negative | 46 | NEG |
| CU074 | Unpreserved | Negative | 46 | NEG |
| CU075 | Unpreserved | 2X LoD | 24.1 | POS |
| CU076 | Unpreserved | Negative | 46 | NEG |
| CU077 | Unpreserved | 2X LoD | 23.25 | POS |
| CU078 | Unpreserved | 50X LoD | 19.7 | POS |
| CU079 | Unpreserved | 2X LoD | 22.96 | POS |
| CU080 | Unpreserved | Negative | 46 | NEG |
| CU081 | Unpreserved | 2X LoD | 25.73 | POS |
| CU082 | Unpreserved | Negative | 46 | NEG |
| CU083 | Unpreserved | Negative | 46 | NEG |
| CU084 | Unpreserved | Negative | 46 | NEG |
| CU085 | Unpreserved | 10X LoD | 21.41 | POS |
| CU086 | Unpreserved | Negative | 46 | NEG |
| CU087 | Unpreserved | Negative | 46 | NEG |
| CU088 | Unpreserved | 4X LoD | 23.59 | POS |
| CU089 | Unpreserved | 100X LoD | 18.47 | POS |
| CU090 | Unpreserved | 2X LoD | 23.44 | POS |
| CU091 | Unpreserved | Negative | 46 | NEG |
| CU092 | Unpreserved | Negative | 46 | NEG |
| CU093 | Unpreserved | Negative | 46 | NEG |
| CU094 | Unpreserved | 2X LoD | 24.55 | POS |
| CU095 | Unpreserved | 50X LoD | 18.7 | POS |
| CU096 | Unpreserved | 2X LoD | 24.53 | POS |
| CU097 | Unpreserved | Negative | 46 | NEG |
| CU098 | Unpreserved | Negative | 46 | NEG |
| CU099 | Unpreserved | 10X LoD | 21.36 | POS |
| CU100 | Unpreserved | Negative | 46 | NEG |

As shown in Table 13, 100% of the spiked specimens were positive and 100% of non-spiked specimens were negative. As such, it is contemplated that methods of detecting E. histolytica nucleic acid in accordance with some embodiments herein are robust and accurate among samples that contain E. histolytica, as well as samples that do not contain E. histolytica.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

While the present invention has been described in some detail for purposes of clarity and understanding, one skilled in the art will appreciate that various changes in form and detail can be made without departing from the true scope of the invention.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

The foregoing description and Examples detail certain embodiments. It will be appreciated, however, that no matter how detailed the foregoing may appear in text, the invention may be practiced in many ways and the invention should be construed in accordance with the appended claims and any equivalents thereof

What is claimed is:

1. A method of detecting the presence of an E. histolytica polynucleotide sequence in a sample, the method comprising:
contacting the sample with a first primer consisting essentially of SEQ ID NO: 1 (GTACAAAATGGCCAATTCATTCAATG);
contacting the sample with a second primer consisting essentially of SEQ ID NO: 2 (ACTACCAACTGATTGATAGATCAG);
extending the first and second primer, thereby producing at least one amplicon if the E. histolytica polynucleotide sequence is present in the sample; and
contacting the sample with an oligonucleotide probe comprising a polynucleotide consisting essentially of SEQ ID NO: 3 (ATTGTCGTGGCATCCTAACTCA) or its complement,
wherein the probe provides detectable signal when it is bound to a substantially complementary nucleic acid, but does not provide detectable signal when it is single-stranded, and
detecting the signal, if the amplicon is present.

2. The method of claim 1, wherein, if used under standard amplification conditions, the first primer and second primer amplify the E. histolytica polynucleotide sequence, but do not substantially amplify any E. dispar polynucleotide sequence;

3. The method of any one of claims 1-2, wherein the first primer hybridizes to the E. histolytica polynucleotide sequence if contacted with the E. histolytica polynucleotide sequence at a temperature of at least about 50° C. in 5 mM MgCl2, 100 mM Tris, 10 mM NaOH, 0.019% ProClin300, 0.010% Tween-20, 1.96% Trehalose, 0.6 mg/ml BSA, but does not hybridize to any E. dispar polynucleotide sequence if contacted with any E. dispar polynucleotide sequence at a temperature of at least about 60° C. in 5 mM MgCl₂, 100 mM Tris, 10 mM NaOH, 0.019% ProClin300, 0.010% Tween-20, 1.96% Trehalose, 0.6 mg/ml BSA.

4. The method of any one of claims 1-3, wherein the second primer hybridizes to the E. histolytica polynucleotide sequence if contacted with E. histolytica polynucleotide sequence at a temperature of at least about 60° C. in 5 mM MgCl2, 100 mM Tris, 10 mM NaOH, 0.019% ProClin300, 0.010% Tween-20, 1.96% Trehalose, 0.6 mg/ml BSA, and hybridizes to an E. dispar polynucleotide sequence if contacted with the E. dispar polynucleotide sequence at a temperature of at least about 60° C. in 5 mM MgCl2, 100 mM Tris, 10 mM NaOH, 0.019% ProClin300, 0.010% Tween-20, 1.96% Trehalose, 0.6 mg/ml BSA.

5. The method of any of claims 1-3, wherein each of the first primer and second primer hybridizes to the E. histolytica polynucleotide sequence if contacted with the E. histolytica polynucleotide sequence at a temperature of at least about 60° C. in in 5 mM MgCl2, 100 mM Tris, 10 mM NaOH, 0.019% ProClin300, 0.010% Tween-20, 1.96% Trehalose, 0.6 mg/ml BSA, but the second primer does not hybridize to any E. dispar polynucleotide sequence if contacted with any E. dispar polynucleotide sequence at a temperature of at least about 60° C. in 5 mM MgCl2, 100 mM Tris, 10 mM NaOH, 0.019% ProClin300, 0.010% Tween-20, 1.96% Trehalose, 0.6 mg/ml BSA.

6. The method of any one of claims 1-5, wherein the sample comprises E. histolytica and E. dispar.

7. The method of any one of claims 1-6, wherein the sample comprises fecal material of a human.

8. The method of any one of claims 1-7, wherein the sample comprises fixed material.

9. The method of any one of claims 1-7, wherein the sample is non-fixed.

10. The method of any one of claims 1-9, wherein a 95% limit of detection for E. histolytica comprises no more than about 17 E. histolytica genomes per milliliter.

11. The method of any of claims 1-10, wherein if used under standard amplification conditions, the primers and probes do not cross-react with any of the following organisms, if present in the sample: Abiotrophia defectiva, Acinetobacter baumannil, Acinetobacter Iwoffii, Aeromonas hydrophila, Alcaligenes faecalis subsp. faecalis, Anaerococcus tetradius, Arcobacter butzleri, Arcobacter cryaerophilus, Bacillus cereus, Bacteroides caccae, Bacteroides merdae, Bacteroides stercoris, Bifidobacterium adolescentis, Bifidobacterium longum, Camplylobacter coli, Campylobacter concisus, Campylobacter curvus, Campylobacter fetus subsp. fetus, Campylobacter fetus subsp. venerealis, Campylobacter gracilis, Campylobacter hominis, Camplylobacter jejuni, Campylobacter lari, Campylobacter rectus, Campylobacter upsaliensis, Candida albicans, Candida catenulate, Cedecea davisae, Chlamydia trachomatis, Citrobacter amalonaticus, Citrobacter fruendii, Citrobacter koseri, Citrobacter sedlakii, Clostridium difficile 17858, Clostridium difficile 43598, Clostridium difficile CCUG 8864-9689, Clostridium difficile 43255, Clostridium difficile BAA-1805, Clostridium difficile 43593, Clostridium perfringens, Collinsella aerofaciens, Corynebacterium genitalium, Desulfovibrio piger, Edwardsiella tarda, Eggerthella lenta, Enterobacter aerogenes, Enterobacter cloacae, Enterococcus casseliflavus, Enterococcus cecorum, Enterococcus dispar, Enterococus faecalis, Enterococcus faecium, Enterococcus gallinarum, Enterococcus hirae, Enterococcus raffinosus, Escherichia coli, Escherichia fergusonii, Escherichia hermannii, Escherichia vulneris, Fusobacterium varium, Gardnerella vaginalis, Gemella morbillorum, Hafnia alvei, Helicobacter fennelliae, Helicobacter pylori, Klebsiella oxytoca, Klebsiella pneumonia, Lactobacillus acidophilus, Lactobacillus reuteri, Lactococcus lactis, Leminorella grimontii, Listeria grayi, Listeria innocua, Listeria monocytogenes, Morganella morganii, Peptoniphilus asaccharolyticus, Peptostreptococcus anaerobius, Plesiomonas shigelloides, Porphyromonas asaccharolytica, Prevotella melaninogenica, Proteus mirabilis, Proteus penneri, Proteus vulgaris, Providencia alcalifaciens, Providencia rettgeri, Providencia stuartii, Pseudomonas aeruginosa, Pseudomonas fluorescens, Ruminococcus bromii, Salmonella typhimurium, Salmonella enteriditis, Serratia liquefaciens, Serratia marcescens, Shigella sonnei, Shigella flexneri, Staphylococcus aureus, Staphylococcus epidermidis, Stenotrophomonas maltophilia, Streptococcus agalactiae, Streptococcus dysgalactiae, Streptococcus intermedius, Streptococcus uberis, Trabulsiella guamensis, Veillonella parvula, Vibrio cholera, Vibrio parahaemolyticus, Yersinia bercovieri, Yersinia enterocolitica, Yersinia rohdei, Adenovirus type 2, Adenovirus type 14, Adenovirus type 40, Adenovirus type 41, Coxsackie A9, Coxsackie B1, HHV-5, Cytomegalovirus, Enterovirus type 69, Human Papillomavirus Type 16, Human Papillomavirus Type 18, Herpes Simplex Virus I, Herpes Simplex Virus II, Norovirus I, Norovirus II, Rotavirus, Blastocystis hominis, Encephalitozoon intestinalis, Encephalitozoon helium, Encephalitozoon cuniculi, Pentatrichomonas hominis, Entamoeba barrette, Entamoeba dispar, Entamoeba gigivalis, Entamoeba invadens, Entamoeba moshkovskii, Entamobea ranarum, Citrobacter fruendii (rpt), Enterobacter cloacae (rpt), Cryptosporidium parvum, Giardia lamblia, or Cryptosporidium meleagridis.

12. A kit comprising:
a first primer;
a second primer,
wherein, if used under standard amplification conditions, the first primer and second primer amplify a E. histolytica polynucleotide sequence, thereby producing an amplicon, but do not substantially amplify any E. dispar polynucleotide sequence; and
a probe, wherein the probe comprises a polynucleotide consisting essentially of a sequence, wherein the sequence or its complement is present in each of the amplicon, a polynucleotide sequence of E. histolytica, and a polynucleotide sequence of E. dispar.

13. The kit of claim 12, wherein the probe comprises:
a fluorophore; and
a quencher.

14. The kit of any one of claims 12-13, wherein the primers and probes amplify an E. histolytica polynucleotide sequence with a 95% limit of detection of no more than about 17 E. histolytica organisms per mililiter.

15. The kit of any one of claims 12-14, wherein if used under standard amplification conditions, the primers and probes do not cross-react with any of the following organisms, if present in the sample: Abiotrophia defectiva, Acinetobacter baumannii, Acinetobacter Iwoffii, Aeromonas hydrophila, Alcaligenes faecalis subsp. faecalis, Anaerococcus tetradius, Arcobacter butzleri, Arcobacter cryaerophilus, Bacillus cereus, Bacteroides caccae, Bacteroides merdae, Bacteroides stercoris, Bifidobacterium adolescentis, Bifidobacterium longum, Camplylobacter coli, Campylobacter concisus, Campylobacter curvus, Campylobacter fetus subsp. fetus, Campylobacter fetus subsp. venerealis, Campylobacter gracilis, Campylobacter hominis, Camplylobacter jejuni, Campylobacter lari, Campylobacter rectus, Campylobacter upsaliensis, Candida albicans, Candida catenulate, Cedecea davisae, Chlamydia trachomatis, Citrobacter amalonaticus, Citrobacter fruendii, Citrobacter koseri, Citrobacter sedlakii, Clostridium difficile 17858, Clostridium difficile 43598, Clostridium difficile CCUG 8864-9689, Clostridium difficile 43255, Clostridium difficile BAA-1805, Clostridium difficile 43593, Clostridium perfringens, Collinsella aerofaciens, Corynebacterium genitalium, Desulfovibrio piger, Edwardsiella tarda, Eggerthella lenta, Enterobacter aerogenes, Enterobacter cloacae, Enterococcus casseliflavus, Enterococcus cecorum, Enterococcus dispar, Enterococus faecalis, Enterococcus gallinarum, Enterococcus hirae, Enterococcus raffinosus, Escherichia coli, Escherichia fergusonii, Escherichia hermannii, Escherichia vulneris, Fusobacterium varium, Gardnerella vaginalis, Gemella morbillorum, Hafnia alvei, Helicobacter fennelliae, Helicobacter pylori, Klebsiella oxytoca, Klebsiella pneumonia, Lactobacillus acidophilus, Lactobacillus reuteri, Lactococcus lactis, Leminorella grimontii, Listeria grayi, Listeria innocua, Listeria monocytogenes, Morganella morganii, Peptomphilus asaccharolyticus, Peptostreptococcus anaerobius, Plesiomonas shigelloides, Porphyromonas asaccharolytica, Prevotella melaninogenica, Proteus mirabilis, Proteus penneri, Proteus vulgaris, Providencia alcalifaciens, Providencia rettgeri, Providencia stuartii, Pseudomonas aeruginosa, Pseudomonas fluorescens, Ruminococcus bromii, Salmonella typhimurium, Salmonella enteriditis, Serratia liquefaciens, Serratia marcescens, Shigella sonnei, Shigella flexneri, Staphylococcus aureus, Staphylococcus epidermidis, Stenotrophomonas maltophilia, Streptococcus agalactiae, Streptococcus dysgalactiae, Streptococcus intermedius, Streptococcus uberis, Trabulsiella guamensis, Veillonella parvula, Vibrio cholera, Vibrio parahaemolyticus, Yersinia bercovieri, Yersinia enterocolitica, Yersinia rohdei, Adenovirus type 2, Adenovirus type 14, Adenovirus type 40, Adenovirus type 41, Coxsackie A9, Coxsackie B1, HHV-5, Cytomegalovirus, Enterovirus type 69, Human Papillomavirus Type 16, Human Papillomavirus Type 18, Herpes Simplex Virus I, Herpes Simplex Virus II, Norovirus I, Norovirus II, Rotavirus, Blastocystis hominis, Encephalitozoon intestinalis, Encephalitozoon helium, Encephalitozoon cuniculi, Pentatrichomonas hominis, Entamoeba barrette, Entamoeba dispar, Entamoeba gigivalis, Entamoeba invadens, Entamoeba moshkovskii, Entamobea ranarum, Citrobacter fruendii (rpt), Enterobacter cloacae (rpt), Cryptosporidium parvum, Giardia lamblia, or Cryptosporidium meleagridis.

16. A kit comprising:
a first primer comprising a polynucleotide having at least about 90% identity to SEQ ID NO: 1 (GTACAAAATGGCCAATTCATTCAATG);
a second primer comprising polynucleotide having at least about 90% identity to SEQ ID NO: 2 (ACTACCAACTGATTGATAGATCAG); and
a probe comprising:
a polynucleotide having at least about 90% identity to SEQ ID NO: 3 (ATTGTCGTGGCATCCTAACTCA) or its complement;
a flurophore; and
a quencher.

17. The kit of claim 16, wherein
the first primer consists essentially of SEQ ID NO: 1 (GTACAAAATGGCCAATTCATTCAATG),
the second primer consists essentially of SEQ ID NO: 2 (ACTACCAACTGATTGATAGATCAG); and
the probe comprises a polynucleotide consisting essentially of SEQ ID NO: 3 (ATTGTCGTGGCATCCTAACTCA) or its complement.

18. A method of detecting the presence of an E. histolytica polynucleotide sequence in a sample, the method comprising:
contacting the sample with a first primer;
contacting the sample with a second primer,
wherein, under if used standard amplification conditions, the first primer and second primer amplify the E. histolytica polynucleotide sequence, but do not substantially amplify any E. dispar polynucleotide sequence;
extending the first and second primer, thereby producing at least one amplicon if the E. histolytica polynucleotide sequence is present in the sample; and
contacting the sample with an oligonucleotide probe,
wherein the probe provides detectable signal when it is bound to a substantially complementary nucleic acid, but does not provide detectable signal when it is single-stranded, and
wherein the probe comprises a polynucleotide consisting essentially of sequence that is a portion of the E. histolytica polynucleotide sequence, a polynucleotide sequence of E. dispar, and a sequence of the amplicon; and
detecting the signal, if the amplicon is present.

19. The method of claim 18, wherein the first primer hybridizes to the E. histolytica polynucleotide sequence if contacted with the E. histolytica polynucleotide sequence at a temperature of at least about 50° C. in 5 mM MgCl2, 100 mM Tris, 10 mM NaOH, 0.019% ProClin300, 0.010% Tween-20, 1.96% Trehalose, 0.6 mg/ml BSA, but does not hybridize to any E. dispar polynucleotide sequence if contacted with any E. dispar polynucleotide sequence at a temperature of at least about 60° C. in 5 mM MgCl₂, 100 mM Tris, 10 mM NaOH, 0.019% ProClin300, 0.010% Tween-20, 1.96% Trehalose, 0.6 mg/ml BSA.

20. The method of any one of claims 18-19, wherein the second primer hybridizes to the E. histolytica polynucleotide sequence if contacted with E. histolytica polynucleotide sequence at a temperature of at least about 60° C. in 5 mM MgCl2, 100 mM Tris, 10 mM NaOH, 0.019% ProClin300, 0.010% Tween-20, 1.96% Trehalose, 0.6 mg/ml BSA, and hybridizes to an E. dispar polynucleotide sequence if contacted with the E. dispar polynucleotide sequence at a temperature of at least about 60° C. in 5 mM MgCl2, 100 mM Tris, 10 mM NaOH, 0.019% ProClin300, 0.010% Tween-20, 1.96% Trehalose, 0.6 mg/ml BSA.

21. The method of any one of claims 18-20, wherein each of the first primer and second primer hybridizes to the E. histolytica polynucleotide sequence if contacted with the E. histolytica polynucleotide sequence at a temperature of at least about 60° C. in in 5 mM MgCl2, 100 mM Tris, 10 mM NaOH, 0.019% ProClin300, 0.010% Tween-20, 1.96% Trehalose, 0.6 mg/ml BSA, but the second primer does not hybridize to any E. dispar polynucleotide sequence if contacted with any E. dispar polynucleotide sequence at a temperature of at least about 50° C. in 5 mM MgCl2, 100 mM Tris, 10 mM NaOH, 0.019% ProClin300, 0.010% Tween-20, 1.96% Trehalose, 0.6 mg/ml BSA.

22. The method of any one of claims 18-21 or 40-75, wherein the first primer comprises a polynucleotide having at least about 90% identity to SEQ ID NO: 1 (GTACAAAATGGCCAATTCATTCAATG) or its complement.

23. The method of any one of claims 18-22, wherein the first primer consists essentially of SEQ ID NO: 1 (GTACAAAATGGCCAATTCATTCAATG) or its complement.

24. The method of any one of claims 18-23, or 40-75, wherein the second primer comprises a polynucleotide having at least about 90% identity to SEQ ID NO: 2 (ACTACCAACTGATTGATAGATCAG) or its complement.

25. The method of any of claims 18-24, or 40-75, wherein the second primer comprises a polynucleotide having the sequence of SEQ ID NO: 2 (ACTACCAACTGATTGATAGATCAG) or its complement.

26. The method of any of claims 18-25, or 40-75, wherein the probe comprises a polynucleotide having at least about 90% identity to SEQ ID NO: 3 (ATTGTCGTGGCATCCTAACTCA) or its complement.

27. The method of any of claims 18-26, or 40-75, wherein the probe comprises a polynucleotide having the sequence of SEQ ID NO: 3 (ATTGTCGTGGCATCCTAACTCA) or its complement.

28. The method of any of claims 18-27, or 40-75, wherein the amplicon comprises a polynucleotide having at least about 95% identity to SEQ ID NO: 7 (GTACAAAATGGCCAATTCATTCAATGAATTGAGAAATGACATTCTAAGTGAG TTAGGATGCCACGACAATTGTAGAACACACAGTGTTTAACAAGTAACCAATG AGAATTTCTGATCTATCAATCAGTTGGTAGT).

29. The method of any of claims 18-28, or 40-75, wherein the amplicon comprises a polynucleotide having the sequence of SEQ ID NO: 7 (GTACAAAATGGCCAATTCATTCAATGAATTGAGAAATGACATTCTAAGTGAG TTAGGATGCCACGACAATTGTAGAACACACAGTGTTTAACAAGTAACCAATG AGAATTTCTGATCTATCAATCAGTTGGTAGT).

30. The method of any of claims 18-29, or 40-75, wherein the sample comprises E. histolytica and E. dispar.

31. The method of any of claims 18-30, or 40-75, wherein the sample comprises fecal material of a human.

32. The method of any of claims 18-31, or 40-75, wherein the sample comprises fixed material.

33. The method of any of claims 18-32, or 40-75, wherein the sample is non-fixed.

34. The method of any of claims 18-33, or 40-75, wherein a 95% limit of detection for E. histolytica comprises no more than about 17 E. histolytica genomes per milliliter.

35. The method of any of claims 18-34, or 40-75, wherein if used under standard amplification conditions, the primers and probes do not cross-react with any of the following organisms, if present in the sample: Abiotrophia defectiva, Acinetobacter baumannii, Acinetobacter Iwoffii, Aeromonas hydrophila, Alcaligenes faecalis subsp. faecalis, Anaerococcus tetradius, Arcobacter butzleri, Arcobacter cryaerophilus, Bacillus cereus, Bacteroides caccae, Bacteroides merdae, Bacteroides stercoris, Bifidobacterium adolescentis, Bifidobacterium longum, Camplylobacter coli, Campylobacter concisus, Campylobacter curvus, Campylobacter fetus subsp. fetus, Campylobacter fetus subsp. venerealis, Campylobacter gracilis, Campylobacter hominis, Camplylobacter jejuni, Campylobacter lari, Campylobacter rectus, Campylobacter upsaliensis, Candida albicans, Candida catenulate, Cedecea davisae, Chlamydia trachomatis, Citrobacter amalonaticus, Citrobacter fruendii, Citrobacter koseri, Citrobacter sedlakii, Clostridium difficile 17858, Clostridium difficile 43598, Clostridium difficile CCUG 8864-9689, Clostridium difficile 43255, Clostridium difficile BAA-1805, Clostridium difficile 43593, Clostridium perfringens, Collinsella aerofaciens, Corynebacterium genitalium, Desulfovibrio piger, Edwardsiella tarda, Eggerthella lenta, Enterobacter aerogenes, Enterobacter cloacae, Enterococcus casseliflavus, Enterococcus cecorum, Enterococcus dispar, Enterococus faecalis, Enterococcus gallinarum, Enterococcus hirae, Enterococcus raffinosus, Escherichia coli, Escherichia fergusonii, Escherichia hermannii, Escherichia vulneris, Fusobacterium varium, Gardnerella vaginalis, Gemella morbillorum, Hafnia alvei, Helicobacter fennelliae, Helicobacter pylori, Klebsiella oxytoca, Klebsiella pneumonia, Lactobacillus acidophilus, Lactobacillus reuteri, Lactococcus lactis, Leminorella grimontii, Listeria grayi, Listeria innocua, Listeria monocytogenes, Morganella morganii, Peptomphilus asaccharolyticus, Peptostreptococcus anaerobius, Plesiomonas shigelloides, Porphyromonas asaccharolytica, Prevotella melaninogenica, Proteus mirabilis, Proteus penneri, Proteus vulgaris, Providencia alcalifaciens, Providencia rettgeri, Providencia stuartii, Pseudomonas aeruginosa, Pseudomonas fluorescens, Ruminococcus bromii, Salmonella typhimurium, Salmonella enteriditis, Serratia liquefaciens, Serratia marcescens, Shigella sonnei, Shigella flexneri, Staphylococcus aureus, Staphylococcus epidermidis, Stenotrophomonas maltophilia, Streptococcus agalactiae, Streptococcus dysgalactiae, Streptococcus intermedius, Streptococcus uberis, Trabulsiella guamensis, Veillonella parvula, Vibrio cholera, Vibrio parahaemolyticus, Yersinia bercovieri, Yersinia enterocolitica, Yersinia rohdei, Adenovirus type 2, Adenovirus type 14, Adenovirus type 40, Adenovirus type 41, Coxsackie A9, Coxsackie B1, HHV-5, Cytomegalovirus, Enterovirus type 69, Human Papillomavirus Type 16, Human Papillomavirus Type 18, Herpes Simplex Virus I, Herpes Simplex Virus II, Norovirus I, Norovirus II, Rotavirus, Blastocystis hominis, Encephalitozoon intestinalis, Encephalitozoon helium, Encephalitozoon cuniculi, Pentatrichomonas hominis, Entamoeba barrette, Entamoeba dispar, Entamoeba gigivalis, Entamoeba invadens, Entamoeba moshkovskii, Entamobea ranarum, Citrobacter fruendii (rpt), Enterobacter cloacae (rpt), Cryptosporidium parvum, Giardia lamblia, or Cryptosporidium meleagridis.

36. The method of any one of claims 1-11 or 18-35 or 40-75, wherein if used under standard amplification conditions, the primers and probes do not cross-react with any of the following organisms, if present in the sample: Entamoeba coli, Entamoeba dispar, Entamoeba polecki, Entamoeba muris, Entamoeba nuttalli, Entamoeba hartmanni, and Entamoeba bovis.

37. The method of any one of claims 1-11 or 18-36 or 40-75, wherein if used under standard amplification conditions, the primers and probes produce fewer than 1 in 1600 false positives for samples that do not comprise E. histolytica.

38. The kit of any one of claims 12-17, wherein if used under standard amplification conditions, the primers and probes do not cross-react with any of the following organisms, if present in the sample: Entamoeba coli, Entamoeba dispar, Entamoeba polecki, Entamoeba muris, Entamoeba nuttalli, Entamoeba hartmanni, and Entamoeba bovis.

39. The kit of any one of claims 12-17 or 38, wherein if used under standard amplification conditions, the primers and probes produce fewer than 1 in 1600 false positives for samples that do not comprise E. histolytica.

40. A method of determining the presence or absence of an E. histolytica nucleic acid sequence in a sample, the method comprising:
performing a nucleic acid amplification reaction on the sample, the nucleic acid amplification comprising a first oligonucleotide primer and a second oligonucleotide primer,
wherein the first oligonucleotide primer has a length of 15-75 nucleotides and hybridizes under standard conditions to SEQ ID NO:10 or its complement, if present, but does not hybridize under standard conditions to SEQ ID NO: 11 or its complement, if present, and
wherein the second oligonucleotide primer has a length of 15-75 nucleotides and hybridizes under standard conditions to a SEQ ID NO:10 or its complement, if present, and wherein the second oligonucleotide primer hybridizes under standard conditions to SEQ ID NO: 11 or its complement, if present;
detecting a signal, if present, from a detectably labeled probe that hybridizes to an amplicon of the first and second oligonucleotide primers under standard hybridization conditions if the amplicon is present, wherein the signal indicates the presence or absence of the amplicon,
wherein the amplicon has a length of 75-350 nucleotides.

41. The method of claim 40, wherein the first oligonucleotide primer comprises at least 10 consecutive nucleotides of SEQ ID NO: 1, and wherein the first oligonucleotide primer has at least 80% identity to a target sequence of SEQ ID NO: 10 or its complement.

42. The method of claim 40 or claim 41, wherein the second oligonucleotide primer comprises at least 10 consecutive nucleotides of SEQ ID NO: 2, and wherein the second oligonucleotide primer has at least 80% identity to a target sequence of SEQ ID NO: 10 or its complement.

43. The method of claim 41 or claim 42, wherein the first oligonucleotide primer comprises at least 12 consecutive nucleotides of SEQ ID NO: 1.

44. The method of claim 41 or claim 42, wherein the first oligonucleotide primer comprises at least 15 consecutive nucleotides of SEQ ID NO: 1.

45. The method of claim 41 or claim 42, wherein the first oligonucleotide primer comprises at least 20 consecutive nucleotides of SEQ ID NO: 1.

46. The method of any one of claims 41-45, wherein the first oligonucleotide primer has at least 85% identity to a target sequence of SEQ ID NO: 10 or its complement.

47. The method of any one of claims 41-45, wherein the first oligonucleotide primer has at least 90% identity to a target sequence of SEQ ID NO: 10 or its complement.

48. The method of any one of claims 41-45, wherein the first oligonucleotide primer has at least 95% identity to a target sequence of SEQ ID NO: 10 or its complement.

49. The method of any one of claims 41-45, wherein the first oligonucleotide primer has 100% identity to a target sequence of SEQ ID NO: 10 or its complement.

50. The method of any one of claims 42-49, wherein the second oligonucleotide primer comprises at least 12 consecutive nucleotides of SEQ ID NO: 2.

51. The method of any one of claims 42-49, wherein the second oligonucleotide primer comprises at least 15 consecutive nucleotides of SEQ ID NO: 2.

52. The method of any one of claims 42-49, wherein the second oligonucleotide primer comprises at least 20 consecutive nucleotides of SEQ ID NO: 2.

53. The method of any one of claims 42-52, wherein the second oligonucleotide primer has at least 85% identity to a target sequence of SEQ ID NO: 10 or its complement.

54. The method of any one of claims 42-52, wherein the second oligonucleotide primer has at least 90% identity to a target sequence of SEQ ID NO: 10 or its complement.

55. The method of any one of claims 42-52, wherein the second oligonucleotide primer has at least 95% identity to a target sequence of SEQ ID NO: 10 or its complement.

56. The method of any one of claims 42-52, wherein the second oligonucleotide primer has 100% identity to a target sequence of SEQ ID NO: 10 or its complement.

57. The method of any one of claims 40-56, wherein the probe comprises at least 10 consecutive nucleotides of SEQ ID NO: 3, and wherein the probe has at least 80% identity to a target sequence of SEQ ID NO: 10 or its complement.

58. The method of claim 57, wherein the probe comprises at least 12 consecutive nucleotides of SEQ ID NO: 3.

59. The method of claim 57, wherein the probe comprises at least 15 consecutive nucleotides of SEQ ID NO: 3.

60. The method of claim 57 wherein the probe comprises at least 20 consecutive nucleotides of SEQ ID NO: 3.

61. The method of any one of claims 57-60, wherein the probe has at least 85% identity to a target sequence of SEQ ID NO: 10 or its complement.

62. The method of any one of claims 57-60, wherein the probe has at least 90% identity to a target sequence of SEQ ID NO: 10 or its complement.

63. The method of any one of claims 57-60, wherein the probe has at least 95% identity to a target sequence of SEQ ID NO: 10 or its complement.

64. The method of any one of claims 57-60, wherein the probe has 100% identity to a target sequence of SEQ ID NO: 10 or its complement.

65. The method of any one of claims 40-64, wherein the first oligonucleotide primer is about 20-50 nucleotides long.

66. The method of any one of claims 40-64, wherein the first oligonucleotide primer is about 23-45 nucleotides long.

67. The method of any one of claims 40-66, wherein the second oligonucleotide primer is about 20-50 nucleotides long.

68. The method of any one of claims 40-66, wherein the second oligonucleotide primer is about 23-45 nucleotides long.

69. The method of any one of claims 40-68, wherein the detectably labeled probe is about 15-75 nucleotides long.

70. The method of any one of claims 40-68, wherein the detectably labeled probe is about 20-45 nucleotides long.

71. The method of any one of claims 40-70 wherein the detectably labeled probe probe is capable of hybridizing to SEQ ID NO:10 and to SEQ ID NO: 11 under standard hybridization conditions.

72. The method of any one of claims 40-70, wherein the detectably labeled probe probe is capable of hybridizing to SEQ ID NO:10 but not to SEQ ID NO: 11 under standard hybridization conditions.

73. The method of any one of claims 40-72, wherein the detectably labeled probe probe comprises a fluorophore or a quencher.

74. The method of any one of claims 40-73, wherein the amplicon has a length of 100-150 nucleotides.

75. The method of any one of claims 40-74, wherein the amplicon comprises SEQ ID NO: 7.

76. The method of any of claims 18-26, or 40-75, wherein E. dispar, if present, does not inhibit determining the presence or absence of E. histolytica.

77. The method of any of claims 18-26, wherein E. dispar, if present, does not inhibit production of the amplicon if the E. histolytica polynucleotide sequence is present in the sample.

78. The method of any of claims 40-75, wherein E. dispar, if present, does not inhibit production of the amplicon of the first and second oligonucleotide primers.

79. A kit comprising the first oligonucleotide primer, the second oligonucleotide primer, and the detectably labeled probe of any one of claims 40-77.

80. The kit of any of claims 12-17, 38, or 79, wherein E. dispar, if present, does not inhibit determining the presence or absence of E. histolytica.

81. The kit of any of claims 12-17, 38, or 79, wherein E. dispar, if present, does not inhibit production of the amplicon if the E. histolytica polynucleotide sequence is present in the sample.


SEQUENCE LISTING (11 sequences)

SEQ ID NO: 1 - DNA, 26 bp, Artificial Sequence
  EntFP-GT
gtacaaaatggccaattcattcaatg

SEQ ID NO: 2 - DNA, 24 bp, Artificial Sequence
  EntRP-GT
actaccaactgattgatagatcag

SEQ ID NO: 3 - DNA, 22 bp, Artificial Sequence
  EntPr-GT
attgtcgtggcatcctaactca

SEQ ID NO: 4 - DNA, 22 bp, Artificial Sequence
  EntFP
attgtcgtggcatcctaactca

SEQ ID NO: 5 - DNA, 20 bp, Artificial Sequence
  EntRP
gcggacggctcattataaca

SEQ ID NO: 6 - DNA, 22 bp, Artificial Sequence
  EntPr
tcattgaatgaattggccattt

SEQ ID NO: 7 - DNA, 135 bp, Entamoeba histolytica
gtacaaaatggccaattcattcaatgaattgagaaatgacattctaagtgagttaggatgccacgacaattgtagaacacacagtgtttaacaagtaaccaatgagaatttctgatctatcaatcagttggtagt

SEQ ID NO: 8 - DNA, 89 bp, Entamoeba dispar
tttgtgaatgataaagataatacttgagacgatccaatttgtattagtacaaagtggccaatttatgtaagtaaattgagaaatgacat

SEQ ID NO: 9 - DNA, 88 bp, Entamoeba histolytica
tttgtgaatgataaagataatacttgagacgatccagtttgtattagtacaaaatggccaattcattcaatgaattgagaaatgacat

SEQ ID NO: 10 - DNA, 1944 bp, Entamoeba histolytica
tatctggttgatcctgccagtattatatgctgatgttaaagattaagccatgcatgtgtaagtataaagaccaagtaggatgaaactgcggacggctcattataacagtaatagtttctttggttagtaaaatacaaggatagctttgtgaatgataaagataatacttgagacgatccagtttgtattagtacaaaatggccaattcattcaatgaattgagaaatgacattctaagtgagttaggatgccacgacaattgtagaacacacagtgtttaacaagtaaccaatgagaatttctgatctatcaatcagttggtagtatcgaggactaccaagattataacggataacgaggaattggggttcgacatcggagagggagctttacagatggctaccacttctaaggaaggcagcaggcgcgtaaattacccactttcgaattgaagaggtagtgacgacacataactctagagttgagtaaaatcaattcttgaaggaatgagtaggaggtaaattctcctacgaaatcaattggagggcaagtctggtgccagcagccgcggtaattccagctccaatagtgtatattaaagttgctgtgattaaaacgctcgtagttgaattaaaatgtggttttatacattttgaagactttatgtaagtaaagtttctagaaatgttaaattaaaatcaaagaaggaaacaattcaagtaattgagttgttattactttgaataaaataaggtgtttaaagcaaaacattatgttaatgaatattcaagcatgggacaatgctgaggggatgtcaataagacatttcgagagaaggattaaaaggaacaattggggtgattcagaaaataacgggagaggtgaaaatccatgatcgctataagatgcacgagagcgaaagcatttcactcaactgtgtccattaatcaagaacgaaagttaggggatcgaagacgatcagataccgtcgtagtcctaactataaacgatgtcaaccaaggattggatgaaattcagatgtacaaagatagagaagcattgtttctagatctgagtatatcaatattaccttgttcagaacttaaagagaaatcttgagtttatggacttcagggggagtatggtcacaaggctgaaacttaaaggaattgacggaagggcacaccaggagtggagcctgcggcttaatttgactcaacacgggaaaacttaccaagaccgaacagtagaaggaatgacagattaagagttctttcatgatttattgggtagtggtgcatggccgttcttagttggtggagtgatttgtcaggttaattccggtaacgaacgagactgaaacctattaattagttttctgcctataagacagaaatgttcgcaagaacaggtgcgtaagtaccacttcttaaagggacacatttcaattgtcctattttaattgtagttatctaatttcggttagacctcttttaacgtgggaaaaagaaaaaggaagcattcagcaataacaggtctgtgatgcccttagacatcttgggccgcacgcgcgctacaatggagttactagagagtattttatcatttacaccttatttattaggctttgtctaataattaaggatagtaagtggtgtaccgagattgaaatagttaaggaaaactcaaaagaacgtacatgacagggataaatgattggaattatttgttttgaacgaggaattccttgtaatatcgagtcattaactcgagatgaatacgtccctgccctttgtacacaccgcccgtcgctcctaccgattgaataaagaggtgaaattctaggattctgtcttatagatagaaaaatggatttaaatctccttatttagaggaaggagaagtcgtaacaaggtttccgtaggtgaacctgcggaaggatca

SEQ ID NO: 11 - DNA, 2426 bp, Entamoeba dispar
tggatataaatacaaaagagaagtaagaataaagaatccttccttttaaaaaggaagaagaataaaatatctggttgatcctgccagtattatatgctgatgttaaagattaagccatgcatgtgtaagtataaagaccaagtaggatgaaactgcggacggctcattataacagtaatagtttctttggttagtaaagtacaaggatagctttgtgaatgataaagataatacttgagacgatccaatttgtattagtacaaagtggccaatttatgtaagtaaattgagaaatgacattctaagtgagttaggatgccacgacaattgtagaacacacagtgtttaacaagtaaccaatgagaatttctgatctatcaatcagttggtagtatcgaggactaccaagattataacggataacgaggaattggggttcgacatcggagagggagctttacagatggctaccacttctaaggaaggcagcaggcgcgtaaattacccactttcgaattgaagaggtagtgacgacacataactctagagttgagtaaaatcaattcttgaaggaatgagtaggaggtaaattctcctacgaaatcaattggagggcaagtctggtgccagcagccgcggtaattccagctccaatagtgtatattaaagttgctgtgattaaaacgctcgtagttgaattaaaatgtgattttatacattttgaagactttacattaagtgaagtttctagaaatgttaaattaaaatcaaagaaggagacaattcaagtaattgagttgtcattactttgaataaaataaggtgtttaaagcaaaacattatgttaatgaatattcaagcatgggacaatgctgaggagatgtcaattagacatttcgagagaaggattaaaaggaacaattggggtgattcagaaaataacgggagaggtgaaaatccatgatcgctataagatgcacgagagcgaaagcatttcactcaactgggtccattaatcaagaacgaaagttaggggatcgaagacgatcagataccgtcgtagtcctaactataaacgatgtcaaccaaggattggatgaaattcagatgtacaaagatgaagaaacattgtttctaaatccaagtatatcaatactaccttgttcagaacttaaagagaaatcttgagtttatggacttcagggggagtatggtcacaaggctgaaacttaaaggaattgacggaagggcacaccaggagtggagcctgcggcttaatttgactcaacacgggaaaacttaccaagaccgaacagtagaaggaatgacagattaagagttctttcatgatttattgggtagtggtgcatggccgttcttagttggtggagtgatttgtcaggttaattccggtaacgaacgagactgaaacctattaattagttttctgcctataagacagaaatgttcgcaagaacaggtgcgtaagtaccacttcttaaagggacacatttcaattgtcctattttaattgttagttatctaatttcgattagaactcttttaacgtgggaaaaagaaaaaggaagcattcagcaataacaggtctgtgatgcccttagacatcttgggccgcacgcgcgctacaatggagttactagagagcattttatcatttacaccttatttattaggctatgtctaataggtagggatagtaagtggtgtaccgagattgaaatagttaaggaaaactcaaaagaacgtacatgacagggataaatgattggaattatttgttttgaacgaggaattccttgtaatatcgagtcattaactcgagatgaatacgtccctgccctttgtacacaccgcccgtcgctcctaccgattgaataaagaggtgaaattctaggattctgtcttatagatagaaaaatggatttaaatctccttatttagaggaaggagaagtcgtaacaaggtttccgtaggtgaacctgcggaaggatcattaaaagaaaagaaataatcttttaaaataaaacaagaaatttatagaataagataatctacaaagaaaataataaaagtaagaataaaaggaattagaatataagaagaaagaaaaagtataataaaatattactttggatagtttagtttcctgtgcgatgaagaacgcaatgaattgcgataagtgataggaacaataaaatgtgaatatccaaactttgaatgcttgaaagtatacttatgaacttcaaggtatatatgatattcaatatccaaaataaaagagtatattaaaagcaaatattagtagaagtgagaagtagctagtgggtaaaagagagaagaagtaaagagctttaaccagatatctataagtgagttaataaataaagatttgagtatcgtaagag